(19) United States
(12) Patent Application Publication
(10) Pub. No.: US 2009/0227555 A2
(43) Pub. Date: Sep. 10, 2009

(54) Novel Pyridine Compounds

(75) Inventors: Peter Bach, Molndal (SE); Jonas Bostrom, Molndal (SE); Kay Brickmann, Molndal (SE); Leifeng Cheng, Molndal (SE); Fabrizio Giordanetto, Molndal (SE); Robert Groneberg, Boulder, CO (US); Darren Harvey, Westminster, CO (US); Michael O'Sullivan, Boulder, CO (US); Fredrik Zetterberg, Molndal (SE)

Correspondence Address: Pepper Hamilton LLP, 400 Berwyn Park, 899 Cassatt Road, Berwyn, PA, 19312-1183, UNITED STATES, 610.640.5489, 610.640.7835

(73) Assignee: AstraZeneca AB, Sodertalje (SE)

(21) Appl. No.: 11/813,289

(22) Filed: Jun. 23, 2008

(30) Foreign Application Priority Data
Jan. 6, 2005 (GB) 0500140.9
Oct. 21, 2005 (GB) 0521484.6

(51) Int. Cl. A61K 31/496; C07D 487/04; A61K 31/4439; C07D 401/04; C12N 5/06; A61K 31/551; A61K 31/4545

(52) U.S. Cl. 514/210.2; 546/276.7; 514/338; 540/575; 514/218; 514/318; 546/193; 546/276.4; 514/343; 546/268.1; 544/365; 514/253.13; 435/375

(57) ABSTRACT

The present invention relates to certain novel pyridin compounds of Formula (I) to processes for preparing such compounds, to their utility as P2Y12 inhibitors and as anti-thrombotic agents etc, and processes for their preparation, their use as medicaments in cardiovascular diseases as well as pharmaceutical compositions containing them.

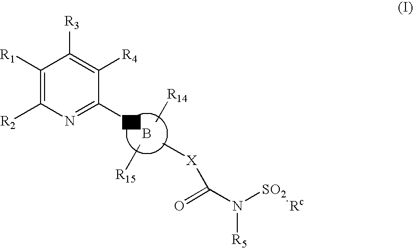

### FIELD OF THE INVENTION

The present invention provides novel pyridine compounds, their use as medicaments, compositions containing them and processes for their preparation.

### BACKGROUND OF THE INVENTION

Platelet adhesion and aggregation are initiating events in arterial thrombosis. Although the process of platelet adhesion to the sub-endothelial surface may have an important role to play in the repair of damaged vessel walls, the platelet aggregation that this initiates can precipitate acute thrombotic occlusion of vital vascular beds, leading to events with high morbidity such as myocardial infarction and unstable angina. The success of interventions used to prevent or alleviate these conditions, such as thrombolysis and angioplasty is also compromised by platelet mediated occlusion or re-occlusion.

Haemostasis is controlled via a tight balance between platelet aggregation, coagulation and fibrinolysis. Thrombus formation under pathological conditions, like e.g. arteriosclerotic plaque rupture, is firstly initiated by platelet adhesion, activation and aggregation. This results not only in the formation of a platelet plug but also in the exposure of negatively charged phospholipids on the outer platelet membrane promoting blood coagulation. Inhibition of the build-up of the initial platelet plug would be expected to reduce thrombus formation and reduce the number of cardiovascular events as was demonstrated by the anti-thrombotic effect of e.g. Aspirin (BMJ 1994; 308: 81-106 Antiplatelet Trialists' Collaboration. Collaborative overview of randomised trials of antiplatelet therapy, I: Prevention of death, myocardial infarction, and stroke by prolonged antiplatelet therapy in various categories of patients.). Platelet activation/aggregation can be induced by a variety of different agonists. However, distinct intracellular signalling pathways have to be activated to obtain full platelet aggregation, mediated via C-proteins G_{q}, G_{12/13} and Gᵢ (Platelets, A D Michelson ed., Elsevier Science 2002, ISBN 0-12-493951-1; 197-213: D Woulfe, et al. Signal transduction during the initiation, extension, and perpetuation of platelet plug formation) In platelets, the G-protein coupled receptor P2Y₁₂ (previously also known as the platelet P_{2T}, P2T_{ac}, or P2Y_{cyc} receptor) signals via Gi, resulting in a lowering of intracellular cAMP and fill aggregation (Nature 2001; 409: 202-207 G Hollopeter, et al. Identification of the platelet ADP receptor targeted by antithrombotic drugs.). Released ADP from dense-granules will positively feedback on the P2Y12 receptor to allow full aggregation.

Clinical evidence for the key-role of the ADP-P2Y₁₂ feedback mechanism is provided by the clinical use of clopidogrel, an thienopyridine prodrug which active metabolite selectively and irreversibly binds to the P2Y₁₂ receptor, that has shown in several clinical trials to be effective in reducing the risk for cardiovascular events in patients at risk (Lancet 1996; 348: 1329-39: CAPRIE Steering committee, A randomised, blinded, trial of clopidogrel versus aspirin in patients at risk of ischaemic events (CAPRIE); N Engl J Med 2001; 345 (7): 494-502): The Clopidogrel in Unstable Angina to prevent Recurrent Events Trial Investigators. Effects of clopidogrel in addition to aspirin in patients with acute coronary syndromes without ST-segment elevation.). In these studies, the clinical benefit with a reduced bleeding risk as compared to thienopyridines (Sem Thromb Haemostas 2005; 31 (2): 195-204 J J J van Giezen & R G Humphries. Preclinical and clinical studies with selective reversible direct P2Y₁₂ antagonists.

Accordingly it is an object of the present invention to provide reversible and selective P2Y₁₂-antagonists as anti-thrombotic agents.

### SUMMARY OF THE INVENTION

We have now surprisingly found that certain pyridine compounds of Formula (I) or a pharmaceutically acceptable salt thereof are reversible and selective P2Y12 antagonists, hereinafter referred to as the compounds of the invention. The compounds of the invention unexpectedly exhibit beneficial properties that render them particularly suitable for use in the treatment of diseases/conditions as described below (See p. 79). Examples of such beneficial properties are high potency, high selectivity, and an advantageous therapeutic window.

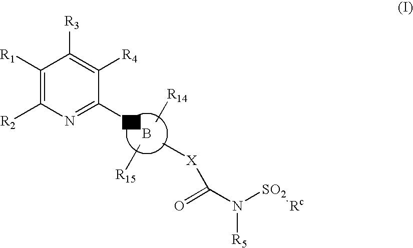

### DETAILED DESCRIPTION OF THE INVENTION

According to the present invention there is provided a novel compound of formula (I) or a pharmaceutically acceptable salt thereof: 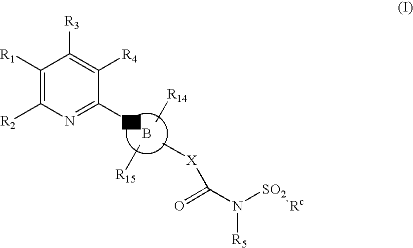
wherein
R₁ represents R₆OC(O), R₇C(O), R₁₆SC(O), R₁₇S, R₁₈C(S) or a group selected from

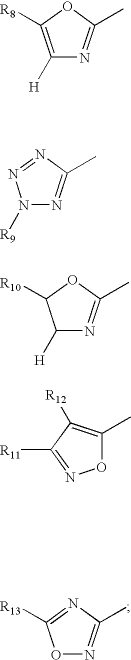

R₂ represents H, CN, NO₂, (C₁₋₁₂)alkyl optionally interrupted by oxygen and/or optionally substituted by OH, aryl, cycloalkyl, heterocyclyl or one or more halogen (F, Cl, Br, I) atoms; further R₂ represents (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxy, (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(2)}R^{b(2)} in which R^{a(2)} and R^{b(2)} independently represent H, (C₁-C₁₂)alkyl, (C₁-C₁₂)alkylC(O) or R^{a2} and R^{b(2)} together with the nitrogen atom represent piperidine, pyrrolidine, azetidine or aziridine;

Further, R₁+R₂ together (with two carbon atoms of the pyridine ring) may form a 5-membered or 6-membered cyclic lactone;

R₃ represents H, CN, NO₂, halogen (F, Cl, Br, I), (C₁-C₁₂)alkyl optionally interrupted by oxygen and/or optionally substituted by OH, aryl, cycloalkyl, heterocyclyl or one or more halogen atoms; further R₃ represents (C₃-C₆)cycloalkyl, hydroxy(C₁-C₁₂)alkyl, (C₁-C₁₂)alkylC(O), (C₁-C₁₂)alkoxy, (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O),heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(3)}R^{b(3)} in which R^{a(3)} and R^{b(3)} independently represent H, (C₁-C₁₂)alkyl, (C₁-C₁₂)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₁₂)alkyl Optionally interrupted by oxygen and/or optionally substituted by OH, COOH, aryl, cycloalkyl, heterocyclyl or one or more halogen atoms; flirter R₄ represents (C₃-C₆)cycloalkyl, hydroxy(C₁-C₁₂)alkyl, (C₁-C₁₂)alkylC(O), (C₁-C₁₂)alkylcycloalkyl, (C₁-C₁₂)alkoxy wherein the alkoxy group may optionally be substituted by OH and/or COOH; further 1R₄ represents (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(4)}R^{b(4)} in which R^{a(4)} and R^{b(4)} independently represent H, (C₁-C₁₂)alkyl, (C₁-C₁₂)alkylC(O) or R^{a(4)} and R^{b(4)} together with the nitrogen atom represent piperidine, pyrrolidine, azetidine or aziridine;

R₅ represents H or (C₁-C₁₂)alkyl;

R₆ represents (C₁-C₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen (F, Cl, Br, I) atoms, further R₆ represents (C₃-C₆)cycloalkyl, hydroxy(C₂-C₁₂)alkyl, aryl or heterocyclyl;

R₇ represents (C₁-C₁₂)alkyl optionally interrupted by oxygen, and/or optionally substituted by OH, aryl, cycloalkyl, heterocyclyl or one or more halogen (F, Cl, Br, I) atoms, further R₇ represents (C₃-C₆)cycloalkyl, hydroxy(C₁-C₁₂)alkyl, (C₁-C₁₂)alkoxy, (C₃-C₆)cycloalkoxy, aryl or heterocyclyl;

R₈ represents H, (C₁-C₁₂)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₁₂)alkyl,(C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;

R₉ represents H, (C₁-C₁₂)alkyl optionally interrupted by oxygen, and/or optionally substituted by aryl, cycloalkyl, heterocyclyl or one or more halogen (F, Cl, Br, I) atoms; further R₉ represents (C₃-C₆)cycloalkyl, hydroxy(C₁—I₂)alkyl, aryl or heterocyclyl;

R₁₀ represents (C₁-C₁₂)alkyl optionally interrupted by oxygen, and/or optionally substituted by aryl, cycloalkyl, heterocyclyl or one or more halogen (F, Cl, Br, I) atoms; further R₁₀ represents (C₃-C₆)cycloalkyl, hydroxy(C₁-C₁₂)alkyl,(C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;

R₁₁ represents H, (C₁-C₁₂)alkyl optionally interrupted by oxygen, and/or optionally substituted by aryl, cycloalkyl, heterocyclyl or one or more halogen (F, Cl, Br, I) atoms; further R₁₁ represents (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;

R₁₂ represents H, (C₁-C₁₂)alkyl optionally interrupted by oxygen, and/or optionally substituted by aryl, cycloalkyl, heterocyclyl or one or more halogen (F, Cl, Br, I) atoms; further R₁₂ represents (C₃₋₆)cycloalkyl, hydroxy(C₁-C₁₂)alkyl,(C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;

R₁₃ represents H, (C₁-C₁₂)alkyl optionally interrupted by oxygen, and/or optionally substituted by aryl, cycloalkyl, heterocyclyl or one or more halogen (F, Cl, Br, I) atoms; further R₁₃ represents (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;

R₁₄ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{d}; wherein R^{d} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl; further R₁₄ represents aryl, cycloalkyl, heterocyclyl, one or more halogen (F, Cl, Br, I) atoms, (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl, a group of formula NR^{a(14)}R^{b(14)} in which R^{a(14)} and R^{b(14)} independently represent H, (C₁-C₁₂)alkyl, (C₁-C₁₂)alkylC(O) or R^{a(14)} and R^{b(14)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{d}; wherein R^{d} represents aryl, cycloalkyl, heterocyclyl or (C₁₋₁₂)alkyl optionally substituted by one or more of halogen (F, Cl, Br, I) atoms, OH, aryl, cycloalkyl and heterocyclyl; further R₁₅ represents aryl, cycloalkyl, heterocyclyl, one or more halogen (F, Cl, Br, I) atoms, (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(15)}R^{b(15)} in which R^{a(15)} and R^{b(15)} independently represent H, (C₁-C₁₂)alkyl, (C₁-C₁₂)alkylC(O) or R^{a(15) and R}^{b(15)} together with the nitrogen atom represent piperidine, pyrrolidine, azetidine or aziridine;

R₁₆ represents (C₁-C₁₂)alkyl optionally interrupted by oxygen and/or optionally substituted by OH, aryl, cycloalkyl, heterocyclyl or one or more halogen (F, Cl, Br, I) atoms, further R₁₆ represents (C₃-C₆)cycloalkyl, hydroxy(C₂-C₁₂)alkyl,(C₁-C₁₂)alkoxy, (C₃-C₆)cycloalkoxy, aryl or heterocyclyl;

R₁₇ represents (C₁-C₁₂)alkyl optionally interrupted by oxygen and/or optionally substituted by OH, aryl, cycloalkyl, heterocyclyl or one or more halogen (F, Cl, Br, I) atoms, further R₁₇ represents (C₃-C₆)cycloalkyl, hydroxy(C₁-C₁₂)alkyl,(C₁-C₁₂)alkoxy, (C₃-C₆)cycloalkoxy, aryl or heterocyclyl;

R₁₈ represents (C₁₋₁₂)alkyl optionally interrupted by oxygen and/or optionally substituted by OH, aryl, cycloalkyl, heterocyclyl or one or more halogen (F, Cl, Br, I) atoms, further R₁₈ represents (C₃-C₆)cycloalkyl, hydroxy(C₁₂)alkyl,(C₁-C₁₂)alkoxy, (C₃-C₆)cycloalkoxy, aryl or heterocyclyl;

R^{c} represents (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rc)}R^{b(Rc)} in which R^{a(Rc)} and R^{b(Rc)} independently represent H, (C₁-C₁₂)alkyl, (C₁₋₁₂)alkylC(O) or R^{a(Rc)} and R^{b(Rc)} together with the nitrogen atom represent piperidine, pyrrolidine, azetidine or aziridine;

X represents a single bond, imino (—NH—), methylene (—CH₂—), iminomethylene (—CH₂—NH—) wherein the carbon is connected to the B-ring/ringsystem, methyleneimino (—NH—CH₂—) wherein the nitrogen is connected to the B-ring/ringsystem and any carbon and/or nitrogen in these groups may optionally be substituted with (C₁-C₆) alkyl; further X may represent a group (—CH₂—)n wherein n=2-6, which optionally is unsaturated and/or substituted by one or more substituent chosen among halogen, hydroxyl or (C₁-C₆)alkyl;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

Preferred values of each variable group are as follows. Such values may be used where appropriate with any of the values, definitions, claims, aspects or embodiments defined hereinbefore or hereinafter. In particular, each may be used as an individual limitation on the broadest definition of formula (I).

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the particular definitions for that group.

It will be understood that when formula I compounds contain a chiral centre, the compounds of the invention may exist in, and be isolated in, optically active or racemic form. The invention includes any optically active or racemic form of a compound of formula I which act as P2Y₁₂ receptor antagonists. The synthesis of optically active forms may be carried out by standard techniques of organic chemistry well known in the art, for example by, resolution of a racemic mixture, by chiral chromatography, synthesis from optically active starting materials or by asymmetric synthesis.

It will also be understood that the compounds of the formula I may exhibit the phenomenon of tautomerism, the present invention includes any tautomeric form of a compound of formula I which is a P2Y₁₂ receptor antagonist.

It will also be understood that in so far as compounds of the present invention exist as solvates, and in particular hydrates, these are included as part of the present invention.

It is also to be understood that generic terms such as “alkyl” include both the straight chain and branched chain groups such as butyl and tert-butyl. However, when a specific term such as “butyl” is used, it is specific for the straight chain or “normal” butyl group, branched chain isomers such as “t-butyl” being referred to specifically when intended.

In one embodiment alkyl is unsubstituted or substituted by one or more halogen (F, Cl, Br, I) atoms and/or one or more of the following groups, OH, ON, NE, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocycyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

One embodiment of alkyl when substituted by one or more halogen atoms (F, Cl, Br, I) include, for example, (C₁-C₆)alkyl substituted by one or more fluorine. atoms, or mixed halogen atoms. Another embodiment of halogen substituted alkyl includes perfluoroalkyl groups such as trifluoromethyl.

The term “cycloalkyl” generally denotes a substituted or unsubstituted (C₃-C₆), unless other chain length specified, cyclic hydrocarbon.

In one embodiment cycloalkyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term aryl denotes a substituted or unsubstituted (C₆-C₁₄) aromatic hydrocarbon and includes, but is not limited to, phenyl, naphthyl, tetrahydronaphtyl, indenyl, indanyl, antracenyl, fenantrenyl, and fluorenyl.

In one embodiment aryl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

The term “heterocyclyl” denotes a substituted or unsubstituted, 4- to 10-membered monocyclic or multicyclic ring system in which one or more of the atoms in the ring or rings is an element other than carbon, for example nitrogen, oxygen or sulfur, especially 4-, 5- or 6-membered aromatic or aliphatic heterocyclic groups, and includes, but is not limited to azetidine, furan, thiophene, pyrrole, pyrroline, pyrrolidine, dioxolane, oxathiolane, oxazolane, oxazole, thiazole, imidazole, imidazoline, imidazolidine, pyrazole, pyrazoline, pyrazolidine, isothiazole, oxadiazole, furazan, triazole, thiadiazole, pyran, pyridine, piperidine, dioxane, morpholine, dithiane, oxathiane, thiomorpholine, pyridazine, pyrimidine, pyrazine, piperazine, triazine, thiadiazine, dithiazine, azaindole, azaindoline, indole, indoline, naphthyridine, benzoxadiazole, dihydrobenzodioxin, benzothiophene, benzothiadiazole, imidazothiazole, 2,3-dihydrobenzofuran, isoxazole, 1,2-benzisoxazole, dihydropyrazole groups, and shall be understood to include all isomers of the above identified groups. For the above groups, e.g. azetidinyl, the term “azetidinyl” as well as “azetidinylene”, etc., shall be understood to include all possible regio isomers. It is further to be understood that the term heterocyclyl may be embodified by one selection among the given possible embodiments for a variable and embodified by another (or the same) selection for another variable, eg. R₄ when selected as heterocyclyl may be a furan, when R^{c} (also when selected as heterocyclyl) may be a pyrrole.

In one embodiment heterocyclyl is substituted by one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, alkylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a}R^{b} in D which R^{a} and R^{b} independently represent H, (C₁-C₁₂)alkyl, (C₁-C₁₂)alkylC(O) or R^{a} and R^{b} together with the nitrogen atom represent piperidine, pyrrolidine, azetidine or aziridine.

In another embodiment of the invention the heterocyclyl group comprises an aromatic 5-membered or 6-membered heterocyclic ring containing one, two or three heteroatoms selected from nitrogen, oxygen and sulphur, and an aromatic 5-membered or 6-membered heterocyclic ring containing one, two or three heteroatoms selected from nitrogen, oxygen and sulphur which is fused to a benzene ring;

In an alternative embodiment of the invention the heterocyclyl group is a non-aromatic 5-membered or 6-membered heterocyclic ring containing one, two or three heteroatoms selected from nitrogen, oxygen and sulphur, fused to a benzene ring.

In a further embodiment of the invention the heterocyclyl group Comprises a group chosen among furyl, pyrrolyl, thienyl, pyridyl, pyrazinyl, pyrimidinyl, pyridazinyl, imidazolyl, oxazolyl, isooxazolyl, thiazolyl, isothiazolyl, oxadiazolyl, 1,2,3-triazolyl, 1,2,4-triazolyl, benzfuranyl, quinolyl, isoquinolyl, benzimidazolyl, indolyl, benzdihydrofuranyl, benzodioxolyl (such as 1,3-benzodioxolyl), benzoxadiazole, dihydrobenzodioxin, benzothiophene, benzothiadiazole, imidazothiazole, 2,3-dihydrobenzofuran, isoxazole, dihydropyrazole and benzdioxanyl (such as 1,4-benzdioxanyl). More particular values include, for example, furyl, pyrrolyl, thienyl, pyridyl, pyrazinyl, pyrimidinyl, pyridazinyl, benzoxadiazole, dihydrobenzodioxin, benzothiophene, benzothiadiazole, imidazothiazole, 2,34-dihydrobenzofuran, isoxazole, 1,2-benzisoxazole, dihydropyrazole or benzdioxanyl (such as 1,4-benzdioxanyl).

In an even further embodiment of the invention the heterocyclyl group is a group chosen among furyl, pyrrolyl, thienyl, pyridyl, pyrazinyl, pyrimidinyl, pyridazinyl, benzoxadiazole, dihydrobenzodioxin, benzothiophene, benzothiadiazole, imidazothiazole, 2,3-dihydrobenzofuran, isoxazole, 1,2-benzisoxazole or dihydropyrazole.

In one embodiment of the invention R₁ represents R₆OC(O).

In another embodiment of the invention R₁ represents R₇C(O).

In yet another embodiment R₁ represents a group selected from

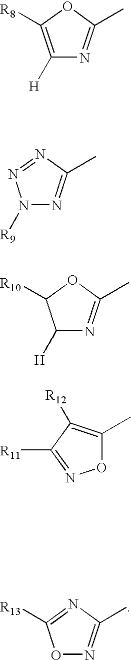

In a further embodiment of the invention R₁ is selected among ROC(O) and R₇C(O) wherein R^{e} can be methyl, ethyl, isopropyl, n-butyl, n-propyl, neopentyl, terbutyl and 2,2-dimethylpropyl and wherein R₇ can be n-propyl or cyclopropyl.

R₁ may also be embodified by a group selected from

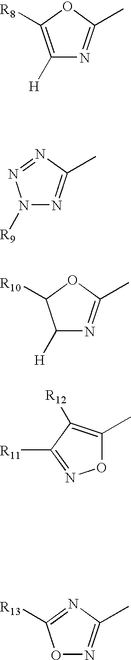

in which R₈, R₉, R₁₁, R₁₂ and R₁₃ are selected from H, (C₁-C₆)alkyl, such as methyl or ethyl; and R₁₀ is selected from (C₁-C₆)alkyl, such as methyl or ethyl.

In another embodiment for the group R₈ this group can be chosen among hydrogen, methyl, ethyl, n-propyl and n-butyl.

Embodiments for R₂ include, for example, H, (C₁-C₄)alkyl and trifluormethyl. Other embodiments for R₂ are trifluoromethyl, methyl, ethyl, iso-propyl, phenyl, methoxy, or amino unsubstituted or optionally substituted with methyl.

Embodiments for R₃ include, for example, H, methyl, methylsulfinyl, hydroxymethyl, methoxy or amino unsubstituted or optionally substituted with one or two methyl groups.

Embodiments for R₄ include H, halogen such as chloro, methyl, cyano, nitro, amino unsubstituted or optionally substituted with one or two methyl groups and (2,2-dimethylpropanoyl)amino.

Another embodiment for R₅ is hydrogen and methyl. Yet another embodiment for R₅ is hydrogen.

Further embodiments for R₈ include, hydrogen, methyl and ethyl.

Further embodiments for R₉ include hydrogen, methyl and ethyl.

Further embodiments for R₁₀ include methyl and ethyl.

A further embodiment for R₁₁ includes methyl.

A further embodiment for R₁₂ includes hydrogen.

Further embodiments for R₁₃ include hydrogen, methyl and ethyl.

Further embodiments for R₁₄ include, for example, hydrogen, methyl, tert-butoxycarbonyl, 2-carboxyethyl, 3-tert-butoxy-3-oxo-propyl.

Other further embodiments for R₁₄ include, for example, methyl, 2-carboxyethyl, and 3-tert-butoxy-3-oxo-propyl.

Further embodiments for R^{c} includes aryl or heterocyclyl, more particularly, aryl or aromatic heterocyclyl.

Another embodiment for R^{c} include, aryl such as phenyl and aromatic heterocyclyl such as thienyl.

Other embodiments of R^{c} include phenyl which optionally may be substituted.

In a special embodiment R^{c} represents aryl, heterocyclyl or (C₃-C₆)cycloalkyl, and anyone of these groups are optionally substituted with one or more halogen (F, Cl, Br, I) atoms and/or one or more of the following groups, OH, CN, NO₂, (C₁-C₁₂)alkyl, (C₁-C₁₂)alkoxyC(O), (C₁-C₁₂)alkoxy, halogen substituted (C₁-C₁₂)alkyl, (C₁-C₆)cycloalkyl, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rc)}R^{b(Rc)} in which R^{a(RC)} and R^{b(Rc)} independently represent H, (C₁-C₁₂)alkyl, (C₁-C₁₂)alkylC(O) or R^{a(Rc)} and R^{b(Rc)} together with the nitrogen atom represent piperidine, pyrrolidine, azetidine or aziridine;

Even further embodiments for R^{c} include phenyl optionally substituted at the 2,3, 4 or 5-positions as well as any combination thereof. Example of substituents are cyano, tetrazol-5-yl, methoxy, trifluoromethoxy, methyl, trifluoromethyl, fluoro, chloro, bromo, methylsulfonyl, nitro, 3-methyl-5-oxo-4,5-dihydro-1H-pyrazol-1-yl. Two adjacent positions (e.g. 2,3) may also be connected to form a ring. Example of such a substituent is 2-naphtyl. Further more specific values for heteroaryls are 2-chloro-5-thienyl, 3-bromo-5-chloro-2-thienyl, 2,1,3-benzoxadiazol-4-yl, 2,4-dimethyl-1,3-thiazol-5-yl, 2,3-dihydro-1,4-benzodioxin-6-yl, 5-chloro-3-methyl-1-benzothien-2-yl, 2,1,3-benzothiadiazol-4-yl, 2,5-di ethyl-3-furyl, 6-chloroimidazo[2,1-b][1,3]thiazol-5-yl, 2,3-dihydro-1-benzofuran-5-yl, 5-chloro-3-thienyl, 5-isoxazol-5-yl-2-thienyl, 5-isoxazol-3-yl-2-thienyl, 4-bromo-5-chloro-2-thienyl, 5-bromo-6-chloropyridin-3-yl, 5-bromo-2-thienyl, 5-pyridin-2-yl-2-thienyl, 2,5-dichloro-3-thienyl, 4,5-dichloro-2-thienyl, benzothien-3-yl, 2,5-dimethyl-3-thienyl, 3-thienyl, 2-thienyl, 5-methylisoxazol-4-yl, pyridin-3-yl, [f-methyl-5-(trifluoromethyl)-1H-pyrazol-3-yl]-2-thienyl, 5-chloro-1,3-dimethyl-1H-pyrazol-4-yl, 4-[(4-chlorophenyl)sulfonyl]-3-methyl-2-thienyl, 5-(methoxycarbonyl)-2-furyl and 4-(methoxycarbonyl)-5-methyl-2-furyl.

Suitable values for the B ring/ring system include, for example, diazepanylene, piperazinylene, piperidinylene, pyrrolidinylene and azetidinylene, wherein anyone of them may be presents in any of their isomeric forms (e.g. piperazin -tetrahydropyridazin-tetrahydropyrimidin).

Embodiments for the B ring/ring system include, for example, diazepanylene, piperazinylene, piperidinylene, pyrrolidinylene and azetidinylene. Further embodiments include these groups which are substituted with R₁₄ having a (C₁-C₆)alkyl group, wherein the (C₁-C₆)alkyl group optionally is substituted with COOR^{d} group, e.g. 12-carboxyethyl group, and wherein R^{d} represents aryl, cycloalkyl, heterocyclyl or (C₁-C₁₂)alkyl optionally substituted by one or more of halogen (F, Cl, Br, I) atoms, OH, aryl, cycloalkyl and heterocyclyl.

In an alternative to the embodiment for the B ring/ring system above, the embodiment include, for example, diazepanylene, piperazinylene, piperidinylene, pyrrolidinylene or azetidinylene groups which are substituted with R₁₄ having a (C₁-C₆)alkyl group, wherein the (C₁-C₆)alkyl group optionally is substituted with COOR^{d} group, e.g. a 2-carboxyethyl group, and wherein R^{d} represents aryl, cycloalkyl, heterocyclyl or (C₁-C₆)alkyl optionally substituted by one or more of halogen (F, Cl, Br, I) atoms, OH, aryl, cycloalkyl and heterocyclyl.

A 2nd embodiment of formula I is defined by;
R₁ represents R₆OC(O), R₇C(O), R₁₆SC(O), R₁₇S, R₁₈C(S) or a groups selected from

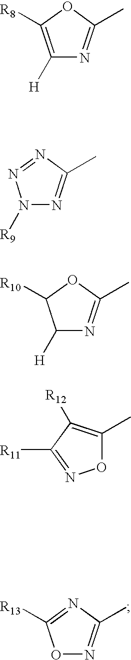

R₂ represents H, CN, NO₂, (C₁-C₆)alkyl optionally interrupted by oxygen and/or optionally substituted by OH, aryl, cycloalkyl, heterocyclyl or one or more halogen (F, Cl, Br, I) atoms; further R₂ represents (C₃-C₆)cycloalkyl, hydroxy(C₁-C₆)alkyl, (C₁-C₆)alkylC(O),(C₁-C₆)alkoxy, (C₁-C₆)alkylthioC(O), (C₁-C₆)alkylC(S), C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine;

Further, R₁+R₂ together (with two carbons from the pyridine ring) may form a 5-membered or 6-membered cyclic lactone;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen atoms; further R₃ represents (C₃-C₆)cycloalkyl, hydroxy(C₁-C₆)alkyl, (C₁-C₆)alkylC(O), (C₁-C₆)alkoxy, (C₁-C₆)alkylthioC(O), (C₁-C₆)alkylC(S), (C₁-q)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₆)alkyl optionally interrupted by oxygen and/or optionally substituted by OH, COOH, aryl, cycloalkyl, heterocyclyl or one or more halogen atoms; further R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by OH and/or COOH; further R₄ represents (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁₋₆)alkylthio, heterocyclyl(C₁-C₆)alkylsulfinyl, heterocyclyl(C₁-C₆)alkylsulfonyl, (C₃-C₆)cycloalkyl(C₁₋₆)alkylthio, (C₃-C₆)cycloalkyl(C₁-C₆)alkylsulfinyl, (C₃)cycloalkyl(C₁-C₆)alkylsulfonyl or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

R₅ represents H or (C₁-C₆)alkyl;

R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocycle or one or more halogen (F, Cl, Br, I) atoms, further R₆ represents (C₃-C₆)cycloalkyl, hydroxy(C₂-C₆)alkyl, aryl or heterocyclyl;

R₇ represents (C₁-C₆)alkyl optionally interrupted by oxygen, and/or optionally substituted by OH, aryl, cycloalkyl, heterocyclyl or one or more halogen (F, Cl, Br, I) atoms, further R₇ represents (C₃-C₆)cycloalkyl, hydroxy(C₁-C₆)alkyl, (C₁-C₆)alkoxy, (C₃-C₆)cycloalkoxy, aryl or heterocyclyl;

R₈ represents H, (C₁-C₆)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)Cycloalkyl(C₁-C₆)alkylthio, (C₃-C₆)cycloalkyl(C₁-C₆)alkylsulfinyl or (C₃-C₆)cycloalkyl(C₁-C₆)alklylsulfonyl;

R₉ represents H, (C₁-C₆)alkyl optionally interrupted by oxygen, and/or optionally substituted by aryl, cycloalkyl, heterocyclyl or one or more halogen (F, Cl, Br, I) atoms; further R₉ represents (C₃-C₆)cycloalkyl, hydroxy(C₁-C₆)alkyl, aryl or heterocyclyl;

R₁₀ represents (C₁-C₆)alkyl optionally interrupted by oxygen, and/or optionally substituted by aryl, cycloalkyl, heterocyclyl or one or more halogen (F, Cl, Br, I) atoms; further R₁₀ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;

R₁₁ represents H, (C₁-C₆)alkyl optionally interrupted by oxygen, and/or optionally substituted by aryl, cycloalkyl, heterocyclyl or one or more halogen (F, Cl, Br, I) atoms; further R₁₁ represents (C₃-C₆)cycloalkyl, hydroxy(C₁-C₆)alkyl,(C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;

R₁₂ represents H, (C₁-C₆)alkyl optionally interrupted by oxygen, and/or optionally substituted by aryl, cycloalkyl, heterocyclyl or one or more halogen (F, Cl, Br, I) atoms; further R₁₂ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₁-C₆)cycloalkyl(C₁-C₆)alkylsulfinyl or (C₃-C₆)cycloalkyl(C₁-C₆)alkylsulfonyl;

R₁₃ represents H, (C₁-C₆)alkyl optionally interrupted by oxygen, and/or optionally substituted by aryl, cycloalkyl, heterocyclyl or one or more halogen (F, Cl, Br, I) atoms; further R₁₃ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₁-C₆)cycloalkyl(C₁-C₆)alkylsulfinyl or (C₃-C₆)cycloalkyl(C₁-C₆)alkylsulfonyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{d}; wherein R^{d} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents aryl, cycloalkyl, heterocyclyl, one or more halogen (F, Cl, Br, I) atoms, (C₃-C₆)cycloalkyl, hydroxy(C₁-C₆)alkyl, (C₁-C₆)alkoxy, (C₃-C₈)cycloalkoxy, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocycyl(C₁-C₆)alkylsulfonyl, (C₃-C₆)Cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(14)}R^{b(14)} in which R^{a(14)} and R^{b(14)} independently represent H, (C₁-C₆)alkyl, (C₁-C₆)alkylC(O) or R^{a(14)} and R^{b(14)} together with the nitrogen atom represent piperidine, pyrrolidine, azetidine or aziridine;

R₁₅ represents H, OH with the proviso that the OH group must Le at least 2 carbon atoms away from any heteroatom in the B ring/ring system, (C₁-C₆)alkyl optionally interrupted by oxygen and/or optionally substituted by one or more of OH, COOH and COOR^{d}; wherein R^{d} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₅ represents aryl, cycloalkyl, heterocyclyl, one or more halogen (F, Cl, Br, I) atoms, (C₃-C₆)cycloalkyl, hydroxy(C₁-C₆)alkyl,(C₁-C₆)alkoxy, (C₃₋₆)cycloalkoxy, aryl heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁₋₆)alkylthio, heterocyclyl(C₁-C₆)alkylsulfinyl, heterocycyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(15)}R^{b(15)} in which R^{a(15) and R}^{b(15)} independently represent H, (C₁-C₆)alkyl, (C₁-C₆)alkylC(O) or R^{a(15)} and R^{b}(15) together with the nitrogen atom represent piperidine, pyrrolidine, azetidine or aziridine;

R₁₆ represents (C₁-C₆)alkyl optionally interrupted by oxygen and/or optionally substituted by OH, aryl, cycloalkyl, heterocyclyl or one or more halogen (F, Cl, Br, I) atoms, further R₁₆ represents (C₃-C₆)cycloalkyl, hydroxy(C₂-C₆)alkyl, (C₁-C₆)alkoxy, (C₃-C₆)cycloalkoxy, aryl, or heterocyclyl;

R₁₇ represents (C₁-C₆)alkyl optionally interrupted by oxygen and/or optionally substituted by OH, aryl, cycloalkyl, heterocyclyl or one or more halogen (F, Cl, Br, I) atoms, further R₁₇ represents (C₃-C₆)cycloalkyl, hydroxy(C₁-C₆)alkyl, (C₁-C₆)alkoxy, (C₃-C₆)cycloalkoxy, aryl or heterocyclyl;

R₁₈ represents (C₁-C₆)alkyl optionally interrupted by oxygen and/or optionally substituted by OH, aryl, cycloalkyl, heterocyclyl or one or more halogen (F, Cl, Br, I) atoms, further R₁₈ represents (C₃-C₆)cycloalkyl, hydroxy(C₁-C₆)alkyl, (C₁-C₆)alkoxy, (C₃-C₆)cycloalkoxy, aryl or heterocyclyl;

R^{c} represents (C₃-C₈)cycloalkyl, aryl or heterocyclyl, and anyone of these groups optionally substituted with one or more halogen (F, Cl, Br, I) atoms and/or one or more of the following groups, OH, CN, NO₂, (C₁-C₆)alkyl, (C₁-C₆)alkoxyC(O), (C₁-C₆)alkoxy, halogen substituted (C₁-C₆)alkyl, (C₃-C₆cycloalkyl, aryl, heterocyclyl, (C₁-C₆alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(Rc)}R^{b(Rc)} in which R^{a(Rc)} and R^{b(Rc)} independently represent H, (C₁-C₆)alkyl, (C₁₋₆)alkylC(O) or R^{a(Rc)} and R^{b(Rc)} together with the nitrogen atom represent piperidine, pyrrolidine, azetidine or aziridine;

X represents a single bond, imino (—NH—), methylene (—CH₂—), iminomethylene (CH₂—NH—) wherein the carbon is connected to the B-ring/ringsystem, methyleneimino (—NH—CH₂—) wherein the nitrogen is connected to the B-ring/ringsystem and any carbon and/or nitrogen in these groups may optionally be substituted with (C₁-C₆) alkyl; further X may represent a group (—CH₂—)n wherein n=2-6, which optionally is unsaturated and/or substituted by one or more substituent chosen among halogen, hydroxyl or (C₁-C₆)alkyl.;

B is a monocyclic or bicyclic, 4 to 11-membered heterocyclic ringing system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

A 3rd embodiment of formula I is defined by;
R₁ represents R₆OC(O), R₇C(O), or a group selected from

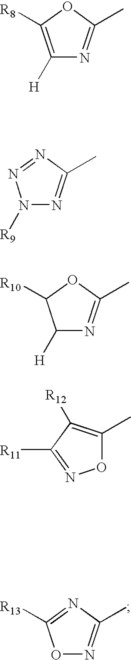

R₂ represents H, CN, NO₂, (C₁-C₆)alkyl optionally interrupted by oxygen and/or optionally substituted by OH, aryl, cycloalkyl, heterocyclyl or one or more halogen (F, Cl, Br, l) atoms; further R₂ represents (C₃-C₆)cycloalkyl, hydroxy(C₁-C₆)alkyl, (C₁-C₆)alkylC(O),(C₁-C₆)alkoxy, (C₁-C₆)alkylthioC(O), (C₁-C₆)alkylC(S), (C₁-C₆)alkoxy(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine;

R₃ represents H, CN, NO₂, halogen (F, Cl, Br, I), (C₁-C₆)alkyl optionally interrupted by oxygen and/or optionally substituted by OH, aryl, cycloalkyl, heterocyclyl or one or more halogen atoms; further R₃ represents (C₃-C₆)cycloalkyl, hydroxy(C₁-C₆)alkyl, (C₁-C₆)alkylC(O),(C₁-C₆)alkoxy, (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;

R₄ represents H, CN, NO₂, halogen (F, Cl, Br, I), (C₁-C₆)alkyl optionally interrupted by oxygen and/or optionally substituted by OH, COOH, aryl, cycloalkyl, heterocyclyl or one or more halogen atoms; further R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by OH and/or COOH; further R₄ represents (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;

R₅ represents H or (C₁-C₆)alkyl;

R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen (F, Cl, Br, I) atoms, further R₆ represents (C₃-C₆)cycloalkyl, hydroxy(C₂-C₆)alkyl, aryl or heterocyclyl;

R₇ represents (C₁-C₆)alkyl optionally interrupted by oxygen, and/or optionally substituted by OH, aryl, cycloalkyl, heterocyclyl or one or more halogen (F, Cl, Br, I) atoms, further R₇ represents (C₃-C₆)cycloalkyl, hydroxy(C₁-C₆)alkyl,(C₁-C₆)alkoxy, (C₃-C₆)cycloalkoxy, aryl or heterocyclyl;

R₈ represents H, (C₁₋₆)alkyl optionally interrupted by oxygen, and/or optionally substituted by aryl, cycloalkyl, heterocyclyl or one or more halogen (F, Cl, Br, I) atoms; further R₈ represents (C₃-C₆)cycloalkyl, hydroxy(C₁-C₆)alkyl, (C₁-C₆)alkoxy, (C₃-C₆)cycloalkoxy, aryl or heterocyclyl;

R₉ represents H, (C₁-C₆)alkyl optionally interrupted by oxygen, and/or optionally substituted by aryl, cycloalkyl, heterocyclyl or one or more halogen (F, Cl, Br, I) atoms; further R₉ represents (C₃-C₆)cycloalkyl, hydroxy(C₁-C₆)alkyl, aryl or heterocyclyl;

R₁₀ represents (C₁-C₆)alkyl optionally interrupted by oxygen, and/or optionally substituted by aryl, cycloalkyl, heterocyclyl or one or more halogen (F, Cl, Br, I) atoms; further R₁₀ represents (C₃-C₆)cycloalkyl, hydroxy(C₁-C₆)alkyl,(C₁-C₆)alkoxy, (C₃-C₆)cycloalkoxy, aryl or heterocyclyl;

R₁₁ represents H, (C₁-C₆)alkyl optionally interrupted by oxygen, and/or optionally substituted by aryl, cycloalkyl, heterocyclyl or one or more halogen (F, Cl, Br, I) atoms; further R₁₁ represents (C₃-C₆)cycloalkyl, hydroxy(C₁-C₆)alkyl,(C₁-C₆)alkoxy, (C₃-C₆)cycloalkoxy, aryl or heterocyclyl;

R₁₂ represents H, (C₁-C₆)alkyl optionally interrupted by oxygen, and/or optionally substituted by aryl, cycloalkyl, heterocyclyl or one or more halogen (F, Cl, Br, I) atoms; further R₁₂ represents (C₃-C₆)cycloalkyl, hydroxy(C₁-C₆)alkyl, (C₁-C₆)alkoxy, (C₃-C₆)cycloalkoxy, aryl or heterocyclyl;

R₁₃ represents H, (C₁-C₆)alkyl optionally interrupted by oxygen, and/or optionally substituted by aryl, cycloalkyl, heterocyclyl or one or more halogen (F, Cl, Br, I) atoms; further R₁₃ represents (C₃-C₆)cycloalkyl, hydroxy(C₁₋₆)alkyl, (C₁-C₆)alkoxy, (C₃-C₆)cycloalkoxy, aryl or heterocyclyl;

R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{d}; wherein R^{d} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₄ represents aryl, cycloalkyl, heterocyclyl, one or more halogen (F, Cl, Br, I) atoms, (C₃-C₆)cycloalkyl, hydroxy(C₁-C₆)alkyl,(C₁-C₆)alkoxy, (C₃₋₆)cycloalkoxy, aryl, heterocycyl or a group of formula NR^{a(15)}R^{b(15)} in which R^{a(15)} and R^{b(15)} independently represent H, (C₁-C₆)alkyl, (C₁-C₆)alkylC(O) or R^{a(15)} and R^{b(15)} together with the nitrogen atom represent piperidine, pyrrolidine, azetidine or aziridine;

R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{d}; wherein R^{d} represents aryl, cycloalkyl, heterocyclyl or (C₁-C₆)alkyl optionally substituted by one or more of halogen (F, Cl, Br, I) atoms, OH, aryl, cycloalkyl and heterocyclyl; further R₁₅ represents aryl, cycloalkyl, heterocyclyl, one or more halogen (F, Cl, Br, I) atoms, (C₃-C₆)cycloalkyl, hydroxy(C₁-C₆)alkyl,(C₁-C₆)alkoxy, (C₃-C₆)cycloalkoxy, aryl, heterocyclyl or a group of formula NR^{a(15)}R^{b(15)} in which R^{a(15)} and R^{b(15)} independently represent H, (C₁-C₆)alkyl, (C₁-C₆)alkylC(O) or R^{a(15)} and R^{b(15)} together with the nitrogen atom represent piperidine, pyrrolidine, azetidine or aziridine;

R^{c} represents (C₃-C₈)cycloalkyl, aryl or heterocyclyl, and anyone of these groups optionally substituted with one or more halogen (F, Cl, Br, I) atoms and/or one or more of the following groups, CN, NO₂, (C₁-C₆)alkyl, (C₁-C₆)alkoxy, halosubstituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;

X represents a single bond, imino (—NH—), methylene (—CH₂—), iminomethylene (—CH₂—NH—) wherein the carbon is connected to the B-ring/ringsystem, methyleneimino (—NH—CH₂—) wherein the nitrogen is connected to the B-ring/ringsystem and any carbon and/or nitrogen in these groups may optionally be substituted with (C₁-C₆) alkyl; further X may represent a group (—CH₂—)n wherein n=2-6, which optionally is unsaturated and/or substituted by one or more substituent chosen among halogen, hydroxyl or (C₁-C₆)alkyl.;

B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions. The substituents R₁₄ and R₁₅ are connected to the B ring/ring system in such a way that no quarternary ammonium compounds are formed (by these connections).

A 4th embodiment of formula I is defined by that;
R₁ is chosen from a group consisting of methoxycarbonyl, ethoxycarbonyl, (n-propyl)-oxycarbonyl, (iso-propyl)-oxycarbonyl, (n-butyl)-oxycarbonyl, (tert-butyl)-oxycarbonyl, (3-methyl-butyl)-oxycarbonyl, (2,2-dimethyl-propyl)-oxycarbonyl, n-propylcarbonyl, (cyclo-propyl)-carbonyl, 3-methylisoxazol-5-yl, 2-ethyl-2H-tetrazol-5-yl, 5-ethyl-4,5-dihydro-1,3-oxazol-2-yl, 5-methyl-1,3-oxazol-2-yl, 5-ethyl-1,3-oxazol-2-yl, 5-propyl-1,3-oxazol-2-yl and 5-butyl-1,3-oxazol-2-yl;
R₂ is chosen from a group consisting of H, methyl, ethyl, isopropyl, trifluoromethyl, methoxy, phenyl, amino and methylamino;
R₃ is chosen from a group consisting of H, amino, methyl, methylamino, dimethylamino, methoxy, methylsulfinyl and hydroxymethyl;
R₄ is chosen from a group consisting of H, methyl, chloro, cyano, amino, methylamino, dimethylamino, isopropylamino, acetylamino, (2,2-dimethylpropanoyl)amino and nitro;
R₅ is chosen from a group consisting of H and methyl;
R₁₄ is chosen from a group consisting of H, methyl, t-butyl carboxylate, 2-carboxyethyl and 3-tert-butoxy-3-oxopropyl;
R₁₅ is H;
R^{c} is chosen from a group consisting of phenyl, 2-methylphenyl, 3-methylphenyl, 4-methylphenyl, 3-(trifluoromethyl)phenyl, 4-(trifluoromethyl)phenyl, 2-(trifluoromethoxy)phenyl, 4-(trifluoromethoxy)phenyl, 2-fluorophenyl, 3-fluorophenyl, 4-fluorophenyl, 2-chlorophenyl, 3-chlorophenyl, 4-chlorophenyl, 3-bromophenyl, 3-cyanophenyl, 4-cyanophenyl, 3-methoxyphenyl, 4-methoxyphenyl, 3-nitrophenyl, 3-(3-methyl-5-oxo-4,5-dihydro-1H-pyrazol-1-yl)phenyl, 2,4-dichlorophenyl, 3,4-dichlorophenyl, 3,5-difluorophenyl, 3,4-dimethoxyphenyl, 2-methyl-5-(methylsulfonyl)phenyl, 2-thienyl, 3-thienyl, 5-bromo-2-thienyl, 5-chloro-2-thienyl, 5-chloro-3-thienyl, 2,5-dichloro-3-thienyl, 2,5-dimethyl-3-thienyl, 4,5-dichloro-2-thienyl, 3-bromo-5-chloro-2-thienyl, 4-bromo-5-chloro-2-thienyl, 5-pyridin-2-yl-2-thienyl, 5-isoxazol-3-yl-2-thienyl, 5-isoxazol-5-yl-2-thienyl, 5-[1-methyl-5-(trifluoromethyl)-1H-pyrazol-3-yl]-2-thienyl, 5-(2-methyl-1,3-thiazol-4-yl)-2-thienyl, 5-chloro-3-methyl-1-benzothien-2-yl, 2,4-dimethyl-1,3-thiazol-5-yl, 2,5-dimethyl-3-furyl, 5-(methoxycarbonyl)-2-furyl, 4-(methoxycarbonyl)-5-methyl-2-furyl, 5-methylisoxazol-4-yl, 5-chloro-1,3-dimethyl-1H-pyrazol-4-yl, pyridin-3-yl, 5-bromo-6-chloropyridin-3-yl, 2-naphtyl, 2,3-dihydro-1,4-benzodioxin-6-yl, 4-(1H-tetrazol-5-yl)phenyl, 2,1,3-benzoxadiazol-4-yl, 2,1,3-benzothiadiazol-4-yl, 6-ethoxy-1,3-benzothiazol-2-yl, 1-benzothien-3-yl, 2,3-dihydro-1,4-benzodioxin-6-yl, 6-chloroimidazo[2,1-b][1,3]thiazol-5-yl and 2,3-dihydro-1-benzofuran-5-yl;
X represents a single bond, imino (—NH—), methylene (—CH₂—) and iminomethylene (—CH₂—NH—) wherein the carbon is connected to the B-ring/ring system;

B is chosen from the group consisting of 4-piperazin-1-ylene, 4-piperidin-1-ylene, 3-piperidin-1-ylene, 3-azetidin-1-ylene, 3-pyrrolidin-1-ylene, 4-(1,4-diazepan)-1-ylene, 5-hexahydropyrrolo[3,4-c]pyrrol-2(1H)-ylene and 5-(2,5-diazabicyclo[2.2.1]hept)-2-ylene, and the substituents R₁₄ and R₁₅ are connected to the B ring/ring system, in such a way that no quarternary ammonium compounds are formed (by these connections).

In a 5th embodiment of formula (I), it is defined as being any compound(s) of formula (Ia)-(Il): 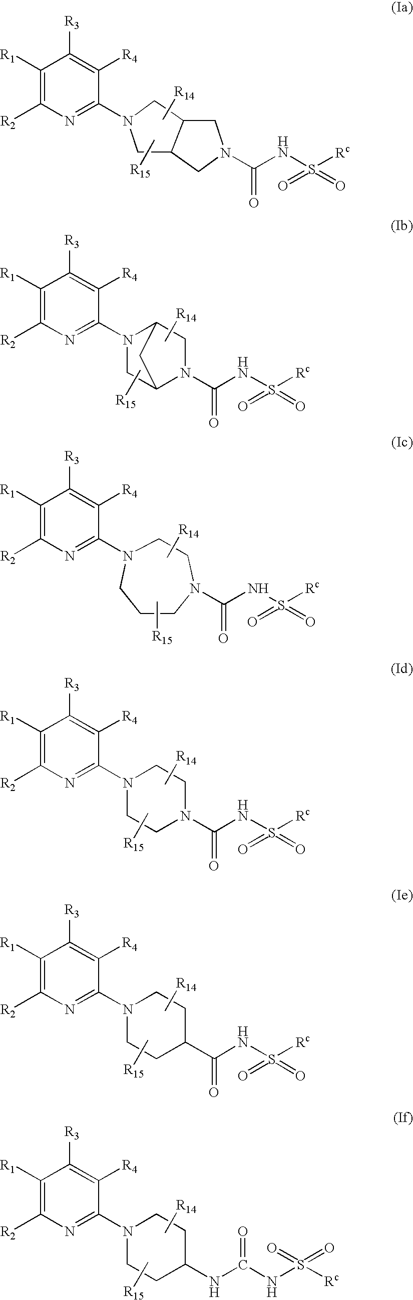 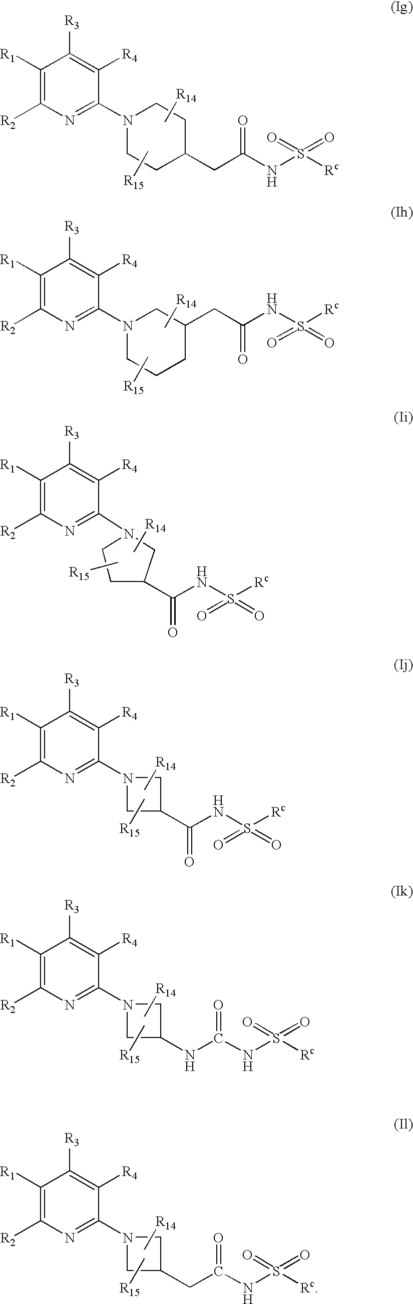
In a 6^{th} embodiment formula (I) is defined as being any compound(s) of formula (Iaa)-(Ipq);

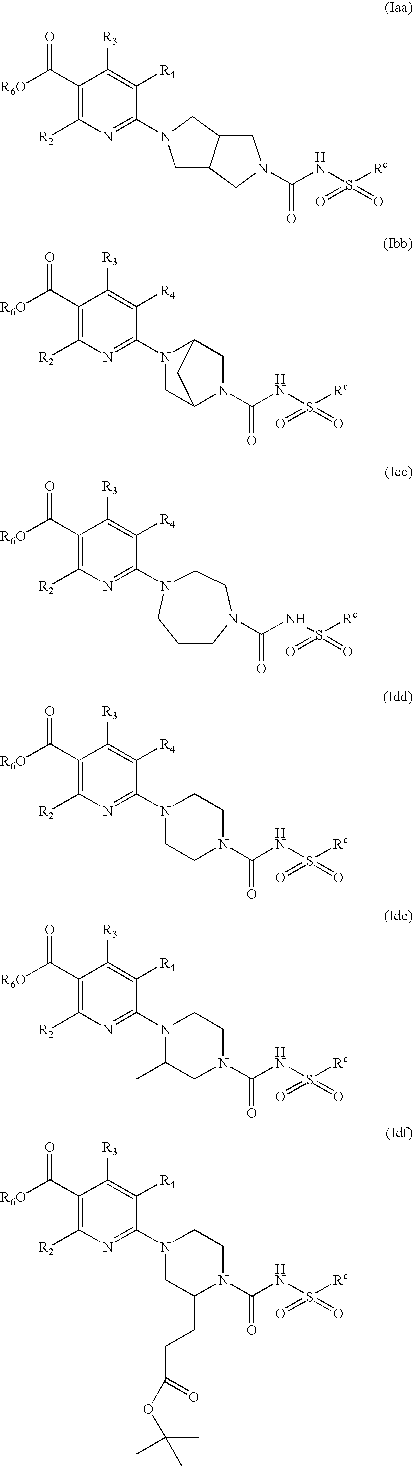

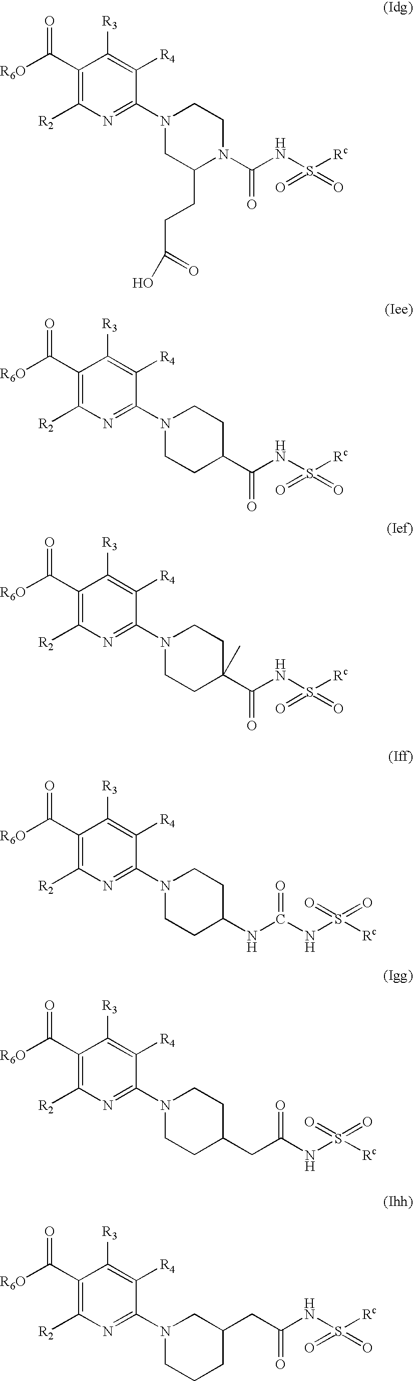

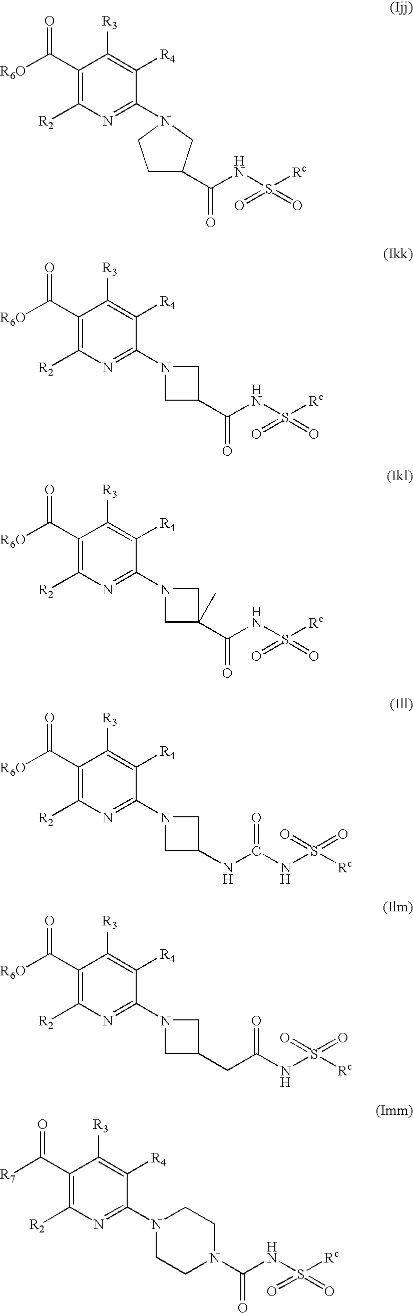

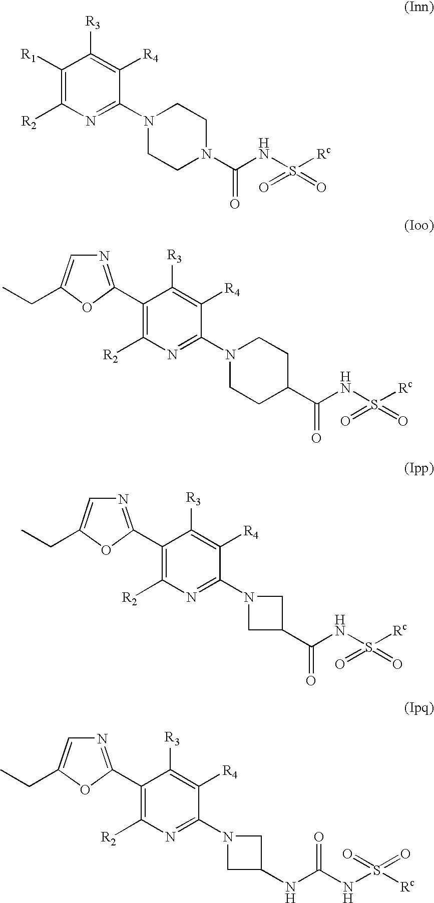

In compound (Inn) R₁ represents a group selected from

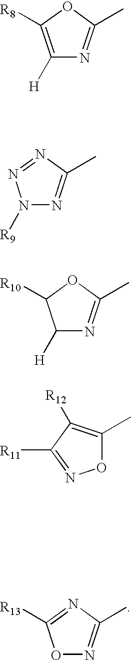

In the above Iaa to Ipq the various values of R (except R₅, R₁₄ and R₁₅, all being H) are as defined above and include the previously mentioned embodiments.

Processes

The following processes together with the intermediates are provided as a further feature of the present invention.

Compounds of formula (I) may be prepared by the following processes a1-a6;

a1) Compounds of formula (I) in which R₁, R₂, R₃, R₄, B, R₅, R₁₄, R₁₅ and R^{c} are defined as above, X is a single bond or a carbon, can be formed by reacting a compound of formula (II), in which R₁, R₂, R₃, R₄, B, R₁₄, and R₁₅ are defined 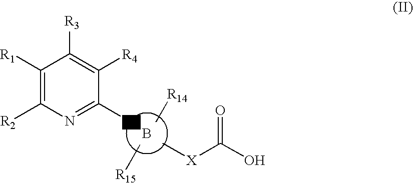
as above, X is a single bond or a carbon, with a compound of formula (III) in which R₅ and R^{c} are defined as above.
R₅—NHSO₂—R^{c} (III)
The reaction is generally carried out in an inert organic solvent such as dichloromethane at ambient temperature. The reaction may be carried out using standard conditions or in the presence of EDCI or HOBT. Optionally, the reaction may be carried out in the presence of an organic base such as triethylamine or DIPEA.

a2) Compounds of formula (I) in which R₁, R₂, R₃, R₄, B, R₅, R₁₄, R₁₅ and R^{c} are defined as above, X is a nitrogen or a single bond connected to a nitrogen which is a member of the B ring, can be formed by reacting a compound of formula (IV), in which R₁, R₂, R₃, R₄, R₁₄, and R₁₅ are defined as above and X is a nitrogen or a hydrogen, with a compound of the general 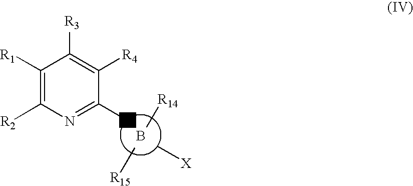
formula (III) which is defined in a) above.
The reaction is generally carried out in an inert solvent such as DCM. The reaction may be carried out in the presence of CDI and a suitable organic base such as triethylamine or DIPEA.

a3) Compounds of formula (I) in which R₁, R₂, R₃, R₄, B, R₁₄, R₁₅, and R^{c} are defined as above, R₅ is a hydrogen, X is a nitrogen or a single bond connected to a nitrogen which is a member of the B ring, can be formed by reacting a compound of formula (IV) which is defined in b) above, with a compound of formula (V)
O═C═N—SO₂—R^{c} (V)
in which R^{c} is as defined above.

The reaction is generally carried out in an inert solvent such as THF. The reaction may also be carried out in the presence of an organic base such as triethylamine or DIPEA.

a4) Compounds of formula (I) in which R₁, R₂, R₃, A, B, R₅, R₁₄, R₁₅, and R^{c} are defined as above, X is a nitrogen or a single bond connected to a nitrogen which is a member of the B ring, can be formed by reacting a compound of formula (IV) which is defined in b) above, with a compound of formula (VI),
R^{c}—SO₂NR₅—COOCH₂CCl₃ (VI)
in which R₅ and R^{c} are as defined above.

The reaction is generally carried out in a solvent such as DMA. This reaction may also be carried out in the presence of an organic base such as triethylamine or DIPEA

a5) Compounds of formula (I) may also be prepared by reacting a compound of formula (VII) in which R₁, R₂, R₃, R₄ are defined as above and L is a suitable leaving group (such as chloro, bromo, iodo, triflate or tosyl), 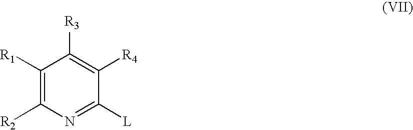
with a compound the general formula (VIII) in which B, R₅, R₁₄, R₁₅, and R^{c} are defined as in formula (I). 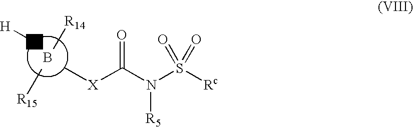 R₅ (VIII)
The reaction is generally carried out in a solvent such as DMA. This reaction may also be carried out in the presence of an organic base such as triethylamine or DIPEA

a6) Compounds of formula (I) where R₁ represents R₆OC(O) and R₂, R₃, R₄, B, R₅, R₁₄, R₁₅, X and R^{c} are defined as for formula (I), can be transesterified using standard procedures or by reacting with R₆—O⁻Li⁺ reagent, to become another compound of the general formula (I) wherein R₁ becomes R₆—OC(O).

The intermediates referred to above may be prepared by, for example, the methods/processes outlined below.

b1) The compounds of formula (II) in which R₁, R₂, R₃, R₄, B, R₁₄, and R₁₅ are defined as above, X is a single bond or a carbon, may be prepared by reacting a compound of formula (IX) 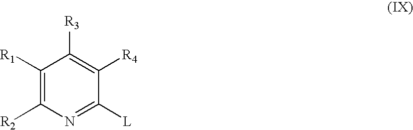
, in which R₁, R₂, R₃, R₄ are defined as for formula (I) above and L is a suitable leaving group (such as chloro, bromo, iodo, triflate or tosyl), with a compound of the general formula (X), 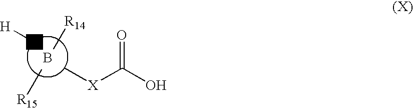
in which B, R₁₄, R₁₅ are defined as above and X is a single bond or a carbon.

The reaction is generally carried out at elevated temperatures using standard equipment or in a single-node microwave oven. Optionally the reaction may be carried out in the presence of an organic base such as TEA or DIPEA.

c) Compounds of formula IV which are defined as above may be prepared by reacting the corresponding compound of formula (IX) which is defined above, with a compound of formula (XI) in which B, R₁₄, R₁₅ are defined as above, X is a nitrogen or a single bond connected to a nitrogen which is a member of the B ring.

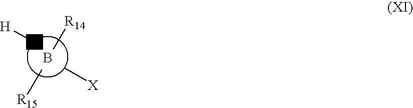

The reaction is generally carried out at elevated temperatures using standard equipment or in a single-node microwave oven. Optionally the reaction may be carried out in the presence of an organic base such as TEA or DIPEA

d) Compounds of formula (XII)

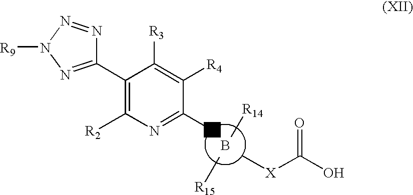

in which R₂, R₃, R₄, B, R₉, R₁₄ and R₁₅ are defined as above and X is a carbon or a single bond may be prepared by a process that comprises the steps d1-d3 below;

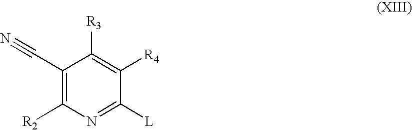

d1) Reacting a compound of the general formula (XIII) in which R₂, R₃, R₄ are defined as for formula I and L is a suitable leaving group, such as chloro, bromo, iodo, triflate or tosyl with a compound of the general formula (X) which is defined as above. The reaction is generally carried out at elevated temperatures using standard equipment or in a single-node microwave oven. Optionally the reaction may be carried out in the presence of an organic base such as TEA or DIPEA, to give a compound of the general formula (XIV).

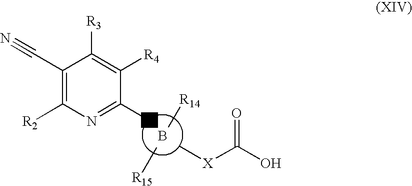

d2) The compound of the general formula (XIV) is then transformed to a compound of the general formula (XV). 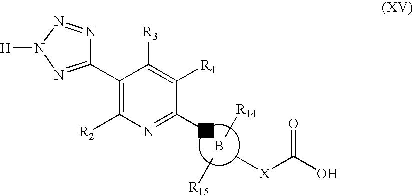
using a suitable reagent such as sodium azide.

d3) The compound of the general formula (XV) can then be reacted with a compound of the general formula (XVI)
R₉-L (XVI)

In which R₉ is defined as above and L is a suitable leaving group such as chloro, bromo, iodo, triflate, tosyl or diazo, to give compounds of the general formula (XII).

e) The preparation of compounds with the general formula (XVII), 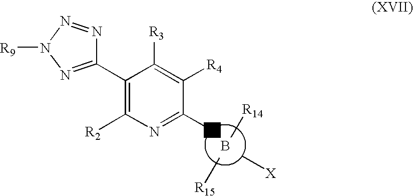
in which R₂, R₃, R₄, B, R₉, R₁₄, R₁₅ are defined as above and X is a nitrogen or a hydrogen connected to a nitrogen which is a member of The B ring, comprises the following steps (e1-e3);

e1) Compounds of the general formula (XIII), defined as above, can be reacted with a compound of the general formula (XI). The reaction is normally performed at elevated temperatures using standard equipment or in a single-node microwave oven, to give a compound of the general formula (XVIII),

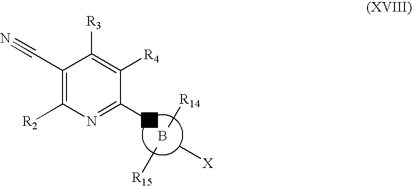

In which R₂, R₃, R₄, B, R₁₄, R₁₅, are defined as above, X is a nitrogen or a hydrogen connected to a nitrogen which is a member of the B ring. Optionally the reaction may be carried out in the presence of an organic base such as TEA or DIPEA.

e2) This compound can be transformed to a compound of the general formula (XIX) 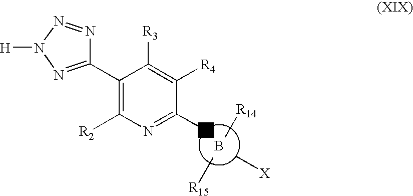
under standard conditions using a suitable reagent such as sodium azide.

e3) Compounds of the general formula (XIX) can thereafter be reacted with compounds of the general formula (XVI), which is defined as above, to give compounds of the general formula (XVII).

f) The preparation of compounds with the general formula (XX), 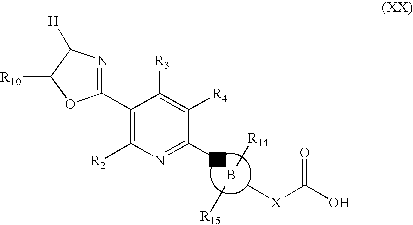
in which R₂, R₃, R₄, B, R₁, R₁₄ and R₁₅ are defined as above and X is a carbon or a single bond comprises the steps (f1-f3) below;

f1) Reacting the corresponding compounds of the general formula (X) which is defined as above with a compound of the general formula (XXI) 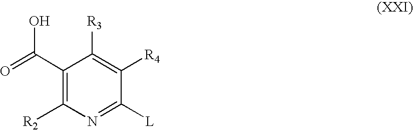
in which R₂, R₃ and R₄ are defined as for formula I, and L is a suitable leaving group, such as chloro, bromo, iodo, triflate or tosyl, to give a compound of formula (XXII).
The reactions are carried at elevated temperatures using standard equipment or a single-node microwave oven. Optionally the reaction may be carried out in the presence of an organic base such as TEA or DIPEA.

f2) The compounds of formula (XXII) can then be reacted 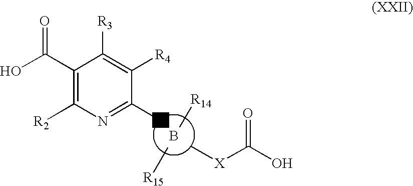
with a compound of the general formula (XXIII), 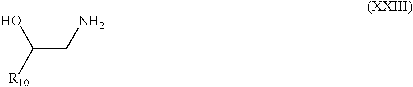
in which R₁₀ is defined as above, to give compounds of the general formula (XXIV). The reactions are carried out using standard conditions or in the presence of EDCI and HOBT. Optionally the reaction may be carried out in the presence of an organic base such as TEA or DIPEA.

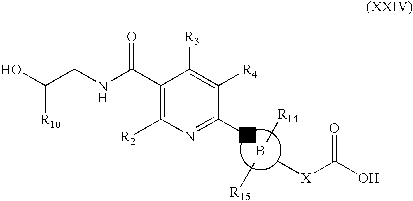

f3) This compound (XXIV) can then be transformed to a compound of the general formula (XX) using known methods or a known reagent such as methanesulfonyl chloride. Optionally the reaction may be carried out in the presence of an organic base such as TEA.
The preparation of compounds of the general formula (XXVI) in which R₂, R₃, R₄, B, R₁₀, R₁₄ and R₁₅, are defined as above, 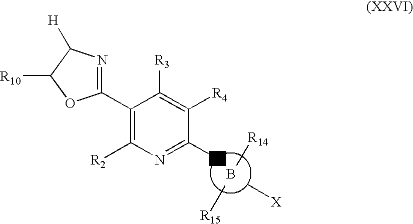
X is a nitrogen or a hydrogen connected to a nitrogen which is a member of the B ring, comprises the following steps (g1-g3);

g1) Reacting a compound of the general formula (XI) which is defined as above with a compound of the general formula (XXI) which is defined as above, to give a compound of the general formula (XXVIII).

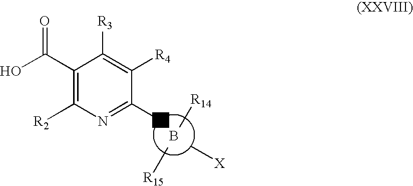

The reactions are carried out at elevated temperatures using standard equipment or a single-node microwave oven. Optionally the reaction may be carried out in the presence of an organic base such as TEA or DIPEA.

g2) The compound of formula (XXVIII) can be reacted with a compound of formula (XXIII), which is defined as above, to give compounds of the general formula (XXIX). The reactions are carried out using standard conditions or in the presence of EDCI and HOBT. Optionally the reactions may be carried out in the presence of an organic base such as TEA or DIPEA.

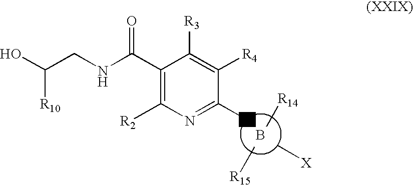

g3) This compound can then be transformed to a compound of the general formula (XXVI) using known methods or a sufficient reagent such as methanesulfonyl chloride. Optionally the reaction may be carried out in the presence of an organic base such as TEA.

h) Compounds of the general formula (XXX), 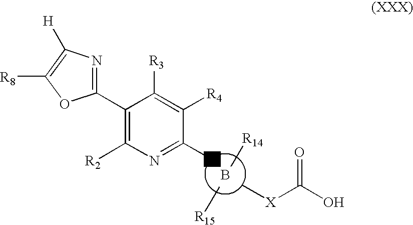
in which R₂, R₃, A, B, R₈, R₁₄ and R₁₅ are defined as above and X is a carbon or a single bond, can be made by oxidising the corresponding compound of the general formula (XX) wherein R₁₀ is the same substituent as to R₈, using a known oxidation reagent such as DDQ.

i) The preparation of compounds of the general formula (XXX) also comprises the steps (i1-i4) below;

i1) Reacting a compound the general formula (XXXI), 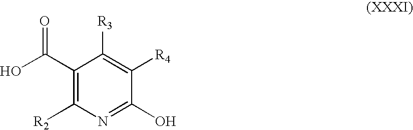
in which R₂, R₃ and R₄ are defined as for compound (I) above, with a compound of the general formula (XXXII), in which R₈ is defined as above, 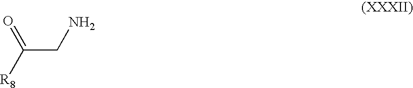
using standard conditions or in the presence of EFCI and HOBT. Optionally the reaction may be carried out in the presence of an organic base such as TEA. This reaction gives a compound of the general formula (XXXIII).

f2) The compound of the general formula (XXXIII) obtained 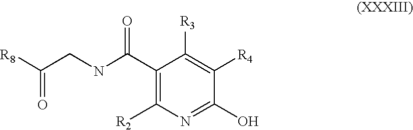
can then be transformed to a compound of the general formula (XXXIV), in which R₂, R₃, R₄ and R₈ are defined as above, using known techniques or using a known reagent such as POCl₃.

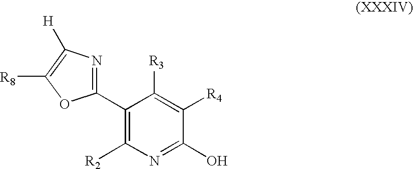

i3) A compound of the general formula (XXXIV) can then be transformed to a compound of the general formula (XXXV), 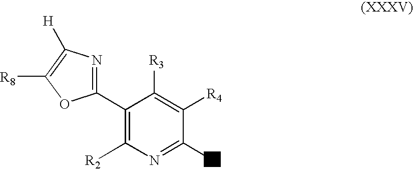
in which R₂, R₃, R₄, R₅ are defined as above and L is a sufficient leaving group, such as chloro, bromo, iodo, triflate or tosyl, using a known techniques or a reagent such as oxalyl chloride or thionyl chloride.

i4) The compound of formula (XXXV) can then be reacted with a compound of the general formula (X), which is defined as above, to give a compound of the general formula (XXX), defined as above. The reactions are carried out at elevated temperatures using standard equipment or a single-node microwave oven. Optionally the reactions may be carried out in the presence of an organic base such as TEA or DIPEA.

j) Compounds of the general formula (XXXVI), 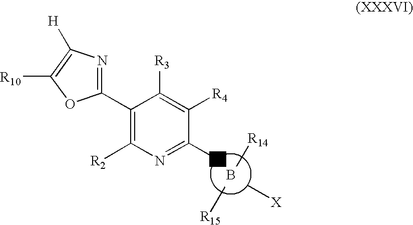
in which R₂, R₃, R₄, B, R₁₀, R₁₄ and R₁₅ are defined as above, X is a nitrogen or a single bond connected to a nitrogen which is a member of the B ring, can be prepared by oxidising a compound of the general formula (XXVI), which is defined as above. The reaction can be performed using standard conditions or a reagent like DDQ.

k) Preparation of compounds with the general formula (XXXVII), 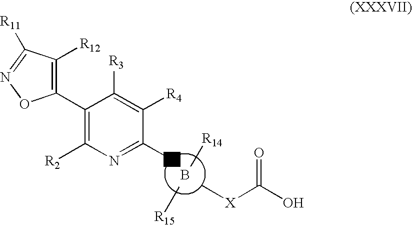
in which R₂, R₃, R₄, B, R₁₁, R₁₂, R₁₄ and R₁₅ are defined as above and X is a carbon or a single bond as above, comprises the steps (k1-k2) below;

k1) Reacting a compound of the general formula (XXII), described above, with N,O-dimethylhydroxylamine. The reaction can be performed using known reagents like CDI to give a compound of the general formula (XXXVIII).

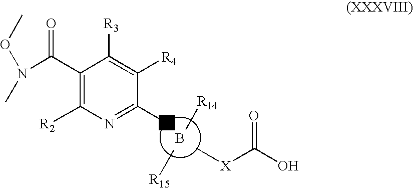

k2) The compounds of formula (XXXVIII) can be reacted with a compound of the general formula (XXXIX), in which R₁₁ and R₁₂ are defined as above. The reaction can be performed 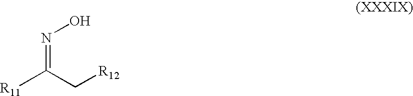
using a known base such as n-butyl lithium.

l) The preparation of compounds of the general formula (XL ), in which R₂, R₃, R₄, B, R₁₁, R₁₂, R₁₄ and R₁₅ are defined as above, X is a nitrogen or a single bond connected to a nitrogen which is a member of the B ring, comprises the steps (l1-l2) below;

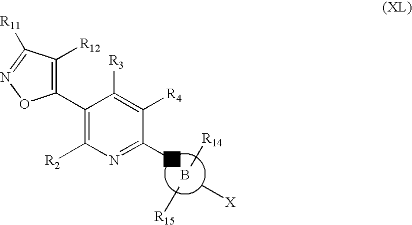

l1) Reacting a compound of the general formula (XXVIII), defined as above, with N,O-dimethylhydroxylamine. The reaction can be performed using known reagents like CDI to give a compound of the general formula (XLI).

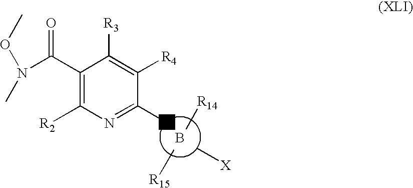

l2) The compounds of the general formula (XLI) above can be reacted with a compound of the general formula (XXXIX) defined as above. This reaction can be performed using known conditions or using a known base such as n-butyl lithium to give a compound of the general formula (XL).

m) The preparation of compounds of the general structure (XLII) in which R₂, R₃, R₄, B, R₁₃R₁₄ and R₁₅ are defined as above and X is a carbon or a single bond, 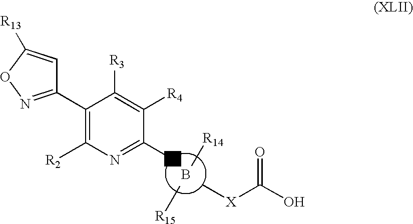
comprises the steps (m1-m2) below;

m1) Reacting a compound of the general formula (XXII), defined as above with hydroxylamine to give compounds of the general formula (XLIII).

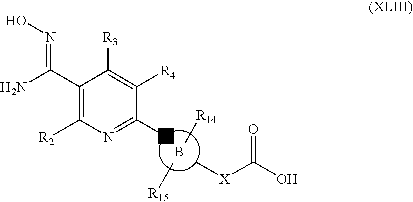

m2) This compound (XLIII)can then be reacted with a reagent mixture like, acetyl chloride/pyridine, propionyl chloride/pyridine or triethyl orthoformiate/BF₃*Et₂O, to give the compound of the general formula (XLII).

n) The preparation of compounds of the general formula (XLIV), 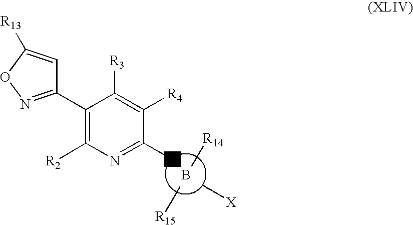
in which R₂, R₃, R₄, B, R₅, R₁₃, R₁₄ and R₁₅ are defined as above, X is a nitrogen or a single bond connected to a nitrogen which is a member of the B ring, comprises the steps (n1-n2) below;

n1) Reacting a compound of the general formula (XVIII), which is defined as above, with hydroxyl amine to give a compound of the general formula (XLV).

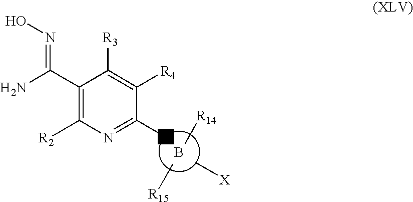

2) This compound (XLV) can then be reacted with a reagent mixture like, acetyl chloride/pyridine, propionyl chloride/pyridine or triethyl orthoformiate/BF₃*Et₂O to give a compound of the general formula (XLIV).

o) Compounds of the general formula (II), in which R₁ is R₇C(O), R₂, R₃, R₄, B, R₁₄ and R₁₅ are defined as above, X is a single bond, may be prepared by reacting a compound of the general formula (XXXVIII), defined as above, with a reagent of the general formula R₇—MgX, in which R₇ is defined as above and X is a halogen, or a reagent of the formula R₇-M, in which M is a metal exemplified by Zn and Li.

p) Compounds of the general formula (IV), in which R₁ is R₇C(O), R₂, R₃, R₄, B, R₁₄ and R₁₅ are defined as above, X is a nitrogen or a single bond connected to a nitrogen which is a member of the B ring, can be prepared by reacting a compound of the general formula (XLI), which is defined as above with a reagent of the general formula R₇—MgX, in which R₇ is defined as above and X is a halogen, or a reagent of the formula R₇-M, in which M is a metal exemplified by Zn and Li.

Compounds of the general formula (VIII) can be formed in one of the processes (q1-q3).

q1) Compounds of the general formula (VIII) in which B, R₅, R₁₄, R₁₅ and R^{c} are defined as above, X is a single bond or a carbon, may be formed by reacting a compound of formula (X) with a compound of formula (III). The reaction is generally carried out in an inert organic solvent such as dichloromethane at ambient temperature. The reaction may be carried out using standard conditions or in the presence of EDCI or HOBT. Optionally, the reaction may be carried out in the presence of an organic base such as triethylamine or DIPEA.

q2) Compounds of the general formula (VIII) in which R₅ is hydrogen, B, R₁₄, R₁₅, and R^{c} are defined as above, X is a nitrogen or a single bond connected to a nitrogen which is a member of the B ring, can be formed by reacting a compound of formula (XI) defined as above with a compound of formula (V), defined as above. The reaction is generally carried out in an inert solvent such as THF. The reaction may also be carried out in the presence of an organic base such as triethylamine or DIPEA.

q3) Compounds of the general formula (VIII) in which B, R₅, R₁₄, R₁₅, and R^{c} are defined as above, X is a nitrogen or a single bond connected to a nitrogen which is a member of the B ring, can also be formed by reacting a compound of formula (XI) with a compound of formula (VI) which is defined as above. The reaction is generally carried out in a solvent such as DMA. This reaction may also be carried out in the presence of an organic base such as triethylamine or DIPEA

(r)Compounds of the general formula (VII) which are defined as above can be formed by reacting a compound of formula (XLVI) using standard conditions or with a reagent such as thionyl chloride or POCl₃.

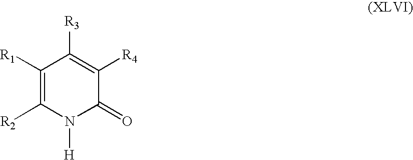

The preparation of compounds of the general formula (XLVII which is defined as above comprises the steps (s1-s3) below;

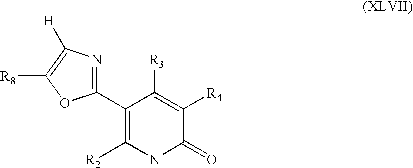

s1) Reacting a compound of the general formula (XLVIII) 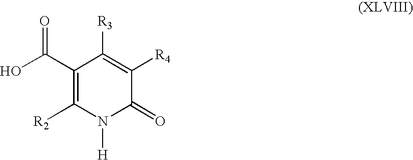
with a compound of the general formula (XXIII), which is having R₈ instead of R₁₀, otherwise defined as above, to give a compound of the formula (IL). The reaction is generally carried out in DCM at ambient temperature. The reaction may be carried out using standard conditions or in the presence of EDCI and HOBt. Optionally the reaction may be carried out in the presence of an organic base such as TEA or DIPEA.

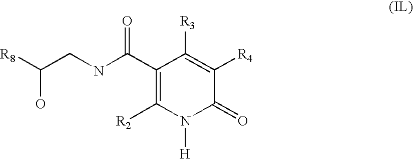

s2) The compound of formula (IL) can be transformed to a compound (L) using standard conditions or an oxidising agent such as the mixture of oxalylchloride and DMSO.

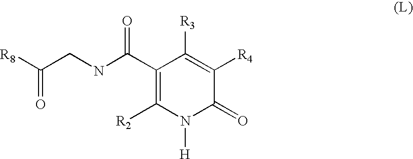

s3) The compound of formula (L) can then be transformed into a compound of the general formula (XLVII), using standard conditions or in the presence of (Methoxycarbonylsulfamoyl)triethylammonium hydroxide (Burgess reagent). The reaction is generally performed in an inert solvent such as THF. The reaction is carried out at elevated temperatures using standard equipment or a single-node microwave oven.

Compounds of the general formula (III) can be formed by reacting the corresponding sulfonyl chloride using known methods with ammonia in an inert solvent such as methanol.

t) Preparation of compounds of the general formula (XLVIII) which is defined as above except for R₃ which is hydrogen, comprises the following steps (t₁-t₃);

t1) Reacting a compound of the formula (LI), in which R₂ and R₆ are defined as for formula (I) with dimethoxy-N,N-dimethylmethaneamine to form a 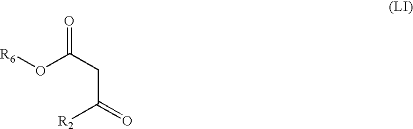
compound of formula (LII).

t2) This compound (LII) can then be reacted further with a compound of the 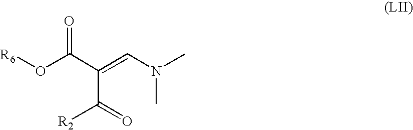
general formula R₄CH₁₂C(O)NH₂, in which R₄ is defined as for formula (I) to give a compound of the general formula (LIII).

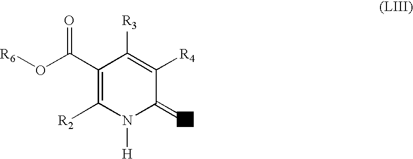

(t3) A compound of the general formula (LIII) can then be transformed to a compound of the general formula (XLVIII). The reaction is generally performed in a protic solvent such as water together with a co-solvent such as THF or methanol. The reaction can be performed using standard reagents or in the presence of LiOH, NaOH or KOH.

(u) The formation of a compound of the general formula (XXX), which is defined as above can be made the below synthesis;

u1) A compound of the general formula (LIV) where R₈ is defined as for formula (I) above can be 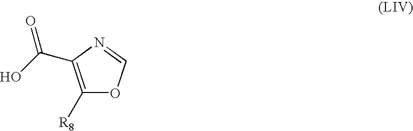
transformed in to a compound of the formula (LV) 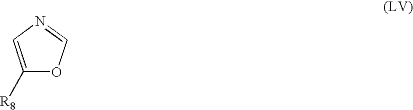
using standard conditions or using Cu(II)O and quinoline.

u2) The compound of the general formula (LV) can be reacted with a compound of the general formula (LVI) in 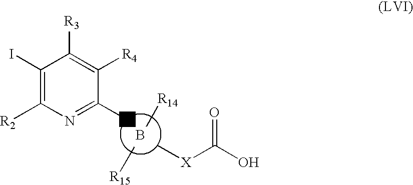
which R₂, R₃, R₄, B, R₁₄ and R₁₅ are defined as for formula (I) and X is a carbon or a single bond, to give compounds of the general formula (XXX). The reaction is generally performed in an inert solvent such as THF under inert atmosphere. The reaction can be performed using standard conditions or in the presence of AlkylLi such as BuLi, ZnCl₂₇ Pd(Ph₃)₄.

(v) Compounds of the general formula (XXXVI) can also be made by the step below;

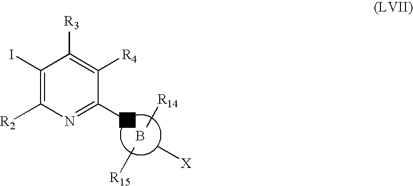

v1) Reacting a compound of the general formula (LV), which is defined as above, with a compound of the general formula (LVII), in which R₂, R₃, R₄, X, R₁₄ and R₁₅ are defined as in formula (I) above, X is a nitrogen or a single bond connected to a nitrogen which is a member of the B ring.

x) The preparation of compounds of the general formula (LVIII), in which R₁₄ and R₁₅ are defined as for formula (I) with the exception that R₁₄ is connected to the same atom as X, and X is defined as a single bond, comprises the below step;

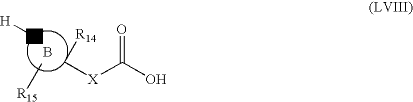

x1) Reacting the corresponding (LIX) with R₁₄-L, wherein L is a suitable leaving group, such as chloro, bromo, iodo, 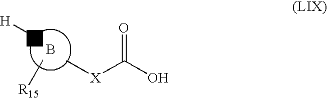
triflate or tosyl to form compounds of the general formula (LVIII), using standard conditions or in the presence of with BuLi and diisopropylamine mixture.

In the reaction schemes described, R₁₄ and R₁₅ can be interchangeably replaced by each other.

At any stage in the synthesis of amine substituted pyridines, a chlorine substituent in the 2, 4 or 6 position of the pyridine can be substituted with azide using known techniques. The azide can be reduced to the corresponding amine. These amines can subsequently be alkylated or acylated using known methods or with an alkylhalide or acylhalide, respectively.

Persons skilled in the art will appreciate that an acid can be transformed to the corresponding activated ester such as an acid chloride, followed by reaction with a thiol, R₁₆SH to give thioesters, R₁₆SC(O).

Persons skilled in the art will appreciate that a nitrogen substituent at the 3 position of a pyridine could be replaced by a thioether chain, R₁₇S—, using known techniques or R₁₇SSR₁₇ and tert-Butylnitrite.

Persons skilled in the art will appreciate that a thioketone could be made from the corresponding ketone using known techniques or using Lawessons reagent.

The compounds of the invention may be isolated from their reaction mixtures using conventional techniques.

Persons skilled in the an will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

It will be appreciated that by those skilled in the art that the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkyl silyl or diarylalkylsilyl groups (e.g. t-butyldimethylsilyl, t-butyldiphenylsilyl or trimethylsilyl) and tetrahydropyranyl. Suitable protecting groups for carboxylic acids include (C₁-C₆)alkyl or benzyl esters. Suitable protecting groups for amino include t-butyloxycarbonyl, benzyloxycarbonyl, 2-(trimethylsilyl)ethoxymethyl or 2-trimethylsilylethoxycarbonyl (Teoc).

The protection and deprotection of functional groups may take place before or after any reaction in the above mentioned process.

Persons skilled in the art will appreciate that, in order to obtain compounds of the invention in an alternative, and on some occasions, more convenient, manner, the individual process steps mentioned hereinbefore may be performed in different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

Persons skilled in the art will appreciate that starting materials for any of the above processes can in some cases be commercially available.

Persons skilled in the art will appreciate that processes above could for some starting materials above be found in the general common knowledge.

The type of chemistry involved will dictate the need for protecting groups as well as sequence for accomplishing the synthesis.

The use of protecting groups is fully described in “Protective groups in Organic Chemistry”, edited by J W F McOrnie, Plenum Press (1973), and “Protective Groups in Organic Synthesis”, 3^{rd} edition, T. W. Greene & P. G. M Wutz, Wiley-Interscience (1999).

Protected derivatives of the invention may be converted chemically to compounds of the invention using standard deprotection techniques (e.g. under alkaline or acidic conditions). The skilled person will also appreciate that certain compounds of Formula (II)-(LIX) may also be referred to as being “protected derivatives”

Compounds of the invention may also contain one or more asymmetric carbon atoms and may therefore exhibit optical and/or diastereoisomerism. Diastereoisomers may be separated using conventional techniques, e.g. chromatography. The various stereoisomers may be isolated by separation of a racemic or other mixture of the compounds using conventional, e.g. HPLC techniques. Alternatively the desired optical isomers may be made by reaction of the appropriate optically active starting materials under conditions which will not cause racemisation or epimerisation, or by derivatisation, for example with a homochiral acid followed by separation of the diasteromeric derivatives by conventionals means (e.g. HPLC, chromatography over silica). Stereocenters may also be introduced by asymmetric synthesis, (e.g metalloorganic reactions using chiral ligands). All stereoisomers are included within the scope of the invention.

All novel intermediates form a flirter aspect of the invention.

Salts of the compounds of formula (I) may be formed by reacting the free acid, or a salt thereof, or the free base, or a salt or a derivative thereof, with one or more equivalents of the appropriate base (for example ammonium hydroxide optionally substituted by C₁-C₆-alkyl or an alkali metal or alkaline earth metal hydroxide) or acid (for example a hydrohalic (especially HCl), sulphuric, oxalic or phosphoric acid). The reaction may be carried out in a solvent or medium in which the salt is insoluble or in a solvent in which the salt is soluble, e.g. water, ethanol, tetrahydrofuran or diethyl ether, which may be removed in vacuo, or by freeze drying. The reaction may also carried out on an ion exchange resin. The non-toxic physiologically acceptable salts are preferred, although other salts may be useful, e.g. in isolating or purifying the product.

Compounds of the Invention

The invention includes any compound(s) selected from;

Ethyl 5-chloro-6-[4-({[(2-methylphenyl)sulfonyl]amino}carbonyl)piperazin-1-yl]nicotinate,
Ethyl 5-chloro-6-[4-({[(4-methylphenyl)sulfonyl]amino}carbonyl)piperazin-1-yl]nicotinate,
Ethyl 5-cyano-6-[4-({[(4-fluorophenyl)sulfonyl]amino}carbonyl)piperazin-1-yl]-2-(trifluoromethyl)nicotinate,
Ethyl 5-chloro-6-[4-({[(4-chlorophenyl)sulfonyl]amino}carbonyl)piperazin-1-yl]nicotinate,
Ethyl 5-chloro-6-[4-({[(5-chloro-2-thienyl)sulfonyl]amino}carbonyl)piperazin-1-yl]nicotinate,
Ethyl-6-(4-{[phenylsulfonyl)amino]carbonyl}piperazine-1-yl)-2-(trifluoromethyl)nicotinate,
Ethyl 5-cyano-6-(4-{[phenylsulfonyl)amino]carbonyl}piperazin-1-yl)-2-(trifluoromethyl)nicotinat,
Ethyl 6-[4-(I{[(2-chlorophenyl)sulfonyl]amino}carbonyl)piperazin-1-yl]-5-cyano-2-(trifluoromethyl)nicotinate,
Ethyl 5-cyano-6-[4-({[(4-methylphenyl)sulfonyl]amino}carbonyl)piperazin-1-yl]-2-(trifluoromethyl)nicotinate,
Ethyl 5 chloro-6-(4-{[(phenylsulfonyl)amino]carbonyl}piperazin-1-yl)nicotinate,
Ethyl 5-cyano-2-methyl-6-(4-{[(phenylsulfonyl)amino]carbonyl}piperazin-1-yl)nicotinate,
Ethyl 6-[4-({[(5-chloro-2-thienyl)sulfonyl]amino}carbonyl)piperazin-1-yl]-5-cyano-2-(trifluoromethyl)nicotinate,
Ethyl 5-chloro-6-[4-({[(4-fluorophenyl)sulfonyl]amino}carbonyl)piperazin-1-yl]nicotinate,
Ethyl 5-chloro-6-[4-({[(2-chlorophenyl)sulfonyl]amino}carbonyl)piperazin-1-yl]nicotinate,
Ethyl 6-[4-({[(4-chlorophenyl)sulfonyl]amino}carbonyl)piperazin-1-yl]-5-cyano-2-(trifluoromethyl)nicotinate,
Ethyl 5-cyano-6-[4-({[(2-methylphenyl)sulfonyl]amino}carbonyl)piperazin-1-yl]-2-(trifluoromethyl)nicotinate,
Ethyl 6-[4-({[(5-chloro-2-thienyl)sulfonyl]amino}carbonyl)piperazin-1-yl]-5-cyano-2-methylnicotinate,
Isopropyl 5-chloro-6-[4-({[(5-chloro-2-thienyl)sulfonyl]amino}carbonyl)piperazin-1-yl]nicotinate,
Butyl 5-chloro-6-[4-({[(5-chloro-2-thienyl)sulfonyl]amino}carbonyl)piperazin-1-yl]nicotinate,
Methyl 5-chloro-6-[4-({[(5-chloro-2-thienyl)sulfonyl]amino}carbonyl)piperazin-1-yl]nicotinate,
Propyl 5-chloro-6-[4-({[(5-chloro-2-thienyl)sulfonyl]amino}carbonyl)piperazin-1-yl]nicotinate,
3-Methylbutyl 5-chloro-6-[4-({[(5-chloro-2-thienyl)sulfonyl]amino}carbonyl)-piperazin-1-yl]nicotinate,
Ethyl 5-chloro-6-(4-{[(phenylsulfonyl)amino]carbonyl}piperidin-1-yl)nicotinate,
Ethyl 5-chloro-6-[4-({[(5-chloro-2-thienyl)sulfonyl]amino}carbonyl piperidin-1-yl]nicotinate,
Ethyl 5-chloro-6-[3-({[(phenylsulfonyl)amino]carbonyl}amino)azetidin-1-yl]nicotinate,
Ethyl 5-chloro-6-[3-({[(5-chloro-2-thienyl)sulfonyl]amino}carbonyl)azetidin-1-yl]nicotinate,
Ethyl 5-chloro-6-[3-({[(phenylsulfonyl)amino]carbonyl}amino)azetidin-1-yl]nicotinate,
Ethyl 5-chloro-6-[3-({[(5-chloro-2-thienyl)sulfonyl]amino}carbonyl)pyrrolidin-1-yl]nicotinate,
Ethyl 6-[3-(3-tert-butoxy-3-oxopropyl)-4-({[(5-chloro-2-thienyl)sulfonyl]amino}carbonyl)piperazin-1-yl]-5-cyano-2-(trifluoromethyl)nicotinate,
3-{1-({[(5-chloro-2-thienyl)sulfonyl]amino}carbonyl)-4-[3-cyano-5-[ethoxy(hydroxy)methyl]-6-(trifluoromethyl)pyridin-2-yl]piperazin-2-yl}propanoic acid,
Ethyl 6-(3-(3-tert-butoxy-3-oxopropyl)-4-{[(phenylsulfonyl)amino]carbonyl}piperazin-1-yl)-5-cyano-2-(trifluoromethyl)nicotinate,
3-(4-[3-Cyano-5-(ethoxycarbonyl)-6-(trifluoromethyl)pyridin-2-yl]-1-{[(phenylsulfonyl)amino]carbonyl}piperazin-2-yl)propanoic acid,
Ethyl 6-(3-(3-tert-butoxy-3-oxopropyl)-4-{[(phenylsulfonyl)amino]carbonyl}piperazin-1-yl)-5-chloronicotinate,
3-(4-[3-Chloro-5-(ethoxycarbonyl)pyridin-2-yl]-1-{[(phenylsulfonyl)amino]carbonyl}piperazin-2-yl)propanoic acid,
Ethyl 5-Chloro-6-[4-({[(phenylsulfonyl)amino]carbonyl}amino)piperidin-1-yl]nicotinate,
4-(5-Butyryl-3-chloropyridin-2-yl)-N-[(5-chloro-2-thienyl)sulfonyl]piperazine-1-carboxamide,
4-[3-Chloro-5-(2-ethyl-2H-tetrazol-5-yl)pyridin-2-yl]-N-[(5-chloro-2-thienyl)sulfonyl]piperazine-1-carboxamide,
4-[3-Chloro-5-(5-ethyl-4,5-dihydro-1,3-oxazol-2-yl)pyridin-2-yl]-N-(phenylsulfonyl)piperazine-1-carboxamide,
4-[3-Chloro-5-(5-methyl-1,3-oxazol-2-yl)pyridin-2-yl]-N-(phenylsulfonyl)piperazine-1-carboxamide,
4-[3-Chloro-5-(5-methyl-1,3-oxazol-2-yl)pyridin-2-yl]-N-[(5-chloro-2-thienyl)sulfonyl]piperazine-1-carboxamide,
4-[3-Chloro-5-(5-ethyl-1,3-oxazol-2-yl)pyridin-2-yl]-N-(phenylsulfonyl)piperazine-1-carboxamide,
4-[3-Chloro-5-(5-ethyl-1,3-oxazol-2-yl)pyridin-2-yl]-N-[(5-chloro-2-thienyl)sulfonyl]piperazine-1-carboxamide,
4-[3-Chloro-5-(3-methylisoxazol-5-yl)pyridin-2-yl]-N-[(5-chloro-2-thienyl)sulfonyl]piperazine-1-carboxamide,
4-[3 Chloro-5-(5-ethyl-1,2,4-oxadiazol-3-yl)pyridin-2-yl]-N-[(5-chloro-2-thienyl)sulfonyl]piperazine-1-carboxamide,
Isopropyl 5-cyano-2-methyl-6-[4-({[(4-methylphenyl)sulfonyl]amino}carbonyl)piperidin-1-yl]nicotinate,
Isopropyl 5-cyano-2-methyl-6-(4-{[(2-naphthylsulfonyl)amino]carbonyl}piperidin-1-yl)nicotinate,
Ethyl 6-{3-[({[(4-chlorophenyl)sulfonyl]amino}carbonyl)amino]azetidin-1-yl}-5-cyano-2-methylnicotinate,
Ethyl 6-{3-[({[(5-chloro-2-thienyl)sulfonyl]amino}carbonyl)amino]azetidin-1-yl}-5-cyano-2-methylnicotinate,
Ethyl 6-[4-({[(5-chloro-2-thienyl)sulfonyl]amino}carbonyl)piperidin-1-yl]-5-cyano-2-isopropylnicotinate,
Ethyl 6-[4-({[(5-chloro-2-thienyl)sulfonyl]amino}carbonyl)piperidin-1-yl]-5-cyano-2-phenylnicotinate,
Ethyl 6-[4-({[(5-chloro-2-thienyl)sulfonyl]amino}carbonyl)piperidin-1-yl]-5-cyano-2-ethylnicotinate,
tert-Butyl 6-[4-({[(5-chloro-2-thienyl)sulfonyl]amino}carbonyl)piperidin-1-yl]-5-cyano-2-methylnicotinate,
2,2-Dimethylpropyl 6-{3-[({[(5-chloro-2-thienyl)sulfonyl]amino}carbonyl)amino]azetidin-1-yl}-5-cyano-2-methylnicotinate,
2,2-Dimethylpropyl 6-[4-({[(5-chloro-2-thienyl)sulfonyl]amino}carbonyl)piperidin-1-yl]-5-cyano-2-methylnicotinate,
Isopropyl 5-cyano-2-methyl-6-[4-({[(5-methyl-2-thienyl)sulfonyl]amino}carbonyl)piperidin-1-yl]nicotinate,
Ethyl 5-cyano-2-methyl-6-[3-(({[(3-methylphenyl)sulfonyl]amino}carbonyl)azetidin-1-yl]nicotinate,
Ethyl 5-cyano-2-methyl-6-[3-({[(phenylsulfonyl)amino]carbonyl}amino)azetidin-1-yl]nicotinate,
1-[3-Chloro-5-(5-ethyl-1,3-oxazol-2-yl)-6-(methylamino)pyridin-2-yl]-N-[(5-chloro-2-thienyl)sulfonyl]piperidine-4-carboxamide,
Ethyl 5-cyano-2-methyl-6-(4-{2-oxo-2-[(phenylsulfonyl)amino]ethyl}piperidin-1-yl)nicotinate,
Ethyl 4-amino-5-chloro-6-[4-({[(5-chloro-2-thienyl)sulfonyl]amino}carbonyl)piperidin-1-yl]nicotinate,
Ethyl 6-[4-(2-{[(5-chloro-2-thienyl)sulfonyl]amino}-2-oxoethyl)piperidin-1-yl]-5-cyano-2-methylnicotinate,
Ethyl 6-[4-({[(5-chloro-2-thienyl)sulfonyl]amino}carbonyl)-1,4-diazepan-1-yl]-5-cyano-2-methylnicotinate,
Ethyl 6-[4-({[(5-chloro-2-thienyl)sulfonyl]amino}carbonyl)-2-methylpiperazin-1-yl]-5-cyano-2-methylnicotinate,
Ethyl 5-cyano-2-methyl-6-(4-{[(phenylsulfonyl)amino]carbonyl}-1,4-diazepan-1-yl)nicotinate,
1-[3-Chloro-5-(5-ethyl-1,3-oxazol-2-yl)-4-(methylamino)pyridin-2-yl]-N-[(5-chloro-2-thienyl)sulfonyl]piperidine-4-carboxamide,
Ethyl 6-[4-({[(5-chloro-2-thienyl)sulfonyl]amino}carbonyl)-4-methylpiperidin-1-yl]-5-cyano-2-methylnicotinate,
Ethyl 6-(3-{[({[(5-chloro-2-thienyl)sulfonyl]amino}carbonyl)amino]methyl}azetidin-1-yl)-5 cyano-2-methylnicotinate,
Ethyl 5-cyano-2-methyl-6-{3-[({[(phenylsulfonyl)amino]carbonyl}amino)methyl]azetidin-1-yl}nicotinate,
Ethyl 5-cyano-6-[3-({[(4-cyanophenyl)sulfonyl]amino}carbonyl)azetidin-1-yl]-2-methylnicotinate,
Ethyl 6-(3-{[(2,1,3-benzoxadiazol-4-ylsulfonyl)amino]carbonyl}azetidin-1-yl)-5-cyano-2-methylnicotinate,
Ethyl 5-cyano-2-methyl-6-(3-[({[4-(1H-tetrazol-5-yl)phenyl]sulfonyl}amino)carbonyl]azetidin-1-yl 3 nicotinate,
Ethyl 5-cyano-6-[3-({[(4-methoxyphenyl)sulfonyl]amino}carbonyl)azetidin-1-yl]-2-methylnicotinate,
Ethyl 5-cyano-6-[3-({[(3-cyanophenyl)sulfonyl]amino}carbonyl)azetidin-1-yl]-2-methylnicotinate,
Ethyl 5-cyano-2-methyl-6-(3-{[(2-naphthylsulfonyl)amino]carbonyl}azetidin-1-yl)nicotinate,
Ethyl 5-cyano-6-[3-({[(2,4-dimethyl-1,3-thiazol-5-yl)sulfonyl]amino}carbonyl)azetidin-1-yl]-2-methyl nicotinate,
Ethyl 5-cyano-6-(3-{[(2,3-dihydro-1,4-benzodioxin-6-ylsulfonyl)amino]carbonyl}azetidin-1-yl)-2-methylnicotinate,
Ethyl 5-cyano-2-methyl-6-[3-({methyl[(4-methylphenyl)sulfonyl]amino }carbonyl)azetidin-1-yl]nicotinate,
Ethyl 5-cyano-6-[3-({[(2,4-dichlorophenyl)sulfonyl]amino}carbonyl)azetidin-1-yl]-2-methylnicotinate,
Ethyl 6-[3-({[(5-chloro-3-methyl-1-benzothien-2-yl)sulfonyl]amino}carbonyl)azetidin-1-yl]-5-cyano-2-methylnicotinate,
Ethyl 5-cyano-2-methyl-6-[3-(f{[(4-methylphenyl)sulfonyl]amino}carbonyl)azetidin-1-yl]nicotinate,
Ethyl 5-cyano-2-methyl-6-{3-[({[4-(trifluoromethyl)phenyl]sulfonyl}amino)carbonyl]azetidin-1-yl}nicotinate,
Ethyl 5-cyano-2-methyl-6-[3-({[(3-nitrophenyl)sulfonyl]amino}carbonyl)azetidin-1-yl]nicotinate,
Ethyl 6-[3-({[(3-bromophenyl)sulfonyl]amino}carbonyl)azetidin-1-yl]-5-cyano-2-methylnicotinate,
Ethyl 6-[3-({[(5-chloro-2-thienyl)sulfonyl]amino}carbonyl)-3-methylazetidin-1-yl]-5-cyano-2-methylnicotinate,
1-[6-amino-3-chloro-5-(5-ethyl-1,3-oxazol-2-yl)pyridin-2-yl]-N-[(5-chloro-2-thienyl)sulfonyl]piperidine-4-carboxamide,
Ethyl 6-[3-({[(3-bromo-5-chloro-2-thienyl)sulfonyl]amino}carbonyl)azetidin-1-yl]-5-cyano-2-methylnicotinate,
Ethyl 6-(3-{[(2,1,3-benzothiadiazol-4-ylsulfonyl)amino]carbonyl}azetidin-1-yl)-5-cyano-2-methylnicotinate,
Ethyl 5-cyano-6-[3-({[(2,5-dimethyl-3-furyl)sulfonyl]amino}carbonyl)azetidin-1-yl]-2-methylnicotinate,
Ethyl 6-[3-({[(6-chloroimidazo[2,1-b][1,3]thiazol-5-yl)sulfonyl]amino}carbonyl)azetidin-1-yl]-5-cyano-2-methylnicotinate,
Ethyl 5-cyano-6-(3-{[(2,3-dihydro-1-benzofuran-5-ylsulfonyl)amino]carbonyl}azetidin-1-yl)-2-methylnicotinate,
Ethyl 5-cyano-6-[3-({[(4-fluorophenyl)sulfonyl]amino}carbonyl)azetidin-1-yl]-2-methylnicotinate,
Ethyl 6-[3-({[(5-chloro-3-thienyl)sulfonyl]amino}carbonyl)azetidin-1-yl]-5-cyano-2-methylnicotinate,
Ethyl 5-cyano-6-[3-({[(5-isoxazol-5-yl-2-thienyl)sulfonyl]amino}carbonyl)azetidin-1-yl]-2-methylnicotinate,
Ethyl 6-[3-({[(3-chlorophenyl)sulfonyl]amino}carbonyl)azetidin-1-yl]-5-cyano-2-methylnicotinate,
Ethyl 5-cyano-6-[3-({[(2-fluorophenyl)sulfonyl]amino}carbonyl)azetidin-1-yl]-2-methylnicotinate,
Ethyl 5-cyano-6-[3-({[(5-isoxazol-3-yl-2-thienyl)sulfonyl]amino}carbonyl)azetidin-1-yl]-2-methylnicotinate,
Ethyl 5-cyano-6-[3-({[(3-fluorophenyl)sulfonyl]amino}carbonyl)azetidin-1-yl]-2-methylnicotinate,
Ethyl 5-cyano-2-methyl-6-(3-{[(phenylsulfonyl)amino]carbonyl}azetidin-1-yl)nicotinate,
Ethyl 6-[3-({[(4-bromo-5-chloro-2-thienyl)sulfonyl]amino}carbonyl)azetidin-1-yl]-5-cyano-2-methylnicotinate,
Ethyl 6-[3-({[(5-bromo-6-chloropyridin-3-yl)sulfonyl]amino}carbonyl)azetidin-1-yl]-5-cyano-2-methylnicotinate,
Ethyl 6-[3-({[(5-bromo-2-thienyl)sulfonyl]amino}carbonyl)azetidin-1-yl]-5-cyano-2-methylnicotinate,
Ethyl 5-cyano-2-methyl-6-[3-({[(5-pyridin-2-yl-2-thienyl)sulfonyl]amino}carbonyl)azetidin-1-yl]nicotinate,
Ethyl 5-cyano-6-[3-({[(2,5-dichloro-3-thienyl)sulfonyl]amino}carbonyl)azetidin-1-yl]-2-methylnicotinate,
Ethyl 5-cyano-6-[3-({[(4,5-dichloro-2-thienyl)sulfonyl]amino}carbonyl)azetidin-1-yl]-2-methylnicotinate,
Ethyl 5-cyano-2-methyl-6-{3-[({[3-(trifluoromethyl)phenyl]sulfonyl}amino)carbonyl]azetidin-1-yl}nicotinate,
Ethyl 6-(3-{[(1-benzothien-3-ylsulfonyl)amino]carbonyl}azetidin-1-yl)-5-cyano-2-methylnicotinate,
Ethyl 6-[3-({[(2-chlorophenyl)sulfonyl]amino}carbonyl)azetidin-1-yl]-5-cyano-2-methylnicotinate,
Ethyl 5-cyano-6-[3-({[(2,5-dimethyl-3-thienyl)sulfonyl]amino}carbonyl)azetidin-1-yl]-2-methylnicotinate,
Ethyl 5-cyano-6-[3-({[(3-methoxyphenyl)sulfonyl]amino}carbonyl)azetidin-1-yl]-2-methylnicotinate,
Ethyl 5-cyano-2-methyl-6-(3-{[(3-thienylsulfonyl)amino]carbonyl}azetidin-1-yl)nicotinate,
Ethyl 5-cyano-2-methyl-6-(3-{[(2-thienylsulfonyl)amino]carbonyl}azetidin-1-yl)nicotinate,
1-[4-Amino-3-chloro-5-(5-ethyl-1,3-oxazol-2-yl)pyridin-2-yl]-N-[(5-chloro-2-thienyl)sulfonyl]piperidine-4-carboxamide,
tert-Butyl 5-chloro -6-[4-({[5-chloro-2-thienyl)sulfonyl]amino}carbonyl)piperidin-1-yl]nicotinate,
N-[(5-chloro-2-thienyl)sulfonyl]-1-[5-(5-ethyl-1,3-oxazol-2-yl)-3-(isopropylamino)pyridin-2-yl]piperidine-4-carboxamide,
N-[(5-chloro-2-thienyl)sulfonyl]-1-[3-(dimethylamino)-5-(5-ethyl-1,3-oxazol-2-yl)pyridin-2-yl]piperidine-4-carboxamide,
N-[(5-chloro-2-thienyl)sulfonyl]-1-[5-(5-ethyl-1,3-oxazol-2-yl)-3-(methylamino)pyridin-2-yl]piperidine-4-carboxamide,
Ethyl 6-[4-({[(5-chloro-2-thienyl)sulfonyl]amino}carbonyl)piperidin-1-yl]-5-cyano-2-methylnicotinate,
Ethyl 5-cyano-2-methyl-6-[3-({[(5-methylisoxazol-4-yl)sulfonyl]amino}carbonyl)azetidin-1-yl]nicotinate,
1-[3-Chloro-5-(5-ethyl-1,3-oxazol-2-yl)A4-(methylsulfinyl)pyridin-2-yl]-N-[(5-chloro-2-thienyl)sulfonyl]piperidine-4-carboxamide,
Ethyl 6-[4-({[(5-chloro-2-thienyl)sulfonyl]amino}carbonyl)piperidin-1-yl]-2,4-dimethylnicotinate,
1-[3-(Acetylamino)-5-(5-ethyl-1,3-oxazol-2-yl)pyridin-2-yl]-N-[(5-chloro-2-thienyl)sulfonyl]piperidine-4-carboxamide,
1-[3-Chloro-5-(5-ethyl-1,3-oxazol-2-yl)-4-(hydroxymethyl)pyridin-2-yl]-N-[(5-chloro-2-thienyl)sulfonyl]piperidine-4-carboxamide,
1-[3-amino-5-(5-ethyl-1,3-oxazol-2-yl)pyridin-2-yl]-N-[(5-chloro-2-thienyl)sulfonyl]piperidine-4-carboxamide,
4-[3-chloro-5-(cyclopropylcarbonyl)pyridin-2-yl]-N-[(5-chloro-2-thienyl)sulfonyl]piperazine-1-carboxamide,
N-[({1-[3-cyano-5-(5-ethyl-1,3-oxazol-2-yl)-6-methylpyridin-2-yl]azetidin-3-yl}amino)carbonyl]-4-meethylbenzenesulfonamide,
N-[(5-chloro-2-thienyl)sulfonyl]-1-[5-(5 ethyl-1,3-oxazol-2-yl)-3-nitropyridin-2-yl]piperidine-4-carboxamide,
N-[(5-chloro-2-thienyl)sulfonyl]-1-[3-cyano-5-(5-ethyl-1,3-oxazol-2-yl)-6-methylpyridin-2-yl]azetidine-3-carboxamide,
N-[(5-chloro-2-thienyl)sulfonyl]-1-[3-cyano-5-(5-ethyl-1,3-oxazol-2-yl)-6-methylpyridin-2-yl]piperidine-4-carboxamide,
1-[3-Chloro-5-(5-ethyl-1,3-oxazol-2-yl)-4-methylpyridin-2-yl]-N-[(5-chloro-2-thienyl)sulfonyl]piperidine-4-carboxamide,
Ethyl 6-[3-({[(5-chloro-2-thienyl)sulfonyl]amino}carbonyl)azetidin-1-yl]-5-cyano-2-methylnicotinate,
N-[(5-chloro-2-thienyl)sulfonyl]-1-[5-(5-ethyl-I ,3-oxazol-2-yl)-3-methylpyridin-2-yl]piperidine-4-carboxamide,
1-[3-Chloro-5-(5-ethyl-1,3-oxazol-2-yl)pyridin-2-yl]-N-[(5-chloro-2-thienyl)sulfonyl]piperidine-4-carboxamide,
1-[3-Chloro-5-(5-propyl-1,3-oxazol-2-yl)pyridin-2-yl]-N-[(5-chloro-2-thienyl)sulfonyl]piperidine-4-carboxamide,
1-[5-(5-Butyl-1,3-oxazol-2-yl)-3-chloropyridin-2-yl]-N-[(5-chloro-2-thienyl)sulfonyl]piperidine-4-carboxamide,
5-Chloro-N-[({1-[3-cyano-5-(5-ethyl-1,3-oxazol-2-yl)-6-methylpyridin-2-yl]azetidin-3-yl}amino)carbonyl]thiophene-2-sulfonamide,
N-[(5-chloro-2-thienyl)sulfonyl]-4-[3-cyano-5-(5-ethyl-1,3-oxazol-2-yl)-6-methylpyridin-2-yl]piperazine-1-carboxamide,
1-[3-Chloro-5-(5-ethyl-1,3-oxazol-2-yl)pyridin-2-yl]-N-[(5-chloro-2-thienyl)sulfonyl]azetidine-3-carboxamide,
Ethyl 5-chloro-6-[4-({[(5-chloro-2-thienyl)sulfonyl]amino}carbonyl)piperidin-1-yl]-2,4-dimethylnicotinate,
1-[3-Chloro-5-(5-ethyl-1,3-oxazol-2-yl)-4-methoxypyridin-2-yl]-N-[(5-chloro-2-thienyl)sulfonyl]piperidine-4-carboxamide,
1-[3-Chloro-5-(5-ethyl-1,3-oxazol-2-yl)-6-methoxypyridin-2-yl]-N-[(5-chloro-2-thienyl)sulfonyl]piperidine-4-carboxamide,
1-[3-Chloro-4-(dimethylamino)-5-(5-ethyl-1,3-oxazol-2-yl)pyridin-2-yl]-N-[(5-chloro-2-thienyl)sulfonyl]piperidine-4-carboxamide,
Ethyl 5-cyano-2-methyl-6-(3-{[(pyridin-3-ylsulfonyl)amino]carbonyl}azetidin-1-yl)nicotinate,
Ethyl 5-cyano-2-methyl-6-(3-{[({5-[1-methyl-5-(trifluoromethyl)-1H-pyrazol-3-yl]-2-thienyl}sulfonyl)amino]carbonyl}azetidin-1-yl)nicotinate,
N-[(5-chloro-2-thienyl)sulfonyl]-1-[3-[(2,2-dimethylpropanoyl)amino]-5-(5-ethyl-1,3-oxazol-2-yl)pyridin-2-yl]piperidine-4-carboxamide,
Ethyl 6-[3-({[(5-chloro-1,3-dimethyl-1H-pyrazol-4-yl)sulfonyl]amino}carbonyl)azetidin-1-yl]-5-cyano-2-methylnicotinate,
Ethyl 5-cyano-2-methyl-6-{3-[({[3-(3-methyl-5-oxo-4,5-dihydro-1H-pyrazol-1-yl)phenyl]sulfonyl}amino)carbonyl]azetidin-1-yl}nicotinate,
Ethyl 6-(3-{[({4-[(4-chlorophenyl)sulfonyl]-3-methyl-2-thienyl}sulfonyl)amino]carbonyl}azetidin-1-yl)-5-cyano-2-methylnicotinate,
Ethyl 5-cyano-2-methyl-6-{3-[({[2-(trifluoromethoxy)phenyl]sulfonyl}amino)carbonyl]azetidin-1-yl}nicotinate,
Ethyl 5-cyano-6-[3-({[(3,5-difluorophenyl)sulfonyl]amino}carbonyl)azetidin-1-yl]-2-methylnicotinate,
Ethyl 5-cyano-2-methyl-6-{3-[({[4-(trifluoromethoxy)phenyl]sulfonyl}amino)carbonyl]azetidin-1-yl}nicotinate,
Ethyl 6-[3-(2-{[(5-chloro-2-thienyl)sulfonyl]amino}-2-oxoethyl)piperidin-1-yl]-5-cyano-2-methylnicotinate,
Ethyl 5-cyano-6-{3-[({[5-(methoxycarbonyl)-2-furyl]sulfonyl}amino)carbonyl]azetidin-1-yl}-2-methylnicotinate,
Ethyl 5-cyano-6-{3-[({[4-(methoxycarbonyl)-5-methyl-2-furyl]sulfonyl}amino)carbonyl]azetidin-1-yl}-2-methylnicotinate,
Ethyl 6-[3-({[(4-chlorophenyl)sulfonyl]amino}carbonyl)azetidin-1-yl]-5-cyano-2-methylnicotinate,
Ethyl 5-cyano-6-[3-({[(3,4-dichlorophenyl)sulfonyl]amino}carbonyl)azetidin-1-yl]-2-methylnicotinate,
Ethyl 5-cyano-6-[3-({[(3,4-dimethoxyphenyl)sulfonyl]amino}carbonyl)azetidin-1-yl]-2-methylnicotinate,
Ethyl 5-cyano-2-methyl-6-{3-[({[2-methyl-5-(methylsulfonyl)phenyl]sulfonyl}amino)carbonyl]azetidin-1-yl}nicotinate,
N-[(5-chloro-2-thienyl)sulfonyl]-1-[3-cyano-5-(cyclopropylcarbonyl)-6-methylpyridin-2-yl]piperidine-4-carboxamide,
Isopropyl 6-[3-({[(5-chloro-2-thienyl)sulfonyl]amino}carbonyl)azetidin-1-yl]-5-ethynyl-2-methylnicotinate,
Ethyl 6-{4-[({[(4-chlorophenyl)sulfonyl]amino}carbonyl)amino]piperidin-1-yl}-5-cyano-2-methylnicotinate,
Ethyl 6-{4-[({[(5-chloro-2-thienyl)sulfonyl]amino}carbonyl)amino]piperidin-1-yl}-5-cyano-2-methylnicotinate,
Ethyl 6-[4-({[(5-chloro-3-thienyl)sulfonyl]amino}carbonyl)piperidin-1-yl)-]-5-cyano-2-methylnicotinate,
Ethyl 5-cyano-2-methyl-6-(4-{[(2-naphthylsulfonyl)amino]carbonyl}piperidin-1-yl)nicotinate,
Ethyl 5-cyano-2-methyl-6-[4-({[(4-methylphenyl)sulfonyl]amino}carbonyl)piperidin-1-yl]nicotinate,
Ethyl 5-cyano-2-methyl-6-[5-{[(phenylsulfonyl)amino]carbonyl}hexahydropyrrolo[3,4-c]pyrrol-2(1H)-yl]nicotinate,
Ethyl 5-cyano-2-methyl-6-{3-[({[5-(2-methyl-1,3-thiazol-4-yl)-2-thienyl]sulfonyl}amino)carbonyl]azetidin-1-yl}nicotinate,
Ethyl 6-[(1S,4S)-5-({[(5-chloro-2-thienyl)sulfonyl]amino}carbonyl)-2,5-diazabicyclo[2.2.1]hept-2-yl]-5-cyano-2-methylnicotinate,
Ethyl 5-cyano-2-methyl-6-(4-{[(phenylsulfonyl)amino]carbonyl}piperidin-1-yl)nicotinate,
Ethyl 5-cyano-6-[4-({[(2,4-dichlorophenyl)sulfonyl]amino}carbonyl)piperidin-1-yl]-2-methylnicotinate,
Isopropyl 6-[4-({[(3-bromophenyl)sulfonyl]amino}carbonyl)piperidin-1-yl]-5-cyano-2-methylnicotinate,
Ethyl 5-cyano-2-methyl-6-{4-[({[4-(trifluoromethoxy)phenyl]sulfonyl}amino)carbonyl]piperidin-1-yl}nicotinate,
Ethyl 5-cyano-6-[3-{[(6-ethoxy-1,3-benzothiazol-2-yl)sulfonyl]amino}carbonyl)azetidin-1-yl]-2-methylnicotinate,
Ethyl 5-cyano-2-methyl-6-(3-{2-oxo-2-[(phenylsulfonyl)amino]ethyl}piperidin-1-yl)nicotinate,
Ethyl 5-cyano-6-(4-{[(2,3-dihydro-1,4-benzodioxin-6-ylsulfonyl)amino]carbonyl}piperidin-1-yl)-2-methylnicotinate,
Ethyl 5-cyano-6-[4-({[(4-methoxyphenyl)sulfonyl]amino}carbonyl)piperidin-1-yl]-2-methylnicotinate,
Ethyl 6-(4-{[(2,1,3-benzoxadiazol-4-ylsulfonyl)amino]carbonyl}piperidin-1-yl)-5-cyano-2-methylnicotinate,
Ethyl 5-cyano-2-methyl-6-[4-({[(3-nitrophenyl)sulfonyl]amino}carbonyl)piperidin-1-yl]nicotinate,
Isopropyl 5-cyano-2-methyl-6-(4-{[(phenylsulfonyl)amino]carbonyl}piperidin-1-yl)nicotinate,
Isopropyl 5-cyano-2-methyl-6-{3-[({[4-(trifluoromethyl)phenyl]sulfonyl}amino)carbonyl]azetidin-1-yl}nicotinate,
Isopropyl 6-[4-({[(4-chlorophenyl)sulfonyl]amino}carbonyl)piperidin-1-yl]-5-cyano-2-methylnicotinate,
Ethyl 5-cyano-6-[4-({[(3-cyanophenyl)sulfonyl]amino}carbonyl)piperidin-1-yl]-2-methylnicotinate,
Isopropyl 5-cyano-2-methyl-6-(3-{[(2-naphthylsulfonyl)amino]carbonyl}azetidin-1-yl)nicotinate,
Ethyl 5-cyano-2-methyl-6-{4-[({[2-(trifluoromethoxy)phenyl]sulfonyl}amino)carbonyl]piperidin-1-yl}nicotinate,
Isopropyl 5-cyano-6-[4-({[(4-methoxyphenyl)sulfonyl]amino}carbonyl)piperidin-1-yl]-2-methylnicotinate,
Ethyl 5-cyano-2-methyl-6-(3-{2-oxo-2-[(phenylsulfonyl)amino]ethyl}azetidin-1-yl)nicotinate and
Ethyl 6-[3-(2-{[(5-chloro-2-thienyl)sulfonyl]amino}-2-oxoethyl)azetidin-1-yl]-5-cyano-2-methylnicotinate.
   Another Embodiment of the Invention Comprises the Following Compounds;
ethyl 6-[4-(({[(5-chloro-2-thienyl)sulfonyl]amino}carbonyl)piperazin-1-yl]-5-cyano-2-(trifluoromethyl)nicotinate
ethyl 6-[4-({[(4-chlorophenyl)sulfonyl]amino}carbonyl)piperazin-1-yl]-5-cyano-2-(trifluoromethyl)nicotinate
ethyl 6-[4-({[(5-chloro-2-thienyl)sulfonyl]amino}carbonyl)piperazin-1-yl]-5-cyano-2-methylnicotinate
3-{1-({[(5-chloro-2-thienyl)sulfonyl]amino}carbonyl)-4-[3-cyano-5-(ethoxycarbonyl)-6-(trifluoromethyl)pyridin-2-yl]piperazin-2-yl}propanoic acid
ethyl 6-{3-[({[(4-chlorophenyl)sulfonyl]amino}carbonyl)amino]azetidin-1-yl}-5-cyano-2-methylnicotinate
ethyl 6-{3-[({[(5-chloro-2-thienyl)sulfonyl]amino}carbonyl)amino]azetidin-1-yl}-5-cyano-2-methylnicotinate
ethyl 6-[4-({[(5-chloro-2-thienyl)sulfonyl]amino}carbonyl)piperidin-1-yl]-5-cyano-2-isopropylnicotinate
ethyl 6-[4-({[(5-chloro-2-thienyl)sulfonyl]amino}carbonyl)piperidin-1-yl]-5-cyano-2-ethylnicotinate
2,2-dimethylpropyl 6-{3-[({[(5-chloro-2-thienyl)sulfonyl]amino}carbonyl)amino]azetidin-1-yl}-5-cyano-2-methylnicotinate
ethyl 4-amino-5-chloro-6-[4-({[(5-chloro-2-thienyl)sulfonyl]amino}carbonyl)piperidin-1-yl]nicotinate
ethyl 6-[4-({[(5-chloro-2-thienyl)sulfonyl]amino}carbonyl)-2-methylpiperazin-1-yl]-5-cyano-2-methylnicotinate
ethyl 5-cyano-6-[3-({[(4-methoxyphenyl)sulfonyl]amino}carbonyl)azetidin-1-yl]-2-methylnicotinate
ethyl 5-cyano-6-[3-({[(2,4-dichlorophenyl)sulfonyl]amino}carbonyl)azetidin-1-yl]-2-methylnicotinate
ethyl 5-cyano-2-methyl-6-[3-({[(4-methylphenyl)sulfonyl]amino}carbonyl)azetidin-1-yl]nicotinate
ethyl 6-[3-({[(3-bromophenyl)sulfonyl]amino}carbonyl)azetidin-1-yl]-5-cyano-2-methylnicotinate
ethyl 6-[3-({[(3-bromo-5-chloro-2-thienyl)sulfonyl]amino}carbonyl)azetidin-1-yl]-5-cyano-2-methylnicotinate
ethyl 5-cyano-6-[3-({[(2,5-dimethyl-3-furyl)sulfonyl]amino}carbonyl)azetidin-1-yl]-2-methylnicotinate
ethyl 6-[3-({[(5-chloro-3-thienyl)sulfonyl]amino}carbonyl)azetidin-1-yl]-5-cyano-2-methylnicotinate
ethyl 6-[3-({[(3-chlorophenyl)sulfonyl]amino}carbonyl)azetidin-1-yl]-5-cyano-2-methylnicotinate
ethyl 6-[3-({[(5-bromo-2-thienyl)sulfonyl]amino}carbonyl)azetidin-1-yl]-5-cyano-2-methylnicotinate
ethyl 5-cyano-6-[3-({[(2,5-dimethyl-3-thienyl)sulfonyl]amino}carbonyl)azetidin-1-yl]-2-methylnicotinate
ethyl 6-[4-({[(5-chloro-2-thienyl)sulfonyl]amino}carbonyl)piperidin-1-yl]-5-cyano-2-methylnicotinate
ethyl 6-[3-({[(5-chloro-2-thienyl)sulfonyl]amino}carbonyl)azetidin-1-yl]-5-cyano-2-methylnicotinate
N-[(5-chloro-2-thienyl)sulfonyl]-4-[3-cyano-5-(5-ethyl-1,3-oxazol-2-yl)-6-methylpyridin-2-yl]piperazine-1-carboxamide
ethyl 6-{4-[({[(5-chloro-2-thienyl)sulfonyl]amino}carbonyl)amino]piperidin-1-yl}-5-cyano-2-methylnicotinate
ethyl 6-[4-({[(5-chloro-3-thienyl)sulfonyl]amino}carbonyl)piperidin-1-yl]-5-cyano-2-methylnicotinate
ethyl 5-cyano-2-methyl-6-(3-{2-oxo-2-[(phenylsulfonyl)amino]ethyl}azetidin-1-yl)nicotinate
ethyl 6-[3-(2-{[(5-chloro-2-thienyl)sulfonyl]amino}-2-oxoethyl)azetidin-1-yl]-5-cyano-2-methylnicotinate
   Pharmacological Data

Functional inhibition of—the P2Y₁₂ receptor can be measured by in vitro assays using cell membranes from P2Y₁₂ transfected CHO-cells. Inhibition of platelet aggregation as a result of P2Y₁₂ antagonism is best measured by ADP-induced aggregation of washed human platelets, activation of the platelet fibrinogen receptor (GPIIb/IIIa), or aggregation in whole blood via residual platelet counting. Detailed methodology is indicated below.

Functional inhibition of 2-Me-S-ADP induced P2Y₁₂ signalling: 5 μg of membranes were diluted in 200 μl of 200 mM NaCl, 1 mM MgCl₂, 50 mM HEPES (pH 7.4), 0.01% BSA, 30 μg/ml saponin and 10 μM GDP. To this was added an EC₈₀ concentration of agonist (2-methyl-thio-adenosine diphosphate), the required concentration of test compound and 0.1 μCi ³⁵S-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of ³⁵S-GTPγS retained by the filter. Maximum activity was that determined in the presence of the agonist and minimum activity in the absence of the agonist following subtraction of the value determined for non-specific activity. The effect of compounds at various concentrations was plotted according to the equation
y=A+((B−A)/(1+((C/x)ˆD))
and IC₅₀ estimated where
A is the bottom plateau of the curve i.e. the final minimum y value
B is the top of the plateau of the curve i.e. the final maximum y value
C is the x value at the middle of the curve. This represents the log EC₅₀ value when A+B=100
D is the slope factor.
x is the original known x values.
Y is the original known y values.
Most of the compounds of the invention have an activity, when tested in the functional inhibition of 2-Me-S-ADPinduced P₂Y₁₂ signalling assay described, at a concentration of around 4 μM or below.

For example the compounds described in Examples 18 and 10 gave the following test result in the functional inhibition of 2-Me-S-ADPinduced P₂Y₁₂ signalling assay described.
IC₅₀(μM)^{a)}
Example 18 0.52
Example 10 0.61

Platelet aggregation in washed platelet suspension: Citrated blood was centrifuged for 15 min at 240×g. The supernatant containing the platelet rich plasma (PRP) was transferred to new tubes and PGI₂ was added to a final concentration of 0.8 μM. The PRP was centrifuged for 10 min at 125×g in order to pellet and discard the remaining RBC. The platelets in the PRP (supernatant) were centrifuged for 10 min at 640×g and re-suspended in PBS without Ca and Mg, supplemented with 10 mM Hepes, 2.7 mM KCl, 1 mM MgCl₂, 0.1% D-glucose, and 0.8 M PGI₂ (37° C.). Platelets were pelleted by centrifugation for 15 min at 640×g and re-suspended in PBS without PGI₂ to 200×10⁹/L. Washed platelet suspension was kept at 4° C. for 2 h prior to used in the experiments in order for the inhibitory effect of remaining PGI₂ to ablate.

Compounds were diluted in DMSO and 0.5 μL was added per well in a 96-well plate. 150 μL platelet suspension (with CaCl₂ and fibrinogen added to a final concentration of 1 mM and 10 mg/mL clottable protein, respectively) was added to each well. Light absorption at 650 nm was recorded before and after a 5 min plate-shake and referred to recording 0 (R0) and R1. 10 μL ADP in 0.9% NaCl (20 μM final concentration) was added to each well prior to an additional 5 min plate shake and light absorption recording; R2. All measurements were made in triplicates and light absorbances in wells with PBS buffer alone were subtracted from all readings before percent aggregation was calculated according to the formula below: [(R1−R2)/R1]×100=% aggregation. Spontaneous aggregation or possible pro-aggregatory effects of the compounds was evaluated by the same formula, [(R0−R1)/R0]×100=% aggregation.

GPIIb/IIIa receptor activation assay: A venous blood sample was taken via vena puncture from the forearm of a healthy volunteer, using citrate as anticoagulant (1 part 0.109 M citrate in 9 parts blood). The citrated blood was diluted 1:10 with modified Tyrodes buffer (TB; 137 mM NaCl, 2.8 mM KCl, 1 mM MgCl2, 12 mM NaHCO., 0.4 mM Na₂HPO4, 0.35% BSA, 10 mM HEPES, 5.5 mM glucose, pH 7.4) within 1 min of collection and used within 15 min of collection. A two-colour antibody panel was used: PAC-1-FITC and CD42a-PerCP.

CD42a was used as a general platelet marker. The α_{IIb}β₃ (GPIIb/IIIa) antibody PAC-1 specifically recognises the active conformation of The α_{IIb}β₃ integrin and was therefore used as a marker of platelet activation. All incubations were performed at room temperature in the dark.

Compound was diluted into DMSO and ADP was diluted in TB. One μl of diluted compound or DMSO was added to each tube and pre-incubated for 2 min with a mix constituting the following reagents: 172.5 μl diluted human whole blood, 11.25 μl mouse antihuman CD42a-PerCP, 18.75 μl PAC-1-FITC, and 97.5 μl of TB. Ten μl of ADP, final concentration 20 μM, or TB was added to each tube and the samples were incubated for 10 min. The reaction was stopped by fixing the cells for 30 min with 300 μl of 1.5% formaldehyde in TB. Thirty 4 of sample was then diluted with 1 mL TB prior to analysis on a flow cytometer. Samples were analysed with a FACSCalibur using the CellQuest software (Becton Dickinson, Palo Alto, Calif., USA) within 2 hrs. The threshold was set on fluorescence 3 (FL3; CD42a-PerCP), and platelets were defined as CD42a positive and within the platelet cluster in a log forward-scatter (FSC) versus log CD42a-PerCP dot plot. Data on 5000 platelets were acquired in each sample. The data were analysed using the WinList 5.0 software (Verity Software House, Topsham, Me., USA), and the platelet population was analysed with respect to PAC-1 mean fluorescence intensity (MFI). Background, defined as PAC-1 MFI in the absence of ADP and antagonist, was deducted from all samples. PAC-1 MFI in the presence of ADP but absence of antagonist was defined as 100% activation and percent inhibition of 20 μM ADP-induced platelet activation was calculated. IC₅₀ values were calculated by Exfit according to the equation, y=100/[1+(x/b)^{s}], where y=response; s=the slope of the concentration response curve; x=antagonist concentration; and b=antagonist IC₅₀ concentration.

Whole blood platelet aggregation assay: A venous blood sample was taken via vena puncture from the forearm of a healthy volunteer, using hirudin as anticoagulant (0.01 part hirudin (5 mg/ml) in 9.99 parts blood) turned upside-down 6 times and left at 37° C. for at least 30 (but no longer than 60) minutes to rest the platelets. 500 μl of the hirudinated blood was pipetted into to a 3 ml PE-test tube, containing 1.5 μl compound/vehicle, and stiffed at 1000 rpm for 2 minutes before a sample of 10 μl was withdrawn and also presamples were taken. Again after 1 minute, platelet aggregation was induced by addition of 10 μl ADP solution for human blood (30 μM final concentration). Additional samples of 10 μl1 were taken 1 and 10 minutes after the ADP addition. The samples were mixed with 100 μl of a fixing solution (150 mM NaCl containing 0.16% (w/v) formaldehyde, 4.6 mM disodium EDTA, 4.5 mM disodium-hydrogen-phosphate, 1.6 mM potassium-dihydrogen-phosphate and 1% PE anti-human CD 426). The samples were then further diluted before analysed by flow cytometry by mixing 10 μl of the sample with 1 ml of the fixing solution. The amount of single platelets was determined by flow cytometry via counting the amount of single platelets per 50,000 red blood cells. Samples were analysed with a FACSArray using the CellQuest software (Becton Dickinson, Palo Alto, Calif., USA). Percentage aggregation was determined by dividing the amount of platelets left after ADP stimulation minus the amount of platelets in non ADP-stimulated samples.

The compounds of the invention act as P2Y₁₂ receptor antagonists and are therefore useful in therapy. Thus, according to a further aspect of the invention there is provided a compound of formula (I), or a pharmaceutically acceptable salt thereof, for use in therapy.

In a further aspect there is provided the use of a compound of formula (I), or a pharmaceutically acceptable salt thereof for the manufacture of a medicament for treatment of a platelet aggregation disorder. In another aspect of the invention there is provided the use of a compound of formula (I), or a pharmaceutically acceptable salt thereof, for the manufacture of a medicament for the inhibition of the P2Y₁₂ receptor.

The compounds are useful in therapy, especially adjunctive therapy, particularly they are indicated for use as: inhibitors of platelet activation, aggregation and degranulation, promoters of platelet disaggregation, anti-thrombotic agents or in the treatment or prophylaxis of unstable angina, coronary angioplasty (PTCA), myocardial infarction, perithrombolysis, primary arterial thrombotic complications of atherosclerosis such as thrombotic or embolic stroke, transient ischaemic attacks, peripheral vascular disease, myocardial infarction with or without thrombolysis, arterial complications due to interventions in atherosclerotic disease such as angioplasty, endarterectomy, stent placement, coronary and other vascular graft surgery, thrombotic complications of surgical or mechanical damage such as tissue salvage following accidental or surgical trauma, reconstructive surgery including skin and muscle flaps, conditions with a diffuse thrombotic/platelet consumption component such as disseminated intravascular coagulation, thrombotic thrombocytopaenic purpura, haemolytic uraemic syndrome, thrombotic complications of septicaemia, adult respiratory distress syndrome, anti-phospholipid syndrome, heparin-induced thrombocytopaenia and pre-eclampsia/eclampsia, or venous thrombosis such as deep vein thrombosis, venoocclusive disease, haematological conditions such as myeloproliferative disease, including thrombocythaemia, sickle cell disease; or in the prevention of mechanically-induced platelet activation in vivo, such as cardio-pulmonary bypass and extracorporeal membrane oxygenation (prevention of microthromboembolism), mechanically-induced platelet activation in vitro, such as use in the preservation of blood products, e.g. platelet concentrates, or shunt occlusion such as in renal dialysis and plasmapheresis, thrombosis secondary to vascular damage/inflammation such as vasculitis, arteritis, glomerulonephritis, inflammatory bowel disease and organ graft rejection, conditions such as migraine, Raynaud's phenomenon, conditions in which platelets can contribute to the underlying inflammatory disease process in the vascular wall such as atheromatous plaque formation/progression, stenosis/restenosis and in other inflammatory conditions such as asthma, in which platelets and platelet-derived factors are implicated in the immunological disease process.

According to the invention there is further provided the use of a compound according to the invention in the manufacture of a medicament for the treatment of the above disorders. In particular the compounds of the invention are useful for treating myocardial infarction, thrombotic stroke, transient ischaemic attacks, peripheral vascular disease and angina, especially unstable angina. The invention also provides a method of treatment of the above disorders which comprises administering to a patient suffering from such a disorder a therapeutically effective amount of a compound according to the invention.

In a further aspect the invention provides a pharmaceutical composition comprising a compound of formula (I), or a pharmaceutically acceptable salt thereof, together with a pharmaceutically acceptable diluent, adjuvant and/or carrier.

The compounds may be administered topically, e.g. to the lung and/or the airways, in the form of solutions, suspensions, HFA aerosols and dry powder formulations; or systemically, e.g. by oral administration in the form of tablets, pills, capsules, syrups, powders or granules, or by parenteral administration in the form of sterile parenteral solutions or suspensions, by subcutaneous administration, or by rectal administration in the form of suppositories or transdermally.

The compounds of the invention may be administered on their own or as a pharmaceutical composition comprising the compound of the invention in combination with a pharmaceutically acceptable diluent, adjuvant or carrier. Particularly preferred are compositions not containing material capable of causing an adverse, e.g. an allergic, reaction.

Dry powder formulations and pressurised HFA aerosols of the compounds of the invention may be administered by oral or nasal inhalation. For inhalation the compound is desirably finely divided. The compounds of the invention may also be administered by means of a dry powder inhaler. The inhaler may be a single or a multi dose inhaler, and may be a breath actuated dry powder inhaler.

One possibility is to mix the finely divided compound with a carrier substance, e.g. a mono-, di- or polysaccharide, a sugar alcohol or another polyol. Suitable carriers include sugars and starch. Alternatively the finely divided compound may be coated by another substance. The powder mixture may also be dispensed into hard gelatine capsules, each containing the desired dose of the active compound.

Another possibility is to process the finely divided powder into spheres, which break up during the inhalation procedure. This spheronized powder may be filled into the drug reservoir of a multidose inhaler, e.g. that known as the Turbuhaler® in which a dosing unit meters the desired dose which is then inhaled by the patient. With this system the active compound with or without a carrier substance is delivered to the patient.

The pharmaceutical composition comprising the compound of the invention may conveniently be tablets, pills, capsules, syrups, powders or granules for oral administration; sterile parenteral or subcutaneous solutions, suspensions for parenteral administration or suppositories for rectal administration.

For oral administration the active compound may be admixed with an adjuvant or a carrier, e.g. lactose, saccharose, sorbitol, mannitol, starches such as potato starch, corn starch or amylopectin, cellulose derivatives, a binder such as gelatine or polyvinylpyrrolidone, and a lubricant such as magnesium stearate, calcium stearate, polyethylene glycol, waxes, paraffin, and the like, and then compressed into tablets. If coated tablets are required, the cores, prepared as described above, may be coated with a concentrated sugar solution which may contain e.g. gum arabic, gelatine, talcum, titanium dioxide, and the like. Alternatively, the tablet may be coated with a suitable polymer dissolved either in a readily volatile organic solvent or an aqueous solvent.

For the preparation of soft gelatine capsules, the compound may be admixed with e.g. a vegetable oil or polyethylene glycol. Hard gelatine capsules may contain granules of the compound using either the above mentioned excipients for tablets, e.g. lactose, saccharose, sorbitol , mannitol, starches, cellulose derivatives or gelatine. Also liquid or semisolid formulations of the drug may be filled into hard gelatine capsules.

Liquid preparations for oral application may be in the form of syrups or suspensions, for example solutions containing the compound, the balance being sugar and a mixture of ethanol, water, glycerol and propylene glycol. Optionally such liquid preparations may contain colouring agents, flavouring agents, saccharine and carboxymethylcellulose as a thickening agent or other excipients known to those skilled in art.

The invention will be further illustrated with the following non-limiting examples:

### EXAMPLES

### General Experimental Procedure

Mass spectra was recorded on a Finnigan LCQ Duo ion trap mass spectrometer equipped with an electrospray interface (LC-ms) or LC-ms system consisting of a Waters ZQ using a LC-Agilent 1100 LC system.

1H NMR measurements were performed on a Varian Mercury VX 400 spectrometer, operating at a 1H frequency of 400 and Varian UNITY plus 400, 500 and 600 spectrometers, operating at 1H frequencies of 400,500 and 600 respectively. Chemical shifts are given in ppm with the solvent as internal standard. Chromatography was performed using Biotage silica gel 40S, 40M, 12i or Merck silica gel 60 (0.063-0.200 mm). Flash chromatography was performed using either standard glass- or plastic-columns column or on a Biotage Horizon system. HPLC separations were performed on a Waters YMC-ODS AQS-3 120 Angstrom 3×500 mm or on a Waters Delta Prep Systems using Kromasil CS, 10 μm columns. Reactions performed in a microwave reactor were performed in a Personal Chemistry Smith Creator, Smith synthesizer or an Emrys Optimizer.

### LIST OF USED ABBREVIATIONS

| Abbreviation | Explanation |
|---|---|
| AcOH | Acetic acid |
| Aq | Aqueous |
| br | Broad |
| Brine | A saturated solution of sodium chloride in water |
| BSA | Bovine Serum Albumine |
| CDI | Carbonyldiimidazole |
| d | Doublet |
| DCE | 1,2-Dichloroethane |
| DCM | Dichloromethane |
| DDQ | 2,3-Dichloro-5,6-dicyano-1,4-benzoquinone |
| DIEA | N,N-Diisopropylethylamine |
| DIPEA | N,N-Diisopropylethylamine |
| DMA | N,N-Dimethylacetamide |
| DMAP | N,N-dimethylpyridin-4-amine |
| DMF | N,N-dimethylformamide |
| DMSO | Dimethylsulphoxide |
| EDCI | N-[3-(dimethylamino)propyl]-N′-ethylcarbodiimide |
| | hydrochloride |
| EtOAc | Ethyl acetate |
| EtOH | Ethanol |
| HATU | O-(7-Azabenzotriazol-1-yl)-1,1,3,3- |
| | tetramethyluronium hexafluorophosphate |
| HEPES | [4-(2-hydroxyethyl)-1-piperazineethanesulfonic acid |
| HFA | Hydrofluoroalkanes |
| HOAc | Acetic acid |
| HOBT | 1-Hydroxybenzotriazole |
| HPLC | High-performance liquid chromatography |
| Hz | Hertz |
| J | Coupling constant |
| LDA | Litiumdiisopropyl amide |
| M | Multiplet |
| MeOH | Methanol |
| MHz | Megahertz |
| mL | Millilitre |
| MS | Mass spectra |
| NBS | 1-Bromopyrrolidine-2,5-dione(N-bromo |
| | succinimide) |
| q | Quartet |
| r.t | Room temperature |
| s | Singlet |
| t | triplet |
| TB | Tyrodes Buffer |
| TBTU | N[(1H-1,2,3-benzotriazol-1- |
| | yloxy)(dimethylamino)methylene]-N- |
| | methylmethanaminium tetrafluoroborate |
| TEA | Triethylamine |
| TFA | Trifluoroacetic acid |
| THF | Tetrahydrofuran |

Sulphone Amides
Synthesis of Sulfone Amides

Each of the following substances were made by reacting the corresponding sulfonyl chloride (0.75 mmol) with a saturated solution of ammonia in MeOH (5 mL). After evaporation of the ammonia and MeOH the residues were dissolved in MeOH (5 mL) and to a few samples DMF (2 mL) was also added to dissolve the reaction mixtures. The solutions where then separately filtered through ISOLUTE SCX-2, (25 mL cartridge) containing acidic ion exchange resin (5g). MeOH (16 mL) was used to rinse the product from the resin. After removal of the solvent each of the products were used without further purification as described in Method A below.

The sulfonamides made by this procedure are listed in table 1.

| | | Crude | |
|---|---|---|---|
| Products | Compound name | yield | |
| Structure | Compound name | mg | % |
| 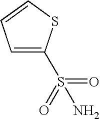 | thiophene-2-sulfonamide | 174 | 142 |
| 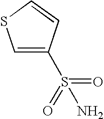 | thiophene-3-sulfonamide | 132 | 108 |
| 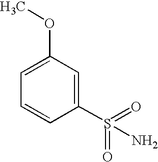 | 3-methoxybenzenesulfonamide | 166 | 118 |
| 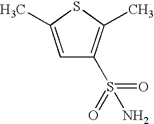 | 2,5-dimethylthiophene-3- sulfonamide | 140 | 97 |
| 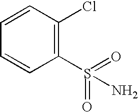 | 2-chlorobenzenesulfonamide | 152 | 106 |
| 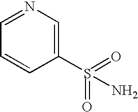 | pyridine-3-sulfonamide | 144 | 121 |
| 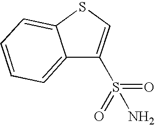 | 1-benzothiophene-3-sulfonamide | 162 | 101 |
| 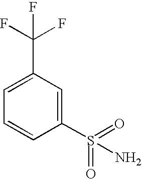 | 3- (trifluoromethyl)benzene- sulfonamide | 184 | 109 |
| 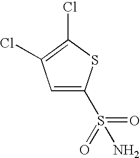 | 4,5-dichlorothiophene-2- sulfonamide | 216 | 124 |
| 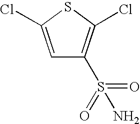 | 2,5-dichlorothiophene-3- sulfonamide | 164 | 94 |
| 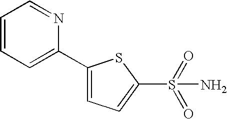 | 5-pyridin-2-ylthiophene-2- sulfonamide | 146 | 81 |
| 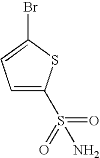 | 5-bromothiophene-2-sulfonamide | 194 | 107 |
| 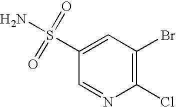 | 5-bromo-6-chloropyridine-3- sulfonamide | 232 | 114 |
| 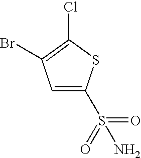 | 4-bromo-5-chlorothiophene-2- sulfonamide | 210 | 101 |
| 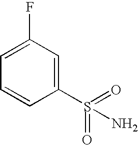 | 3-fluorobenzenesulfonamide | 152 | 116 |
| 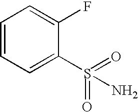 | 2-fluorobenzenesulfonamide | 148 | 113 |
| 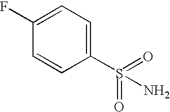 | 4-fluorobenzenesulfonamide | 146 | 111 |
| 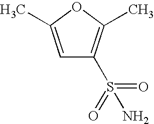 | 2,5-dimethylfuran-3-sulfonamide | 172 | 131 |
| 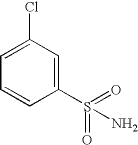 | 3-chlorobenzenesulfonamide | 86 | 60 |
| 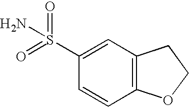 | 2,3-dihydro-1-benzofuran-5- sulfonamide | 144 | 96 |
| 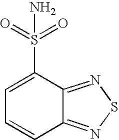 | 2,1,3-benzothiadiazole-4- sulfonamide | 180 | 111 |
| 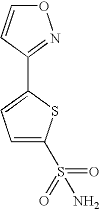 | 5-isoxazol-3-ylthiophene-2- sulfonamide | 174 | 101 |
| 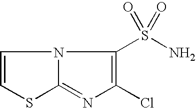 | 6-chloroimidazo[2,1- b][1,3]thiazole-5-sulfonamide | 188 | 105 |
| 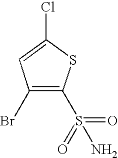 | 3-bromo-5-chlorothiophene-2- sulfonamide | 206 | 99 |
| 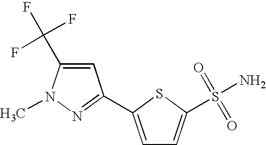 | 5-[1-methyl-5-(trifluoromethyl)- 1H-pyrazol-3-yl]thiophene-2- sulfonamide | 278 | 119 |
| 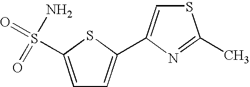 | 5-(2-methyl-1,3-thiazol-4- yl)thiophene-2-sulfonamide | 140 | 72 |
| 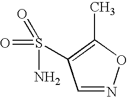 | 5-methylisoxazole-4-sulfonamide | 96 | 79 |
| 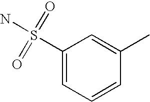 | 3-methylbenzenesulfonamide | 170 | 132 |
| 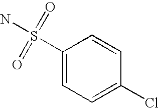 | 4-chlorobenzenesulfonamide | 396 | 275 |
| 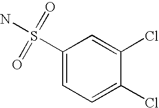 | 3,4-dichlorobenzenesulfonamide | 134 | 79 |
| 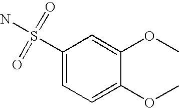 | 3,4- dimethoxybenzenesulfonamide | 126 | 77 |
| 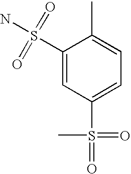 | 2-mehtyl-5- (methylsulfonyl)benzenesulfonamide | 106 | 57 |

Sulphoneamides included which are not commercially available or described in the table above, were made by an analogous method from the corresponding commercially available sulfonyl chloride.

Method A

1-[3-Cyano-5-(ethoxycarbonyl)-6-methylpyridine-2-yl]azetidine-3-carboxylic acid (0.072 g, 0.25 mmol), see example 56(d), sulfone amide (the amount and structure of the sulfonamide used is specified in each of the examples below) and DIPEA (5 Eq) was stirred in DMF (8 mL/mmol of the acid used). HATU (1.05 Eq) was dissolved in DMF (4 mL/mmol of the acid used) added and the reaction was stirred at r.t over night. The solvent was removed in vacuo and the crude reaction mixture was dissolved in DMSO (1 mL) and purified by preparative HPLC (Kromasil C8, 5 μm particles, 100×21.2 mm column, Eluent A: 100% CH₃CN, Eluent B: 0.1M NH₄OAc (aq)containing 5% CH₃CN, flow 30 mL/min, using a increasing gradient of CH₃CN over 8 minutes to afford the products after evaporation of the solvents).

Method B

To a solution of 1-[3-cyano-5-(ethoxycarbonyl)-6-methylpyridin-2-yl]piperidine-4-carboxylic acid (0.21 mmol) DCM (2 mL) was added TBTU 0.25 mmol) and DIPEA (1.05) mmol. The reaction mixture was stirred for 10 minutes followed by addition of sulfoneamide (0.25 mmol) e.g. 5-chlorothiophene-3-sulfonamide. The reaction mixture was stirred over night followed by addition of 0.1 M KHSO₄ (2 mL), the organic phase was isolated and the crude reaction mixture was submitted to preparative HPLC (see below for details) in order to isolate the wanted product, e.g. ethyl 6-[4-({[(5-chloro-3-thienyl)sulfonyl]amino}carbonyl)piperidin-1-yl]-5-cyano-2-methylnicotinate.

The preparative HPLC system used was a Waters Fraction Lynx Purification System with Kromasil CS 5 mm 20×100 mm columns. The mobile phase used was varying gradients of CH₃CN and 0.1 M NH₄OAc(aq) buffer. The flow was 30 mL/minute. MS triggered fraction collection was used. Mass spectra were recorded on either a Micromass ZQ single quadrupole or a Micromass Quattro micro, both equipped with a pneumatically assisted electro spray interface.

Method C

A solution of 1-[3-cyano-5-(isopropoxycarbonyl)-6-methylpyridin-2-yl]azetidine-3-carboxylic acid (0.091 g, 0.3 mmol), DIPEA 0.074 g, 0.6 mmol) and TBTU (0.039 g, 0.3 mmol) in 1 eq. DCM/1 eq . DMF (2 mL) was added to sulfonamide (0.4 mmol), e.g. 4-(trifluoromethyl)benzenesulfonamide. The reaction mixture was stirred for 48 h followed by addition of TBTU (0.013 g, 0.1 mmol). After 20 h the solvents were removed in vacuo. The crude reaction mixture was added NaHSO₄ (2 mL, 1M) and due to differences in solubility between products DCM and DCM/ethyl acetate was used for extraction. The organic phase was isolated and the solvents were removed in vacuo. The crude material was purified using preparative HPLC (see below for details) in order to isolate the desired product, e.g. isopropyl 5-cyano-2-methyl-6-{3-[({[4-(trifluoromethyl)phenyl]sulfonyl}amino)carbonyl]azetidin-1-yl}nicotinate.

The preparative HPLC system used was a Waters Fraction Lynx Purification System with Kromasil C8 5 mm 20×100 mm columns. The mobile phase used was varying gradients of CH₃CN and 0.1 M NH₄OAc buffer. The flow was 30 mL/minute. MS triggered fraction collection was used. Mass spectra were recorded on either a Micromass ZQ single quadrupole or a Micromass Quattro micro, both equipped with a pneumatically assisted electro spray interface.

### Example 1

### Ethyl 5-chloro-6-[4-({[(2-methylphenyl)sulfonyl]amino}carbonyl)piperazin-1-yl]nicotinate

### (a) Ethyl 5-chloro-6-piperazin-1-ylnicotinate

Ethyl 5,6-dichloronicotinate (2.20 g, 10.0 mol) was weighed into an Erlenmeyer flask. piperazine (1.03 g, 12.0 mol), triethylamine (1.21 g, 12.0 mol), and absolute ethanol (20.0 mL) were added. The mixture was stirred until a clear solution appeared. This solution was divided into 10 microwave vials. Each vial was heated in the microwave reactor, at 120° C. for 10 minutes. The combined reaction mixtures were extracted with ethylacetate (3×80 mL) from a 10% potassium carbonate solution (80 mL). The combined organic extracts were evaporated in vacuo. The crude material was purified by flash chromatography (DCM/MeOH/triethylamine 9:1:0.1) to give Ethyl 5-chloro-6-piperazin-1-ylnicotinate. Yield: 1.60 g (61%).

¹H NMR (400 MHz, CDCl₃): 1.38 (3H, t, J=7.2 Hz), 1.77 (1H, br s), 3.01-3.05 (4H, m), 3.51-3.55 (4H, m), 4.36 (2H, t, J=7.2 Hz), 8.12 (1H, d, J=2.0 Hz), 8.75 (1H, d, J=2.0 Hz).

MS^{m}/z: 270 (M+1).

### (b)Ethyl 5-chloro-6-[4-({[(2-methylphenyl)sulfonyl]amino}carbonyl)piperazin-1-yl]nicotinate

Ethyl 5-chloro-6-piperazin-1-ylnicotinate (0.067 g, 0.25 mmol) was dissolved in DCM (1.5 mL) and 2-methylbenzenesulfonyl isocyanate (0.074 g, 0.375 mmol) was added at room temperature. The reaction mixture was stirred at room temperature under nitrogen for 14 h and then evaporated. The crude material was purified by preparative HPLC (Acetonitrile/ammonium acetate buffer 19-48%) followed by removal of solvents from the relevant fractions in vacuo to a volume of 5 ml followed by extraction using DCM (3*5 ml). The combined organic phases were dried over sodium sulphate, filtered and evaporated to give Ethyl 5-chloro-6-[4-({[(2-methylphenyl)sulfonyl]amino}carbonyl)piperazin-1-yl]nicotinate. Yield: 0.038 g (33%).

¹H NMR (400 MHz, CD₃OD): δ 1.41 (3H, t, J=7.1 Hz), 2.71 (3H, s), 3.38-3.42 (4H, m), 3.48-3.53 (4H, m), 4.38 (2H, q, J=7.1 Hz), 7.30-7.36 (2H, m), 7.43-7.48 (1H, m), 8.04-8.08 (1H, m), 8.14-8.16 (1H, m), 8.70-8.72 (1H, m).

MS^{m}/z: 467 (M+1).

### Example 2

### Ethyl 5-chloro-6-[4-({[(4-methylphenyl)sulfonyl]amino}carbonyl)piperazin-1-yl]nicotinate

Ethyl 5-chloro-6-piperazin-1-ylnicotinate (0.108 g, 0.40 mmol) was dissolved in DCM (3.0 mL) and 4-methylbenzenesulfonyl isocyanate (0.095 g, 0.48 mmol) was added at room temperature. The reaction mixture was stirred at room temperature under nitrogen for 14 h and then evaporated. The crude material was purified by preparative HPLC using a gradient (Acetonitrile/ammonium acetate buffer(0.1M) 1948%) followed by removal of solvents by freeze-drying to give Ethyl 5-chloro-6-[4-({[(4-methylphenyl)sulfonyl]amino}carbonyl)piperazin-1-yl]nicotinate. Yield: 0.077 g (41%)

¹H NMR (400 MHz, CDCl₃): δ 1.37 (3H, t, J=7.2 Hz), 2.34 (3H, s), 3.36-3.42 (4H, m), 3.50-3.56 (4H, m), 4.35 (2H, q, J=7.2 Hz), 7.16-7.21 (2H, m), 7.84-7.88 (2H, m), 8.07 (1H, d, J=1.9 Hz), 8.68 (1H, d, J=1.9 Hz).

MS^{m}/z: 467 (M+1).

### Example 3

### Ethyl 5-cyano-6-[4-({[(4-fluorophenyl)sulfonyl]amino}carbonyl)piperazin-1-yl]-2-(trifluoromethyl)nicotinate

### (a)Ethyl 5-cyano-6-piperazin-1-yl-2-(trifluoromethyl)nicotinate

Ethyl 6-chloro-5-cyano-2-(trifluoromethyl)nicotinate (1.00 g, 3.41 mmol) and piperazine (0.928 g, 10.77 mmol) was taken in ethanol (3 ml). Triethylamine (727 mg, 7.18 mmol) was added. The mixture was heated in a microwave reactor at 170° C. for 20 min. The mixture was diluted with dichloromethane (200 mL) and washed in succession with saturated sodium hydrogen carbonate solution and brine respectively. The organics were dried (Na₂SO₄), filtered and evaporated. Flash chromatography (CH₂Cl₂/MeOH 100:1 to 30:1) gave ethyl 5-cyano-6-piperazin-1-yl-2-(trifluoromethyl)nicotinate. Yield: 751 mg (67%).

¹H NMR (400, CD₃OD): δ 1.36 (3H, t J=7.14 Hz), 2.93-2.99 (4H, m), 3.92-3.98 (4H, m), 4.34 (2H, q, J=7.22 Hz), 8.42 (1H, s).

MS^{m}/z: 329 (M+1).

### (b)Ethyl 5-cyano-6-[4-({[(4-fluorophenyl)sulfonyl]amino}carbonyl)piperazin-1-yl]-2-(trifluoromethyl)nicotinate

Ethyl 5-cyano-6-piperazin-1-yl-2-(trifluoromethyl)nicotinate (0.066 g, 0.20 mmol) and 4-fluorobenzenesulfonyl isocyanate (0.048 g, 0.24 mmol) were mixed at room temperature in DCM (1.0 mL) and triethylamine (0.08 mL, 0.60 mmol) was added. The reaction mixture was stirred at room temperature under nitrogen for 14 h followed by removal of solvents in vacuo. The crude material was purified by preparative HPLC using a gradient (Acetonitrile/ammonium acetate buffer 1948%) followed by removal of solvents by freeze-drying to give Ethyl 5-cyano-6-[4-({[(4-fluorophenyl)sulfonyl]amino}carbonyl)piperazin-1-yl]-2-(trifluoromethyl)nicotinate. Yield: 0.066 g (62%).

¹H NMR (400 MHz, CDCl₃): δ 1.36 (3H, t, JA 7.2 Hz), 3.54-3.60 (4H, m), 3.80-3.86 (4H, m), 4.35 (2H, q, J=7.2 Hz), 7.00-7.06 (2H, m), 7.91-7.96 (2H, m), 8.31 (1H, s).

MS^{m}/z: 530 (M+1).

### Example 4

### Ethyl 5-chloro-6-[4-({[(4-chlorophenyl)sulfonyl]amino}carbonyl)piperazin-1-yl]nicotinate

Ethyl 5-chloro-6-piperazin-1-ylnicotinate (0.081 g, 0.30 mmol) was dissolved in DCM (3.0 mL) and 4-chlorobenzenesulfonyl isocyanate (0.078 g, 0.36 mmol) was added at room temperature. The reaction mixture was stirred at room temperature under nitrogen for 22 h and then evaporated. The crude material was purified by preparative HPLC using a gradient (Acetonitrile/ammonium acetate buffer 19-48%) followed by removal of solvents by freeze-drying to give Ethyl 5-chloro-6-[4-({[(4-chlorophenyl)sulfonyl]amino}carbonyl)piperazin-1-yl]nicotinate. Yield: 0.085 g (58%)

¹H NMR (400 MHz, CD₃OD): δ 1.40 (3H, t J=7.1 Hz), 3.47-3.52 (4H, m), 3.60-3.66 (4H, m), 4.37 (2H, q, J=7.1 Hz), 7.41-7.46 (2H, m), 7.89-7.95 (2H, m), 8.13 (1H, d, J=2.0 Hz), 8.71 (1H, d, J=2.0 Hz).

MS^{m}/z: 488 (M+1).

### Example 5

### Ethyl 5-chloro-6-[4-({[(5-chloro-2-thienyl)sulfonyl]amino}carbonyl)piperazin-1-yl]nicotinate

5-chlorothiophene-2-sulfonamide (0.079 g, 0.4 mmol) and 1,1′-[carbonylbis(oxy)]dipyrrolidine-2,5-dione (0.123 g, 0.48 mmol) were mixed in MeCN (2.5 mL) and 1,8-diazabicyclo[5.4.0]undec-7-ene (0.122 g, 0.3 mmol) was added. The reaction mixture was stirred in a sealed vial for 1 h at 40° C. Ethyl 5-chloro-6-piperazin-1-ylnicotinate (0.135 g, 0.4 mmol) was added and the reaction mixture was stirred at 40° C. in a sealed vial for 3 days followed by evaporation of the solvents. The crude material was purified by preparative HPLC using a gradient (Acetonitrile/ammonium acetate buffer 1948%) followed by removal of solvents by freeze-drying to give Ethyl 5-chloro-6-[4-({[(5-chloro-2-thienyl)sulfonyl]amino}carbonyl)piperazin-1-yl]nicotinate. Yield: 0.025 g (13%/0).

¹H NMR (400 MHz, CDCl₃): δ 1.38 (3H, t, J=7.0 Hz), 3.54-3.64 (S8H m), 4.37 (2H, q, J=7.0 Hz), 6.93 (1H, d, J=4.1 Hz), 7.65 (1H, d, J=4.1 Hz), 8.15 (1H, d, J=1.9 Hz), 8.74 (1H, d, J=1.9 Hz).

MS^{m}/z: 494 (M+1).

### Example 6

### Ethyl-6-(4-{[phenylsulfonyl)amino]carbonyl}piperazine-1-yl)-2-(trifluoromethyl)nicotinate

### (a) Ethyl 6-piperazin-1-yl-2-(trifluoromethyl)nicotinate

Ethyl 6-oxo-2-(trifluoromethyl)-1,6-dihydropyridine-3-carboxylate (400 mg, 1.70 mmol) and triethylamine (861 mg, 8.50 mmol) was dissolved in dry THF (20 ml) under a nitrogen atmosphere. It was cooled to 0° C. and trifluoromethanesulfonic anhydride (480 mg, 1.70 mmol) was added via a syringe. Stirring was continued under nitrogen atmosphere for 20 hrs. Piperazine (440 mg, 5.10 mmol) was added and the mixture was refluxed for 22 hrs followed by cooling to r.t. The reaction mixture was diluted with dichloromethane (200 ml) and washed in succession with saturated sodium hydrogen carbonate and brine respectively. The organic layer was dried over Na₂SO₄, filtered and concentrated under reduced pressure. Flash chromatography (DCM-MeOH 50:1 to 10:1) gave Ethyl 6-piperazin-1-yl-2-(trifluoromethyl)nicotinate. Yield 160 mg (30%).

¹H NMR (CDCl₃): δ 1.36 (3H, t, J=7.1 Hz), 2.90-3.06 (4H, m), 3.62-3.74 (4H, m), 4.34 (2H, q, J=7.1 Hz), 6.69 (1H, d, J=9.1 Hz), 7.98 (I, d, 9.1 Hz).

MS^{m}/z: 304 (M+1).

### (b) Ethyl 6-(4-{[phenylsulfonyl)amino]carbonyl}piperazine-1-yl)-2-(trifluoromethyl)nicotinate

Ethyl 6-piperazin-1-yl-2-(trifluoromethyl)nicotinate (160 mg, 0.50 mmol) was dissolved in dry dichloromethane (5.0 ml) at r.t. under N₂ followed by addition of benzenesulfonylisocyanate (138 mg, 0.75 mmol). The mixture was stirred overnight at r.t. followed by addition of two drops of water. Toluene was added and the solvents were removed under reduced pressure. The residue was purified by flash chromatography (DCM-ethylacetate 4:1 to 1:1) to give Ethyl 6-piperazin-1-yl-2-(trifluoromethyl)nicotinate. Yield 136 mg (56%).

¹H NMR (300 MHz d₆-DMSO): δ 1.26 (3H, t, J=7.1 Hz), 3.38-3.49 (4H, m), 3.51-3.56 (4H, m), 4.24 (2H, q, J=7.1 Hz), 7.08 (1H, d, J=9.0 Hz), 7.32-7.40 (31t, m), 7.72-7.78 (2H, m), 7.96 (1H, d, J=9.0 Hz).

MS^{m}/z: 484 (M−1).

### Example 7

### Ethyl 5-cyano-6-(4-{1-[phenylsulfonyl)amino]carbonyl}piperazin-1-yl)-2-(trifluoromethyl)nicotinat

Ethyl 5-cyano-6-piperazin-1-yl-2-(trifluoromethyl)nicotinate (210 mg, 0.640 mmol) was dissolved in dry dichloromethane (10.00 ml) under a nitrogen atmosphere followed by addition of benzenesulfonylisocyanate (352 mg, 1.92 mmol). After 2 h, triethylamine (0.10 ml, 1.00 mmol) was added and the mixture was stirred overnight at r.t. The mixture was evaporated and was purified by preparative hplc (acetonitrile 20% to 95% gradient containing 0.1% formic acid) to give Ethyl 5-cyano-6-(4-{[phenylsulfonyl)amino]carbonyl}piperazin-1-yl)-2-(trifluoromethyl)nicotinat. Yield 93 mg (27%).

¹H NMR (400 MHz ,CDCl₃): δ 1.37 (3H, t, J=7.2 Hz), 3.59-3.64 (4H, m), 3.93-4.01 (4H, m), 4.37 (2H, q, J=7.2 Hz), 7.52-7.60 (2H, m), 7.61-7.68 (1H, m), 8.05-8.10 (1H, s).

MS^{m}/z: 510 (M−1).

### Example 8

### Ethyl 6-[4-({[(2-chlorophenyl)sulfonyl]amino}carbonyl)piperazin-1-yl]-5-cyano-2-(trifluoromethyl)nicotinate

Ethyl 5-cyano-6-piperazin-1-yl-2-(trifluoromethyl)nicotinate (0.066 g, 0.20 mmol) was dissolved in DCM (1.5 mL) and 2-chlorobenzenesulfonyl isocyanate (0.065 g, 0.30 mmol) was added. The reaction mixture was stirred at room temperature under nitrogen for 14h followed by removal of the solvent under reduced pressure. The crude material was purified by preparative HPLC using a gradient (Acetonitrile/ammonium acetate buffer 1948%) followed by removal of solvents by freeze-drying to give Ethyl 6-[4-({[(2-chlorophenyl)sulfonyl]amino}carbonyl)piperazin-1-yl]-5-cyano-2-(trifluoromethyl)nicotinate. Yield: 0.077 g (71%).

¹H NMR (300 MHz, CDCl₃): δ 1.36 (3H, t, J=7.2 Hz), 3.51-3.60 (4H, m), 3.74-3.82 (4H, m), 4.35 (2H, q, J=7.2 Hz), 7.18-7.36 (31H, m), 8.02-8.09 (1H, m), 8.29 (1H, s).

MS^{m}/z: 546 (M+1).

### Example 9

### Ethyl 5-cyano-6-[4-({[(4-methylphenyl)sulfonyl]amino}carbonyl)piperazin-1-yl]-2-(trifluoromethyl)nicotinate

Ethyl 5-cyano-6-piperazin-1-yl-2-(trifluoromethyl)nicotinate (0.066 g, 0.20 mmol) and 4-methylbenzenesulfonyl isocyanate (0.048 g, 0.24 mmol) were mixed at room temperature in DCM (1.0 mL) and triethylamine (0.08 mL, 0.60 mmol) was added. The reaction mixture was stirred at room temperature under nitrogen for 14 h followed by removal of solvents under reduced pressure. The crude material was purified by flash chromatography (pentane/ethyl acetate 1:2, then 1:3 and finally with pure ethyl acetate) to give Ethyl 5-cyano-6-[4-({[(4-methylphenyl)sulfonyl]amino}carbonyl)piperazin-1-yl]-2-(trifluoromethyl)nicotinate. Yield: 0.026 g (25%).

¹H NMR (400 MHz, d₆-DMSO): a 1.31 (3H, t, J=7.1 Hz), 2.41 (3H, s), 3.52-3.57 (4H, m), 3.86-3.91 (4H, m), 4.31 (2H, q, J=7.1 Hz), 7.34-7.36 (2H, m), 7.76-7.80 (2H, m), 8.58 (1H, s), 10.99 (1H, br s).

MS^{m}/z: 526 (M+1).

### Example 10

### Ethyl 5-chloro-6-(4-{[(phenylsulfonyl)amino]carbonyl}piperazin-1-yl)nicotinate

Ethyl 5-chloro-6-piperazin-1-ylnicotinate (0.054 g, 0.20 mmol) was dissolved in THF (1.0 mL). Triethylamine (0.030 g, 0.30 mmol) was added at 0° C., followed by benzenesulfonyl isocyanate (0.048 g, 0.26 mmol). The reaction mixture was stirred at 0° C. for 1 h and then at room temperature for 17 h. The reaction mixture was then stirred gently with PS-TRIS and PS-NCO at room temperature for 1 h. The resins were filtered off and the solvents were removed in vacuo. The crude product was purified by flash chromatography (pentane/ethyl acetate 1:1, followed by a gradient to pure ethyl acetate) to give Ethyl 5-chloro-6-(4-{[(phenylsulfonyl)amino]carbonyl}piperazin-1-yl)nicotinate. Yield: 0.022 g (24%).

¹H NMR (400 MHz, CDCl₃): δ 1.39 (3H, t, J=7.2 Hz), 3.54-3.57 (8H, m), 4.38 (2H, q, J=7.2 Hz), 7.56-7.59 (2H, m), 7.62-7.67 (2H, m), 8.6-8.12 (1H, m), 8.15 (1H, d, J=2.0 Hz), 8.75 (1H, d, J=2.0 Hz).

MS^{m}/z: 453 (M+1)

### Example 11

### Ethyl 5-cyano-2-methyl-6-(4{[(phenylsulfonyl)amino]carbonyl}piperazin-1-yl)nicotinate

### (a) Ethyl 5-cyano-2-methyl-6-piperazin-1-ylnicotinate

Ethyl 6-chloro-5-cyano-2-methylnicotinate (2.00 g, 8.90 mmol) and piperazine (2.30 g, 26.7 mmol) was taken in ethanol (30 ml). Triethylamine (1.35 g, 13.4 mmol) was added. The mixture was heated in a microwave reactor at 160° C. for 25 min. The mixture was diluted with dichloromethane (300 ml) and washed in succession with saturated sodium hydrogen carbonate solution and brine respectively. The organics were dried over sodium sulphate, filtered and the solvents were removed under reduced pressure to give Ethyl 5-cyano-2-methyl-6-piperazin-1-ylnicotinate which was used crude in the consecutive step.

¹H NMR (CDCl₃): δ 1.37 (3H, t, J=7.2 Hz), 2.71 (3H, s), 2.96-3.02 (4H, m), 3.88-3.95 (4H, m), 4.31 (2H, q, J=7.2 Hz), 8.28 (1H, s).

MS^{m}/z: 275 (M+1).

### (b) Ethyl 5-cyano-2-methyl-6-(4-{[(phenylsulfonyl)amino]carbonyl}piperazin-1-yl)nicotinate

To a solution of Ethyl 5-cyano-2-methyl-6-piperazin-1-ylnicotinate (274 mg, 0.96 mmol) in dry dichloromethane (5.0 ml) at r.t. under N₂ was added benzenesulfonylisocyanate (263 mg, 1.44 mmol). The mixture was kept stirring overnight at r.t. Two drops of water was added followed by toluene and the solvents were removed under reduced pressure. The crude product was purified by flash chromatography (DCM-ethylacetate 4:1 to 0:1 gradient) to give Ethyl 5-cyano-2-methyl-6-(4-{[(phenylsulfonyl)amino]carbonyl}piperazin-1-yl)nicotinate. Yield 61 mg (13%).

¹H NMR (500 MHz, d₆-DMSO): δ 1.31 (3H, t, J=7.3 Hz), 2.64 (3H, s), 3.45-3.53 (4H, m), 3.75-3.80 (4H, m), 4.26 (2H, q, J=7.3 Hz), 7.39-7.45 (311 m), 7.77-7.81 (2H, m), 8.35 (1H, s).

MS^{m}/z: 456 (M−1).

### Example 12

### Ethyl 6-[4-({[(5-chloro-2-thienyl)sulfonyl]amino}carbonyl)piperazin-1-yl]-5-cyano-2-(trifluoromethyl)nicotinate

5-Chlorothiophene-2-sulfonamide (0.181 g, 0.91 mmol) and 1,1′-carbonyldiimidazole (0.148 g, 0.91 mmol) were dissolved in DCM (5 mL) and DIPEA (1.59 mL, 9.14 mmol). The reaction mixture was stirred at room temperature for 4 h. ethyl 5-cyano-6-piperazin-1-yl-2-(trifluoromethyl)nicotinate (0.300 g, 0.91 mmol) was added and the reaction mixture was heated at reflux for 16 h. The reaction mixture was cooled to room temperature and concentrated under reduced pressure to yield the crude product. Flash chromatography (3:7 EtOAc/hexanes to EtOAc, 1% AcOH) gave 3-methylbutyl 5-chloro-6-[4-({[(5-chloro-2-thienyl)sulfonyl]amino}carbonyl)piperazin-1-yl]nicotinate as a solid. Yield: 0.060g (12%).

¹H NMR (400 MHz, d₆-DMSO): δ 1.29 (3H, t, J=7.1 Hz), 3.53-3.60 (4H, m), 3.86-3.93 (4H, m), 4.29 (2H, q, J=7.1 Hz), 7.24 (1H, d, J=4.1 Hz), 7.62 (1H, d, J=4.1 Hz), 8.57 (1H, s).

MS^{m}/z: 552 (M+1).

### Example 13

### Ethyl 5-chloro-6-[4-({[(4-fluorophenyl)sulfonyl]amino}carbonyl)piperazin-1-yl]nicotinate

Ethyl 5-chloro-6-piperazin-1-ylnicotinate (0.067 g, 0.25 mmol) was dissolved in DCM (3.0 mL) and 4-fluorobenzenesulfonyl isocyanate (0.091 g, 0.45 mmol) was added at room temperature. The reaction mixture was stirred at room temperature for 20 h and then evaporated. The crude material was purified by preparative HPLC using a gradient (Acetonitrile/ammonium acetate buffer 19-48% 0.1M) followed by removal of solvents by freeze-drying to give Ethyl 5-chloro-6-[4-({[(4-fluorophenyl)sulfonyl]amino}carbonyl)piperazin-1-yl]nicotinate. Yield: 0.087 g (74%)

¹H NMR (400 MHz, CDCl₃): δ 1.40 (3H, t, J=7.1 Hz), 3.51-3.56 (4H, m), 3.57-3.61 (4H, m), 4.38 (2H, q, J=7.1 Hz), 7.20-7.28 (2H, m), 8.06-8.11 (2H, m), 8.14-8.17 (1H, m), 8.69-8.74 (1H, m).

MS^{m}/z: 471 (M+1).

### Example 14

### Ethyl 5-chloro-6-[4-({[(2-chlorophenyl)sulfonyl]amino}carbonyl)piperazin-1-yl]nicotinate

Ethyl 5-chloro-6-piperazin-1-ylnicotinate (0.054 g, 0.20 mmol) and 2-chlorobenzenesulfonyl isocyanate (0.052 g, 0.24 mmol) were mixed in DCM (1.0 ml) at room temperature and triethylamine (0.80 mL, 0.6 mmol) was added. The reaction mixture was stirred at room temperature for 14 h followed by removal of the solvents under reduced pressure. The crude material was purified by flash chromatography (pentane/ethyl acetate 1:2, then 1:3, and finally with pure ethyl acetate) to give Ethyl 5-chloro-6-[4-({[(2-chlorophenyl)sulfonyl]amino}carbonyl)piperazin-1-yl]nicotinate. Yield: 0.018 g (19%).

¹H NMR (500 MHz, CDCl₃): δ 1.36 (3H, t, J=7.1 Hz), 3.22-3.82 (8H, m), 4.35 (2H, q, J=7.1 Hz), 7.00-7.6 (1H, m), 7.20-7.38 (21 m), 7.96-8.08 (1H, m), 8.10-8.26 (1H, m), 8.58-8.70 (1H, m).

MS^{m}/z: 488 (M+1).

### Example 15

### Ethyl 6-[4-({[(4-chlorophenyl)sulfonyl]amino}carbonyl)piperazin-1-yl]-5-cyano-2-(trifluoromethyl)nicotinate

Ethyl 5-cyano-6-piperazin-1-yl-2-(trifluoromethyl)nicotinate (0.068 g, 0.21 mmol) was dissolved in DCM (1.5 mL) and 4-chlorobenzenesulfonyl isocyanate (0.054 g, 0.25 mmol) was added at room temperature. The reaction mixture was stirred at room temperature under nitrogen for 14 h and then evaporated. The crude material was purified by preparative HPLC using a gradient (Acetonitrile/ammonium acetate buffer 19-48%) followed by concentration of solvents under reduced pressure to 10 mL volume followed by extraction with DCM (3×10 mL). The combined organic phases were dried over sodium sulphate and the solvents were removed under reduced pressure to give Ethyl 6-[4-({[(4-chlorophenyl)sulfonyl]amino}carbonyl)piperazin-1-yl]-5-cyano-2-(trifluoromethyl)nicotinate. Yield: 0.095 g (84%).

¹H NMR (400 MHz, CDCl₃): δ 1.37 (3H, t, J=7.1 Hz), 3.62-3.67 (4H, m), 3.96-4.01 (4H, m), 4.37 (2H, q, J=7.1 Hz), 7.46-7.51 (2H, m), 7.95-8.00 (2H, m), 8.37 (1H, s).

MS^{m}/z: 546 (M+1).

### Example 16

### Ethyl 5-cyano-6-[4-({[(2-methylphenyl)sulfonyl]amino}carbonyl)piperazin-1-yl]-2-(trifluoromethyl)nicotinate

Ethyl 5-cyano-6-piperazin-1-yl-2-(trifluoromethyl)nicotinate (0.066 g, 0.20 mmol) and 2-methylbenzenesulfonyl isocyanate (0.047 g, 0.24 mmol) were mixed at room temperature in DCM (1.0 mL) and triethylamine (0.08 mL, 0.60 mmol) was added. The reaction mixture was stirred at room temperature under nitrogen for 14 h and then evaporated. The crude material was purified by preparative HPLC using a gradient (Acetonitrile/ammonium acetate buffer 1948%) followed by removal of solvents by freeze-drying to give Ethyl 5-cyano-6-[4-({[(2-methylphenyl)sulfonyl]amino}carbonyl)piperazin-1-yl]-2-(trifluoromethyl)nicotinate. Yield: 0.066 g (62%).

¹H NMR (400 MHz, CDCl₃): δ 1.39 (3H, t, J=7.1 Hz), 2.69 (3H, s), 3.61-3.66 (4H, m), 3.93-3.98 (4H, m),4.37 (2H, q, J=7.1 Hz), 7.28-7.38 (2H, m), 7.44-7.51 (1H, m), 8.08-8.12 (1H, m), 8.36 (1H, s).

MS^{m}/z: 526 (M+1).

### Example 17

### ethyl 6-[4-({[(5-chloro-2-thienyl)sulfonyl]amino}carbonyl)piperazin-1-yl]-5-cyano-2-methylnicotinate

1,1′-Carbonylbis-1H-imidazole (CDI) (443 mg, 2.7 mmol), 5-chlorothiophene-2-sulfonamide (407 mg, 2.0 mmol) and DIPEA (1.5 ml) was dissolved in DCM (15 ml) under a nitrogen atmosphere. After 4 hours ethyl 5-cyano-2-methyl-6-piperazin-1-ylnicotinate (407 mg, 1.8 mmol) and diisopropylethylamine (1 ml)dissolved in DCM (10 ml) was added and the reaction mixture was stirred at 40° C. over night. Water was added to the reaction mixture and the organic phase was isolated by filtering through a phase separator. The solvents were removed under reduced pressure to give crude product of 91% purity in according to HPLC. The crude was purified by preparative HPLC (acetonitrile/ammonium acetate buffer (0.1M) 5-32%) followed by concentration of solvents under reduced pressure followed by extraction with DCM (3×10 mL). The combined organic phases were dried through a phase separator and the solvents were removed under reduced pressure to give ethyl 6-[4-({[(5-chloro-2-thienyl)sulfonyl]amino}carbonyl)piperazin-1-yl]-5-cyano-2-methylnicotinate. Yield: 527 mg (55%).

¹H NMR (400 MHz, d₆-DMSO): 1.29 (3H, t, J=7.1 Hz), 2.63 (3H, s), 3.48-3.55 (4H, m), 3.80-3.87 (4H, m),4.23 (2H, q, J=7.1 Hz), 7.21 (1H, d, J=4.0 Hz), 7.60 (1H, J=4.0Hz), 8.34 (1H, s).

MS^{m}/z: 498 (M+1).

### Example 18

### Isopropyl 5-chloro-6-[4-({[(5-chloro-2-thienyl)sulfonyl]amino}carbonyl)piperazin-1-yl]nicotinate

### (a) 6-[4-(tert-Butoxycarbonyl)piperazin-1-yl]-5-chloronicotinic acid

5,6-Dichloronicotinic acid (19.0 g, 99 mmol), 1-boc-piperazine (24.0 g, 129 mmol) and DIPEA (34.5 mL, 198 mmol) were dissolved in DMA (150 mL) and heated to 120° C. for 16 h in a sealed flask. Additional 1-boc-piperazine (1.0 g, 5.4 mmol) was added and heating continued for a further 4 h. The reaction mixture was concentrated under reduced pressure and diluted with EtOAc (500 mL). The resulting precipitate was collected, washed with EtOAc (2×100 mL) and discarded. The combined organics were washed with 1 M HCl (3×100 mL), water (2×100 mL), brine (100 mL), dried over MgSO₄ and passed through a silica gel plug to afford 6-[4-(tert-butoxycarbonyl)piperazin-1-yl]-5-chloronicotinic acid as a solid. Yield: 32.5 g (96%)

¹H NMR (400 MHz, CDCl₃): δ 1.50 (9H, s), 3.59 (8H, s), 8.18 (1H, s), 8.81 (1H, s).

MS^{m}/z: 340 (M−1).

### (b) tert-Butyl -[3-chloro-5-----(isopropoxycarbonyl)pyridin-2-yl]piperazine-1-carboxylate

6-[4-(tert-Butoxycarbonyl)piperazin-1-yl]-5-chloronicotinic acid (0.407 g, 1.2 mmol), EDCI (0.297 g, 1.6 mmol) and HOBT (0.209 g, 1.6 mmol) were dissolved in DCM (20 mL). The reaction mixture was stirred at room temperature for 90 minutes and then isopropyl alcohol (1.43 g, 24 mmol) and DIPEA (0.622 mL, 3.6 mmol) were added drop-wise. Stirring was continued for 2 h. The mixture was concentrated under reduced pressure, diluted with EtOAc (100 mL), washed with saturated NH₄Cl (2×25 mL), saturated NaHCO₃ (2×25 mL), brine, dried (MgSO₄) and concentrated under reduced pressure to afford tert-butyl 4-[3-chloro-5-(isopropoxycarbonyl)pyridin-2-yl]piperazine-1-carboxylate which was used without purification. Yield: 0.420g (92%/).

¹H NMR (400 MHz, CDCl₃): δ 1.31 (6H, d, J=6.2 Hz), 1.45 (9H, s), 3.44-3.46 (4H, m), 3.52-3.55 (4H, m), 5.14-5.24 (1H, m), 8.08 (1H, s), 8.70 (1H, s).

MS^{m}/z: 384 (M+1).

### (c) Isopropyl 5-chloro-6-piperazin-1-ylnicotinate dihydrochloride

tert-Butyl 4-[3-chloro-5-(isopropoxycarbonyl)pyridin-2-yl]piperazine-1-carboxylate (0.400g. 1.0 mmol) was dissolved in MeOH (30 mL) and HCl (4 M in dioxane, 2.6 mL, 10 mmol) was added drop-wise. The reaction mixture was stirred at room temperature for 16 h and concentrated under reduced pressure to yield isopropyl 5-chloro-6-piperazin-1-ylnicotinate dihydrochloride as a solid, which was used crude assuming 100% conversion.

### (d) 2,2,2-Trichloroethyl [(5-chloro-2-thienyl)sulfonyl]carbamate

5-chlorothiophene-2-sulfonamide (15.00 g, 75.89 mmol) was suspended in a bi-phasic mixture of NaOH (9.11 g, 55.41 mmol) in water (100 mL) and DCM (250 mL). The reaction mixture was cooled to 0° C. and then 2,2,2-trichloroethyl chloroformate (30.1 mL, 132.8 mmol) added drop-wise to the rapidly stirred mixture. The reaction mixture was slowly warmed to room temperature and stirred for 18 h. HCl (conc.) was added drop-wise to the mixture until the pH was lowered to pH 1. The reaction mixture was diluted with DCM (500 mL) the layers separated. The organics were dried (MgSO₄) and concentrated under reduced pressure to afford the crude product. Flash chromatography (1:3 EtOAc/hexanes to 1:1 EtOAc/hexanes) gave 2,2,2-trichloroethyl [(5-chloro-2-thienyl)sulfonyl]carbamate as a solid. Yield: 20.67 g (73%).

¹H NMR (400 N4 Hz, CDCl₃): δ 4.76 (2H, s), 6.99 (1H, d, J=4.2 Hz), 7.71 (1H, d, J=4.2 Hz), 7.74 (1H, br s).

MS^{m}/z: 372 (M−1).

### (e) Isopropyl 5-chloro-6-[4-({[(5-chloro-2-thienyl)sulfonyl]amino}carbonyl)piperazin-1-yl]nicotinate

Isopropyl 5-chloro-6-piperazin-1-ylnicotinate dihydrochloride (0.123 g, 0.38 mmol) and 2,2,2-trichloroethyl [(5-chloro-2-thienyl)sulfonyl]carbamate (0.143 g, 0.38 mmol) were dissolved in DMA (5 mL) at room temperature. DMAP (0.002 g, 0.02 mmol) and DIPEA (0.334 mL, 1.9 mmol) were added and the system sealed with a screw cap and heated to 100° C. for 3 h. The reaction mixture was cooled to room temperature and the solvent concentrated under reduced pressure. The material was partitioned between EtOAc (75 mL) and saturated aqueous NH₄Cl (25 mL). The organics were washed with brine (25 mL), dried (MgSO₄) and concentrated under reduced pressure to afford the crude product. Flash chromatography (1:9 EtOAc/hexanes, 0.5% AcOH to 3:7 EtOAc/hexanes, 0.5% AcOH) followed by preparative HPLC gave isopropyl 5-chloro-6-[4-({[(5-chloro-2-thienyl)sulfonyl]amino}carbonyl)piperazin-1-yl]nicotinate as a solid. Yield: 0.047 g (24%).

¹H NMR (400 MHz, CDCl₃): δ 1.36 (6H, d, J=6.2 Hz), 3.55-3.60 (8H, m), 5.21-5.27 (1H, m), 6.95 (1H, d, J=4.1 Hz), 7.67 (1H, d, J=4.1 Hz), 8.15 (1H, s), 8.74 (1H, s).

MS^{m}/z: 507 (M+1).

### Example 19

### Butyl 5-chloro-6-[4-({[(5-chloro-2-thienyl)sulfonyl]amino}carbonyl)piperazin-1-yl]nicotinate

### (a) piperazin-1-ium 5-chloro-6-piperazin-1-ylnicotinate

5,6-Dichloronicotinic acid (5.00 g, 26 mmol), piperazine (14.6 g, 78 mmol) and DIPEA (13.6 mL, 78 mmol) were dissolved in DMA (100 mL) in a sealed flask and heated to 120° C. for 2 h. The reaction mixture was concentrated under reduced pressure, diluted with EtOAc (250 mL), stirred at room temperature for 16 h, and sonicated for 30 minutes. The resulting solid was collected and washed with EtOAc (2×200 mL) to yield piperazin-1-ium 5-chloro-6-piperazin-1-ylnicotinate which was used crude. Yield: 6.64 g, (78%).

### (b) Butyl 5-chloro-piperazin-1-ylnicotinate

Piperazin-1-ium 5-chloro-6-piperazin-1-ylnicotinat (0.600 g, 1.8 mmol), 1-butanol (8.0 mL, 88 mmol), and concentrated sulfuric acid (1 mL) were heated to 80° C. for 16 h in a sealed flask. The reaction mixture was cooled to 0° C., neutralized with saturated aqueous NaHCO₃ and extracted with EtOAc (3×50 mL). The combined organics were washed with brine (25 mL), dried (MgSO₄) and concentrated under reduced pressure to yield the crude product. Flash chromatography using 1:4 EtOAc/hexanes as the eluant, gradually changing the solvent system to 1:19 (10% NH₄OH in methanol)/ethyl acetate) gave butyl 5-chloro-6-piperazin-1-ylnicotinate. Yield: 0.250g (34%).

¹H NMR (400 MHz, CDCl₃): δ 0.98 (3H, t, J=7.4 Hz), 1.39-1.51 (2H, m), 1.70-1.77 (2H, m), 2.22 (1H, br s), 3.01 (4H, br s), 3.58 (4H, br s), 4.31 (2H, t, J=6.6 Hz), 8.14 (1H, s), 8.75 (1H, s).

MS^{m}/z: 298 (M+1).

### (c) Butyl 5-chloro-6-[4-({[(5-chloro-2-thienyl)sulfonyl]amino}carbonyl)piperazin-1-yl]nicotinate

5-Chlorothiophene-2-sulfonamide (0.066 g, 0.34 mmol) and 1,1′-carbonyldiimidazole (0.054 g, 0.34 mmol) were dissolved in DCM (3 mL) and DIPEA (0.5 mL). The reaction mixture was stirred at room temperature for 6 h. Butyl 5-chloro-6-piperazin-1-ylnicotinate (0.100 g, 0.034 mmol) was added and the reaction mixture was heated to reflux for 16 h. The reaction mixture was cooled to room temperature and concentrated under reduced pressure to yield the crude product. Flash chromatography (3:7 EtOAc/hexanes to EtOAc, 1% AcOH) gave butyl 5-chloro-6-[4-({[(5-chloro-2-thienyl)sulfonyl]amino}carbonyl)piperazin-1-yl]nicotinate as a solid. Yield: 0.130g (74%).

¹H NMR (400 MHz, CDCl₃): δ 0.98 (3H, t, J=7.4 Hz), 1.41-1.51 (2H, m), 1.58 (1H, s), 1.70-1.77 (2H, m), 3.57-3.59 (8H, m), 4.32 (2H, t, J=6.6 Hz), 6.95 (1H, d, J=4.0), 7.67 (1H, d, J=4.0), 8.15 (1H, s), 8.75 (1H, s).

MS^{m}/z: 521 (M+1).

### Example 20

### Methyl 5-chloro-6-[4-({[(5-chloro-2-thienyl)sulfonyl]amino}carbonyl)piperazin-1-yl]nicotinate

### (a) Methyl 5-chloro-6-piperazin-1-ylnicotinate

Piperazin-1-ium 5-chloro-6-piperazin-1-ylnicotinate (0.500 g, 1.5 mmol), MeOH (20.0 mL, 49 mmol), and concentrated sulfuric acid (1 mL) were heated at reflux for 3 h. The reaction mixture was cooled to 0° C., neutralized with saturated aqueous NaHCO₃ and extracted with EtOAc (3×50 mL). The combined organics were washed with brine (25 mL), dried (MgSO₄) and concentrated under reduced pressure to afford methyl 5-chloro-6-piperazin-1-ylnicotinate which was used without purification. Yield: 0.300g (57%).

### (b) Methyl 5-chloro-6-[4-({[(5-chloro-2-thienyl)sulfonyl]amino}carbonyl)piperazin-1-yl]nicotinate

5-Chlorothiophene-2-sulfonamide (0.077 g, 0.39 mmol) and 1,1′-carbonyldiimidazole (0.063 g, 0.39 mmol) were dissolved in DCM (10 mL) and DIPEA (0.70 mL, 5.4 mmol). The reaction mixture was stirred at room temperature for 4 h. Methyl 5-chloro-6-piperazin-1-ylnicotinate (0.100 g, 0.039 mmol) was added and the reaction mixture was heated at reflux for 16 h. The reaction mixture was cooled to room temperature and concentrated under reduced pressure to yield the crude product. Flash chromatography (2:3 Et)Ac/hexanes to EtOAc, 1% AcOH) gave methyl 5-chloro-6-[4-({[(5-chloro-2-thienyl)sulfonyl]amino}carbonyl)piperazin-1-yl]nicotinate as a solid. Yield: 0.131 g (70%).

¹H NMR (400 MHz, CDCl₃): δ 3.57-3.61 (8H, m), 3.91 (3H, s), 6.93 (In, d, J=4.4), 7.64 (1H, d, J=4.4), 8.15 (1H, s), 8.74 (1H, s).

MS^{m}/z: 479 (M+1).

### Example 21

### Propyl 5-chloro-6-[4-({[(5-chloro-2-thienyl)sulfonyl]amino}carbonyl)piperazin-1-yl]nicotinate

### (a) Propyl 5-chloro-6-piperazin-1-ylnicotinate

Piperazin-1-ium 5-chloro-6-piperazin-1-ylnicotinate (0.600 g, 1.8 mmol), 1-propanol (8.0 mL, 107 mmol), and concentrated sulfuric acid (1 mL) were heated to 80° C. for 16 h in a sealed flask. The reaction mixture was cooled to 0° C., neutralized with saturated aqueous NaHCO₃ and extracted with EtOAc (3×50 mL). The combined organics were washed with brine (25 mL), dried (MgSO₄) and concentrated under reduced pressure to yield propyl 5-chloro-6-piperazin-1-ylnicotinate, which was used without purification. Yield: 0.050g (7.1%).

### (b) 5-Chlorothiophene-2-sulfonyl isocyanate

A mixture of 5-chlorothiophene-2-sulfonamide (3.79 g, 19 mmol), n-butyl isocyanate (1.94 mL, 17 mmol), and DABCO (0.054 g, 0.48 mmol) in dichloroethane (80 mL) were heated at reflux with a dry ice/acetone condenser for 30 m. Phosgene (20% in toluene, 13.2 mL, 25 mmol) was added slowly over 15 m and heating continued for 2 h. The dry ice/acetone condenser was removed and the reaction vessel was purged with N₂ through a 1 M NaOH trap for 30 m. After cooling to room temperature, the reaction mixture was passed through a celite plug, and concentrated under reduced pressure to yield 5-chlorothiophene-2-sulfonyl isocyanate as an oil which was used immediately without purification assuming 100% conversion.

### (c) Propyl 5-chloro-6-[4-({[(5-chloro-2-thienyl)sulfonyl]amino}carbonyl)piperazin-1-yl]nicotinate

To a solution of propyl 5-chloro-6-piperazin-1-ylnicotinate (0.155 g, 0.55 mmol) in 0CM (5 mL) was added 5-chlorothiophene-2-sulfonyl isocyanate (0.122 g, 0.55 mmol) and the reaction mixture was stirred at room temperature for 1 h. Additional 5-chlorothiophene-2-sulfonyl isocyanate (0.122 g, 0.55 mmol) was added and stirring was continued for a further 1.5 h. The reaction mixture was diluted with EtOAc (70 mL), washed with 1 M HCl (2×25 mL), and brine (25 mL). The organics were dried (MgSO₄) and concentrated under reduced pressure to afford the crude product. Flash chromatography (3:7 EtOAc/hexanes to 3:7 EtOAc/hexanes, 0.5% AcOH) gave propyl 5-chloro-6-[4-({[(5-chloro-2-thienyl)sulfonyl]amino}carbonyl)-piperazin-1-yl]nicotinate as a solid. Yield: 0.130g (47%).

¹H NMR (400 MHz, CDCl₃): δ 1.02 (3H, t, J=7.4 Hz), 1.74-1.83 (2H, m), 3.56-3.57 (5H, m), 4.27 (2H, t, J=6.6 Hz), 6.95 (1H, d, J=4.0 Hz), 7.67 (1H, d, J=4.0 Hz), 8.16 (1H, s), 8.75 (1H, s).

MS^{m}/z: 507 (M+1).

### Example 22

### 3-Methylbutyl 5-chloro-6-[4-({[(5-chloro-2-thienyl)sulfonyl]amino}carbonyl)-piperazin-1-yl]nicotinate

### (a) 3-Methylbutyl 5-chloro-6-piperazin-1-ylnicotinate

Piperazin-1-ium 5-chloro-6-piperazin-1-ylnicotinate (0.600 g, 1.8 mmol), 3-methyl-1-butanol (8.0 mL, 73 mmol), and concentrated sulfuric acid (1 mL) were heated to 80° C. for 16 h in a sealed flask. The reaction mixture was cooled to 0° C., neutralized with saturated aqueous NaHCO₃ and extracted with EtOAc (3×50 mL). The combined organics were washed with brine (25 mL), dried (MgSO₄) and concentrated under reduced pressure to yield the crude product. Flash chromatography (1:4 EtOAc/hexanes to 1:19 (9:1 MeOH NH₄OH)/EtOAc) gave 3-methylbutyl 5-chloro-6-piperazin-1-ylnicotinate. Yield: 0.050g (7.1%).

¹H NMR (400 MHz, CDCl₃): δ 0.97 (6H, d, J=6.6 Hz), 1.62-1.68 (2H, m), 1.74-1.81 (1H, m), 3.19 (4H, br s), 3.66 (4H, br s), 4.34 (2H, t, J=6.6 Hz), 8.14 (1H, s), 8.76 (1H, s).

MS^{m}/z: 312 (M+1).

### (b) 3-Methylbutyl 5-chloro-6-[4-({[(5-chloro-2-thienyl)sulfonyl]amino}carbonyl)-piperazin-1-yl]nicotinate

5-chlorothiophene-2-sulfonamide (0.063 g, 0.32 mmol) and 1,1′-carbonyldiimidazole (0.052 g, 0.32 mmol) were dissolved in DCM (2 mL) and DIPEA (0.50 mL, 3.2 mmol). The reaction mixture was stirred at room temperature for 4 h. 3-Methylbutyl 5-chloro-6-piperazin-1-ylnicotinate (0.100 g, 0.032 mmol) was added and the reaction mixture was heated at reflux for 16 h. The reaction mixture was cooled to room temperature and concentrated under reduced pressure to yield the crude product. Flash chromatography (3:7 EtOAc/hexanes to EtOAc, 1% AcOH) gave 3-methylbutyl 5-chloro-6-[4-({[(5-chloro-2-thienyl)sulfonyl]amino}carbonyl)piperazin-1-yl]nicotinate as a solid. Yield: 0.125 g (73%).

¹H NMR (400 MHz, CDCl₃): δ 0.97 (6H, d, J=6.5 Hz), 1.62-1.67 (2H, m), 1.72-1.82 (1H, m), 3.57-3.59 (8H, m), 4.34 (2H, t, J=6.7 Hz), 6.95 (1H, d, J=3.9), 7.67 (1H, d, J=3.9), 8.15 (1H, s), 8.75 (1H, s).

MS^{m}/z: 535 (M+1).

### Example 23

### Ethyl 5-chloro-6 (4-{[(phenylsulfonyl)amino]carbonyl}piperidin-1-ylnicotinate

### (a) 1-[3-Chloro-S-(ethoxycarbonyl)pyridin-2-yl]piperidine-4-carboxylic acid

5,6-Dichloro-nicotinic acid ethyl ester (5.00 g, 22.7 mmol) and iso-nipecotic acid (4.40 g, 34.1 mmol) were suspended in DMA (50 mL). DIPEA (11.9 mL, 68.2 mmol) was added to the system heated at 120° C. for 2 h. The reaction mixture was cooled to room temperature and concentrated under reduced pressure to afford the crude material. The crude material was partitioned between DCM (300 mL) and 1N 1C6 (150 mL) and the organics separated. The organics were dried (MgSO₄) and concentrated under reduced pressure to afford the crude product. Flash chromatography (1:4 EtOAc/hexanes to 1:3 EtOAc/hexanes, 0.5% AcOH) gave 1-[3-chloro-5-(ethoxycarbonyl)pyridin-2-yl]piperidine-4-carboxylic acid as a solid. Yield: 6.36 g (90%).

¹H NMR (400 MHz, CDCl₃): δ 1.38 (3H, t, J=7.1 Hz), 1.88-1.97 (2H, m), 2.03-1.12 (2H, m), 2.57-2.66 (ill, m), 2.99-3.09 (2H, m), 4.02-4.11 (2H, m), 4.36 (2H, q, J=7.1 Hz), 8.12 (1H, s), 8.74 (1H, s).

MS^{m}/z: 311 (M−1).

### (b) Ethyl 5-chloro-6-(4{[(phenylsulfonyl)amino]carbonyl}piperidin-1-yl)nicotinate

1-[3-Chloro-5-(ethoxycarbonyl)pyridin-2-yl]piperidine-4-carboxylic acid (0.250 g, 0.80 mmol), EDCI (0.199 g, 1.04 mmol) and HOBT (0.140 g, 1.04 mmol) were dissolved in DCM (5 mL) at room temperature. The reaction mixture was stirred at room temperature for 30 minutes and then benzenesulfonamide (0.251 g, 1.60 mmol) and DIPEA (0.42 mL, 2.40 mmol) were added. The reaction mixture was stirred at room temperature for 18 h. The reaction mixture was diluted with DCM (70 mL) and washed with saturated aqueous NH₄Cl (2×40 mL) and brine (40 mL). The organics were dried (MgSO₄) and concentrated under reduced pressure to afford the crude product. Flash chromatography (3:7 EtOAc/hexanes, 0.5% AcOH to 7:3 EtOAc/hexanes, 0.5% AcOH) gave ethyl 5-chloro-6-(4-{[(phenylsulfonyl)amino]carbonyl}piperidin-1-yl)nicotinate as a solid. Yield: 0.359 g (99%).

¹H NMR (400 MHz, CDCl₃): δ 1.37 (3H, t, J=7.1 Hz), 1.77-1.92 (4H, m), 2.39-2.46 (1H, m), 2.88-2.95 (2H, m), 4.06-4.09 (2H, m), 4.35 (2H q, J=7.1 Hz), 7.55-7.59 (2H, m), 7.65.7.69 (1H, m), 8.07-8.09 (2H, m), 8.10 (1H, s), 8.32 (1H, br s), 8.71 (1H, s).

MS^{m}/z: 452 (M+1).

### Example 24

### Ethyl 5-chloro-6-[4-({[(5-chloro-2-thienyl)sulfonyl]amino}carbonyl)piperidin-1-yl]nicotinate

1-[3-Chloro-5-(ethoxycarbonyl)pyridin-2-yl]piperidine-4-carboxylic acid (0.250 g, 0.80 mmol), EDCI (0.199 g, 1.04 mmol) and HOBT (0.140 g, 1.04 mmol) were dissolved in DCM (5 mL) at room temperature. The reaction mixture was stirred at room temperature for 30 minutes and then 5-chlorothiophene-2-sulfonamide (0.316 g, 1.60 mmol) and DIPEA (0.42 mL, 2.40 mmol) were added. The reaction mixture was stirred at room temperature for 18 h. The reaction mixture was diluted with DCM (70 mL) and washed with saturated aqueous NH₄Cl (2×40 mL) and brine (40 mL). The organics were dried (MgSO₄) and concentrated under reduced pressure to afford the crude product. Flash chromatography (3:7 EtOAc/hexanes, 0.5% AcOH to 7:3 EtOAc/hexanes, 0.5% AcOH) gave Ethyl 5-chloro-6-[4-({[(5-chloro-2-thienyl)sulfonyl]amino}carbonyl)piperidin-1-yl]nicotinate as a solid. Yield: 0.095 g (24%).

¹H NMR (400 MHz, CDCl₃): δ 1.38 (3H, t, J=7.1 Hz), 1.83-1.98 (4H, m), 2.42-2.50 (1H, m), 2.92-2.98 (2H, m), 4.09-4.13 (2H, m), 4.36 (2H q, J=7.1 Hz), 6.97 (1H, d, J=4.1 Hz), 7.71 (1H, d, J=4.1 Hz), 8.12 (1H, d, J=1.7 Hz), 8.73 (1H, d, A=1.7 Hz).

MS^{m}/z: 492 (M+1).

### Example 25

### Ethyl 5-chloro-4-[3-({[(phenylsulfonyl)amino]carbonyl}amino)azetidin-1-yl]nicotinate

### (a) 1-[3-Chloro-5-(ethoxycarbonyl)pyridin-2-yl]azetidine-3-carboxylic acid

5,6-Dichloro-nicotinic acid ethyl ester (3.68 g, 16.48 mmol) and azetidinecarboxylic acid (2.50 g, 24.763 mmol) were suspended in DMA (50 mL). DIPEA (8.61 mL, 49.45 mmol) was added to the system heated at 120° C. for 18 h. The reaction mixture was cooled to room temperature and concentrated under reduced pressure to afford the crude material. The crude material was partitioned between DCM (300 mL) and 1N HCl (150 mL) and the organics separated. The organics were dried (MgSO₄) and concentrated under reduced pressure to afford the crude product. Flash chromatography (1:3 EtOAc/hexanes to 1:3 EtOAc/hexanes, 0.5% AcOH) gave 1-[3-chloro-5-(ethoxycarbonyl)pyridin-2-yl]azetidine-3-carboxylic acid as a solid. Yield. 3.44 g (73%).

¹H NMR (400 MHz, CDCl₃): δ 1.37 (3H, t, J=7.1 Hz), 3.54-3.61 (1H, m), 4.34 (2H, q, J=7.1 Hz), 4.53-4.63 (4H, m), 8.00 (1H, s), 8.67 (1H, s).

MS^{m}/z: 283 (M−1).

### (b) Ethyl 5-chloro-6-[3-({[(phenylsulfonyl)amino]carbonyl}-amino)azetidin-1-yl]nicotinate

1-[3-Chloro-5-(ethoxycarbonyl)pyridin-2-yl]azetidine-3-carboxylic acid (0.150 g, 0.53 mmol), EDCI (0.131 g, 0.68 mmol) and HOBT (0.093 g, 0.68 mmol) were dissolved in DCM (5 mL) at room temperature. The reaction mixture was stirred at room temperature for 30 minutes and then benzenesulfonamide (0.166 g, 1.05 mmol) and DIPEA (0.28 mL, 1.58 mmol) were added. The reaction mixture was stirred at room temperature for 18 h. The reaction mixture was diluted with DCM (70 mL) and washed with saturated aqueous NH₄Cl (2×40 mL) and brine (40 mL). The organics were dried (MgSO₄) and concentrated under reduced pressure to afford the crude product. Flash chromatography (3:7 EtOAc/hexanes, 0.5% AcOH to 7:3 EtOAc/hexanes, 0.5% AcOH) gave ethyl 5-chloro-6-[3-({[phenylsulfonyl)amino]carbonyl}amino)azetidin-1-yl]nicotinate as a solid. Yield: 0.141 g (63%).

¹H NMR (400 MHz, CDCl₃): δ 1.36 (3H, t, J=7.3 Hz), 3.39-3.46 (1H, m), 4.33 (2H, q, J=7.3 Hz), 4.41-4.48 (4H, m), 7.57-7.61 (2H, m), 7.68-7.71 (1H, m), 7.98 (1H, s), 8.08-8.10 (2H, m), 8.64 (1H, s).

MS^{m}/z: 424 (M+1).

### Example 26

### Ethyl 5-chloro-6-[3-({[(5-chloro-2-thienyl)sulfonyl]amino}carbonyl)azetidin-1-yl]nicotinate

1-[3-Chloro-5-(ethoxycarbonyl)pyridin-2-yl]azetidine-3-carboxylic acid (0.150 g, 0.53 mmol), EDCI (0.131 g, 0.68 mmol) and HOBT (0.093 g, 0.68 mmol) were dissolved in DCM (5 mL) at room temperature. The reaction mixture was stirred at room temperature for 30 minutes and then 5-chlorothiophene-2-sulfonamide (0.208 g, 1.05 mmol) and DIPEA (0.28 mL, 1.58 mmol) were added. The reaction mixture was stirred at room temperature for 18 h. The reaction mixture was diluted with DCM (70 mL) and washed with saturated aqueous NH₄Cl (2×40 mL) and brine (40 mL). The organics were dried (MgSO₄) and concentrated under reduced pressure to afford the crude product. Flash chromatography (3:7 EtOAc/hexanes, 0.5% AcOH to 7:3 EtOAc/hexanes, 0.5% AcOH) gave ethyl 5-chloro-6-[3-({[(phenylsulfonyl)amino]carbonyl}amino)azetidin-1-yl]nicotinate as a solid. Yield: 0.137 g (56%).

¹H NMR (400 MHz, CDCl₃): δ 1.37 (3H, t, J=7.1 Hz), 3.40-3.47 (1H, m), 4.34 (2H, q, J=7.1 Hz), 4.46-4.53 (4H, m), 6.99 (1H, d J=4.6 Hz), 7.72 (1H, d, J=4.6 Hz), 8.01 (1H, s), 8.66 (1H, s).

MS^{m}/z: 464 (M+1).

### Example 27

### Ethyl 5-chloro-6-[3-({[(phenylsulfonyl)amino]carbonyl}amino)azetidin-1-yl]nicotinate

### (a) Ethyl 6-{3-[(tert-butoxycarbonyl)amino]azetidin-1-yl}-5-chloronicotinate

Ethyl 5,6-dichloronicotinate (1.00 g, 4.5 mmol) and tert-butyl azetidin-3-ylcarbamate (0.765 g, 3.8 mmol) were dissolved in DMA (10 mL) at room temperature. DIPEA (1.66 g, 9.5 mmol) was added and the system heated at reflux for 16 h. The reaction mixture was cooled to room temperature and the solvent concentrated under reduced pressure. The material was partitioned between EtOAc (100 mL) and a 50% mixture of saturated aqueous NaHCO₃ and brine (1×80 mL). The organics were dried (MgSO₄) and concentrated under reduced pressure to afford the crude product. Flash Chromatography (4:1 hexanes/EtOAc) gave ethyl 6-{3-[(tert-butoxycarbonyl)amino]azetidin-1-yl}-5-chloronicotinate. Yield: 1.02 g (50%).

¹H NMR (400 MHz, CDCl₃): δ 1.38 (3H, t, J=7.0 Hz), 1.46 (9H, s), 4.12-4.15 (2H, m), 4.34 (2H, q, J=7.1 Hz), 4.57 (1H, m), 4.65-4.70 (2H, m), 5.00 (1H, m), 7.98 (1H, s), 8.65 (1H, s).

MS^{m}/z: 355 (M+1).

### (b) Ethyl 6-(3-aminoazetidin-1-yl)-5-chloronicotinate dihydrochloride

Ethyl 6-{3-[(tert-butoxycarbonyl)amino]azetidin-1-yl}-5-chloronicotinate. (1.00 g, 2.8 mmol) was dissolved in DCM (4 mL) at room temperature. HCl (1.80 mL, 14 mmol) was added and the system stirred for 16 h. The solvent was concentrated under reduced pressure. The material was azeotroped using hexanes and toluene, and concentrated under reduced pressure to afford Ethyl 6-(3-aminoazetidin-1-yl)-5-chloronicotinate dihydrochloride product as a crude solid. Yield: 0.480g (102%).

### (c) Ethyl 5-chloro-6-[3-({[(phenylsulfonyl)amino]carbonyl}amino)azetidin-1-yl]nicotinate

Ethyl 6-(3-aminoazetidin-1-yl)-5-chloronicotinate dihydrochloride (0.150 g, 0.41 mmol) was suspended in DCM (5 mL) and TEA (0.21 mL, 1.52 mmol) was added. Benzenesulfonyl isocyanate (0.045 mL, 0.335 mmol) was added and the reaction mixture was stirred at room temperature for 16 h. The reaction mixture was diluted with EtOAc (20 mL) and washed with saturated aqueous NH₄Cl (3×20 mL). The organics were dried (MgSO₄) and concentrated under reduced pressure to afford the crude product. Flash chromatography (50% EtOAc/hexanes to 50% EtOAc/hexanes with 1% AcOH) gave Ethyl 5-chloro-6-[3-({[(phenylsulfonyl)amino]carbonyl}amino)azetidin-1-yl]nicotinate. Yield: 0.042 g (31%).

¹H NMR (400 MHz, CDCl₃): δ 1.69 (3H, t, J=7.0 Hz), 4.14-4.15 (2H, m), 4.35 (2H, q, J=7.0 Hz), 4.66-4.72 (3H, m), 7.08 (1H, m), 7.54-7.60 (2H, m), 7.67-7.69 (2H, m), 8.02 (1H, s), 8.68 (1H, s).

MS^{m}/z: 439 (M+1).

### Example 28

### Ethyl 5-chloro-6 [3-({[(5-chloro-2-thienylsulfonyl]amino}carbonyl)pyrrolidin-1-yl]nicotinate

### (a) 1-[3-Chloro-5-(ethoxycarbonyl)pyridin-2-yl]pyrrolidine-3-carboxylic acid

5,6-Dichloro-nicotinic acid ethyl ester (0.765 g, 0.35 mmol) and 3-pyrrolidine carboxylic acid (0.060 g, 0.52 mmol) were suspended in DMA (3 mL). DIPEA (0.18 1.04 l) was added to the system heated at 120° C. for 18. The reaction mixture was cooled to room temperature and concentrated under reduced pressure to afford the crude material. The crude material was partitioned between DCM (70 mL) and 1N HCl (40 mL) and the organics separated. The organics were dried (MgSO₄) and concentrated under reduced pressure to afford 1-[3-chloro-5-(ethoxycarbonyl)pyridin-2-yl]pyrrolidine-3-carboxylic acid as a solid crude product, which was used without further purification.

### (b) Ethyl 5-chloro-6-[3-({[(5-chloro-2-thienyl)sulfonyl]amino}carbonyl)pyrrolidin-1-yl]nicotinate

1-[3-Chloro-5-(ethoxycarbonyl)pyridin-2-yl]pyrrolidine-3-carboxylic acid (0.065 g, 0.22 mmol), EDCI (0.054 g, 0.28 mmol) and HOBT (0.038 g, 0.28 mmol) were dissolved in DCM (5 mL) at room temperature. The reaction mixture was stirred at room temperature for 30 minutes and then 5-chlorothiophene-2-sulfonamide (0.065 g, 0.33 mmol) and DIPEA (0.11 mL, 0.65 mmol) were added. The reaction mixture was stirred at room temperature for 18 h. The reaction mixture was diluted with DCM (70 mL) and washed with saturated aqueous NH₄Cl (2×40 mL) and brine (40 mL). The organics were dried (MgSO₄) and concentrated under reduced pressure to afford the crude product. Flash chromatography (3:7 EtOAc/hexanes, 0.5% AcOH to 7:3 EtOAc/hexanes, 0.5% AcOH) gave ethyl 5-chloro-6-[3-({[(5-chloro-2-thienyl)sulfonyl]amino}carbonyl)pyrrolidin-1-yl]nicotinate as a solid. Yield: 0.054 g (52 I/o).

¹H NMR (400 MHz, CDCl₃): δ 1.38 (3H, t, J=7.2 Hz), 2.12-2.21 (1H, m), 2.25-2.33 (1H, m), 3.02-3.09 (1H, m), 3.74-3.85 (2H, m), 3.88-3.94 (1H, m), 4.11-4.15 (1H, m), 4.36 (2H, q, J=7.2 Hz), 6.96 (1H, d, J=4.1 Hz), 7.69 (1H, d, J=4.1 Hz), 8.11 (1H, s), 8.69 (1H, s).

MS^{m}/z: 478 (M+1).

### Example 29

### Ethyl 6-[3-(3-ter-butoxy-3-oxopropyl)-4-({[(5-chloro-2-thienyl)sulfonyl]amino}carbonyl)piperazin-1-yl]-5-cyano-2-(trifluoromethyl)nicotinate

### (a) Ethyl 6-[3-(3-tert-butoxy-3-oxopropyl)piperazin-1-yl]-5-cyano-2-(trifluoromethyl)nicotinate

Ethyl 6-chloro-5-cyano-2-(trifluoromethyl)nicotinate (250 mg, 0.90 mmol) and tert-butyl 3-piperazin-2-ylpropanoate (192 mg, 0.90 mmol) was dissolved in ethanol (2 ml). Triethylamine (0.15 ml, 1.08 mmol) was added. The solution was heated in a microwave reactor at 150° C. for 20 min. The solvent was evaporated in vacuo and the residue was dissolved in DCM (50 ml). This solution was washed with water (50 ml), dried over MgSO₄ and the solvents were removed under reduced pressure. The residue was purified by flash chromatography (DCM/methanol 50:1) to give ethyl 6-[3-(3-tert-butoxy-3-oxopropyl)piperazin-1-yl]-5-cyano-2-(trifluoromethyl)nicotinate. Yield: 162 mg (40%).

¹H NMR (400 MHz, CDCl₃): δ 1.36 (3H, t, J=7.2 Hz), 1.44 (9H, s), 1.58-1.84 (3H, m), 2.35 (2H, t, J-7.7 Hz), 2.75-2.83 (1H, m), 2.85-2.93 (2H, m), 3.10-3.16 (1H, m), 3.18-3.28 (1H, m), 4.35 (2H, q, J=7.2 Hz), 4.59-4.67 (2H, m), 8.34 (1H, s).

MS^{m}/z: 457 (M+H).

### (b) Ethyl 6-[3-(3-tert-butoxy-3-oxopropyl)-4-({[(5-chloro-2-thienyl)sulfonyl]amino}carbonyl)piperazin-1-yl]-5-cyano-2-(trifluoromethyl)nicotinate

N,N′-carbonyldiimidazole (34 mg, 0.21 mmol), 5-chlorothiophene-2-sulfonamide (27 mg, 0.14 mmol) and N,N-diisopropylethylamine (0.10 ml, 0.58 mmol) was dissolved in DCM (1 ml) under nitrogen. The solution was stirred at room temperature for 3.5 h. A solution of ethyl 6-[3-(3-tert-butoxy-3-oxopropyl)piperazin-1-yl]-5-cyano-2-(trifluoromethyl)nicotinate (63 mg, 0.14 mmol) and N,N-diisopropylethylamine (0.14 ml, 0.81 mmol) in DCM (1 ml) was added and the stirring was continued for 24 h. The reaction mixture was transferred to a vial which was capped and heated to 40° C. in an oil bath over night. The solvent was removed in vacuo. The residue was purified by preparative HPLC (ammonium acetate buffer(0.1 M)/acetonitrile 80:20 to 30:70). The pure fractions were concentrated to a volume of about 20 ml and extracted with DCM (3×20 ml). The combined organic extracts were dried over MgSO₄, filtered and the solvents were removed in vacuo to give ethyl 6-[3-(3-tert-butoxy-3-oxopropyl)-4-({[(5-chloro-2-thienyl)sulfonyl]amino}carbonyl)piperazin-1-yl]-5-cyano-2-(trifluoromethyl)nicotinate. Yield: 56 mg (60%).

¹H NMR (400 MHz, CDCl₃): δ 1.37 (3H, t, J=7.2 Hz), 1.52 (9H, s), 1.69-1.81 (1H, m), 1.83-1.95 (1H, m), 2.34-2.42 (2H, m), 3.09 (dt, 1H, J=3.2 and 12.5 Hz), 3.36-3.50 (2H, m), 4.02-4.10 (1H, m), 4.29 (1H, d, J=13.9 Hz), 4.37 (2H, q, J=7.2 Hz), 4.51-4.66 (2H, m), 6.91 (1H, d, J=4.0 Hz), 7.65 (1H, d, J=4.0 Hz), 8.37 (1H, s).

MS^{m}/z: 680 (M+H).

### Example 30

### 3-{1-({[(5-Chloro-2-thienyl)sulfonyl]amino}carbonyl)-4-[3-cyano-5-[ethoxy(hydroxy)methyl]-6-(trifluoromethyl)pyridin-2-yl]piperazin-2-yl}propanoic acid

Ethyl 6-[3-(3-tert-butoxy-3-oxopropyl)-4-({[(5-chloro-2-thienyl)sulfonyl]amino}carbonyl)piperazin-1-yl]-5-cyano-2-(trifluoromethyl)nicotinate (56 mg, 0.082 mmol) was dissolved in DCM (4 ml) under nitrogen. Trifluoroacetic acid (1 ml) was added. The resulting solution was stirred at room temperature for 1 h. The solvents were removed in vacuo. The residue was purified by preparative HPLC (acetonitrile/ammonium acetate buffer(0.1M) 20-40%), the solvents were removed by freeze-drying to give 3-{1-({[(5-Chloro-2-thienyl)sulfonyl]amino}carbonyl)-4-[3-cyano-5-[ethoxy(hydroxy)methyl]-6-(trifluoromethyl)pyridin-2-yl]piperazin-2-yl}propanoic acid. Yield: 46 mg (90%).

¹H NMR (400 MHz, d₆-DMSO): δ 1.27 (3H, t, J=7.1 Hz), 1.53-1.75 (2H, m), 2.02-2.14 (1H, m), 2.16-2.28 (1H, m), 3.14-3.40 (3H, m), 4.06-4.16 (1H, m), 4.26 (2H, q, J=7.1 Hz), 4.30-4.45 (3H, m), 6.90 (1H, d, J=3.8 Hz), 7.14 (1H, d, J=3.8 Hz), 8.49 (1H, s).

MS^{m}/z: 624 (M+H).

### Example 31

### Ethyl 6-(3-(3-tert-butoxy-3-oxopropyl)-4-{[(phenylsulfonyl)amino]carbonyl}piperazin-1-yl)-5-cyano-2-(trifluoromethyl)nicotinate

Ethyl 6-[3-(3-tert-butoxy-3-oxopropyl)piperazin-1-yl]-5-cyano-2-(trifluoromethyl)nicotinate (59 mg, 0.13 mmol) was dissolved in DCM under nitrogen. Benzenesulfonyl isocyanate (30 μl, 0.19 mmol) was added and the reaction mixture was stirred at room temperature for 20 h. The solvent was removed in vacuo. The residue was dissolved in DCM (25 ml) and washed with water (2*15 ml). The organic phase was evaporated in vacuo to give Ethyl 6-(3-(3-tert-butoxy-3-oxopropyl)-4-{[(phenylsulfonyl)amino]carbonyl}piperazin-1-yl)-5-cyano-2-(trifluoromethyl)nicotinate. Yield: 76 mg (92%).

¹H NMR (400 MHz, CDCl₃): δ 1.38 (3H, t, J=7.1 Hz), 1.55 (9H, s), 1.68-1.80 (1H, m), 1.81-1.93 (1H, m), 2.34-2.41 (2H, m), 3.03 (1H, dt, J=3.3 and 12.5 Hz), 3.33-3.50 (2H, m), 4.07 (1H, s br), 4.23 (1H, d, J=13.7 Hz), 4.38 (2H, q, J=7.1 Hz), 4.52 (1H, d, J=13.5 Hz), 4.61 (1H, d, J=13.9 Hz), 7.49-7.56 (2H, m), 7.57-7.64 (1H, m), 8.06-8.11 (2H, m), 8.37 (1H, s).

MS^{m}/z: 640 (M+@).

### Example 32

### 3-(4-[3-Cyano-5-(ethoxycarbonyl)-6-(trifluoromethyl)pyridin-2-yl]-1-{[(phenylsulfonyl)amino]carbonyl}piperazin-2-yl)propanoic acid

Ethyl 6-(3-(3-tert-butoxy-3-oxopropyl)-4-{[(phenylsulfonyl)amino]carbonyl}piperazin-1-yl)-5-cyano-2-(trifluoromethyl)nicotinate (76 mg, 0.12 mmol) was dissolved in DCM (4 ml) under nitrogen. Trifluoroacetic acid (1 ml) was added. The resulting solution was stirred at room temperature for 1 h. The solvents were removed in vacuo. The residue was purified by preparative HPLC (acetonitrile/ammonium acetate buffer(0.1M) 20-30%). The pure fractions were combined and concentrated to about 10 ml in vacuo followed by extraction using DCM (3×10 ml). The combined organic extracts were dried (MgSO₄) and evaporated in vacuo to give 3-(4-[3-Cyano-5-(ethoxycarbonyl)-6-(trifluoromethyl)pyridin-2-yl]-1-{[(phenylsulfonyl)amino]carbonyl}piperazin-2-yl)propanoic acid. Yield: 54 mg (78%).

¹H NMR (400 MHz, CDCl₃): δ 1.37 (3H, , J=7.2 Hz), 1.75-1.95 (2H, m), 2.42-2.60 (2H, m), 3.04-3.22 (1H, m), 3.32-3.47 (2H, m), 4.05-4.25 (2H, m), 4.37 (2H, q, J=7.2 Hz), 4.47-4.54 (1H, m), 4.62A468 (1H, m), 7.48-7.54 (2H, m), 7.56-7.62 (1H, m), 8.01-8.07 (2H, m), 8.35 (1H, s).

MS^{m}/z: 584 (M+H).

### Example 33

### Ethyl 6-(3-(3-tert-butoxy-3-oxopropyl)-4-{[(phenylsulfonyl)amino]carbonyl}piperazin-1-yl)-5-chloronicotinate

### (a) Ethyl 6-[3-(3-tert-butoxy-3-oxopropyl)piperazin-1-yl]-5-chloronicotinate

5,6-Dichloronicotinic acid ethyl ester (205 mg, 0.93 mmol) and tert-butyl 3-piperazin-2-ylpropanoate (200 mg, 0.93 mmol) was dissolved in ethanol (2 ml). Triethylamine (0.14 ml, 1.03 mmol) was added. The solution was heated in a microwave reactor at 120° C. for 15 min. Ethyl acetate (8 ml) and 10% K₂CO₃ (8 ml) was added. The phases were separated and the aqueous phase was extracted with ethyl acetate (2×8 ml). The combined organic extracts were dried over MgSO₄, filtered and evaporated under reduced pressure. Flash chromatography (methanol/DCM 8%) gave ethyl 6-[3-(3-tert-butoxy-3-oxopropyl)piperazin-1-yl]-5-chloronicotinate . Yield: 256 mg (69%).

¹H NMR (400 MHz, CDCl₃): δ 1.36 (3H, t, J=7.2 Hz), 1.44 (9H, s), 1.62-1.80 (2H, m), 2.34 (2H, dt, J=3.0 and 7.6 Hz), 2.60-2.69 (1H, m), 2.81-2.90 (1H, m), 2.91-3.05 (2H, m), 3.06-3.12 (1H, m), 3.96-4.06 (2H, m), 4.34 (2H, q, J=7.2 Hz), 8.10 (1H, d, J=2.0 Hz), 8.72 (1H, d, J=2.0 Hz).

MS^{m}/z: 398 (M+H).

### (b) Ethyl 6-(3-(3-tert-butoxy-3-oxopropyl)-4-{[(phenylsulfonyl)amino]carbonyl}piperazin-1-yl)-5-chloronicotinate

Benzenesulfonyl isocyanate (10 μl, 0.072 mmol) was added to a solution of ethyl 6-[3-(3-tert-butoxy-3-oxopropyl)piperazin-1-yl]-5-chloronicotinate (24 mg, 0.060 mmol) in acetonitrile (2 ml). The resulting mixture was purged with nitrogen and stirred at room temperature for 4 h. PS-TRIS (50 mg, 4.4 mmol/g) was added and the stirring was continued for 1 h. The suspension was filtered, and the solid material was washed with DCM. The filtrate was evaporated in vacuo, and the residue was purified by flash chromatography (ethyl acetateaheptane 80%). Yield: 10 mg (29%).

¹H NMR (400 MHz, CDCl₃): δ 1.38 (3H, t, J=7.2 Hz), 1.54 (9H, s), 1.76-1.90 (1H, m), 2.10-2.24 (1H, m), 2.30-2.38 (2H, m), 2.92-3.14 (3H, m), 3.90-4.06 (3H, m), 4.16-4.30 (1H, m), 4.37 (2H, q, J=7.2 Hz), 7.49-7.54 (2H, m), 7.56-7.62 (1H, m), 8.07-8.12 (2H, m), 8.14 (1H, d, J=2.0 Hz), 8.74 (1H, d, J=2.0 Hz).

MS^{m}/z: 581 (M+H).

### Example 34

### 3-(4-[3-Chloro-5-(ethoxycarbonyl)pyridin-2-yl]-1-{[(phenylsulfonyl)amino]carbonyl}piperazin-2-yl)propanoic acid

Ethyl 6-(3-(3-tert-butoxy-3-oxopropyl)-4-{[(phenylsulfonyl)amino]carbonyl}piperazin-1-yl)-5-chloronicotinate (132 mg, 0.23 mmol) was dissolved in DCM (8 ml) at room temperature under nitrogen. Trifluoroacetic acid (2 ml) was added. The resulting solution was stirred at room temperature under nitrogen for 1 h. The solvents were removed in vacuo and the residue was coevaporated with toluene (2×5 ml). The residue was purified by preparative HPLC (acetonitrile/ammonium acetate buffer(0.1M) 10-30%), removal of the solvents by freeze-drying gave 3-(4-[3-Chloro-5-(ethoxycarbonyl)pyridin-2-yl]-1-{[(phenylsulfonyl)amino]carbonyl}piperazin-2-yl)propanoic acid. Yield: 30 mg (25%).

¹H NMR (400 MHz, CD₃OD): δ 1.41 (3H, t, J=7.2 Hz), 1.84-2.00 (1H, m), 2.10-2.22 (1H, m), 2.22-2.40 (2H, m), 2.80-3.03 (1H, m), 3.06-3.16 (1H, m), 3.16-3.32 (1H, m), 4.04 (1H, d, J=12.7 Hz), 4.10 (1H, d, J=13.1 Hz), 4.18 (1H, d, J=13.7 Hz), 4.39 (2H, q, J=7.2 Hz), 4.47 (1H, br s), 7.48-7.60 (3H, m), 7.97-8.02 (2H, m), 8.18 (1H, d, J-2.0 Hz), 8.73 (1H, d, J=2.0 Hz).

MS^{m}/z: 525 (M+H).

### Example 35

### Ethyl 5-Chloro-6-[4-({[(phenylsulfonyl)amino]carbonyl}amino)piperidin-1-yl]nicotinate

### (a) Ethyl 6-{4-[(tert-butoxycarbonyl)amino]piperidin-1-yl}-5-chloronicotinate

Ethyl 5,6-dichloronicotinate (1.00 g, 4.5 mmol) and 4-(N-Boc amino)-piperidine (0.765 g, 3.8 mmol) were dissolved in CH₃CN (8 mL) at room temperature. DIPEA (1.66 g, 9.5 mmol) was added and the system heated at reflux for 16 h. The reaction mixture was cooled to room temperature and the solvent concentrated under reduced pressure. The material was partitioned between EtOAc (50 mL) and saturated aqueous NH₄Cl (2×30 mL). The organics were washed with brine (30 mL), dried (MgSO₄) and concentrated under reduced pressure to afford the crude product. Flash chromatography (6:1 hexanes/EtOAc) gave ethyl 6-{4-[(tert-butoxycarbonyl)amino]piperidin-1-yl}-5-chloronicotinate. Yield: 1.04 g (84%/).

¹H NMR (400 MHz, CDCl₃): δ 1.38 (3H, t, J=7.0 Hz), 1.46 (9H, s), 2.01-2.12 (2H, m), 3.04 (2H, m), 3.64-3.78 (1H, s), 4.02-4.06 (2H, m), 4.36 (21, q, J=7.0 Hz), 4.50-4.52 (1H, m), 8.11 (1H, s), 8.73 (1H, s).

MS^{m}/z: 384 (M+1).

### (b) Ethyl 6-(4-aminopiperidin-1-yl)-5-chloronicotinate dihydrochloride

Ethyl 6-{4-[(tert-butoxycarbonyl)amino]piperidin-1-yl}-5-chloronicotinate (1.00 g, 2.8 mmol) was dissolved in DCM (2 mL) at room temperature. HCl (3.50 mL, 14 mmol) was added and the system stirred for 16 h. The solvent was concentrated under reduced pressure. The material was azeotroped using hexanes and toluene, and concentrated under reduced pressure to afford ethyl 6-(4-aminopiperidin-1-yl)-5-chloronicotinate dihydrochloride product as a solid. Yield: 1.00 g (91%).

¹H NMR (400 MHz, CD₃OD): δ 1.38 (3H, t, J=7.1 Hz), 1.76-1.86 (2H, m), 2.13-2.16 (2H, m), 3.11-3.18 (2H, m), 3.40-3.46 (1H, m), 4.21-4.25 (2H, m), 4.37 (2H, q, J=7.1 Hz), 8.28 (1H, s), 8.68 (1H, s).

MS^{m}/z: 284 (M+1).

### (c) Ethyl 5-Chloro-6-[4-({[(phenylsulfonyl)amino]carbonyl}amino)piperidin-1-yl]nicotinate

Ethyl 6-(4-aminopiperidin-1-yl)-5-chloronicotinate dihydrochloride (0.100 g, 0.28 mmol) was suspended in DCM (5 mL) and TEA (0.18 mL, 1.27 mmol) was added. Benzenesulfonyl isocyanate (0.037 mL, 0.28 mmol) was added and the reaction mixture was stirred at room temperature for 18 h. The reaction mixture was diluted with DCM (70 mL) and washed with saturated aqueous NH₄Cl (2×40 mL) and brine (40 mL). The organics were dried (MgSO₄) and concentrated under reduced pressure to afford the crude product. Flash chromatography (3:7 EtOAc/hexanes, 0.5% AcOH to 7:3 EtOAc/hexanes, 0.5% AcOH) gave ethyl 5-chloro-6-[4-({[(phenylsulfonyl)amino]carbonyl}amino)piperidin-1-yl]nicotinate as a solid. Yield: 0.079 g (60%).

¹H NMR (400 MHz, CDCl₃): δ 1.38 (3H, t, J=7.2 Hz), 1.58-1.68 (2H, m), 1.99-2.06 (2H, m), 3.02-3.08 (2H, m), 3.83 (1H, m), 3.96-4.05 (2H, m), 4.37 (2H, q, J=7.2 Hz), 6.58 (1H, d, J=7.7 Hz), 7.55-7.61 (2H, m), 7.64-7.71 (1H, m), 7.87-7.92 (2H, m), 8.13 (1H, s), 8.75 (1H, s).

MS^{m}/z: 467 (M+1).

### Example 36

### 4-(5-Butyrl-3-chloropyridin-2-yl)-N-[(5-chloro-2-thienyl)sulfonyl]piperazine-1-carboxamide

### (a) ter-Butyl 4-(3-chloro-5-{[methoxy(methyl)amino]carbonyl}pyridin-2-yl)piperazine-1-carboxylate

6-[4-(tert-Butoxycarbonyl)piperazin-1-yl]-5-chloronicotinic acid (10.0 g, 29.3 mmol) was dissolved in DCM (250 mL) and CDI (5.70 g, 35.0 mmol) added. The system was stirred at room temperature for 15 minutes and then N,O-dimethylhydroxylamine hydrochloride (3.70 g, 37.93 mmol) was added and the system stirred at room temperature for 18 h. The reaction mixture was diluted with DCM (150 mL) and washed sequentially with 1N HCl (100 mL), saturated aqueous NH₄Cl (100 mL) and saturated aqueous NaHCO₃ (100 mL). The organics were dried (MgSO₄) and concentrated under reduced pressure to afford crude tert-butyl 4-(3-chloro-5-{[methoxy(methyl)amino]carbonyl}pyridin-2-yl)piperazine-1-carboxylate as an oil, which was used without further purification.

¹H NMR (400 MHz, CDCl₃): δ 1.49 (9H, s), 3.37 (3H, s), 3.43-3.48 (4H, m), 3.56-3.60 (4H, m), 3.70 (3H, s), 8.03 (1, d, J=2.0 Hz), 8.62 (1H, d, J=2.0 Hz).

MS^{m}/z: 385 (M+1).

### (b) ter-Butyl 4-(5-butyryl-3-chloropyridin-2-yl)piperazine-1-carboxylate

tert-Butyl 4-(3-chloro-5-{[methoxy(methyl)amino]carbonyl}pyridin-2-yl)piperazine-1-carboxylate (1.50 g, 4.05 mmol) was dissolved in THF (14 mL) and the system cooled to 0° C. Propylmagnesium chloride (2.0 M, 3.0 mL, 6.1 mmol) was added drop-wise and the reaction mixture stirred at 0° C. for 15 minutes. The reaction mixture was warmed to room temperature and stirred for a further 1 h. 2N HCl was added to quench the reaction. The reaction mixture was diluted with EtOAc (70 mL) and washed with saturated aqueous NH₄Cl (40 mL) and then brine (40 mL). The organics were dried (MgSO₄) and concentrated under reduced pressure to afford the crude product. Flash chromatography (1:9 EtOAc/hexanes to 1:7 EtOAc/hexanes)) gave tert-butyl 4-(5-butyryl-3-chloropyridin-2-yl)piperazine-1-carboxylate as a solid, Yield: 0.20 g (81%).

### (c) 1-(5-Chloro-6-piperazin-1-ylpyridin-3-yl)butan-1-one dihydrochloride

tert-Butyl 4-(5-butyryl-3-chloropyridin-2-yl)piperazine-1-carboxylate (0.714 g, 1.941 mmol) was dissolved in DCM (25 mL) and HCl (4M in 1,4-dioxane, 4.00 mL, 16.00 mmol) was added and the reaction mixture stirred at room temperature for 18 h. The solvent was concentrated under reduced pressure to afford crude 1-(5-chloro-6-piperazin-1-ylpyridin-3-yl)butan-1-one dihydrochloride as a solid, which was used without further purification.

### (d) 4-(5-Butyryl-3-chloropyridin-2-yl)-N-[(5-chloro-2-thienyl)sulfonyl]piperazine-1-carboxamide

1-(5-Chloro-6-piperazin-1-ylpyridin-3-yl)butan-1-one dihydrochloride (0.160 g, 0.47 mmol) and 2,2,2-trichloroethyl [(5-chloro-2-thienyl)sulfonyl]carbamate (0.160 g, 0.43 mmol) were dissolved in DMA (5 mL) at room temperature. DMAP (0.006 g, 0.05 mmol) and DIPEA (0.82 mL, 4.70 mmol) were added and the system sealed with a screw cap and heated to 105° C. for 2 h. The reaction mixture was cooled to room temperature and the solvent concentrated under reduced pressure. The material was partitioned between EtoAc (70 mL) and saturated aqueous NH₄Cl (40 mL). The organics were washed with brine (40 mL), dried (MgSO₄) and concentrated under reduced pressure to afford the crude product. Flash chromatography (3:7 EtOAc/hexanes, 0.5% AcOH to 7:3 EtOAc/hexanes, 0.5% AcOH) gave 4-(5-butyryl-3-chloropyridin-2-yl)-N-[(5-chloro-2-thienyl)sulfonyl]piperazine-1-carboxamide as a solid. Yield: 0.085 g (37%).

¹H NMR (400 MHz, CDCl₃): δ 1.00 (3H, t, J=7.4 Hz), 1.72-1.81 (2H, m), 2.86 (2H, t, J=7.3 Hz). 3.56-3.64 (8H, m), 6.95 (1H, d, J=4.1 Hz), 7.67 (1H, d, J=4.1 Hz), 8.14 (1H, d, J=1.9 Hz), 8.70 (1H, d, J=1.9 Hz).

MS^{m}/z: 491 (M+1).

### Example 37

### 4-[3-Chloro-5-(2-ethyl-2H-tetrazol-5-yl)pyridin-2-yl]-N-[(5-chloro-2-thienyl)sulfonyl]piperazine-1-carboxamide

### (a) tert-Butyl 4-(3-chloro-5-cyanopyridin-2-yl)piperazine-1-carboxylate

5,6-Dichloronicotinonitrile (5.00 g, 28.90 mmol, made in according to (JPN patent WO-95-JP587)), 1-Boc-piperazine (8.08 g, 43.4 mmol) and DIPEA (15.1 mL, 86.7 mmol) were suspended in DMA (50 mL) and heated at 120° C. for 18 h. The reaction mixture was cooled to room temperature and concentrated under reduced pressure to afford the crude material. The crude material was partitioned between DCM (300 mL) and saturated aqueous NaHCO₃ (150 mL) and the organics separated. The organics were washed with water (150 mL) and then dried (MgSO₄) and concentrated under reduced pressure to afford the crude product. Flash chromatography (DCM) gave tert-butyl 4-(3-chloro-5-cyanopyridin-2-yl)piperazine-1-carboxylate as a solid. Yield: 11.20 g (120%) The product was contaminated with DMA.

¹H NMR (400 MHz, CDCl₃): δ 1.49 (9H, s), 3.52-3.62 (8H, m), 7.76 (1H, s), 8.39 (1H, s).

### (b) tert-Butyl 4-[3-chloro-5-(2H-tetrazol-5-yl)pyridin-2-yl]piperazine-1-carboxylate

tert-Butyl 4-(3-chloro-5-cyanopyridin-2-yl)piperazine-1-carboxylate (2.00 g, 6.20 mmol) was dissolved in DMF (100 mL). Sodium azide (2.014 g, 30.98 mmol) and NH₄Cl (1.657 g, 30.98 mmol) were added to the reaction mixture and the system heated at 75° C. for 18 h. The reaction mixture was cooled to room temperature and concentrated under reduced pressure to afford crude tert-butyl 4-[3-chloro-5-(2H-tetrazol-5-yl)pyridin-2-yl]piperazine-1-carboxylate as a solid, which was used without further purification.

### (e) tert-Butyl 4-[3-chloro-5-(2-ethyl-2H-tetrazol-5-yl)pyridin-2-yl]piperazine-1-carboxylate

tert-Butyl 4-[3-chloro-5-(2H-tetrazol-5-yl)pyridin-2-yl]piperazine-1-carboxylate (2.267 g, 6.20 mmol) and K₂CO₃ (2.569 g, 18.59 mmol) were suspended in acetone (100 mL) and ethyl iodide (0.56 mL, 8.06 mmol) was added. The reaction mixture was stirred at room temperature for 18 h and then concentrated under reduced pressure to afford the crude material. The crude material was partitioned between EtOAc (150 mL) and water (50 mL) and the organics separated. The organics were dried (MgSO₄) and concentrated under reduced pressure to afford the crude product. Flash chromatography (1:4 EtOAc/hexanes to 1:2 EtOAc/hexanes) gave tert-butyl 4-[3-chloro-5-(2-ethyl-2H-tetrazol-5-yl)pyridin-2-yl]piperazine-1-carboxylate as a solid. Yield: 1.304 g (53%).

¹H NMR (400 MHz, CDCl₃): δ 1.49 (9H, s), 1.64-1.76 (3H, m), 3.39-3.51 (4H, m), 3.56-3.67 (4H, m), 4.66-4.76 (2H, m), 8.31 (1H, s), 8.90 (1H, s).

MS^{m}/z: 394 (M+1).

### (d) 1-[3-Chloro-5-(2-ethyl-2H-tetrazol-5-yl)pyridin-2-yl]piperazine dihydrochloride

tert-Butyl 4-[3-chloro-5-(2-ethyl-2H-tetrazol-5-yl)pyridin-2-yl]piperazine-1-carboxylate (1.304 g, 3.31 mmol) was suspended in 1,4-dioxane (30 mL) and DCM added until the material was in solution. HCl (4M in 1,4-dioxane, 16.55 mL, 66.19 mmol) was added and the reaction mixture stirred at room temperature for 18 h. The solvent was concentrated under reduced pressure to afford crude 1-[3-chloro-5-(2-ethyl-2H-tetrazol-5-yl)pyridin-2-yl]piperazine dihydrochloride as a solid, which was used without further purification. Yield: 1.211 g (100%).

¹H NMR (400 MHz, CD₃OD): δ 1.66 (3H, t, J=7.3 Hz), 3.38-3.45 (4H, m), 3.68-3.75 (4H, m), 4.76 (2H, q, J=7.3 Hz), 8.41 (1H, s), 8.92 (1H, s).

MS^{m}/z: 294 (M+1, free base).

### (e) 4-[3-Chloro-5-(2-ethyl-2H-tetrazol-5-yl)pyridin-2-yl]-N-[(5-chloro-2-thienyl)sulfonyl]piperazine-1-carboxamide

1-[3-Chloro-5-(2-ethyl-2H-tetrazol-5-yl)pyridin-2-yl]piperazine dihydrochloride (0.150 g, 0.41 mmol) and 2,2,2-trichloroethyl [(5-chloro-2-thienyl)sulfonyl]carbamate (0.170 g, 0.41 mmol) were placed in a reactor vial and dissolved in DMA (5 mL) at room temperature. DMAP (0.002 g, 0.02 mmol) and DIPEA (0.71 mL, 4.09 mmol) were added and the system sealed with a screw cap and heated to 80° C. for 18 h. The reaction mixture was cooled to room temperature and the solvent concentrated under reduced pressure. The material was partitioned between EtOAc (70 mL) and saturated aqueous NH₄Cl (40 mL). The organics were washed with brine (40 mL), dried (MgSO₄) and concentrated under reduced pressure to afford the crude product. Flash chromatography (3:7 EtOAc/hexanes, 0.5% AcOH to 7:3 EtOAc/hexanes, 0.5% AcOH) gave 4-[3-chloro-5-(2-ethyl-2H-tetrazol-5-yl)pyridin-2-yl]-N-[(5-chloro-2-thienyl)sulfonyl]piperazine-1-carboxamide as a solid. Yield: 0.141 g (63%).

¹H NMR (400 MHz, CDCl₃): δ 1.69 (3H, t, J=7.3 Hz), 3.49-3.51 (4H, m), 3.62-3.64 (4H, m), 4.71 (2H, q, J=7.3 Hz), 6.94 (1H, d, J=3.5 Hz), 7.66 (1H, d, J=3.5 Hz), 8.33 (1H, s), 8.90 (1H, s).

MS^{m}/z: 517 (M+1).

### Example 38

### 4-[3-Chloro-5-(5-ethyl-4,5-dihydro-1,3-oxazol-2-yl)pyridin-2-yl]-N-(phenylsulfonyl)piperazine-1-carboxamide

### (a) tert-Butyl 4-(3-chloro-5{[(2-hydroxybutyl)amino]carbonyl}pyridin-2-yl)piperazine-1-carboxylate

6-[4-(tert-Butoxycarbonyl)piperazin-1-yl]-5-chloronicotinic acid (5.00 g, 15 mmol), EDCI (3.65 g, 19 mmol) and HOBT (2.57 g, 19 mmol) were dissolved in DCM (100 mL). The reaction mixture was stirred at room temperature for 90 minutes and then 1-amino-2-butanol (2.10 mL, 22 mmol) and DIPEA (7.64, 44 mmol) were added dropwise. The reaction mixture was stirred at room temperature for 3 days. The resulting precipitate was filtered, washed with DCM (50 mL) and discarded. The filtrate was concentrated under reduced pressure, diluted with EtOAc (200 mL), washed with saturated NH₄Cl (2×50 mL), saturated NaHCO₃ (2×50 mL), brine, dried (MgSO₄) and concentrated under reduced pressure to afford tert-butyl 4-(3-chloro-5-{[(2-hydroxybutyl)amino]carbonyl}pyridin-2-yl)piperazine-1-carboxylate which was used crude. Yield: 6.04 g (100%/).

MS^{m}/z: 411 (M−1).

### (b) ten-Butyl 4-[3-chloro-5-(5-ethyl-4,5-dihydro-1,3-oxazol-2-yl)pyridin-2-yl]piperazine-1-carboxylate

tert-Butyl 4-(3 chloro-5-{[(2-hydroxybutyl)amino]carbonyl}pyridin-2-yl)piperazine-1-carboxylate (6.18 g, 15 mmol) and DIPEA (10.4 mL, 60 mmol) were dissolved in DCM (100 mL) and cooled to 0° C. Methanesulfonyl chloride (1.40 mL, 18 mmol) was added slowly over 5 minutes. The reaction mixture was allowed to warm to room temperature and stirred for 16 h followed by heating at reflux for 2 days. The reaction mixture was cooled to room temperature, diluted with DCM (200 mL), washed with saturated NaHCO₃ (3×75 mL), dried (MgSO₄) and concentrated under reduced pressure to yield the crude product. Flash chromatography (1:4 EtOAc/hexanes to 1:1 EtOAc/hexanes) gave tert-butyl 4-[3-chloro-5-(5-ethyl-4,5-dihydro-1,3-oxazol-2-yl)pyridin-2-yl]piperazine-1-carboxylate. Yield: 5.29 g (90%).

¹H NMR (400 MHz, CDCl₃): δ 1.01 (3H, t, J=7.4 Hz), 1.49 (1R^{X}, s), 1.64-1.79 (2K, m), 3.43-3.45 (4H, m), 3.57-3.59 (4H, m), 3.62-3.67 (1H, m), 4.05-4.13 (1H, m), 4.62-4.69 (1H, m), 8.10 (1H, s), 8.66 (1H, s).

MS^{m}/z: 395 (M+1).

### (c) 1-[3-Chloro-5-(5-ethyl-4,5-dihydro-1,3-oxazol-2-yl)pyridin-2-yl]piperazine bis(trifluoroacetate)

tert-Butyl 4-[3-chloro-5-(5-ethyl-4,5-dihydro-1,3-oxazol-2-yl)pyridin-2-yl]piperazine-1-carboxylate (1.06 g, 2.7 mmol) was dissolved in DCM (20 mL) and TFA (10 mL) and stirred at room temperature for 16 h. The reaction mixture was concentrated under reduced pressure to yield 1-[3-chloro-5-(5-ethyl-4,5-dihydro-1,3-oxazol-2-yl)pyridin-2-yl]piperazine bis(trifluoroacetate)as an oil which was used without purification assuming 100% conversion.

### (d) 4-[3-Chloro-5-(5-ethyl-4,5-dihydro-1,3-oxazol-2-yl)pyridin-2-yl]-N-(phenylsulfonyl)piperazine-1-carboxamide

1-[3-Chloro-5-(5-ethyl-4,5-dihydro-1,3-oxazol-2-yl)pyridin-2-yl]piperazine (0.264 g, 0.90 mmol) was dissolved in DCM (10 mL) and DIPEA (3.12 mL, 18 mmol) was added. Benzenesulfonyl isocyanate (0.132 mL, 0.99 mmol) was added and the reaction mixture was stirred at room temperature for 18 h. The reaction mixture was concentrated under reduced pressure, diluted with EtOAc (75 mL) and washed with saturated aqueous NH₄Cl (2×25 mL) and brine (25 mL). The organics were dried (MgSO₄) and concentrated under reduced pressure to afford the crude product. Flash chromatography (2:3 EtOAc/hexanes, 0.5% AcOH to 4:1 EtOAc/hexanes, 0.5% AcOH) gave 4-[3-chloro-5-(5-ethyl-4,5-dihydro-1,3-oxazol-2-yl)pyridin-2-yl]-N-(phenylsulfonyl)piperazine-1-carboxamide as a solid. Yield: 0.248 g (58%).

¹H NMR (400 MHz, CDCl₃): δ 1.00 (3H, t, J=7.4 Hz), 1.65-1.79 (2H, m), 3.44-3.46 (4H, m), 3.54-3.57 (4H, m), 3.62-3.68 (1H, m), 4.06-4.12 (1H, m), 4.63-4.70 (1H, m), 7.53-7.78 (2H, m), 7.62-7.65 (1H, m), 8.08-8.10 (3H, m), 8.64 (1H, s).

MS^{m}/z: 478 (M+1).

### Example 39

### 4-[3-Chloro-5-(5-methyl-1,3-oxazol-2-yl)pyridin-2-yl]-N-(phenylsulfonyl)piperazine-1-carboxamide

### (a) tert-Butyl 4-(3-chloro-5-{[(2-hydroxypropyl)amino]carbonyl}pyridin-2-yl)piperazine-1-carboxylate

6-[4-(tert-Butoxycarbonyl)piperazin-1-yl]-5-chloronicotinic acid (5.00 g, 15 mmol), EDCI (3.65 g, 19 mmol) and HOBT (2.57 g, 19 mmol) were dissolved in DCM (100 mL). The reaction mixture was stirred at room temperature for 90 minutes and then 1-amino-2-propanol (1.72 mL, 22 mmol) and DIPEA (7.64, 44 mmol) were added drop-wise. The reaction mixture was stirred at room temperature for 3 days. The resulting precipitate was filtered, washed with DCM (50 mL) and discarded. The filtrate was concentrated, diluted with EtOAc (200 mL), washed with saturated NH₄Cl (2×50 mL), saturated NaHCO₃ (2×50 mL), brine, dried (MgSO₄) and concentrated under reduced pressure to afford tert-butyl 4-(3-chloro-5-{[(2-hydroxypropyl)amino]carbonyl}pyridin-2-yl)piperazine-1-carboxylate which was used crude. Yield: 5.84 g (100%/).

MS^{m}/z: 397 (M−1).

### (b) tert-Butyl 4-[3-chloro-5-(5-methyl-4,5-dihydro-1,3-oxazol-2-yl)pyridin-2-yl]piperazine-1-carboxylate

tert-Butyl 4-(3-chloro-5-{[(2-hydroxypropyl)amino]carbonyl}pyridin-2-yl)piperazine-1-carboxylate (6.05 g, 15 mmol) and DIPEA (10.6 mL, and 61 mmol) were dissolved in DCM (100 mL) and cooled to 0° C. Methanesulfonyl chloride (1.41 mL, 18 mmol) was added drop-wise over 5 minutes. The reaction mixture was allowed to warm to room temperature and stirred for 16 h followed by heating at reflux for 2 days. The reaction mixture was cooled to room temperature, diluted with DCM (200 mL), washed with saturated NaHCO₃ (3×75 mL), dried (MgSO₄) and concentrated under reduced pressure to yield the crude product. Flash chromatography (1:4 EtOAc/hexanes to 1:1 EtOAc/hexanes) gave tert-butyl 4-[3-chloro-5-(5-methyl-4,5-dihydro-1,3-oxazol-2-yl)pyridin-2-yl]piperazine-1-carboxylate. Yield: 4.86 g (84%).

¹H NMR (400 MHz, CDCl₃): δ 1.42 (3H, d, J=6.2 Hz), 1.49 (1H, s), 3.43-3.45 (4H, m), 3.57-3.62 (4H, m), 4.09-4.15 (1H, m), 4.80-4.89 (1H, m), 8.10 (1H, s), 8.65 (1H, s).

MS^{m}/z: 381 (M+1).

### (c) tert-Butyl 4-[3-chloro-5-(5-methyl-1,3-oxazol-2-yl)pyridin-2-yl]piperazine-1-carboxylate

tert-Butyl 4-[3-chloro-5-(5-methyl-4,5-dihydro-1,3-oxazol-2-yl)pyridin-2-yl]piperazine-1-carboxylate (1.53 g, 4.0 mmol) and DDQ (1.82 g, 8.0 mmol) were dissolved in toluene (200 mL) and heated to 50° C. for 20 h. After cooling to room temperature and the mixture was concentrated under reduced pressure. The reaction mixture was diluted with EtOAc (200 mL), washed with saturated NaHCO₃ (3×75 mL), dried (MgSO₄), passed through a silica gel plug and concentrated under reduced pressure to yield the crude product. Flash chromatography (1:4 EtOAc/hexanes) gave tert-butyl 4-[3-chloro-5-(5-methyl-1,3-oxazol-2-yl)pyridin-2-yl]piperazine-1-carboxylate as a solid. Yield: 0.480g (32%).

¹H NMR (400 MHz, CDCl₃): δ 1.49 (9H, s), 2.39 (3H, s), 3.41-3.44 (4H, m), 3.58-3.61 (4H, m), 6.83 (1H, s), 8.16 (1H, d, J=1.9 Hz), 8.74 (1H, d, J=1.9 Hz).

MS^{m}/z: 379 (M+1).

### (d) 1-[3-chloro-5-(5-methyl-1,3-oxazol-2-yl)pyridin-2-yl]piperazine bis(trifluoroacetate)

tert-Butyl 4-[3-chloro-5-(5-methyl-1,3-oxazol-2-yl)pyridin-2-yl]piperazine-1-carboxylate (0.480 g, 0.1.3 mmol) was dissolved in DCM (30 mL) and TFA (15 mL) and stirred at room temperature for 7 h. The reaction mixture was concentrated under reduced pressure to yield 1-[3-chloro-5-(5-methyl-1,3-oxazol-2-yl)pyridin-2-yl]piperazine bis(trifluoroacetate) as an oil which was used without purification assuming 100% conversion.

### (e) 4-[3-Chloro-5-(5-methyl-1,3-oxazol-2-ylpyridin-2-yl]-N-(phenylsulfonyl)piperazine-1-carboxamide

1-[3-Chloro-5-(5-methyl-1,3-oxazol-2-yl)pyridin-2-yl]piperazine (0.117 g, 0.42 mmol) was dissolved in DCM (10 mL) and DIPEA (1.46 mL, 8.4 mmol) was added. Benzenesulfonyl isocyanate (0.062 mL, 0.46 mmol) was added and the reaction mixture was stirred at room temperature for 18 h. The reaction mixture was concentrated under reduced pressure, diluted with EtOAc (75 mL) and washed with saturated aqueous NH₄Cl (2×25 mL) and brine (25 mL). The organics were dried (MgSO₄) and concentrated under reduced pressure to afford the crude product. Flash chromatography (3:7) EtOAc/hexanes, 0.5% AcOH to 1:1 EtOAc/hexanes, 0.5% AcOH) gave 4-[3-chloro-5-(5-methyl-1,3-oxazol-2-yl)pyridin-2-yl]-N-(phenylsulfonyl)piperazine-1-carboxamide as a solid. Yield: 0.087 g (44%).

¹H NMR (400 MHz, CDCl₃): δ 2.39 (3H, s), 3.44-3.46 (4H, m), 3.56-3.57 (4H, m), 6.84 (In, s), 7.54-7.58 (2H, m), 7.62-7.66 (1H, m), 8.01-8.10 (2H, m), 8.16-8.17 (1H, m), 8.72-8.73 (1H, m).

MS^{m}/z: 462 (M+1).

### Example 40

### 4-[3-Chloro-5-(5-methyl-1,3-oxazol-2-yl)pyridin-2-yl]-N-[(5-chloro-2-thienyl)sulfonyl]piperazin-1-carboxamide

1-[3-Chloro-5-(5-methyl-1,3-oxazol-2-yl)pyridin-2-yl]piperazine (0.117 g, 0.42 mmol) and 2,2,2-trichloroethyl [(5-chloro-2-thienyl)sulfonyl]carbamate (0.157 g, 0.42 mmol) were dissolved in DMA (20 mL) at room temperature. DMAP (0.002 g, 0.02 mmol) and DIPEA (1.46 mL, 8.4 mmol) were added and the system sealed with a screw cap and heated to 100° C. for 3 h. The reaction mixture was cooled to room temperature and the solvent concentrated under reduced pressure. The material was partitioned between EtOAc (25 mL) and saturated aqueous NH₄Cl (25 mL). The organics were washed with brine (25 μL), dried (MgSO₄) and concentrated under reduced pressure to afford the crude product. Flash chromatography (1:4 EtOAc/hexanes to 1:3 EtOAc/hexanes, 1% AcOH) gave 4-[3-chloro-5-(5-methyl-1,3-oxazol-2-yl)pyridin-2-yl]-N-[(5-chloro-2-thienyl)sulfonyl]piperazine-1-carboxamide as a solid. Yield: 0.100 g (44%).

¹H NMR (400 MHz, CDCl₃): δ 2.40 (3H, s), 3.48-3.50 (4H, m), 3.58-3.63 (4H, m), 6.84 (1H, s), 6.95 (1H, d, J=4.3 Hz), 7.67 (1H, d, J=4.3 Hz), 8.17 (1H, s), 8.74 (1H, s).

MS^{m}/z: 502 (M+1).

### Example 41

### 4-[3-Chloro-5-(5-ethyl-1,3-oxazol-2-yl)pyridin-2-yl]-N-(phenylsulfonyl)piperazine-1-carboxamide

### (a) ter-Butyl 4-[3-chloro-5-(5-ethyl-1,3-oxazol-2-yl)pyridin-2-yl]piperazine-1-carboxylate

tert-Butyl 4-[3-chloro-5-(5-ethyl-4,5-dihydro-1,3-oxazol-2-yl)pyridin-2-yl]piperazine-1-carboxylate (1.51 g, 3.8 mmol) and DDQ (1.74 g, 7.7 mmol) were dissolved in toluene (200 nm) and heated to 50° C. for 20 h. After cooling to room temperature, the mixture was concentrated under reduced pressure, diluted with Et(OAc (200 mL), washed with saturated NaHCO₃ (3×75 mL), dried (MgSO₄), passed through a silica gel plug and concentrated under reduced pressure to yield the crude product. Flash chromatography (3:17 EtOAc/hexanes to 1:4 EtOAc/hexanes) gave tert-butyl 4-[3-chloro-5-(5-ethyl-1,3-oxazol-2-yl)pyridin-2-yl]piperazine-1-carboxylate as a solid. Yield: 0.420g (28%).

¹H NMR (400 MHz, CDCl₃): δ 1.31 (31, t, J=7.6 Hz), 10.49 (9H, s), 2.75 (2H, q, J=7.6 Hz), 3.41-3.44 (4H, m), 3.58-3.61 (4H, m), 6.83 (1H, s), 8.17 (1H, d, J=1.9 Hz), 8.75 (1H, d, J=1.9 Hz).

MS^{m}/z: 393 (M+1).

### (b) 1-[3-Chloro-5-(5-ethyl-1,3-oxazol-2-yl)pyridin-2-yl]piperazine bis(trifluoracetate)

tert-Butyl 4-[3-chloro-5-(5-ethyl-1,3-oxazol-2-yl)pyridin-2-yl]piperazine-1 carboxylate (0.480 g, 1.1 mmol) was dissolved in DCM (30 mL) and TFA (15 mL) and stirred at room temperature for 7 h. The reaction mixture was concentrated under reduced pressure to yield 1-[3-chloro-5-(5-ethyl-1,3-oxazol-2-yl)pyridin-2-yl]piperazine bis(trifluoroacetate) as an oil which was used without purification assuming 100% conversion.

### (c) 4-[3-Chloro-(5-ethyl-1,3-oxazol-2-ylpyridin-2-yl]-N-(phenylsulfonyl)piperazin-1-carboxamide

1-[3-Chloro-5-(5-ethyl-1,3-oxazol-2-yl)pyridin-2-yl]piperazine bis(trifluoroacetate) (0.103 g, 0.35 mmol) was dissolved in DCM (10 mL) and DIPEA (1.23 mL, 7.0 mmol) was added. Benzenesulfonyl isocyanate (0.052 mL, 0.39 mmol) was added and the reaction mixture was stirred at room temperature for 18 h. The reaction mixture was concentrated under reduced pressure, diluted with EtOAc (75 mL) and washed with saturated aqueous NH₄Cl (2×25 mL) and brine (25 mL). The organics were dried (MgSO₄) and concentrated under reduced pressure to afford the crude product. Flash chromatography (3:7) EtOAc/hexanes, 0.5% AcOH to 3:2 EtOAc/hexanes, 0.5% AcOH) gave 4-[3-chloro-5-(5-ethyl-1,3-oxazol-2-yl)pyridin-2-yl]-N-(phenylsulfonyl)piperazine-1-carboxamide as a solid. Yield: 0.054 g (94%).

¹H NMR (400 MHz, CDCl₃): δ 1.30 (3H, t, J=7.5 Hz) 2.75 (2H, q, J=7.5 Hz), 3.44-3.46 (4H, m), 3.56-3.57 (4H, m), 6.93 (1H, s), 7.54-7.58 (2H, m), 7.62-7.66 (1H, m), 8.09-8.10 (2H, m), 8.16-8.17 (1H, m), 8.73-8.74 (1H, m).

MS^{m}/z: 476 (M+1).

### Example 42

### 4-[3-Chloro-5-(5-ethyl-1,3-oxazol-2-yl)pyridin-2-yl]-N-[(5-chloro-2-thienyl)sulfonyl]piperazine-1-carboxamide

1-[3-Chloro-5-(5-ethyl-1,3-oxazol-2-yl)pyridin-2-yl]piperazine bis(trifluoroacetate) (0.103 g, 0.35 mmol) and 2,2,2-trichloroethyl [(5-chloro-2-thienyl)sulfonyl]carbamate (0.131 g, 0.35 mmol) were dissolved in DMA (20 mL) at room temperature. DMAP (0.002 g, 0.02 mmol) and DIPEA (1.23 mL, 7.0 mmol) were added and the system sealed with a screw cap and heated to 100° C. for 3 h. The reaction mixture was cooled to room temperature and concentrated under reduced pressure. The material was partitioned between EtOAc (75 mL) and saturated aqueous NH₄Cl (25 mL). The organics were washed with brine (25 mL), dried (MgSO₄) and concentrated under reduced pressure to afford the crude product. Flash chromatography (2:3 EtOAc/hexanes to 99% EtOAc, 1% AcOH) gave 4-[3-chloro-5-(5-ethyl-1,3-oxazol-2-yl)pyridin-2-yl]-N-[(5-chloro-2-thienyl)sulfonyl]piperazine-1-carboxamide as a solid. Yield: 0.089 g (46%).

¹H NMR (400 MHz, CDCl₃): δ 1.31 (3H, t, J=7.6 Hz), 2.75 (2H, q, J=7.6 Hz), 3.48-3.50 (4H, m), 3.58-3.62 (4H, m), 6.84 (1H, s), 6.95 (1H, d, J=4.3 Hz), 7.67 (1H, d, J=4.3 Hz), 8.18 (1H, s), 8.74 (1H, s).

MS^{m}/z: 516 (M+1).

### Example 43

### 4-[3-Chloro-5-(3-methylisoxazol-5-yl)pyridin-2-yl]-N-[(5-chloro-2-thienyl)sulfonyl]piperazine-1-carboxamide

### (a) tert-Butyl 4-[3-chloro-5-(3-methylisoxazol-5-yl)pyridin-2-yl]piperazine-1-carboxylate

To a cooled (0° C.) solution of 2-butanone oxime (0.456 g, 6.2 mmol) in THF (12 mL) was added drop-wise over 5 minutes n-BuLi (2.5 M in hexanes, 4.99 mL, 13 mmol). After 30 minutes, tert-butyl 4-(3-chloro-5-{[methoxy(methyl)amino]carbonyl}-pyridin-2-yl)piperazine-1-carboxylate (2.00 g, 5.2 mmol) in THF (20 mL) was added drop-wise over 20 minutes. After 30 minutes, the solution was poured into concentrated H₂SO₄ (1.0 mL) in THF/water (4:1, 14 mL) and refluxed for 1 h. The reaction mixture was cooled to 0° C. and neutralized with saturated NaHCO₃ (50 mL), diluted with water (100 mL) and extracted with ether (2×50 mL). The combined ethereal extracts were washed with brine (25 mL), dried (MgSO₄), passed through a silica gel plug and concentrated to yield tert-butyl 4-[3-chloro-5-(3-methylisoxazol-5-yl)pyridin-2-yl]piperazine-1-carboxylate which was used without further purification. Yield: 1.17 g (59%).

¹H NMR (400 MHz, CDCl₃): δ 1.49 (9H, s), 2.53 (3H, s), 3.43-3.45 (4H, m), 3.58-3.61 (4H, m), 6.31 (1H, s), 7.94 (1H, br s), 8.53 (1H, br s).

MS^{m}/z: 379 (M+1).

### (b) 1-[3-Chloro-5-(3-methylisoxazol-5-yl)pyridin-2-yl]piperazine dihydrochloride tert-Butyl 4-[3-chloro-5-(3-methylisoxazol-5-yl)pyridin-2-yl]piperazine-1-carboxylate (0.117 g, 3.1 mmol) was dissolved in DCM (30 mL) and HCl (4 M in dioxane, 15.4 mL, 62 mmol) was added and stirred at room temperature for 16 h. The reaction mixture was concentrated under reduced pressure to yield 1-[3-chloro-5-(3-methylisoxazol-5-yl)pyridin-2-yl]piperazine dihydrochloride as a solid which was used without purification. Yield: was not determined, full conversion was assumed.

¹H NMR (400 MHz, d₆-DMSO: δ 2.29 (3H, s), 3.24 (411, br s), 3.60-3.62 (4H, m), 5.04 (2H, br s), 6.96 (1H, s), 8.29 (1H, d, J=11.9 Hz), 8.71 (1H, d, J=1.9 Hz), 9.17 (1H, br s).

MS^{m}/z: 279 (M+1 of freebase).

### (c) 4-[3-Chloro-5-(3-methylisoxazol-5-yl)pyridin-2-yl]-N-[(5-chloro-2-thienyl)sulfonyl]piperazine-1-carboxamide

1-[3-Chloro-5-(3-methylisoxazol-5-yl)pyridin-2-yl]piperazine dihydrochloride (0.150 g, 0.42 mmol) and 2,2,2-trichloroethyl [(5-chloro-2-thienyl)sulfonyl]carbamate (0.159 g, 0.42 mmol) were dissolved in DMA (20 mL) at room temperature. DMAP (0.002 g, 0.02 mmol) and DIPEA (0.742 mL, 4.3 mmol) were added and the system sealed with a screw cap and heated to 100° C. for 3 h. The reaction mixture was cooled to room temperature and concentrated under reduced pressure. The material was partitioned between EtOAc (75 mL) and saturated aqueous NH₄Cl (25 mL). The organics were washed with brine (25 mL), dried (MgSO₄) and concentrated under reduced pressure to afford the crude product. Flash chromatography (2:3 EtOAc/hexanes to 99% EtOAc, 1% AcOH) gave 4-[3-chloro-5-(3-methylisoxazol-5-yl)pyridin-2-yl]-N-[(5-chloro-2-thienyl)sulfonyl]piperazine-1-carboxamide as a solid. Yield: 0.050g (23%).

¹H NMR (400 MHz, d₆-DMSO): 3 2.28 (3H, s), 3.36-3.39 (4H, m), 3.51-3.53 (4H, m), 6.92 (1H, s), 7.24 (1H, d, J=4.1 Hz), 7.62 (1H, d, J=4.1 Hz), 8.23 (1H, d, J=2.0 Hz), 8.67 (1H, d, J=2.0 Hz),

MS^{m}/z: 502 (M+1).

### Example 44

### 4-[3-Chloro-5-(5-ethyl-1,2,4-oxadiazol-3-yl)pyridin-2-yl]-N-[(5-chloro-2-thienyl)sulfonyl]piperazine-1-carboxamide

### (a) tert-Butyl 4-{3-chloro-5-[(hydroxyamino)(imino)methyl]pyridin-2-yl}piperazine-1-carboxylate

tert-Butyl 4-(3-chloro-5-cyanopyridin-2-yl)piperazine-1-carboxylate (3.14 g, 9.7 mmol) and aqueous hydroxylamine (50% by weight, 2.98 mL, 49 mmol) were stirred in EtOH (100 mL) at room temperature for 16 h. The reaction mixture was concentrated under reduced pressure, diluted with EtOAc (200 mL), washed with brine (3×50 mL), dried (MgSO₄), passed through a silica gel plug and concentrated under reduced pressure to afford tert-butyl 4-{3-chloro-5-[(hydroxyamino)(imino)methyl]pyridin-2-yl}piperazine-1-carboxylate. Yield: 2.84 g (84%).

¹H NMR (400 MHz, CDCl₃): δ 1.48 (9H, s), 3.37-3.39 (4H, m), 3.57-3.50 (4H, m), 4.82 (2H, br s), 7.43 (1H, br s), 7.87 (1H, s), 8.40 (1H, s).

MS^{m}/z: 356 (M+1).

### (b) tert-Butyl 4-[3-chloro-5-(5-ethyl-1,2,4-oxadiazol-3-yl)pyridin-2-yl]piperazine-1-carboxylate

tert-Butyl 4-{3-chloro-5-[(hydroxyamino)(imino)methyl]pyridin-2-yl}piperazine-1-carboxylate (0.897 g, 2.5 mmol) was dissolved in pyridine (25 mL) and propionyl chloride (4.4 mL, 5.0 mmol) was added drop-wise. The reaction mixture was heated at reflux for 16 h, cooled to room temperature and concentrated under reduced pressure. The resulting mixture was diluted with EtOAc (200 mL), washed with saturated NH₄Cl (2×50 mL), saturated NaHCO₃ (2×50 mL), dried (MgSO₄), passed through a silica gel plug, and concentrated under reduced pressure to the crude product. Flash chromatography (1:9 EtOAc/hexanes to 1:4 EtOAc/hexanes) gave tert-butyl 4-[3-chloro-5-(5-ethyl-1,2,4-oxadiazol-3-yl)pyridin-2-yl]piperazine-1-carboxylate as a solid. Yield: 0.0.244 g (25%).

¹H NMR (400 MHz, CDCl₃): δ 1.45 (3H, t, J=7.6 Hz), 1.49 (9H, s), 2.97 (2H, q, J=7.6 Hz), 3.45-3.47 (4H, m), 3.58-3.61 (4H, m), 8.23 (1H, s), 8.82 (1H, s).

MS^{m}/z: 394 (M+1).

### (c) 1-[3-Chloro-5-(5-ethyl-1,2,4-oxadiazol-3-yl)pyridin-2-yl]piperazine bis(trifluoroacetate)

tert-Butyl 4-[3-chloro-5-(5-ethyl-1,2,4-oxadiazol-3-yl)pyridin-2-yl]piperazine-1-carboxylate (0.244 g, 0.62 mmol) was dissolved in DCM (20 mL) and TFA (10 mL) and stirred at room temperature for 16 h. The reaction mixture was concentrated under reduced pressure to yield 1-[3-Chloro-5-(5-ethyl-1,2,4-oxadiazol-3-yl)pyridin-2-yl]piperazine bis(trifluoroacetate)as an oil which was used without purification assuming 100% conversion.

### (d) 4-[3-Chloro-5-(5-ethyl-1,2,4-oxadiazol-3-yl)pyridin-2-yl]-N-[(5-chloro-2-thienyl)sulfonyl]piperazine-1-carboxamide

1-[3-Chloro-5-(5-ethyl-1,2,4-oxadiazol-3-yl)pyridin-2-yl]piperazine bis(trifluoroacetate) (0.0910 g, 0.31 mmol) and 2,2,2-trichloroethyl [(5-chloro-2-thienyl)sulfonyl]carbamate (0.115 g, 0.31 mmol) were dissolved in DMA (20 mL) at room temperature. DMAP (0.002 g, 0.02 mmol) and DIPEA (1.08 mL, 6.2 mmol) were added and the system sealed with a screw cap and heated to 100° C. for 3 h. The reaction mixture was cooled to room temperature and the solvent concentrated under reduced pressure. The material was partitioned between EtOAc (75 mL) and saturated aqueous NH₄Cl (25 mL). The organics were washed with brine (25 mL), dried (MgSO₄) and concentrated under reduced pressure to afford the crude product. Flash chromatography (2:3 EtOAc/hexanes to 99% EtOAc, 1% AcOH) gave 4-[3-chloro-5-(5-ethyl-1,2,4-oxadiazol-3-yl)pyridin-2-yl]-N-[(5-chloro-2-thienyl)sulfonyl]piperazine-1-carboxamide as a solid. Yield: 0.088 g (55%).

¹H NMR (400 MHz, CDCl₃): δ 1.45 (3H, t J=7.6 Hz), 2.98 (21 q, J=7.6 Hz), 3.51-3.62 (8H, m), 6.95 (1H, d, J=3.8 Hz), 7.68 (1H, d, J=3.8 Hz), 8.25 (1H, s), 8.83 (1H, s).

MS^{m}/z: 517 (M+1).

### Example 45

### Isopropyl 5-cyano-2-methyl-6-[4-({[(4-methylphenyl)sulfonyl]amino}carbonyl)piperidin-1-yl]nicotinate

### (a) Isopropyl 2-((dimethylamino)methylene)-3-oxobutanoate

Isopropyl 3-oxobutanoate (200 ml, 1365 mmol) was stirred at r.t and dimethoxy-N,N-dimethylmethanamine (242 ml, 1706 mmol) was added drop-wise. The reaction mixture was allowed to stir at r.t overnight. The reaction mixture was concentrated under vacuum and then azeotroped with toluene (3×300 mL) and placed under high vacuum to afford isopropyl 2-((dimethylamino)methylene)-3-oxobutanoate as an oil, which was used without further purification. Yield: 272g (100%).

¹H NMR (400 MHz, CDCl₃): δ 1.30 (6H, d, J=6.2 Hz), 2.32 (3H, s), 5.07-5.17 (1H, m), 7.64 (1H, s).

### (b) Isopropyl 5-cyano-2-methyl-6-oxo-1,6-dihydropyridine-3-carboxylate

NaH (33.359 g, 834.07 mmol) was suspended in THF (700 mL) and 2-cyanoacetamide (58.905 g, 700.62 mmol) added portion-wise at r.t. When gas evolution had stopped a solution of isopropyl 2-(dimethylamino)methylene)-3-oxobutanoate (147.72 g, 667.25 mmol) in THE (300 mL) was added and the system stirred at r.t overnight. The reaction mixture was concentrated under reduced pressure and the solids dissolved in the minimum amount of to hot water. 1N HCl was added to the solution until pH 1 and the solids isolated by filtration. The solids were dried under high vacuum to afford isopropyl 5-cyano-2-methyl-6-oxo-1,6-dihydropyridine-3-carboxylate as a solid, which was used without further purification. Yield: 123g (84%).

¹H NMR (400 MHz, CDCl₃): δ 1.37 (6H, d, J=6.2 Hz), 2.84 (3H, s), 5.18-5.28 (1H, m), 8.50 (1H, s), 13.04 (1H, s).

MS^{m}/z: 221 (M+1).

### (c) Isopropyl 6-chloro-cyano-2-methylnicotinate

Isopropyl 5-cyano-2-methyl-6-oxo-1,6-dihydropyridine-3-carboxylate (123.04 g, 558.70 mmol) was suspended in POCl₃ (204.58 ml, 2234.8 mmol) and heated at 100° C. for 5 h. The reaction mixture was cooled to r.t and concentrated under reduced pressure. The residue was diluted with DCM and poured onto ice. The bi-phasic mixture was stirred at r.t and slowly quenched with solid K₂CO₃ until all the POCl₃ had hydrolysed. The aqueous was extracted into DCM and the organics, dried (MgSO₄) and passed through a silica plug. The organics were concentrated under reduced pressure to afford isopropyl 6-chloro-5-cyano-2-methylnicotinate as a solid, which was used without further purification. Yield: 106g (79%/).

¹H NMR (400 MHz, CDCl₃): δ 1.40 (6H, d, J=6.2 Hz), 2.90 (3H, s), 5.23-5.30 (1H, m), 7.26 (1H, s), 8.46 (1H, s).

MS^{m}/z: 239 (M+1).

### (d) 1-(3-Cyano-5-(isopropoxycarbonyl)-6-methylpyridin-2-yl)piperidine-4-carboxylic acid

Isopropyl 6-chloro-5-cyano-2-methylnicotinate (25.00 g, 104.75 mmol), piperidine-4-carboxylic acid (14.205 g, 109.98 mmol) and DIPEA (54.735 ml, 314.24 mmol) were suspended in EtOH (200 mL) and heated at reflux for 1 h. The reaction mixture was cooled to r.t and added dropwise to KHSO₄ (71.316 g, 523.74 mmol) in water (2000 mL). The solids were collected by filtration and dried under vacuum to afford 1-(3-cyano-5-(isopropoxycarbonyl)-6-methylpyridin-2-yl)piperidine-4-carboxylic acid as a solid, which was used without further purification. Yield: 35g (100%/).

¹H NMR (400 MHz, CDCl₃): δ 1.35 (6H, d, J=6.2 Hz), 1.81-1.93 (2H, m), 2.04-2.12 (2H, m), 2.67-2.74 (4H, m), 3.26-3.36 (2H, m), 4.53-4.62 (2H, m), 5.15-5.23 (1H, m), 8.32 (1H, s).

MS^{m}/z: 332 (M+1).

### (e) Isopropyl 5-cyano-2-methyl-6-[4-({[(4 methylphenyl)sulfonyl]amino}carbonyl)piperidin-1-yl]nicotinate

To 1-[3-cyano-5-(isopropoxycarbonyl)-6-methylpyridin-2-yl]piperidine-4-carboxylic acid (0.100 g, 0.302 mmol)) were added TBTU (0.097 g, 0.302 mmol), dry DCM (2 ml), DIPEA (0.1 ml, 0.57 mmol) and the mixture was stirred at room temperature for 2.5 h. The mixture was added to 4-methylbenzenesulfonamide (0.0616 g, 0.359 mmol), dry DCM (2 ml) was added and the reaction mixture was stirred at room temperature for 18 h. NaHCO₃ (aq) was added and the mixture was extracted with DCM (×3). The combined organic layer was run through a phase separator and solvents were removed in vacuo. The crude product was purified by preparative HPLC (Column: Kromasil C8 10 μm, 21.5×250 mm, Mobilephase A: 100% CH₃CN, Mobilephase B: 5% CH₃CN, 95% 0.1M NH₄OAc(aq) (pH7), Gradient: 20=>50%).

tions was evaporated and freezedried yielding the product isopropyl 5-cyano-2-methyl-6-[4-({[(4-methylphenyl)sulfonyl]amino}carbonyl)piperidin-1-yl]nicotinate as a solid, Yield 0.112 mg (77%)

1H NMR (500 MHz, d₆-DMSO): 1.29 (6H, d), 1.47 (2H, m), 1.82 (2H, m), 2.40 (3H, s), 2.61 (1H, m), 2.62 (3H, s), 3.12 (2H, m), 4.46 (2H, m), 5.07 (1H, m), 7.42 (2H, m), 7.79 (2H, m), 8.29 (1H, s), 12.11 (1H, s).

MS^{m}/z: 485 (M+1), 483 (M-I)

### Example 46

### Isopropyl 5-cyano-2-methyl-6-(4-{[(2-naphthylsulfonyl)amino]carbonyl}piperidin-1-yl)nicotinate

To 1-[3-cyano-5-(isopropoxycarbonyl)-6-methylpyridin-2-yl]piperidine-4-carboxylic acid

(0.100 g, 0.302 mmol)), see example 45, were added TBTU (0.097 g, 0.302 mmol), dry DCM (2 ml), DIPEA (0.1 ml, 0.57 mmol) and the mixture was stirred at room temperature for 2.5 h. The mixture was added to naphthalene-2-sulfonamide (0.0746 g, 0.359 mmol), dry DCM (2 ml) was added and the reaction mixture was stirred at room temperature for 18 h. NaHCO₃ (aq) was added and the mixture was extracted with DCM(x3). The combined organic layer was run through a phase separator and solvents were removed in vacuo. The crude product was purified by preparative HPLC (Column: Kromasil C8 10 μm, 21.5×250 mm, Mobilephase A: 100% CH₃CN, Mobilephase B: 5% CH₃CN, 95% 0.1M NH₄OAc(aq) (pH7), Gradient: 20=>50% B).

tions was evaporated and freezedried yielding the product isopropyl 5-cyano-2-methyl-6-(4-{[(2-naphthylsulfonyl)amino]carbonyl}piperidin-1-yl)nicotinate as a solid, Yield 0.080 m (51%)

¹H NMR (500 MHz, d₆DMSO): 1.28 (6H, d), 1.45 (2H, m), 1.83 (2H, m), 2.60 (3H, s), 2.64 (1H, m), 3.12 (2H, m), 4.45 (2H, m), 5.07 (1H, m), 7.70-8.22 (6H, m), 8.27 (1H, s), 8.60 (1H, s), 12.28 (1H, s).

MS^{m}/z: 521 (M+1), 519 (M−1).

### Example 47

### Ethyl 6-{3-[({[(4-chlorophenyl)sulfonyl]amino}carbonyl)amino]azetidin-1-yl}-5-cyano-2-methylnicotinate

### (a) Ethyl 6-{3-[(tert-butoxycarbonyl)amino]azetidin-1-yl}-5-cyano-2-methylnicotinate

Ethyl 6-chloro-5-cyano-2-methylnicotinate (6.20 g, 29.4 mmol), tert-butyl azetidin-3-ylcarbamate (5.07 g, 29.4 mmol), and DIPEA (5.13 mL, 29.4 mmol) were dissolved in DE (40 mL) and stirred at r.t for 1 h. The reaction mixture was concentrated under reduced pressure and diluted with EtOAc (40 mL). The combined organics were washed with saturated NaHCO₃ (2×30 mL), dried (MgSO₄) and concentrated under reduced pressure to afford the crude product. Flash chromatography (1:6 EtOAc/hexanes) gave ethyl 6-{3-[(tert-butoxycarbonyl)amino]azetidin-1-yl}-5-cyano-2-methylnicotinate as a solid. Yield: 7.00 g (66.0%)

¹H NMR (400 MHz, CDCl₃): δ 1.37 (3H, t, J=7.2 Hz), 1.46 (9H, s), 2.70 (1H, s), 4.18-4.22 (2H₂ m), 4.30 (2H, q, J=7.2 Hz), 4.59 (1H, s), 4.67-4.72 (2H, m), 5.00 (1H, s), 8.26 (1H, s).

MS^{m}/z: 361 (M+1).

### (b) Ethyl 6-(3-aminoazetidin-1-yl)-5-cyano-2-methylnicotinate dihydrochloride

Ethyl 6-(3-(tert-butoxycarbonylamino)azetidin-1-yl)-5-cyano-2-methylnicotinate (1.00 g, 2.77 mmol) was dissolved in DCM (10 mL). HCl (4 M, 13.9 mL, 55.5 mmol) was added slowly. The reaction mixture was stirred at room temperature for 16 h. The mixture was concentrated under reduced pressure to afford ethyl 6-(3-aminoazetidin-1-yl)-5-cyano-2-methylnicotinate dihydrochloride as a solid, which was used crude assuming a 100% conversion.

### (c) Ethyl 6-{3-[({[(4-chlorophenyl)sulfonyl]amino}carbonyl)amino]azetidin-1-yl}-5-cyano-2-methylnicotinate

Ethyl 6-(3-aminoazetidin-1-yl)-5-cyano-2-methylnicotinate dihydrochloride (0.150 g, 0.576 mmol) and DIPEA (0.502 mL, 2.88 mmol) were dissolved in DCM (2 mL), at room temperature. The reaction mixture was cooled to 0° C. 4-chlorobenzenesulfonyl isocyanate (0.103 mL, 0.692 mmol), was slowly added and the system stirred for 2 h at room temperature. EtOAc (40 mL) was added and the combined organics were washed with saturated NaHCO₃ (1×30 mL) and saturated NH₄Cl (1×30 mL). The organics were then dried (MgSO₄) and concentrated under reduced pressure. Flash Chromatography (30 to 50% EtOAc in Hexanes then 50% EtOAc in hexanes with 0.5% AcOH) gave ethyl 6-{3-[({[(4-chlorophenyl)sulfonyl]amino}carbonyl)amino]azetidin-1-yl}-5-cyano-2-methylnicotinate as a solid. Yield: 0.020g (7.26%).

¹H NMR (400 MHz, d₆-DMSO): δ 1.29 (3H, t, J=7.1 Hz), 2.61 (3H, s), 4.07-4.16 (2H, m), 4.23 (2H, q, J=7.1 Hz), 4.39-4.55 (3H, m), 7.34-7.40 (1H, m), 7.70 (21, d, J=8.6 Hz), 7.91 (2H, d, J=8.6 Hz), 8.28 (1H, m), 11.1 (1H, s).

MS^{m}/z: 478 (+1).

### Example 48

### Ethyl 6-{3-[({[(5-chloro-2-thienyl)sulfonyl]amino}carbonyl)amino]azetidin-1-yl}-5-cyano-2-methylnicotinate

Ethyl 6-(3-aminoazetidin-1-yl)-5-cyano-2-methylnicotinate dihydrochloride (0.200 g, 0.600 mmol), see example 47, and 2,2,2-trichloroethyl [(5-chloro-2-thienyl)sulfonyl]carbamate (0.336 g, 0.900 mmol) were dissolved in DMA (2 mL) at room temperature. DIPEA (1.05 mL, 6.00 mmol) were added and the system heated to 100° C. for 1 h. The reaction mixture was cooled to room temperature and the solvent concentrated under reduced pressure. The material was partitioned between EtOAc (40 mL) and saturated aqueous NH₄Cl (2×40 mL). The organics were dried (MgSO₄) and concentrated under reduced pressure to afford the crude product. Flash Chromatography (30 to 50% EtOAc in Hexanes then 50% EtOAc in hexanes with 0.5% AcOH) gave ethyl 6-{3-[({[(5-chloro-2-thienyl)sulfonyl]amino}carbonyl)amino]azetidin-1-yl}-5-cyano-2-methylnicotinate Yield: 0.034 g (10.5%).

¹H NMR (400 MHz, CDCl₃): δ 1.29 (3H, t, J=7.1 Hz), 2.61 (3H, s), 4.11-4.19 (2H, m), 4.23 (2H, A, 1=7.1 Hz), 4.46-4.58 (3H, m), 7.26 (1H, d, J=4.1 Hz), 7.40-7.49 (1H, m), 7.63 (1H, d, J=4.1 Hz).

MS^{m}/z: 512 (M+1).

### Example 49

### Ethyl 6-[4-({[(5-chloro-2-thienyl)sulfonyl]amino}carbonyl)piperidin-1-yl]-5-cyano-2-isopropylnicotinate

### (a) Ethyl 2-((dimethylamino)methylene)-4-methyl-3-oxopentanoate

1,1-Dimethoxy-N,N-dimethylmethanamine (4.96 mL, 37.2 mmol) was added drop-wise to ethyl 4-methyl-3-oxopentanoate (5.00 mL, 31.0 mmol) while stirring at r.t. The reaction mixture was allowed to stir at r.t for 18 h and was then concentrated under reduced pressure and azeotroped with toluene (2×20 mL) producing ethyl 2-((dimethylamino)methylene)-4-methyl-3-oxopentanoate as an oil which was used without purification. Yield: 6.61 g (100%).

¹H NMR (400 MHz, CDCl₃): δ 1.09 (6H, d, J=6.9 Hz), 1.31 (3H, t, J=7.3 Hz), 3.00 (6H, br s), 3.26 (1H, br s), 4.21 (2H, q, J=7.3Hz), 7.60 (1H, s).

### (b) Ethyl 5-cyano-2-isopropyl-4-oxo-1,6-dihydropyridine-3-carboxylate

To a suspension of 2-cyanoacetamide (2.74 g, 32.6 mmol) in THF (100 mL) was added NaH (60% dispersion in mineral oil, 1.36 g, 34.1 mmol. The system was stirred at r.t until gas evolution ceased, at which point ethyl 2-((dimethylamino)methylene)-4-methyl-3-oxopentanoate (6.61 g, 31.0 mmol) was added in one portion. The reaction mixture was stirred at r.t for 18 h and concentrated under reduced pressure to afford a crude intermediate. The solids were dissolved in a minimum amount of warm water and then acidified to pH I with 5 N HCl. Filtration followed by drying under vacuum produced ethyl 5-cyano-2-isopropyl-6-oxo-1,6-dihydropyridine-3-carboxylate. Yield: 6.46 g (89%).

¹H NMR (400 MHz, d₆-DMSO): δ 1.25 (6H, d, J=7.1 Hz), 1.29 (34, t, J=7.3 Hz), 4.01-4.12 (1H, m), 4.23 (2H, q, J=7.3 Hz), 8.43 (1H, s), 12.56 (1H, br s).

MS^{m}/z: 235 (M+1).

### (c) Ethyl 6-chloro-5-cyano-2-isopropylnicotinate

A suspension of ethyl 5-cyano-2-isopropyl-6-oxo-1,6-dihydropyridine-3-carboxylate (6.46 g, 27.6 mmol) in POCl₃ (10.1 mL, 110 mmol) was heated at 100° C. for 6 h. The reaction mixture was poured onto ice and then basified with solid K₂CO₃. The aqueous phase was extracted with DCM (3×100 mL) and the organics was dried (MgSO₄) and concentrated under reduced pressure to afford ethyl 6-chloro-5-cyano-2-ispropylnicotinate, which was used without further purification. Yield: 6.54 g (93%).

¹H NMR (400 MHz, CDCl₃): δ 1.29 (6H, d, J=6.8 Hz), 1.42 (3H, t, J=7.2 Hz), 3.88-3.98 (1H, m), 4.41 (2H, q, J=7.2 Hz), 8.37 (1H, s).

MS^{m}/z: 254 (M+1).

### (d) Ethyl 6-[4-({[(5-chloro-2-thienyl)sulfonyl]amino}carbonyl)piperidin-1-yl]-5-cyano-2-isopropylnicotinate

Ethyl 6-chloro-5-cyano-2-isopropylnicotinate (0.100 g, 0.396 mmol), N-(5-chlorothiophen-2-ylsulfonyl)piperidine-4-carboxamide hydrochloride (0.143 g, 0.415 mmol),see example 159, and DIPEA (0.34 ml, 2.0 mmol) were dissolved in DMA (10 ml) and the reaction was heated to 60° C. overnight. The reaction mixture was diluted with EtOAc (125 mL) and washed sequentially with saturated aqueous NH₄Cl (2×50 ml), water (3×40 ml) and brine (40 ml). The organics were dried (MgSO₄) and concentrated under reduced pressure to afford the crude material which was purified by column chromatography (25% EtOAc/hexanes then 0.05% AcOH added) to provide ethyl 6-[4-({[(5-chloro-2-thienyl)sulfonyl]amino}carbonyl)piperidin-1-yl]-5-cyano-2-isopropylnicotinate as a solid. Yield: 0.082 g (39%).

¹H NMR (400 MHz, CDCl₃): δ 1.20 (6H, d, J=6.7 Hz), 1.38 (31H, t, J=7.1 Hz), 1.76-1.86 (2H, m), 1.95-1.99 (2H, m), 2.50-2.57 (1H, m), 3.18-3.25 (2H, m), 3.94-4.04 (1H, m), 4.32 (2H, q, J=7.1 Hz), 4.65468 (2H, d), 6.97 (1H, d, J=4.1 Hz), 7.70 (1H, d, J=4.1 Hz), 8.14 (I ,br) 58.31 (1H, s).

MS^{m}/z: 525 (M+1).

### Example 50

### Ethyl 6-[4-({[(5-chloro-2-thienyl)sulfonyl]amino}carbonyl)piperidin-1-yl]-5-cyano-2-phenylnicotinate

Employing the same methodology which produced ethyl 6-[4-({[(5-chloro-2-thienyl)sulfonyl]amino}carbonyl)piperidin-1-yl]-5-cyano-2-ethylnicotinate (example 51) from ethyl 3-oxopentanoate, ethyl 6-[4-({[(5-chloro-2-thienyl)sulfonyl]amino}carbonyl)piperidin-1-yl]-5-cyano-2-phenylnicotinate was generated from ethyl 3-oxopentanoate.

¹H NMR (400 MHz, CDCl₃): δ 1.09 (3H, t, J=7.1 Hz), 1.78-1.88 (2H, m), 1.95-2.00 (2H, m), 2.49-2.56 (1H, m), 3.20-3.27 (2H, m), 4.14 (2H, q, J=7.1 Hz), 4.64-4.67 (2H, m), 6.96 (1H, d, J=4.1 Hz), 7.39-7.45 (3H, m), 7.48-7.50 (2H, m), 7.69 (1H, d, J=4.1 Hz), 8.32 (1H, s), 8.36 (1H, br s),

MS^{m}/z: 559 (M+1).

### Example 51

### Ethyl 6-[4-({[(5-chloro-2-thienyl-6-sulfonyl]amino}carbonyl piperidin-1-yl]-5-cyano-2-ethylnicotinate

### (a) Ethyl 2-((dimethylamino)methylene)-3-oxopentanoate

1,1-Dimethoxy-N,N-dimethylmethanamine (5.09 mL, 42.0 mmol) was added drop-wise to ethyl 3-oxopentanoate (5.0 mL, 35.0 mmol) while stirring at r.t. The reaction mixture was stirred at r.t for 18 b and then was concentrated under reduced pressure and azeotroped with toluene (2×20 mL) producing ethyl 2-(dimethylamino)methylene)-3-oxopentanoate as an oil which was used without purification. Yield. 6.98 g (100%).

¹H NMR (400 MHz, CDCl₃): δ 1.10 (3H, t, J=7.7 Hz), 1.32 (3H, t, J=7.7 Hz), 2.67-2.69 (2H, m), 3.01 (6H, br s), 4.22 (2H, q, J=7.2 Hz), 7.64 (1H, s).

### (b) Ethyl 5-cyano-2-ethyl-6-oxo-1,6-dihydropyridine-3-carboxylate

To a suspension of 2-cyanoacetamide (3.09 g, 36.8 mmol) in THF (100 mL) was added NaH (60% dispersion in mineral oil, 1.54 g, 38.5 mmol). The mixture was stirred at r.t until gas evolution ceased, at which point ethyl 2-((dimethylamino)methylene)-3-oxopentanoate (6.98 g, 35.0 mmol) was added in one portion. The reaction mixture was stirred at r.t for 18 h and concentrated under reduced pressure to afford crude intermediate. The solids were dissolved in a minimum amount of warm water and then acidified to pH 1 with 5 M HCl. Filtration followed by drying under vacuum produced ethyl 5-cyano-2-ethyl-6-oxo-1,6-dihydropyridine-3-carboxylate as a solid. Yield: 6.28 g (81%).

¹H NMR (400 MHz, DMSO-d₆): δ 1.18 (3H, t, J=7.3 Hz), 1.29 (3H, t, J=7.0 Hz), 2.95 (2H, q, J=7.3 Hz), 4.24 (2H, q, J=7.0 Hz), 8.45 (1H, s), 12.79 (1H, br s).

MS^{m}/z: 221 (M+1).

### (c) Ethyl 6-chloro-5-cyano-2-ethylnicotinate

A suspension of ethyl 5-cyano-2-ethyl-6-oxo-1,6-dihydropyridine-3-carboxylate (6.28 g, 28.5 mmol) in POCl₃ (10.4 mL, 114 mmol) was heated to 100° C. for 6 h. The reaction mixture was poured onto ice and then basified with solid K₂CO₃. The aqueous phase was extracted with DCM (3×100 mL) and the organics dried (MgSO₄) and concentrated under reduced pressure to afford ethyl 6-chloro-5-cyano-2-ethylnicotinate as a solid, which was used without further purification. Yield: 6.17 g (91%). ¹H NM (400 MHz, CDCl₃): δ 1.32 (3H, t, J=7.4 Hz), 1.42 (3H, t, J=7.4 Hz), 3.23 (2H, q, J=7.4 Hz), 4.42 (2H, q, J=7.4 Hz), 8.45 (1H, s).

MS^{m}/z: 239 (M+1).

### (d) Ethyl 6-[4-({[(5-chloro-2-thienyl)sulfonyl]amino}carbonyl)piperidin-1-yl]-5-cyano-2-ethylnicotinate

A solution of ethyl 6-chloro-5-cyano-2-ethylnicotinate (0.100 g, 0.419 mmol), N-(5-chlorothiophen-2-ylsulfonyl)piperidine-4-carboxamide hydrochloride (0.152 g, 0.440 mmol), see example 159, and DIPEA (0.365 mL, 2.10 mmol) in DMA (10 mL) was heated to 60° C. for 20 h. Following concentration, the mixture was diluted with EtOAc (100 mL), washed with saturated NH₄Cl (2×50 mL), water (3×50 mL), brine (50 mL), dried (MgSO₄) and concentrated. Flash chromatography (25% EtOAc/hexanes with 1% AcOH) furnished Ethyl 6-[4-({[(5-chloro-2-thienyl)sulfonyl]amino}carbonyl)piperidin-1-yl]-5-cyano-2-ethylnicotinate as a solid. Yield: 0.185 g (86%).

¹H NMR (400 MHz, CDCl₃): δ 1.23 (3H, t, J=7.4 Hz), 1.37 (3H, t, J=7.1 Hz), 1.77-1.87 (2H, m), 1.95-1.99 (2H, m), 2.50-2.57 (1H, m), 3.12 (2H, q, J=7.4 Hz), 3.18-3.24 (2H, m), 4.32 (2H, q, J=7.1 Hz), 4.66-4.69 (2H, m), 6.97 (1H, d, J-4.1 Hz), 7.70 (1H, d, J=4.1 Hz), 8.27 (1H, br s), 8.33 (1H, s).

MS^{m}/z: 511 (M+1).

### Example 52

### tert-Butyl 6-[4-({[(5-chloro-2-thienyl)sulfonyl]amino}carbonyl)piperidin-1-yl]-5-cyano-2-methylnicotinate

### (a) Benzyl 2-[(dimethylamino)methylene]-3-oxobutanoate

Benzyl 3-oxobutanoate (82 ml, 475 mmol) was stirred at r.t and 1,1-dimethoxy-N,N-dimethylmethanamine (76 ml, 570 mmol) was added drop-wise. The reaction mixture was allowed to stir at r.t overnight. The reaction mixture was concentrated under vacuum and then azeotroped with toluene (3×200 mL) and placed under high vacuum to afford Benzyl 2-[(dimethylamino)methylene]-3-oxobutanoate as an oil, which was used without further purification. Yield: 117g (100%).

¹H NMR (400 MHz, CDCl₃): δ 2.32 (3H, s), 3.02 (6H, br s), 5.22 (2H, s), 7.29-7.43 (5H, m), 7.70 (1H, s).

### (b) Benzyl 5-cyano-2-methyl-6-oxo-1,6-dihydropyridine-3-carboxylate

NaH (19.9 g, 498 mmol) was added to a stirred r.t suspension of 2-cyanoacetamide (39.9 g, 475 mmol) in THF (1000 mL). The reaction mixture was stirred at r.t until gas evolution stopped. Benzyl 2-[(dimethylamino)methylene]-3-oxobutanoateas (117.4 g, 474.7 mmol) was added portion-wise and the reaction mixture stirred at r.t overnight. 1N HCl was added and the system stirred at r.t for 1 h and then the reaction mixture was diluted with EtOAc and extracted. The organics were dried (MgSO₄) and concentrated under reduced pressure to afford benzyl 5-cyano-2-methyl-6-oxo-1,6-dihydropyridine-3-carboxylate as a solid, which was used without further purification. Yield: 111 g (88%).

¹H NMR (400 MHz, d₆-DMSO): δ 2.63 (3H, s), 5.29 (2H, s), 7.34-7.47 (5H, m), 8.72 (1H, s), 12.82 (1H, s).

MS^{m}/z: 267 (M−1).

### (c) 6-Chloro-5-cyano-2-methylnicotinic acid

Benzyl 5-cyano-2-methyl-6-oxo-1,6-dihydropyridine-3-carboxylate was suspended in POCl₃ (43.44 ml, 474.5 mmol) and heated at 100° C. overnight. The reaction mixture was cooled to r.t and poured onto ice. The aqueous was neutralized with solid NaHCO₃ and extracted into DCM. The organics were dried (MgSO₄) and concentrated under reduced pressure to afford the material. Flash chromatography (gradient elution 30-50% EtOAc/Hexanes, 0.5% AcOH) gave crude 6-Chloro-5-cyano-2-methylnicotinic acid as a solid. Yield: 24.2 g (26%).

¹H NMR (400 MHz, CDCl₃): δ 3.00 (3H, s), 8.50 (1H, s).

MS^{m}/z: 195 (M−1).

### (d) tert-Butyl 6-chloro-5-cyano-2-methylnicotinate

A solution of 6-Chloro-5-cyano-2-methylnicotinic acid (6.10g, 31.0 mmol) and tert-butyl N,N′-diisopropylcarbamimidate (18.6 g, 93.1 mmol) in THF (150 mL) was heated to reflux for 20 h. The reaction mixture was cooled to room temperature, concentrated and diluted with DCM (300 mL). The resulting precipitate was removed by filtration through silica gel and discarded. The supernatant was concentrated, diluted with EtOAc (400 mL), washed with saturated NH₄Cl (2×200 mL), saturated NaHCO₃ (2×200 mL), brine (200 mL), dried (MgSO₄), passed through silica gel and concentrated. Flash chromatography (50% DCM/hexanes) furnished tert-butyl 6-chloro-5-cyano-2-methylnicotinate as a solid. Yield: 2.75 g (35%).

¹H NMR (400 MHz, CDCl₃): δ 1.61 (9H, s), 2.87 (3H, s), 8.39 (1H, s).

MS^{m}/z: 254 (M+1).

### (e) tert-Butyl 6-[4-({[(5-chloro-2-thienyl)sulfonyl]amino}carbonyl)piperidin-1-yl]-5-cyano-2-methylnicotinate

A solution of tert-butyl 6-chloro-5-cyano-2-methylnicotinate (0.0977 g, 0.387 mmol), N-(5-chlorothiophen-2-ylsulfonyl)piperidine-4-carboxamide hydrochloride (0.133 g, 0.387 mmol), see example 158, and DIPEA (0.278 mL, 1.55 mmol) in DMF (5 mL) was heated to 80° C. for 20 h. Following concentration, the mixture was diluted with EtOAc (100 mL), washed with saturated NH₁₄Cl (2×50 mL), brine (50 mL), dried (MgSO₄) and concentrated. Flash chromatography (50% EtOAc/hexanes with 1% AcOH) furnished tert-Butyl 6-[4-({[(5-chloro-2-thienyl)sulfonyl]amino}carbonyl)piperidin-1-yl]-5-cyano-2-methylnicotinate as a solid. Yield: 0.180g (84%).

¹H NMR (400 MHz, CDCl₃): δ 1.57 (9H, s), 1.76-1.36 (2H, m), 1.95-1.99 (2H, m), 2.50-2.57 (1H, m), 2.68 (3H, s), 3.14-3.21 (21, m), 4.61-4.64 (2H, m), 6.96 (1H, d, J=4.0 Hz), 7.69 (1H, d, J=4.0 Hz), 8.25 (1H, s), 8.42 (11K, br s).

MS^{m}/z: 526 (M+1).

### Example 53

### 2,2-Dimethylpropyl 6-{3-[({[(5-chloro-2-thienyl)sulfonyl]amino}carbonyl)amino]azetidin-1-yl}-5-cyano-2-methylnicotinate

### (a) 6-(3-[(tert-Butoxycarbonyl)amino]azetidin-1-yl)-5-cyano-2-methylnicotinic acid

Ethyl 6-(3-(tert-butoxycarbonyl)azetidin-1-yl)-5-cyano-2-methylnicotinate (1.50 g, 4.16 mmol), see example 47 and lithium hydroxide (3.00 g, 8.32 mmol) were suspended in MeOH (40 mL) and heated at 90° C. for 1 h. HCl (conc.) was added drop-wise to the mixture until the pH was lowered to pH 2. The precipitate was filtered and collected. The mother liquor was washed with EtOAc (1×60 mL), dried (MgSO₄), concentrated under reduced pressure and combined with the solid to afford 6-{3-[(tert-butoxycarbonyl)amino]azetidin-1-yl}-5-cyano-2-methylnicotinic acid as a solid, which was used crude

### (b) 2,2-dimethylpropyl 6-(3-(tert-butoxycarbonylamino)azetidin-1-yl)-5-cyano-2-methylnicotinate

2,2-dimethylpropyl 6-{3-[6-(3-[(tert-Butoxycarbonyl)amino]azetidin-1-yl}-5-cyano-2-methylnicotinic acid (0.400 g, 1.20 mmol), 1-iodo-2,2-dimethylpropane (0.320 mL, 2.40 mmol), and potassium carbonate (0.216 g, 1.57 mmol) were dissolved in DMA (5 mL). The reaction mixture was heated at 90° C. for 56 h. The reaction mixture was diluted with EtoAc (40 mL). The combined organics were washed with saturated NaHCO₃ (2×40 mL), dried (MgSO₄) and concentrated under reduced pressure to afford 2,2-dimethylpropyl 6-(3-(tert-butoxycarbonylamino)azetidin-1-yl)-5-cyano-2-methylnicotinate as a solid, which was used crude assuming a 100% conversion.

¹H NMR (400 MHz, CDCl₃): δ 1.02 (9H, s), 1.46 (9H, s), 2.72 (3H, s), 3.95 (2H, s), 4.16-4.26 (2H, m), 4.54-4.77 (3H, m), 4.99 (1H, s), 8.25 (1H, s).

MS^{m}/z: 403 (M+1).

### (c) 2,2-Dimethylpropyl 6-(3-aminoazetidin-1-yl)-5-cyano-2-methylnicotinate bis(trifluoroacetate)

2,2-Dimethylpropyl 6-(3-(tert-butoxycarbonylamino)azetidin-1-yl)-5-cyano-2-methylnicotinate (0.388 g, 0.964 mmol) was dissolved in DCM (5 mL). TFA (1.11 mL, 14.5 mmol) was added slowly. The reaction mixture was stirred at room temperature for 30 minutes. The mixture was concentrated and azeotroped (Toluene, Hexanes) to afford ethyl 6-(3-aminoazetidin-1-yl)-5-cyano-2-methylnicotinate bis(trifluoroacetate) as a solid, which was used crude assuming a 100% conversion.

### (d) 2,2-Deimethylpropyl 6-{3-[({[(5-chloro-2-thienyl)sulfonyl]amino}carbonyl)amino]azetidin-1-yl}-5-cyano-2-methylnicotinate

2,2-dimethylpropyl 6-(3-aminoazetidin-1-yl)-5-cyano-2-methylnicotinate bis(trifluoroacetate) (0.421 g, 0.292 mmol), 2,2,2-trichloroethyl [(5-chloro-2-thienyl)sulfonyl]carbamate (0.131 g, 0.350 mmol) and DIPEA (0.762 mL, 4.38 mmol) were dissolved in DMA (2 mL) and heated at 100° C. for 3 h. The reaction mixture was cooled to room temperature and the reaction mixture concentrated under reduced pressure, EtOAc (40 mL) was added and the organics were washed with saturated aqueous NH₄Cl (2×30 mL), brine (40 mL), dried (MgSO₄) and concentrated under reduced pressure to afford the crude product. Flash chromatography (20-50% EtOAc in Hexanes then 20-50% EtOAc in Hexanes with 0.5% AcOH) gave 2,2-dimethylpropyl 6-{3-[({[(5-chloro-2-thienyl)sulfonyl]amino}carbonyl)amino]azetidin-1-yl}-5-cyano-2-methylnicotinate as a solid. Yield: 0.022 g (14.3%).

¹H NMR (400 MHz, d₆-DMSO): d 0.977 (9H, s), 2.63 (3H, s), 3.91 (2H, s), 4.09-4.21 (2H, m), 4.46-4.58 (3H, m), 7.26 (1H, d, J=4.1 Hz), 7.41-7.47 (1H, m), 7.62 (1H, d, J=4.1 Hz), 8.28 (1H, s), 11.29-11.48 (1H, m).

MS^{m}/z: 526 (M+1).

### Example 54

### 2,2-Dimethylpropyl 6-[4-({[(5-chloro-2-thienyl)sulfonyl]amino}carbonyl)piperidin-1-yl]-5-cyano-2-methylnicotinate

### (a) Ethyl 6-(4-(tert-butoxycarbonyl)piperidin-1-yl)-5-cyano-2-methylnicotinate

A solution of ethyl 6-chloro-5-cyano-2-methylnicotinate (6.00 g, 26.7 mmol), tert-butyl piperidine-4-carboxylate hydrochloride (6.51, 29.4 mmol) and DIPEA (23.3 mL, 134 mmol) in DMA (50 mL) were heated to 80° C. for 2 h. After cooling to room temperature, the reaction mixture was diluted with EtOAc (300 mL), washed with saturated NH₄Cl (4×50 mL), brine (50 mL), dried (MgSO₄), passed through silica gel and concentrated. Flash chromatography produced Ethyl 6-(4-(tert-butoxycarbonyl)piperidin-1-yl)-5-cyano-2-methylnicotinate as a solid. Yield 8.85 g (89%).

¹H NMR (400 MHz, CDCl₃): δ 1.37 (3H, t, J=7.1 Hz), 1.45 (9H, s), 1.75-1.84 (2H, m), 1.99-2.03 (2H, m), 2.49-2.57 (1H, m), 2.72 (3H, s), 3.24-3.31 (2H, m), 4.31 (2H, q, J=7.1 Hz), 4.55-4.60 (2H, m), 8.34 (1H, s).

MS^{m}/z: 374 (M+1).

### (b) 6-(4-(ter-Butoxycarbonyl)piperidin-1-yl)-5-cyano-2-methylnicotinic acid

To a solution of ethyl 6-(4-(tert-butoxycarbonyl)piperidin-1-yl)-5-cyano-2-methylnicotinate (6.65 g, 17.8 mmol) in THF 50 mL was added aqueous LiOH (1.0 M, 107 mL, 107 mmol) and the mixture was heated to reflux for 5 h. After cooling to room temperature, the reaction was acidified to pH 3.5 with 2 M HCl and extracted into EtOAc (4×50 mL). The organic extracts were washed with brine, dried (MgSO₄), passed trough silica gel and concentrated. Flash chromatography (20% EtOAc/hexanes with 1% AcOH) furnished 6-(4-(tert-Butoxycarbonyl)piperidin-1-yl)-5-cyano-2-methylnicotinic acid as a solid. Yield 1.8 g (29%)

¹H NMR (400 MHz, d₆-DMSO): δ 1.41 (9H, s), 1.53-1.63 92H, m), 1.90-1.94 (2H, m), 2.55-2.60 (1H, m), 2.64 (3H, s), 3.21-3.28 2H, m), 4.40-4.44 (2H, m), 8.30 (1H, s), 12.91 (1H, br s).

MS^{m}/z: 350 (M+1).

### (c) 2,2-Dimethylpropyl 6-[4-(ten-butoxycarbonyl)piperidin-1-yl]-5-cyano-2-methylnicotinate

A solution of 6-(4-(tert-butoxycarbonyl)piperidin-1-yl)-5-cyano-2-methylnicotinic acid (0.845 g, 2.45 mmol), 2,2-dimethylpropan-1-ol (1.30 g, 14.7 mmol), EDCI (2.11 g, 11.0 mmol), HOBt (0.496 g, 3.67 mmol) and DIPEA (0.852 mL, 4.89 mmol) were heated to 80° C. for 2 days. The reaction mixture was diluted with EtOAc (50 ml), washed with saturated NH₄Cl (3×30 mL), brine, dried (MgSO₄), passed through silica gel and concentrated. Flash chromatography (3% EtOAc/hexanes) yielded 2,2-dimethylpropyl 6-[4-(tert-butoxycarbonyl)piperidin-1-yl]-5-cyano-2-methylnicotinate as a solid. Yield: 1.02 g (41%).

¹H NMR (400 MHz, CDCl₃): δ 1.02 (9H, s), 1.46 (9H, s), 1.76-1.85 (2H, m), 2.00-2.03 (2H, m), 2.49-2.57 (1H, m), 2.73 (3H, M), 3.25-3.31 (2H, m), 3.96 (2H, s), 4.56-4.60 (2H, m), 8.32 (1H, s).

MS^{m}/z: 416 (N+1).

### (d) 1-{3-Cyano-5-[(2,2-dimethylpropoxy)carbonyl]-4-methylpyridin-2-yl}piperidine-4-carboxylic acid

To a solution of 2,2-dimethylpropyl 6-[4-(tert-butoxycarbonyl)piperidin-1-yl]-5-cyano-2-methylnicotinate (0.415 g, 0.999 mmol) in DCM (10 mL) at 0° C. was added TFA (10 mL) and the reaction mixture was stirred for 2 h. Concentration produced 1-{3-cyano-5-[(2,2-dimethylpropoxy)carbonyl]-6-methylpyridin-2-yl}piperidine-4-carboxylic acid which was used crude assuming 100% conversion.

MS^{m}/z: 513 (M+1).

### (e) 2,2-Dimethylpropyl 6-[4-({[(5-chloro-2-thienyl)sulfonyl]amino}carbonyl)piperidin-1-yl]-5-cyano-2-methylnicotinate

A solution of 1-{3-cyano-5-[(2,2-dimethylpropoxy)carbonyl]-6-methylpyridin-2-yl}piperidine-4-carboxylic acid (0.120 g, 0.334 mmol), EDCI (0.083 g, 0.434 mmol), and HOBt (0.052 g, 0.334 mmol), 5-chlorothiophene-2-sulfonamide (0.080 g, 0.401 mmol) and DIPEA (0.291 mL, 1.67 mmol) in DCM (7.0 mL) was stirred at room temperature for 20 h. Following concentration, the mixture was diluted with EtOAc (100 mL), washed with saturated NH₄Cl (2×50 mL), saturated NaHCO₃ (2×50 mL), brine (50 mL), dried (MgSO₄) and concentrated. Flash chromatography (20% EtoAc/hexanes with 1% AcOH) furnished neopentyl 2,2-dimethylpropyl 6-[4-({[(5-chloro-2-thienyl)sulfonyl]amino}carbonyl)piperidin-1-yl]-5-cyano-2-methylnicotinate as a solid. Yield: 0.068 g (36%).

¹H NMR (400 MHz, CDCl₃): δ 1.02 (9H, s), 1.77-1.87 (2H, m), 1.96-2.00 (2H, m), 2.51-2.58 (1H, m), 2.73 (3H, s), 3.17-3.24 (2H, m), 3.97 (2H, s), 4.64-4.67 (2H, m), 6.97 (1H, d, J=4.1 Hz), 7.70 (1H, d, J=4.1 Hz), 8.29 (1H, br s), 8.32 (1H, s).

MS^{m}/z: 540 (M+1).

### Example 55

### Isopropyl 5-cyano-2-methyl-[4-({[(5-methyl-2-thienyl)sulfonyl]amino}carbonyl)piperidin-1-yl]nicotinate

Using the methodology that produced 2,2-dimethylpropyl 6-[4-({[(5-chloro-2-thienyl)sulfonyl]amino}carbonyl)piperidin-1-yl]-5 cyano-2-methylnicotinate (example 67) from 2,2-dimethylpropan-1-ol, isopropyl 5-cyano-2-methyl-6-[4-({[(5-methyl-2-thienyl)sulfonyl]amino}carbonyl)piperidin-1-yl]nicotinate was generated from isopropyl alcohol.

¹H NMR (400 MHz, CDCl₃): δ 1.35 (6H, d, J=6.3 Hz), 1.76-1.86 (2H, m), 1.95-1.99 (2H, m), 2.49-2.56 (1H, m), 2.71 (31, s), 3.15-3.22 (2H, m), 4.64-4.67 (2H, m), 5.16-5.22 (1H, m), 6.97 (1H, d, J=4.1 Hz), 7.70 (1H, d, J=4.1 Hz), 8.07 (1H, br s), 8.32 (1H, s).

MS^{m}/z: 512 (M+1).

### Example 56

### Ethyl 5-cyano-2-methyl-6-[3-{[(3-methylphenyl)sulfonyl]amino}carbonyl)azetidin-1-yl]nicotinate

### (a) Ethyl 2-((dimethylamino)methylene)-3-oxobutanoate

Ethyl 3-oxobutanoate (250 ml, 1961 mmol) was stirred at r.t and 1,1-dimethoxy-N,N-dimethylmethanamine (327 ml, 2452 mmol) was added drop-wise. The reaction mixture was allowed to stir at r.t overnight. The reaction mixture was concentrated under vacuum and then azeotroped with toluene (3×300 mL) and placed under high vacuum to afford ethyl 2-((dimethylamino)methylene)-3-oxobutanoate as an oil, which was used without further purification. Yield: 363g (100%).

¹H NMR (400 MHz, CDCl₃): δ 2.32 (3H, s), 3.02 (6H, br s), 5.22 (2H, s), 7.29-7.43 (5H, m), 7.70 (1H, s).

MS^{m}/z: 186 (M+1).

### (b) Ethyl 5-cyano-2-methyl-6-oxo-1,6-dihydropyridine-3-carboxylate

2-Cyanoacetamide (33.0 g, 392 mmol) was suspended in THF (250 mL) and slowly added to a suspension of NaH (60% dispersion in mineral oil, 16.5 g, 412 mmol) in THF (500 mL). The mixture was stirred for 2 h at r.t followed by the drop-wise addition of ethyl 2-((dimethylamino)methylene)-3-oxobutanoate (72.6 g, 392 mmol) suspended in THF (250 mL). The reaction mixture was stirred at r.t for 16 h and then acidified to pH 6 with acetic acid. Concentration under reduced pressure afforded crude material, which was suspended in 1 N HCl (1000 mL) and stirred for 30 minutes. The suspension was filtered and the product collected as a solid, which was azeotroped with Toluene (3×1000 mL) to afford ethyl 5-cyano-2-methyl-6-oxo-1,6-dihydropyridine-3-carboxylate as a solid. Yield: 75.3 g (93%).

¹H NMR (400 MHz, d₆-DMSO): δ 1.36 (3H, t, J=7.1 Hz), 2.62 (3H, s), 4.25 (2H, q, J=7.1 Hz), 8.71 (1H, s), 12.79 (1H, br s).

### (c) Ethyl 6-chloro-5-cyano-2-methylnicotinate

Ethyl 5-cyano-2-methyl-6-oxo-1,6-dihydropyridine-3-carboxylate (70.33 g, 341.1 mmol) was suspended in POCl₃ (124.5 ml, 1364 mmol) and the system heated at 100° C. overnight. The reaction mixture was cooled to r.t and concentrated under reduced pressure. The residue was diluted with DCM and poured onto ice. The bi-phasic mixture was stirred at r.t and slowly quenched with solid K₂CO₃ until all the POCl₃ had hydrolysed. The aqueous was extracted into DCM and the organics, dried (MgSO₄) and passed through a silica plug. The organics were concentrated under reduced pressure to afford ethyl 6-chloro-5-cyano-2-methylnicotinate as a solid, which was used without further purification. Yield: 61g (80%).

¹H NMR (400 MHz, CDCl₃): δ 1.42 (3H, t, J=7.1 Hz), 2.91 (3H, s), 4.40 (2H, q, J=7.1 Hz), 8.49 (1H, s).

### (d) 1-[3-Cyano-5-(ethoxycarbonyl)-6-methylpyridine-2-yl]azetidine-3-carboxylic acid

Ethyl 6-chloro-5-cyano-2-methylnicotinate (50.98 g, 227 mmol), azetidine-3-carboxylic acid (24.09 g, 238 mmol) and DIPEA (118.9 mL, 681 mmol) were suspended in EtOH (250 mL) and heated at reflux for 1 h. The reaction mixture was cooled to r.t and added drop-wise to KHSO₄ (154.5 g, 1135 mmol) in water (3000 mL). The solids were collected by filtration and dried under vacuum to afford 1-[3-Cyano-5-(ethoxycarbonyl)-6-methylpyridine-2-yl]azetidine-3-carboxylic acid as a solid, which was used without further purification. Yield: 65.33 g (100%).

¹H NMR (400 MHz, CDCl₃): δ 1.37 (3H, t, J=7.1 Hz), 2.72 (31H, s), 3.59-3.68 (1H, m), 4.31 (2H, q, J=7.1 Hz), 4.55-4.68 (4H, m), 8.28 (1H, s).

MS^{m}/z: 290 (M+1).

### (e) Ethyl 5-cyano-2-methyl-6-[3-({[(3-methylphenyl)sulfonyl]amino}carbonyl)azetidin-1-yl]nicotinate

Prepared according to method A starting from 3-methylbenzenesulfonamide (0.100 g, 0.38 mmol). Yield: 0.028 g (25%).

¹H NMR (400 MHz, d₆₆-DMSO) 8, 1.22 (3H, t J=7.2 Hz), 2.34 (3H, s), 2.53 (3H, s), 3.49 (1H, m), 4.13 (2H, m), 4.16 (2H, q, J=7.1 Hz), 4.35 (2H, t, J=9.1 Hz), 7.66 (2H, s), 7.46 (2H, m), 8.20 (1H, s),

MS^{m}/z: 443 (M+1)

### Example 57

### Ethyl 5-cyano-2-methyl-6-[3-({[(phenylsulfonyl)amino]carbonyl}amino)azetidin-1-yl]nicotinate

Ethyl 6-(3-aminoazetidin-1-yl)-5-cyano-2-methylnicotinate dihydrochloride (0.150 g, 0.576 mmol) and DIPEA (0.502 mL, 2.88 mmol) were dissolved in CH₂Cl₂ (2 mL), at room temperature. The reaction mixture was cooled to 0° C. 4-chlorobenzenesulfonyl isocyanate (0.103 mL, 0.692 mmol), was slowly added and the system stirred for 2 h at room temperature. EtOAc (40 mL) was added and the combined organics were washed with saturated NaHCO₃ (1×30 mL) and saturated NH₄Cl (1×30 mL). The organics were then dried (MgSO₄) and concentrated under reduced pressure. Flash Chromatography (40% EtOAc in Hexanes then 50% EtOAc in hexanes with 0.5% AcOH) gave ethyl 5-cyano-2-methyl-6-[3-({[(phenylsulfonyl)amino]carbonyl}amino)azetidin-1-yl]nicotinate product as a solid. Yield: 0.224 g (62.5%).

¹H NMR (400 MHz, CDCl₃): δ 1.38 (3H, t J=7.1 Hz), 2.72 (3H, s), 4.15-4.27 (2H, m), 4.32 (2H, q, J=7.1 Hz), 4.61-4.77 (3H, m), 7.11 (1H, s), 7.53-7.62 (2H, m), 7.64-7.73 (1H, m), 7.84-7.93 (2H, m), 8.29 (1H, s).

MS^{m}/z: 444 (M+1).

### Example 58

### 1-[3-Chloro-5-(5-ethyl-1,3-oxazol-2-yl %6-(methylamino)pyridin-2-yl]-N-[(5-chloro-2-thienyl)sulfonyl]piperidine-4-carboxamide

### (a) 5-Ethyl-1,3-oxazole-4-carboxylic acid

Ethyl 5-ethyl-1,3-oxazole-4-carboxylate [European Journal of Med. Chem. 1987, 22, 283] (56.9 g, 336 mmol) was suspended in EtOH (700 ml) and a solution of NaOH (33.6 g, 841 mmol) in water (300 ml) was added with ice bath cooling and the system was stirred at room temperature for 14 h. The reaction mixture was concentrated under reduced pressure. The concentrated water solution was acidified to pH 1 with conc. HCl and extracted into DCM. The organics were dried (MgSO₄) and concentrated under reduced pressure to afford the crude material (45.2 g) which was used without further purification.

### (b) 5-Ethyl-1,3-oxazole

5-Ethyl-1,3-oxazole-4-carboxylic acid (45.1 g, 320 mmol) and copper(II) oxide (1.3 g, 16 mmol) were combined with quinoline (46 mL). The product was distilled from the reaction mixture under slightly reduced pressure at a distillation-head temperature less than 100° C. Distillation fractions containing clean product (as determined by NMR) were combined to provide 5-ethyl-1,3-oxazole as a clear liquid. Yield: 27g (87%).

¹H NMR (400 MHz, CDCl₃): δ 1.26 (3H, I, J=7.6 Hz), 2.69 (2H, q, J=7.6 Hz), 6.75 (1H, s), 7.76 (1H, s).

### (c) Methyl 1-(6-chloropyridin-2-yl)piperidine-4-carboxylate

2,6-Dichloropyridine (45.00 g, 304 mmol), methyl piperidine-4-carboxylate (43.1 mL, 319 mmol) and DIPEA (106 mL, 608 mmol) were suspended in DMF (350 mL) and heated at 120° C. until complete consumption of starting material was observed by HPLC analysis. The reaction mixture was concentrated under reduced pressure to afford the crude material. The crude material was partitioned between DCM (500 mL) and 1N HCl (250 mL) and the organics separated, dried (MgSO₄) and concentrated under reduced pressure to afford the crude material. Flash chromatography (eluant 10% EtOAc/Hexanes) gave methyl 1-(6-chloropyridin-2-yl)piperidine-4-carboxylate as a solid. Yield: 54.51 g (70%).

¹H NMR (400 MHz, CDCl₃): δ 1.68-1.82 (211 m), 1.94-2.04 (2H, m), 2.50-2.60 (1H, m), 2.92-3.02 (2H, m), 4.15-4.25 (2H, m), 6.50 (1H, d, J=8.4 Hz), 6.57 (1H, d, J=7.5 Hz), 7.34-7.41 (1H, m).

MS^{m}/z: 255 (M+1).

### (d) Methyl 1-(6-chloro-5-iodopyridin-2-yl)piperidine-4-carboxylate

Methyl 1-(6-chloropyridin-2-yl)piperidine-4-carboxylate (24.16 g, 94.85 mmol) was dissolved in MeCN (400 mL) and N-Iodosuccinimide (21.34 g, 94.85 mmol) added. The reaction mixture was stirred at room temperature overnight. HPLC analysis showed incomplete reaction. More N-Iodosuccinimide was added until HPLC analysis showed complete reaction conversion. The reaction mixture was concentrated under reduced pressure and the residue partitioned between EtOAc (500 mL) and sat. aqueous NaHCO₃ (300 mL). The organics were dried (MgSO₄) and concentrated under reduced pressure to afford the crude material. Flash chromatography (eluant 10-20% EtOAc/Hexanes) gave methyl 1-(6-chloro-5-iodopyridin-2-yl)piperidine-4-carboxylate as a solid. Yield: 25.77 g (71%/0).

¹H NMR (400 MHz, CDCl₃): δ 1.68-1.81 (2H, m), 1.95-2.05 (2H, m), 2.52-2.62 (1H, m), 2.94-3.05 (2H, m), 3.71 (3H, s), 4.11-4.21 (2H, m), 6.32 (1H, d, J=8.7 Hz), 7.73 (1H, d, J=8.7 Hz).

MS^{m}/z: 381 (M+1).

### (e) Methyl 1-(3,6-dichloro-5-iodopyridin-2-yl)piperidine-4-carboxylate

Methyl 1-(6-chloro-5-iodopyridin-2-yl)piperidine-4-carboxylate (24.76 g, 65.05 mmol) and NV-chlorosuccinimide (9.56 g, 71.56 mmol) were suspended in MeCN (500 mL) and stirred at reflux until complete consumption of starting material was observed by HPLC analysis. The reaction mixture was concentrated under reduced pressure and the residue partitioned between EtOAc (500 mL) and saturated aqueous NaHCO₃ (300 mL). The organics were dried (MgSO₄) and concentrated under reduced pressure to afford the crude material. Flash chromatography (eluant 7.5% EtOAc/Hexanes) gave methyl 1-(3,6-dichloro-5-iodopyridin-2-yl)piperidine-4-carboxylate as a solid. Yield: 12.93 g (48%).

¹H NMR (400 MHz, CDCl₃): δ 1.81-1.95 (2H, m), 1.99-2.07 (2H, m), 2.46-2.57 (1H, m), 2.86-2.98 (2H, m), 3.71 (3H, s), 3.81-3.90 (2H, m), 7.89 (1H, s).

MS^{m}/z: 415 (M+1).

### (f) Methyl 1-(3,6-dichloro-5-(5-ethyl-1,3-oxazol-2-yl)pyridin-2-yl)piperidine-4-carboxylate

5-ethyloxazole (3.31 g, 34.0 mmol) was dissolved in THF (1M, 40 mL) and cooled to −78° C. n-Butyllithium (24.1 mL, 38.6 mmol) was added drop-wise to the reaction mixture while maintaining an internal temperature below −60° C. The reaction mixture was stirred for 20 minutes and then ZnCl₂ (9.28 g, 68.1 mmol) was added in one portion. The reaction mixture was then warmed to room temperature and placed under an Ar_{(g)} balloon. Sonication was used to make the solution homogenous. Methyl 1-(3,6-dichloro-5-iodopyridin-2-yl)piperidine-4-carboxylate (9.42 g, 22.7 mmol) as a solution in THF (40 mL), and Pd(PPh₃)₄ (2.62 g, 2.27 mmol) were added to the reaction mixture and heated to 60° C. EtOAc (200 mL) was added and the combined organics were washed with saturated aqueous NH₄Cl (2×100 mL) and brine (1×100 mL). The organics were dried (MgSO₄) and concentrated under reduced pressure to afford the crude product. Flash chromatography (10% EtOAc in hexanes to 15% EtOAc in hexanes) gave methyl 1-(3,6-dichloro-5-(5-ethyl-1,3-oxazol-2-yl)pyridin-2-yl)piperidine-4-carboxylate as a solid.

¹H NMR (400 MHz, CDCl₃): δ 1.23-1.36 (3H, m), 1.83-1.97 (2H, m), 1.99-2.09 (2H, m), 2.50-2.62 (1H, m), 2.71-2.81 (2H, m), 2.94-3.07 (2H, m), 3.72 (3H, s), 3.98-4.09 (2H, m), 6.90 (1H, s), 8.16 (1H, s).

MS^{m}/z: 384+I).

(g) Methyl 1-(6-azido-3-chloro-5-(5-ethyl-1,3-oxazol-2-ylpyridin-2-yl)piperidin-4-carboxylate

Methyl 1-(3,6-dichloro-5-(5-ethyl-1,3-oxazol-2-yl)pyridin-2-yl)piperidine-4-carboxylate (0.840 g, 2.20 mmol) and sodium azide (0.210 g, 3.30 mmol) were dissolved in DMA (15 mL) and the reaction was heated to 70° C. for 14 h. EtOAc (50 mL) was added and the combi8ned organics were washed with water (1×30 mL) and saturated NaHCO₃ (1×30 mL), dried (MgSO₄) and concentrated under reduced pressure. No purification was done.

### (h) Methyl 1-(amino-3-chloro-5-(5-ethyl-1,3-oxazol-2-yl)pyridin-2-yl)piperidine-4-carboxylate

Methyl 1-(6-azido-3-chloro-5-(5-ethyl-1,3-oxazol-2-yl)pyridin-2-yl)piperidine-4-carboxylate (0.266 g, 0.681 mmol) was dissolved in THF (10 mL) and H₂O (2.5 mL). Zinc dust (0.445 g, 6.81 mmol) was added. NH₄Cl (10 mL) was added slowly to the solution. The solution was stirred at room temperature for 1.5 h. The reaction mixture was filtered (celite) and diluted with EtOAc (40 mL) and the combined organics were washed with saturated with NH₄OAc (2×30 mL) and brine (1×30 mL), dried (MgSO₄) and concentrated under reduced pressure to afford the crude product. Flash chromatography (10% EtOAc in hexanes to 20% EtOAc in hexanes) gave methyl 1-(6-amino-3-chloro-5-(5-ethyl-1,3-oxazol-2-yl)pyridin-2-yl)piperidine-4-carboxylate as a solid.

¹H NMR (400 MHz, d₆-DMSO): δ 1.23 (3H, t, J=7.5 Hz), 1.61-1.75 (2R^{X}, m), 1.87-1.96 (2H, m), 2.54-2.63 (1H, m), 2.72 (2H, q, J=7.5 Hz), 2.83-2.94 (2H, m), 3.63 (3H, s), 3.78-3.88 (2H, m), 6.99 (1H, s), 7.20-7.37 (2H, m), 7.83 (1H, s).

MS^{m}/z: 365 (M+1).

### (i) Methyl 1-(3-chloro-5-(5-ethyl-1,3-oxazol-2-yl)-6-(methylamino)pyridin-2-yl)piperidine-4-carboxylate

Methyl 1-(6-amino-3-chloro-5-(5-ethyl-1,3-oxazol-2-yl)pyridin-2-yl)piperidine-4-carboxylate (0.077 g, 0.210 mmol) was dissolved in DMF (1 mL) at room temperature. The reaction mixture was cooled to 0° C. and NaH (95%, 0.005 g, 0.210 mmol) was added and stirred for 10 minutes. Methyl iodide (0.039 mL, 0.629 mmol) was added and the reaction mixture was warmed to room temperature and stirred at room temperature for 16 h. EtOAc (40 mL) was added and the reaction mixture was washed with saturated NaHCO₃ (2×30 mL) dried (MgSO₄) and concentrated under reduced pressure. Flash Chromatography (15% EtOAc in hexanes) gave methyl 1-(3-chloro-5-(5-ethyloxazol-2-yl)-6-(methylamino)pyridin-2-yl)piperidine-4-carboxylate as a solid.

¹H NMR (400 MHz, d₆-DMSO): δ 1.28 (3H, t, J=7.5 Hz), 1.84-2.05 (4H, m), 2.48-2.59 (1H, m), 2.71 (2H, q, J=7.5 Hz), 2.91-3.03 (2H, m), 3.04-3.10 (3H, m), 3.71 (3H, s), 4.00-4.09 (2H, m), 6.74 (1H, s), 7.86 (1H, s), 7.98-8.08 (1H, m).

MS^{m}/z: 379 (M+1).

### (j) 1-(3-Chloro-5-(5-ethyl-1,3-oxazol-2-yl)-6-(methylamino)pyridin-2-yl)piperidine-4-carboxylic acid

Methyl 1-(3-chloro-5-(5-ethyl-1,3-oxazol-2-yl)-6-(methylamino)pyridin-2-yl)piperidine-4-carboxylate (0.052 g, 0.140 mmol), and lithium hydroxide (1 M, 2.75 mL, 2.75 mmol) were dissolved in MeOH (2 mL) and THF (2 mL), and stirred at room temperature for 20 h. The reaction mixture was concentrated under reduced pressure. H₂O (10 mL) was added to the reaction mixture and HCl (conc.) was added drop-wise until the pH was lowered to pH 2. The solution was washed with EtOAc (3×40 mL), dried (MgSO₄), and concentrated under reduced pressure to afford 1-(3-Chloro-5-(5-ethyl-1,3-oxazol-2-yl)-6-(methylamino)pyridin-2-yl)piperidine-4-carboxylic acid as a solid, which was used crude assuming a 100% yield.

¹H NMR (400 MHz, CDCDl₃): δ 1.29 (3H, t, J=7.5 Hz), 1.88-2.09 (4H, m), 2.53-2.64 (1H, m), 2.71 (2H, q, J=7.5 Hz), 2.93-3.03 (2H, m), 3.07 (3H, d, J=4.7 Hz), 3.99-4.10 (2H, m), 6.74 (1H, s), 7.87 (1H, s), 8.00-8.09 (1H, m).

MS^{m}/z: 365 (M+1).

### (k) 1-[3-Chloro-5-(5-ethyl-1,3-oxazol-2-yl)-6-(methylamino)pyridin-2-yl]-N-[(5-chloro-2-thienyl)sulfonyl]piperidine-4-carboxamide

1-(3-Chloro-5-(5-ethyl-1 ,3-oxazol-2-yl)-6-(methylamino)pyridin-2-yl)piperidine-4-carboxylic acid (0.043 g, 0.117 mmol), EDCI (0.029 g, 0.152 mmol) and HOST (0.021 g, 0.152 mmol) were dissolved in DCM (2 mL) at room temperature. The reaction mixture was stirred at room temperature for 10 minutes and then 5-chlorothiophene-2-sulfonamide (0.028 g, 0.141 mmol) and DIPEA (0.102 mL, 0.585 mmol) were added. The reaction mixture was stirred at room temperature for 16 h. The reaction mixture was diluted with EtOAc (40 mL) and the combined organics were washed with saturated NH₄Cl (2×30 mL), dried (MgSO₄) and concentrated under reduced pressure to afford the crude product. Flash chromatography (10% EtOAc in Hexanes with 0.5% AcOH) gave 1-[3-chloro-5-(5-ethyl-1,3-oxazol-2-yl)-6-(methylamino)pyridin-2-yl]-N-[(5-chloro-2-thienyl)sulfonyl]piperidine-4-carboxamide as a solid. Yield: 0.055 g (85.8%).

¹H NMR (400 MHz, CDCl₃): δ 1.21-1.37 (3H, m), 1.83-1.99 (4H, m), 2.36-2.48 (1H, m), 2.66-2.79 (2H, m), 2.85-2.98 (2H, m), 3.03-3.14 (3H, m), 4.04-4.19 (2H, m), 6.73-6.80 (1H, s), 6.93-7.02 (1H, d, J=4.2 Hz), 7.68-7.76 (1H, d, J-4.2 Hz), 7.84-7.93 (1H, s), 8.02-8.17 (2H, m).

MS^{m}/z: 544 (M+1).

### Example 59

### Ethyl 5-cyano-2-methyl-6-(4-{2-oxo-2-[(phenylsulfonyl)amino]ethyl}piperidin-1-yl)nicotinate

2-(1-(3-cyano-5-(ethoxycarbonyl)-6-methylpyridin-2-yl)piperidin-4-yl)acetic acid (0.100 g, 0.302 mmol), see example 61, EDCI (0.069 g, 0.302 mmol) and HOBT (0.049 g, 0.360 mmol) were dissolved in DCM (2 mL) at room temperature. The reaction mixture was stirred at room temperature for 10 minutes and then benzenesulfonamide (0.047 g, 0.302 mmol) and DIPEA (0.160 mL, 0.91 mmol) were added. The reaction mixture was stirred at room temperature for 18 h. The reaction mixture was diluted with EtOAc (40 mL) and the combined organics were washed with saturated NH₄Cl (2×30 mL), dried (MgSO₄) and concentrated under reduced pressure to afford the crude product. Reverse Phase HPLC purification gave ethyl 5-cyano-2-methyl-6-(4-{2-oxo-2-[(phenylsulfonyl)amino]ethyl}piperidin-1-yl)nicotinate as a solid. Yield: 0.072 g (51%).

¹H NMR (400 MHz, CDCl₃): δ 1.20-1.40 (5H, m), 1.71-1.84 (2H, m), 1.94-2.17 (1H, m), 2.18-2.26 (2H, m), 2.70 (3H, s), 2.93-3.06 (2H, m), 4.27-4.35 (2H, m), 4.63-4.73 (2H, m), 7.53-7.62 (2H, m), 7.64-7.71 (1H, m), 7.99-8.11 (2H, m), 8.32 (1H, s).

MS^{m}/z: 471 (M+1).

### Example 60

### Ethyl 4-amino-5-chloro-6-[4-({[(5-chloro-2-thienyl)sulfonyl]amino}carbonyl)piperidin-1-yl]nicotinate

### (a) Ethyl 4-azido-5,6-dichloronicotinate

4,5,6-Trichloronicotinic acid (1.28 g, 5.65 mmol) and sodium azide (0.370 g, 5.69 mmol) were dissolved in DMA (10 mL) and stirred at r.t for 16 h. Iodoethane (0.670 mL, 6.60 mmol) and potassium carbonate (3.90 g, 28.25 mmol) were added to the reaction mixture and stirred at r.t for 16 h. The reaction mixture was diluted with EtOAc (40 mL) and the combined organics were washed with water (2×40 mL), brine (1×30 mL), dried (MgSO₄) and concentrated under reduced pressure to yield ethyl 4-azido-5,6-dichloronicotinate as a solid, which was used crude assuming 100% conversion.

### (b) Ethyl 4-amino-5,6-dichloronicotinate

Ethyl 4-azido-5,6-dichloronicotinate (0.700 g, 2.68 mmol) was dissolved in 1:1 THF/MeOH (10 mL). Zinc dust (0.109 g, 1.66 mmol) was added and the solution was cooled to 5° C. NH₄Cl (2 mL) was added slowly to the solution. The solution was warmed to r.t for 2 h. The reaction mixture was filtered (celite), washed with MeOH (50 mL) and concentrated to yield ethyl 4-amino-5,6-dichloronicotinate as a solid, which was used crude assuming a 100% conversion.

### (c) 1-[4-amino-3-chloro-5-(ethoxycarbonyl)pyridin-2-yl]piperidine-4-carboxylic acid

Ethyl 4-amino-5,6-dichloronic (0.320 g, 1.36 mmol), piperidine 4-carboxylic acid (0.352 g, 2.72 mmol) and DIPEA (11.9 mL, 68.2 mmol) were dissolved in DMA (2.5 mL) and heated at 120° C. for 2 h. The reaction mixture was cooled to r.t and concentrated under reduced pressure. The crude material was dissolved in EtOAc (40 mL), washed with NH₄Cl (1×40 mL), dried (MgSO₄) and concentrated under reduced pressure to afford the crude product. Flash chromatography EtOAc/hexanes 1/3 to EtOAc/hexanes 2/3 with 0.5% AcOH) gave 1-[4-amino-3-chloro-5-(ethoxycarbonyl)pyridin-2-yl]piperidine-4-carboxylic acid as a solid. Yield: 0.154 g (34.5%).

¹H NMR (400 MHz, CDCl₃): δ 1.37 (2H, t, J=7.1 Hz), 1.88-2.07 (4H, m), 2.55-2.62 (1H, m), 2.92-3.01 (2H, m), 3.87-3.90 (2H, m), 4.33 (3H, q, J=7.1 Hz), 8.60 (1H, s).

MS^{m}/z: 328 (M+1).

### (d) Ethyl 4-amino-5-chloro-4-[4-({[(5-chloro-2-thienyl)sulfonyl]amino}carbonyl)piperidin-1-yl]nicotinate

Ethyl 4-amino-5,6-dichloronicotinate (0.176 g, 0.711 mmol), N-(5-chlorothiophen-2-ylsulfonyl)piperidine-4-carboxamide hydrochloride (0.25 g. 0.71 mmol), see example 158, and DIPEA (0.31 ml, 2.5 mmol) were combined in DMA (1.7 ml) and heated at 95° C. for 24 hr. The reaction was cooled and diluted with EtOAc (75 ml) and washed with a saturated solution of NH₄Cl (2×50 mL) followed by water (30 mL). The organic phase was dried (MgSO₄) and then concentrated in vacuo. The crude reaction mixture was purified by column chromatography (30% EtOAc/hexanes to 50% EtOAc then add AcOH slowly up to 0.2%) to provide the desired product, ethyl 4-amino-5-chloro-6-[4-({[(5-chloro-2-thienyl)sulfonyl]amino}carbonyl)piperidin-1-yl]nicotinate, as a solid. Yield: 0.037 g (10%).

¹H NMR (400 MHz, CDCl₃): δ 1.37 (3H, t, J=7.1 Hz), 1.85-1.95 (4H, m), 2.37-2.45 (1H, m), 2.89 (2H, t, J=11.4 Hz), 3.93 (2H, d, J=13.1 Hz), 4.33 (2H, q, J=7.1 Hz), 6.96 (1H, d, J=4.1 Hz), 7.70 (1H, d, J=4.1 Hz), 8.57 (1H, s).

MS^{m}/z: 507 (M+1).

### Example 61

### Ethyl 6-[4-(2-{[(5-chloro-2-thienyl)sulfonyl]amino}-2-oxoethyl)piperidin-1-yl]-5-cyano-2-methylnicotinate

### (a) 2-(1-(3-cyano-5-(ethoxycarbonyl)-6-methylpyridin-2-yl)piperidin-4-yl)acetic acid

Ethyl 6-chloro-5-cyanonicotinate (0.500 g, 2.4 mmol), 2-piperidin-4-yl)acetic acid (0.410 g, 2.80 mmol), and DIPEA (2.10 mL, 12.0 mmol) were dissolved in DCM (4 mL) and stirred at room temperature for 8 h. The reaction mixture was cooled to room temperature and the solvent concentrated under reduced pressure. The material was partitioned between EtoAc (50 mL) and saturated aqueous NaHCO₃ (2×30 mL). The organics were washed with brine (30 mL), dried (MgSO₄) and concentrated under reduced pressure to afford the crude product. No purification was done.

¹H NMR (400 MHz, CDCl₃): δ 1.34-1.42 (5H, m), 1.87-1.98 (2H, m), 2.08-2.22 (1H, m), 2.31-2.38 (2H, m), 2.71 (3H, s), 3.03-3.15 (2H, m), 4.31 (2H, q, J=7.1 Hz), 4.71-4.81 (2H, m), 8.34 (1H, s).

MS^{m}/z: 332 (M+1).

### (b) Ethyl 6-[4-(2-{[(5-chloro-2-thienyl)sulfonyl]amino}-2-oxoethyl)piperidin-1-yl]-5-cyano-2-methylnicotinate

2-(1-(3-cyano-5-(ethoxycarbonyl)-6-methylpyridin-2-yl)piperidin-4-yl)acetic acid (0.100 g, 0.302 mmol), EDCI (0.069 g, 0.302 mmol) and HOBT (0.049 g, 0.360 mmol) were dissolved in DCM (2 mL) at room temperature. The reaction mixture was stirred at room temperature for 10 minutes and then 5-chlorothiophene-2-sulfonamide (0.060 g, 0.302 mmol) and DIPEA (0.160 mL, 0.91 mmol) were added. The reaction mixture was stirred at room temperature for 16 h. The reaction mixture was diluted with EtOAc (40 mL) and the combined organics were washed with saturated NH₄Cl (2×30 mL), dried (MgSO₄) and concentrated under reduced pressure to afford the crude product. Flash chromatography (30% EtOAc in Hexanes with 0.5% AcOH) gave ethyl 6-[4-(2-{[(5-chloro-2-thienyl)sulfonyl]amino}-2-oxoethyl)piperidin-1-yl]-5-cyano-2-methylnicotinate as a solid. Yield: 0.052 g (35.0%).

¹H NMR (400 MHz, CDCl₃): δ 1.24-1.41 (5H, m), 1.81-1.89 (2H, m), 2.08-2.23 (1H, m), 2.23-2.28 (2H, m), 2.71 (3H, s), 2.97-3.09 (2H, m), 4.31 (2H, q, J=7.1 Hz), 4.68-4.77 (2H, m), 6.98 (1H, d, J=4.1 Hz), 7.70 (1H, d, J=4.1 Hz), 8.07-8.19 (In, m), 8.31-8.36 (1H, s).

MS^{m}/z: 511 (M+1).

### Example 62

### Ethyl 6-[4-({[(5-chloro-2-thienyl)sulfonyl]amino}carbonyl)-1,4-diazepan-1-yl]-5-cyano-2-methylnicotinate

### (a) Ethyl 5-cyano-4-(1,4-diazepan-1-yl)-2-methylnicotinate

Ethyl 6-chloro-5-cyanonicotinate (0.500 g, 2.4 mmol) and homopiperazine (0.240 g, 2.40 mmol) were dissolved in DCM (4 mL) at room temperature. DIPEA (0.41 mL, 2.40 mmol) was added and the system heated at reflux for 16 h. The reaction mixture was cooled to room temperature and the solvent concentrated under reduced pressure. The material was partitioned between EtOAc (50 mL) and saturated aqueous N₄Cl (2×30 mL). The organics were washed with brine (30 mL), dried (MgSO₄) and concentrated under reduced pressure to afford the crude product. No purification was done. Yield: 1.04 g (84%).

MS^{m}/z: 289 (M+1).

### (b) Ethyl 6-[4-({[(5-chloro-2-thienyl)sulfonyl]amino}carbonyl)-1,4-diazepan-1-yl]-5-cyano-2-methylnicotinate

Ethyl 5-cyano-6-(1,4-diazepan-1-yl)-2-methylnicotinate (0.150 g, 0.520 mmol) and 2,2,2-trichloroethyl [(5-chloro-2-thienyl)sulfonyl]carbamate (0.250 g, 0.680 mmol) were dissolved in DMA (4 mL) at room temperature. DMAP (0.003 g, 0.026 mmol) and DIPEA (0.450 mL, 2.60 mmol) were added and the system heated to 100° C. for 1 h. The reaction mixture was cooled to room temperature and the solvent concentrated under reduced pressure. The material was partitioned between EtOAc (40 mL) and saturated aqueous NH₄Cl (2×40 mL). The organics were dried (MgSO₄) and concentrated under reduced pressure to afford the crude product. Reverse-Phase HPLC gave ethyl 6-[4-({[(5-chloro-2-thienyl)sulfonyl]amino}carbonyl)-1,4-diazepan-1-yl]-5-cyano-2-methylnicotinate Yield: 0.125 g (47%).

¹H NMR (400 MHz, CDCl₃): δ 1.39 (3H, t, J=7.1 Hz), 1.98-2.147 (2H, m), 2.73 (31H, s), 3.36-3.56 (1H, m),3.67-3.74 (2H, m), 4.00-4.14 (4H, m), 4.33 (2H, q, J=7.1 Hz), 6.89 (1H, s), 7.60 (1H, s), 8.34 (1H, s).

MS^{m}/z: 512 (M+1).

### Example 63

### Ethyl 6-[4 -({[(5-chloro-2-thienyl)sulfonyl]amino}carbonyl)-2-methylpiperazin-1-yl]-5-cyano-2-methylnicotinate

### (a) tert-Butyl 4-(3-cyano-5-(ethoxycarbonyl)-6-methylpyridin-2-yl)-3-methylpiperazine-1-carboxylate

Ethyl 6-chloro-5-cyanonicotinate (0.500 g, 2.4 mmol), tert-butyl 3-methylpiperazine-1-carboxylate (0.480 g, 2.40 mmol), and DIPEA (0.41 mL, 2.40 mmol) were dissolved in DMF (4 mL) and heated at 100° C. for 8 h. The reaction mixture was cooled to room temperature and the solvent concentrated under reduced pressure. The material was partitioned between EtOAc (50 mL) and saturated aqueous NaHCO₃ (2×30 mL). The organics were washed with brine (30 mL), dried (MgSO₄) and concentrated under reduced pressure to afford the crude product. No purification was done.

### (b) Ethyl 5-cyano-2-methyl-6-(2-methylpiperazin-1-yl)nicotinate bis(trifluoroacetate)

tert-Butyl 4-(3-cyano-5-(ethoxycarbonyl)-6-methylpyridin-2-yl)-3-methylpiperazine-1-carboxylate (0.200 g, 0.515 mmol) was dissolved in DCM (1 mL). TEA (0.595 mL, 7.72 mmol) was added slowly. The reaction mixture was stirred at room temperature for 1 h. The mixture was concentrated under reduced pressure to afford ethyl 6-(3-aminoazetidin-1-yl)-5-cyano-2-methylnicotinate bis(trifluoroacetate) as a solid, which was used crude assuming a 100% conversion.

¹H NMR (400 MHz, CDCl₃): δ 1.39 (3H, t, J=7.1 Hz), 1.56 (3H, d, J=7.0 Hz), 2.77 (3H, s), 3.12-3.25 (1H, m), 3.26-3.36 (2H, m), 3.45-3.55 (1H, m), 3.62-3.74 (1H, m), 4.35 (2H, q, J=7.1 Hz), 4.58-4.68 (1H, m), 5.01-5.12 (1H, m), 8.42 (1H, s).

### (c) Ethyl 6-[4-({[(5-chloro-2-thienyl)sulfonyl]amino}carbonyl)-2-methylpiperazin-1-yl]-5-cyano-2-methylnicotinate

6-(3-aminoazetidin-1-yl)-5-cyano-2-methylnicotinate bis(trifluoroacetate) (0.230 g, 0.480 mmol) and 2,2,2-trichloroethyl [(5-chloro-2-thienyl)sulfonyl]carbamate (0.230 g, 0.620 mmol) were dissolved in DMA (4 mL) at room temperature. DMAP (0.003 g, 0.024 mmol) and DIPEA (0.420 mL, 2.40 mmol) were added and the system heated to 100° C. for 1 h. The reaction mixture was cooled to room temperature and the solvent concentrated under reduced pressure. The material was partitioned between EtOAc (40 mL) and saturated aqueous NH₄Cl (2×40 mL). The organics were dried (MgSO₄) and concentrated under reduced pressure to afford the crude product. Reverse-Phase HPLC gave Ethyl 6-[4-({[(5-chloro-2-thienyl)sulfonyl]amino}carbonyl)-2-methylpiperazin-1-yl]-5-cyano-2-methylnicotinate. Yield: 0.128 g (52%).

¹H NMR (400 M , CDCl₃): δ 1.35 (3H, d, J=6.6 Hz), 1.39 (3H, I, J=7.1 Hz), 2.73 (3H, s), 3.20-3.32 (1H, m), 3.38-3.45 (2H, m), 3.47-3.58 (1H, m), 3.68-3.82 (1H, m), 3.91-4.03 (1H, m), 4.34 (2H, q, J=7.1 Hz), 4.45-4.55 (1H, m), 4.89-4.98 (1H, m), 6.96 (1H, d, J=4.2 Hz), 7.68 (1H, d, J=4.2 Hz), 8.38 (1H, s).

MS^{m}/z: 512 (M+1).

### Example 64

### Ethyl 5-cyano-2-methyl-6-(4{[(phenylsulfonyl)amino]carbonyl}-1,4-diazepan-1-yl)nicotinate

### Ethyl 5-cyano-6-(1,4-diazepan-1-yl)-2-methylnicotinate (0.100 g, 0.35 mmol), see example 62, was dissolved in DCM (2 mL) and DIPEA (0.30 mL, 1.7 mmol) was added. Benzenesulfonyl isocyanate (0.046 mL, 0.35 mmol) was added and the reaction mixture was stirred at room temperature for 16 h. The reaction mixture was concentrated under reduced pressure, diluted with EtOAc (40 mL) and washed with saturated aqueous NH₄Cl (2×25 mL) and brine (25 mL). The organics were dried (MgSO₄) and concentrated under reduced pressure to afford the crude product. Flash chromatography (20-50% EtOAc in hexanes then 50% EtOAc in hexanes with 0.5% AcOH) gave ethyl 5-cyano-2-methyl-6-(4-{[(phenylsulfonyl)amino]carbonyl}-1,4-diazepan-1-yl)nicotinate as a solid. Yield: 0.134 g (82%).

¹H NMR (400 MHz, CDCl₃): δ 1.34-1.43 (31, m), 1.94-2.10 (2H, m), 2.71 (3H, s), 3.33-3.46 (2H, m), 3.59-3.70 (2H, m), 3.92-4.08 (4H, m), 4.26-4.37 (2H, m), 7.45-7.65 (3H, m), 7.94-8.08 (2H, m), 8.33 (1H, s).

MS^{m}/z: 472 (M+1).

### Example 65

### 1-[3-Chloro-5-(5-ethyl-1,3-oxazol-2-yl)-4-(methylamino)pyridin-2-yl]-N-[(5-chloro-2-thienyl)sulfonyl]piperidine-4-carboxamide

### (a) Methyl 1-(3-chloro-5-(5-ethyl-1,3-oxazol-2-yl)-4-(methylamino)pyridin-2-yl)piperidine-4-carboxylate

Methyl 1-(3-chloro-5-(5-ethyl-1,3-oxazol-2-yl)-4-(methylsulfonyl)pyridin-2-yl)piperidine-4-carboxylate (0.100 g, 0.230 mmol), see example 141, DIPEA (0.81 mL, 4.70 mmol), and methylamine (2 M, 1.2 mL, 2.3 mmol), were dissolved into THF (2 mL) and heated at 60° C. for 48 h. The reaction mixture was concentrated under reduced pressure to afford methyl 1-(3-chloro-5-(5-ethyl-1,3-oxazol-2-yl)-4-(methylamino)pyridin-2-yl)piperidine-4-carboxylate as a solid. No purification was done.

### (b) 1-(3-chloro-5-(5-ethyl-1,3-oxazol-2-yl)-4-(methylaminopyridin-2-yl)piperidin-4-carboxylic acid

Methyl 1-(3-chloro-5-(5-ethyl-1,3-oxazol-2-yl)-4-(methylamino)pyridin-2-yl)piperidine-4-carboxylate (0.089 g, 0.230 mmol), and sodium hydroxide (6 M, 1.78 mL, 10.7 mmol) were dissolved in MeOH (1 mL) and stirred at room temperature 16 h. The reaction mixture was concentrated under reduced pressure. H₂O (10 mL) was added to the reaction mixture and HCl (conc.) was added drop-wise until the pH was lowered to pH 2. The solution was washed with EtOAc (4×50 mL), dried (MgSO₄), and concentrated under reduced pressure to afford 1-(3-chloro-5-(5-ethyl-1,3-oxazol-2-yl)-4-(methylamino)pyridin-2-yl)piperidine-4-carboxylic acid as a solid, which was used crude assuming a 100% yield.

### (c) 1-[3-Chloro-5-(5-ethyl-1,3-oxazol-2-yl)-4-(methylamino)pyridin-2-yl]-N-[(5-chloro-2-thienyl)sulfonyl]piperidine-4-carboxamide

1-(3-chloro-5-(5-ethyl-1,3-oxazol-2-yl)-4-(methylamino)pyridin-2-yl)piperidine-4-carboxylic acid (0.086 g, 0.240 mmol), EDCI (0.054 g, 0.280 mmol) and HOBT (0.038 g, 0.280 mmol) were dissolved in DCM (2 mL) at room temperature. The reaction mixture was stirred at room temperature for 10 minutes and then 5-chlorothiophene-2-sulfonamide (0.056 g, 0.280 mmol) and TEA (0.160 mL, 1.20 mmol) were added. The reaction mixture was stirred at room temperature for 16 h. The reaction mixture was diluted with EtOAc (40 mL) and the combined organics were washed with saturated NH₄Cl (2×30 mL), dried (MgSO₄) and concentrated under reduced pressure to afford the crude product. Flash chromatography (30% EtOAc in Hexanes with 0.5% AcOH) gave 1-[3-Chloro-5-(5-ethyl-1,3-oxazol-2-yl)-4-(methylamino)pyridin-2-yl]-N-[(5-chloro-2-thienyl)sulfonyl]piperidine-4-carboxamide as a solid. Yield: 0.104 g (31%).

¹H NMR (400 MHz, CDCl₃): δ 1.29 (3H, t, J=7.5 Hz), 1.84-2.00 (4H, m), 2.32-2.47 (1H, m), 2.66-2.78 (2H, m), 2.82-2.93 (2H, m), 3.18-3.25 (3H, m), 3.79-3.92 (2H, m), 6.77 (1H, s), 6.96 (1H, d, J=4.1 Hz), 7.70 (1H, d, J=4.1 Hz), 8.29-8.41 (1H, m), 8.46 (1H, s).

MS^{m}/z: 545 (M+1).

### Example 66

### Ethyl 6-[4-({[(5-chloro-2-thienyl)sulfonyl]amino}carbonyl)-4-methylpiperidin-1-yl]-5-cyano-2-methylnicotinate

### (a) 1-tert-Butyl 4-methyl piperidine-1,4-dicarboxylate

1-(tert-Butoxycarbonyl)piperidine-4-carboxylic acid (3.00 g, 13 mmol) was dissolved in MeOH (50 mL) and TMSCHN₂ (32.7 mL of a 2 M solution in hexanes, 65 mmol) was added drop-wise at r.t. TMSCHN₂ was added until a persistent yellow color was produced indicating excess reagent. AcOH was added drop-wise to quench the excess TMSCHN₂ and the reaction mixture was concentrated under reduced pressure and azeotroped with Toluene (3×30 mL) to remove any trace MeOH or AcOH. The crude 1-tert-Butyl 4-methyl piperidine-1,4-dicarboxylate was used without further purification.

### (b) 1-tert-Butyl 4-methyl 4-methylpiperidine-1,4-dicarboxylate

Diisopropylamine (2.40 mL, 17 mmol) was dissolved in THF (60 mL) and cooled to 0° C. Butyl lithium 1.6 M in Hexanes (9.81 mL, 16 mmol) was added drop-wise and the system stirred at 0° C. for 1 h. The reaction mixture was cooled to −78° C. and a solution of 1-tert-butyl 4-methyl piperidine-1,4-dicarboxylate (3.18 g, 13 mmol) in THF (30 mL) was added drop-wise over 30 minutes. The reaction mixture was stirred at −78° C. for 2 h and then Iodomethane (1.31 mL, 21 mmol) in THF (10 mL) was added in one portion and the reaction mixture stirred for 2 h. The system was allowed to warm to r.t overnight. The reaction mixture was quenched with saturated NH₄Cl (100 mL) and extracted into EtOAc (100 mL). The combined organics were washed with brine (70 mL) and dried (MgSO₄) and concentrated under reduced pressure to afford the crude 1-tert-butyl 4-methyl 4-methylpiperidine-1,4-dicarboxylate as a solid, which was used without further purification.

### (c) Methyl 4-methylpiperidine-4-carboxylate

1-tert-Butyl 4-methyl 4-methylpiperidine-1,4-dicarboxylate (3.37 g, 13.1 mmol) was suspended in THF (15 mL) and 4 M HCl in 1,4-dioxane (65.4 mL, 262 mmol) was added and the reaction mixture stirred at r.t until complete consumption of the starting material was observed by TLC analysis. The reaction mixture was concentrated under reduced pressure to afford the crude material. The solids were partitioned between saturated NaHCO₃ and DCM. The organics were dried (MgSO₄) and concentrated under reduced pressure to afford the crude product. Purification by flash chromatography, (eluant 0.5% TEA, 2% MeOH/DCM −1% TEA, 5% MeOH/DCM) gave methyl 4-methylpiperidine-4-carboxylate as an oil. Yield: 0.910g (44%).

¹H NMR (400 MHz, CDCl₃): δ 1.23 (3H, s), 1.44-1.55 (2H, m), 2.09-2.20 (2H, m), 2.69-2.80 (2H, m), 2.98-3.08 (2H, m), 3.72 (3H, s), 3.99 (1H, br s).

MS^{m}/z: 158 (M+1).

### (d) 4-Methylpiperidine-4-carboxylic acid hydrochloride

Methyl 4-methylpiperidine-4-carboxylate (0.300 g, 1.9 mmol) was suspended in THF (30 mL) and potassium trimethylsilanolate (2.4 g, 19 mmol) was added. The system was heated at reflux overnight and then cooled to r.t. 4 M HCl in 1,4-dioxane (12 mL, 48 mmol) was added and the system concentrated under reduced pressure to afford crude 4-methylpiperidine-4-carboxylic acid hydrochloride as a solid, which was used without further purification.

### (e) 1-[3-Cyano-5-(ethoxycarbonyl)-6-methylpyridin-2-yl]-4-methylpiperidine-4-carboxylic acid

Ethyl 6-chloro-5-cyano-2-methylnicotinate (0.28 g, 1.3 mmol) and 4-methylpiperidine-4-carboxylic acid hydrochloride (0.34 g, 1.9 mmol) were suspended in DMF (20 mL) and DIPEA (1.1 mL, 6.3 mmol) was added. The reaction mixture was stirred at r.t until complete consumption of the starting material was observed by HPLC analysis. The reaction mixture was diluted with EtOAc (100 mL) and washed with saturated NH₄Cl (70 mL), water (2×70 mL) and brine (50 mL). The organics were dried (MgSO₄) and concentrated under reduced pressure to afford the crude material. Flash column chromatography (1:3 EtOAc/hexanes, 0.5% AcOH to 1:2 EtOAc/hexanes, 0.5% AcOH) gave 1-[3-cyano-5-(ethoxycarbonyl)-6-methylpyridin-2-yl]-4-methylpiperidine-4-carboxylic acid as a solid. Yield: 0.179 g (43%).

¹H NMR (400 MHz, DMSO-d₄): δ 1.20 (31, s), 1.30 (3H, t, J=7.1 Hz), 1.44-1.54 (2H, m), 2.02-2.11 (2H, m), 2.63 (3H, s), 3.39-3.48 (2H, m), 4.15-4.29 (4H, m), 8.32 (1H, s), 12.52 (1H, br s).

MS^{m}/z: 332 (W+1).

### (f) Ethyl 6-[4-({[(5-chloro-2-thienyl)sulfonyl]amino}carbonyl)-4-methylpiperidin-1-yl]-5-cyano-2-methylnicotinate

1-(3-Cyano-5-(ethoxycarbonyl)-6-methylpyridin-2-yl)-4-methylpiperidine-4-carboxylic acid (0.100 g, 0.30 mmol), EDCI (0.075 g, 0.39 mmol) and HOBT (0.053 g, 0.39 mmol) were dissolved in DCM (20 mL) at room temperature. The reaction mixture was stirred at room temperature for 30 minutes and then 5-chlorothiophene-2-sulfonamide (0.084 g, 0.42 mmol) and DIPEA (d 0.742) (0.32 ml, 1.8 mmol) were added. The reaction mixture was stirred at room temperature until complete consumption of starting material was observed by HPLC analysis. The reaction mixture was diluted with DCM (50 mL) and washed with saturated NH₄Cl (1×30 mL). The combined organics were dried (MgSO₄) and concentrated under reduced pressure to afford the crude material. Flash chromatography (gradient elution 3:7 EtOAc/hexanes, 0.5% AcOH to 7:3 EtOAc/hexanes, 0.5% AcOH) gave ethyl 6-[4-({[(5-chloro-2-thienyl)sulfonyl]amino}carbonyl)-4-methylpiperidin-1-yl]-5-cyano-2-methylnicotinate as a solid. Yield: 0.153 g (99%).

¹H NMR (400 MHz, d₆-DMSO): δ 1.18 (3H, s), 1.30 (3H, t, J=7.1 Hz), 1.46-1.57 (2H, m), 2.03-2.12 (2H, m), 2.62 (3H, s), 3.39-3.47 (2H, m), 3.99-4.08 (2H, m), 4.24 (2H, q, J=7.1 Hz), 7.23-7.28 (1H, m), 7.62-7.68 (1H, m), 8.31 (1H, s).

MS^{m}/z: 511 (M+1).

### Example 67

### Ethyl 6-(3-{[({[(5-chloro-2-thienyl)sulfonyl]amino}carbonyl)amino]methyl}azetidin-1-yl)-5-cyano-2-methylnicotinate

Ethyl 6-(3-(aminomethyl)azetidin-1-yl)-5-cyano-2-methylnicotinate dihydrochloride (0.200 g, 0.580 mmol),see example 68, 2,2,2-trichloroethyl 5-chlorothiophen-2-ylsulfonylcarbamate (0.260, 0.690 mmol) and DIPEA (0.500 mL, 2.90 mmol) were dissolved in DMA (5 mL) and heated at room temperature for 100° C. for 3 h. EtoAc (50 mL) added and the combined organics were washed with saturated NH₄Cl (2×40 mL), dried (MgSO₄) and concentrated under reduced pressure to afford the crude product. Flash Chromatography (40-60% EtOAc in hexanes then with 0.5% AcOH) followed by Trituration (DCM) gave ethyl 6-(3-{[({[(5-chloro-2-thienyl)sulfonyl]amino}carbonyl)amino]methyl}azetidin-1-yl)-5-cyano-2-methylnicotinate as a solid. Yield: 0.062 g (21%)

¹H NMR (400 MHz, d₆-DMSO): δ 1.29 (3H, t, J=7.1 Hz), 2.60 (3H, s), 2.76-2.88 (1H, m), 3.92-4.06 (2H, m), 4.17-4.37 (4H, m), 6.90-7.00 (1H, m), 7.23 (1H, d, J=4.1 Hz), 7.60 (1H, d, J=4.1 Hz), 8.26 (1H, s).

MS^{m}/z: 496 (M−1).

### Example 68

### Ethyl 5-cyano-2-methyl-4-{3-[({[(phenylsulfonyl)amino]carbonyl}amino)methyl]azetidin-1-yl}nicotinate

### (a) Ethyl 6-(3-((tert-butoxycarbonylamino)methyl)azetidin-1-yl)-5-cyano-2-methylnicotinate

Ethyl 6-chloro-5-cyano-2-methylnicotinate (1.00 g, 4.5 mmol), tert-butyl azetidin-3-ylmethylcarbamate (0.99 g, 5.30 mmol), and DIPEA (3.90 mL, 22.0 mmol) were dissolved in DCM (20 mL) and stirred at room temperature for 2 h. The reaction mixture was concentrated under reduced pressure and diluted with EtOAc (40 mL). The combined organics were washed with saturated NH₄Cl (2×30 mL), H₂O (1×20 mL), brine (1×30 mL), dried (MgSO₄) and concentrated under reduced pressure to afford the crude product. Flash chromatography (25 to 35% EtOAc in hexanes) gave ethyl 6-(3-((tert-butoxycarbonylamino)methyl)azetidin-1-yl)-5-cyano-2-methylnicotinate as a solid. Yield: 1.49 g (90%)

¹H NM (400 MHz, CDCl₃): δ 1.37 (3H, t, J=7.2 Hz), 1.45 (9H, s), 2.70 (3H, s), 2.88-2.99 (1H, m), 3.35-3.46 (2H, m), 4.02-4.14 (2H, m), 4.30 (2H, q, J=7.2 Hz), 4.39-4.50 (2H, m), 4.64-4.76 (1H, m), 8.26 (1H, s).

MS^{m}/z: 375 (M+1).

### (b) Ethyl 6-(3-(aminomethyl)azetidin-1-yl)-5-cyano-2-methylnicotinate dihydrochloride

Ethyl 6-(3-((tert-butoxycarbonylamino)methyl)azetidin-1-yl)-5-cyano-2-methylnicotinate (1.50 g. 4.00 mmol) was dissolved HCl (4 M, 20.0 mL, 80.0 mmol). The reaction mixture was stirred at room temperature for 16 h and concentrated under reduced pressure to yield ethyl 6-(3-(aminomethyl)azetidin-1-yl)-5-cyano-2-methylnicotinate dihydrochloride as a solid, which was used crude assuming 100% conversion.

¹H NMR (400 MHz, CDCl₃): δ 1.30 (3H, t, J=7.1 Hz), 2.60 (3H, s), 2.94-3.05 (1H, m), 3.10-3.20 (2H, m), 4.11-4.19 (2H, m), 4.23 (2H, q, J=7.1 Hz), 4.34-4.57 (2H, m), 7.93-8.04 (2H, m), 8.29 (1H, s).

MS^{m}/z: 275 (M+1).

### (c) Ethyl 5-cyano-2-methyl-6-{3-[({[(phenylsulfonyl)amino]carbonyl}amino)methyl]azetidin-t-yl}nicotinate

Ethyl 6-(3-(aminomethyl)azetidin-1-yl)-5-cyano-2-methylnicotinate dihydrochloride (0.200 g, 0.580 mmol), benzenesulfonyl isocyanate (0.092 mL, 0.690 mmol) and DIPEA (0.500 mL, 2.90 mmol) were dissolved in DCM (10 mL) and stirred at room temperature for 3 h. DCM (50 mL) added and the combined organics were washed with saturated NaHCO₃ (2×40 mL), dried (MgSO₄) and concentrated under reduced pressure to afford the crude product. Flash Chromatography (40-60% EtOAc in hexanes then with 0.5% AcOH) gave ethyl 5-cyano-2-methyl-6-{3-[({[(phenylsulfonyl)amino]carbonyl}amino)methyl]azetidin-1-yl}nicotinate as a solid. Yield: 0.249 g (94% o)

¹H NMR (400 MHz, d₆-DMSO): δ 1.29 (3F4, t, J=7.1 Hz), 2.60 (3H, s), 2.70-2.83 (1H, m), 3.21-3.28 (2H, m), 3.87-4.00 (2H, m), 4.17-4.32 (4H, m), 6.80-6.90 (1H, m), 7.53-7.70 (3H, m), 7.84-7.93 (2H, m), 8.25 (1H, s).

MS^{m}/z: 458 (M+1).

### Example 69

### Ethyl S-cyano-6-[3-({[(4-cyanophenyl)sulfonyl]amino}carbonyl)azetidin-1-yl]-2-methylnicotinate

Prepared according to method A starting from 4-cyano-benzenesulfonamid (0.070 g, 0.38 mmol). Yield: 0.047 g (41%1%)

¹H NMR (400 MHz, d₆-DMSO) δ 1.25 (3H, t, J=7.2 Hz,), 2.56 (3H, s), 3.55 (1H, ddd, J=14.5, 8.9, 5.6 Hz), 4.17 (2H, m), 4.18 (2H, q, J=7.1 Hz), 4.36 (2H, t, J=9.0 Hz), 8.07 (4H, t, J=9.3 Hz) 8.23 (1H, s)

MS^{m}/z: 454 (M+1)

### Example 70

### Ethyl 6-(3-{[(2,1,3-benzoxadiazol-4-ylsulfonyl)amino]carbonyl}azetidin-1-yl)-5-cyano-2-methylnicotinate

Prepared according to method A starting from benzo[1,2,5]oxadiazole-4-sulfonic acid amide (0.078 g, 0.38 mmol). Yield: 0.074g(63%)

¹H NMR (400 MHz, d₆-DMSO) δ 1.21 (3H, t, J=7.1 Hz), 2.51 (3H, s), 3.33 (1H, m), 4.14 (4H, m), 4.28 (2H, m), 7.64 (1H, m), 8.01 (1H, m), 8.17 (1H, m), 8.17 (1H, s),

MS^{m}/z: 471 (M+1)

### Example 71

### Ethyl 5-cyano-2-methyl-6-{3-[({([4-(1H-tetrazol-5-yl)phenyl]sulfonyl}amino)carbonyl]azetidin-1-yl}nicotinate

Prepared according to method A starting from 4-(2H-Tetrazol-5-yl)-benzenesulfonamide (0.089 g, 0.38 mmol). Yield: 0.020g (16%).

¹H NMR (400 MHz, d₆-DMSO) 8, 1.21 (31, t, J=7.1 Hz), 2.51 (3H, s), 3.46 (1H, ddd, J=14.5, 9.0, 5.7 Hz,), 4.14 (4H, m,), 4.32 (2H, , J=8.7 Hz), 8.01 (2H, d, J=8.5 Hz), 8.15 (2H, d, J=8.5 Hz,), 8.18 (s, 1H)

MS^{m}/z: 497 (M+1)

### Example 72

### Ethyl 5-cyano-6-[3-({[(4-methoxyphenyl)sulfonyl]amino}carbonyl)azetidin-1-yl]-2-methylnicotinate

Prepared according to method A starting from 4-methoxy-benzenesulfonamid (0.078 g, 0.38 mmol). Yield: 0.064 g (56%).

¹H NMR (400 MHz, d₆-DMSO) δ 8.23 (s, 1H), 7.84 (d, J=8.9 Hz, 2H), 7.10 (d, J=8.9 Hz, 2H), 4.35 (J=9.2 Hz, 2H), 4.18 (q, J=7.1 Hz, 2H), 4.14 (m, 2H), 3.81 (s, 3H), 3.52 (m, 1H), 2.55 (s, 3H, 1.25 (t, J=7.1 Hz, 3H)

MS^{m}/z: 459 (M+1)

### Example 73

### Ethyl 5-cyano-6-[3-({[(3-cyanophenyl)sulfonyl]amino}carbonyl)azetidin-1-yl]-2-methylnicotinate

Prepared according to method A starting from 3-cyano-benzenesulfonamide (0.068 g, 0.38 mmol). Yield: 0.074 g (65%).

¹H NMR (400 MHz, d₆-DMSO) δ 1.24 (3H, t, J=7.1 Hz), 2.55 (3H, s), 3.45 (1H, m), 4.17 (4H, m), 4.31 (2H, m), 7.66 (1H, t, J=7.8 Hz), 7.95 (1H, d, J=7.7 Hz), 8.07 (1H, d, J=7.9 Hz), 8.14 (1H, s), 8.21 (1H, s).

MS^{m}/z: 454 (M+1)

### Example 74

### Ethyl 5-cyano-2-methyl-6-(3-{[(2-naphthylsulfonyl)amino]carbonyl}azetidin-1-yl)nicotinate

Prepared according to method A starting from naphthalene-2-sulfonic acid amide (0.076 g, 0.38 mmol). Yield: 0.082 g (68%).

¹H NMR (400 MHz, d₆-DMSO) δ 1.20 (3H, t, J=7.1 Hz), 2.50 (3H, s), 3.50 (1H, m), 4.14 (4H, m), 4.31 (2H, m), 7.66-7.61 (2H, m), 7.83 (1H, dd, J=8.7, 1.8 Hz), 7.98 (1H, d, J=8.1 Hz), 8.07 (1H, d, J=8.7 Hz), 8.16 (2H, q, J=4.3 Hz), 8.54 (1H, s),

MS^{m}/z: 479 (M+1)

### Example 75

### Ethyl 5-cyano-[3-({[(2,4-dimethyl-1,3-thiazol-5-yl)sulfonyl]amino}carbonyl)azetidin-1-yl]-2-methylnicotinate

Prepared according to method A starting from 2,4-dimethyl-thiazole-5-sulfonamide (0.072 g, 0.38 mmol). Yield: 0.073 g (63%).

¹H NMR (400 MHz, d₆-DMSO) δ 1.25 (3H, t, J=7.2 Hz), 2.57 (3H, s), 2.58 (3H, s), 3.46 (1H, m), 4.19 (4H, m), 4.36 (2H, t, J=8.4 Hz), 8.23 (1H, s)

MS^{m}/z: 464 (M+1)

### Example 76

### Ethyl 5-cyano-6-(3-{[(2,3-dihydro-1,4-benzodioxin-4-ylsulfonyl)amino]carbonyl}azetidin-1-yl)-2-methylnicotinate

Prepared according to method A starting from 2,3-dihydro-benzo[1,4]dioxine-6-sulfonamid (0.083 g, 0.38 mmol). Yield: 0.082 g (67%).

¹H NMR (400 MHz, DMSO) 6, 1.25 (3H, t, J=7.1 Hz), 2.56 (3H, s), 3.52 (1H, dd, J=14.2, 3.1 Hz), 4.18 (4H, q, J=7.2 Hz), 4.28 (411, dd, J=10.9, 4.8 Hz), 4.36 (2H, t, J=9.0 Hz), 7.03 (1H, d, J=8.5 Hz), 7.36 (2H, td, J=8.3, 2.1 Hz), 8.23 (1H, s)

MS^{m}/z: 487 (M+1)

### Example 77

### Ethyl 5-cyano-2-methyl-6-[3-({methyl[(4-methylphenyl)sulfonyl]amino}carbonyl)azetidin-1-yl]nicotinate

Prepared according to method A starting from 4,N-dimethyl-benzenesulfonamide (0.069 g, 0.38 mmol). Yield: 0.036 g (31%).

¹H NMR (400 MHz, d₆-DMSO) δ 1.25 (3H, t, J=7.2 Hz), 2.38 (3H, s), 2.57 (3H, s), 3.19 (3H, s), 4.09 (1H, m), 4.19 (2H, q, J=7.1 Hz), 4.31 (2H, m), 4.42 (2H, t, J=9.0 Hz), 7.43 (2H, d, J=8.3 Hz), 7.84 (21, d, J=8.3 Hz), 8.25 (1H, s)

MS^{m}/z: 457 (M+1)

### Example 78

### Ethyl 5-cyano-6-[3-({[(2,4-dichlorophenyl)sulfonyl]amino}carbonyl)azetidin-1-yl]-2-methylnicotinate

Prepared according to method A starting from 2,4-dichloro-benzenesulfonamid (0.085 g, 0.38 mmol). Yield: 0.049 g (39%).

¹H NMR (400 MHz, d₆-DMSO) δ 1.24 (3H, t, J=7.1 Hz), 2.55 (34, s), 3.26 (1H, m), 4.18 (4H, m), 4.33 (2H, m), 7.51 (1H, d, J=8.7 Hz), 7.66 (1H, m), 7.96 (1H, d, J₁=8.7 Hz), 8.21 (1H, s)

MS^{m}/z: 497 (M+1)

### Example 79

### Ethyl 6-[3-({[(5-chloro-3-methyl-1-benzothien-2-yl)sulfonyl]amino}carbonyl)azetidin-1-yl]-5-cyano-2-methylnicotinate

Prepared according to method A starting from 5-chloro-3-methyl-benzo[b]thiophene-2-sulfonic acid amide 0.102 g, 0.38 mmol). Yield: 0.113 g (85%).

¹H NMR (400 MHz, d₆-DMSO) δ 1.24 (3H, t, J=7.1 Hz), 2.55 (3H, s), 2.56 (3H, s), 3.43 (1H, m), 4.18 (4H, m), 4.35 (2H, m), 7.50 (1H, d, J=8.3 Hz), 7.94 (1H, s), 8.01 (1H, d, =8.5 Hz), 8.21 (1H, s)

MS^{m}/z: 533 (M+1)

### Example 80

### Ethyl 5-cyano-2-methyl-6-[3-({[(4-methylphenyl)sulfonyl]amino}carbonyl)azetidin-1-yl]nicotinate

Prepared according to method A starting from toluene-4-sulfonamide (0.064 mg, 0.38 mmol). Yield 0.060g (54%).

¹H NMR (400 MHz, d₆-DMSO) 61.24 (3H, t, J=7.1 Hz), 2.36 (3H, s), 2.55 (3H, s), 3.52 (1H, ddd, J=14.4, 8.9, 5.6 Hz), 4.16 (4H, m), 4.35 (2H, t, J=8.9 Hz), 7.39 (2H, d, J=8.1 Hz), 7.78 (2H, d, J=8.3 Hz), 8.23 (1H, s)

MS^{m}/z: 443 (M+1)

### Example 81

### Ethyl 5-cyano-2-methyl-6-{3-[({[4 (trifluoromethyl)phenyl]sulfonyl}amino)carbonyl]azetidin-1-yl}nicotinate

Prepared according to method A starting from 4-trifluoromethyl-benzenesulfonamide (0.084 g, 0.38 mmol). Yield: 0.084 g (68%).

¹H NMR (400 MHz, d₆-DMSO) δ 1.24 (3H, t, J=7.1 Hz,), 2.55 (3H, s), 8.23 (1H, s), 3.57 (1H, ddd, J=14.4, 8.7, 5.7 Hz), 4.17 (2H, m), 4.18 (2H, q, J=7.1 Hz), 4.36 (2H, t J=9.0 Hz), 7.99 (2H, d, J=7.9 Hz), 8.12 (2H, d, J=8.3 Hz)

MS^{m}/z: 497 (M+1)

### Example 82

### Ethyl 5-cyano-2-methyl-6-[3-({[(3-nitrophenyl)sulfonyl]amino}carbonyl)azetidin-1-yl]nicotinate

Prepared according to method A starting from 3-nitro-benzenesulfonamide (0.082 g, 0.38 mmol). Yield: 0.058 g (49%).

¹H NMR (400 MHz, d₆-DMSO) δ 1.21 (3H, , J=7.1 Hz), 2.52 (31H, s), 3.34 (1H, m), 4.15 (4H, m), 4.28 (2H, m), 7.75 (1H, m), 8.18 (1H, s), 8.19 (1H, m), 8.33 (1H, m), 8.51 (1H, s)

MS^{m}/z: 474 (M+1)

### Example 83

### Ethyl 6-[3-({[(3-bromophenyl)sulfonyl]amino}carbonyl)azetidin-1-yl]-5-cyano-2-methylnicotinate

Prepared according to method A starting from 3-bromo-benzenesulfonamide (0.089 g, 0.38 mmol). Yield: 0.083 g (65%).

¹H NMR (400 MHz, d₆-DMSO) δ 1.22 (3H, t, J=7.1 Hz,), 2.53 (3H, s), 3.49 (1H, m), 4.16 (2H, m), 4.16 (2H, q, J=7.1 Hz), 4.33 (2H, t, J=8.8 Hz), 7.52 (1H, t J=7.7 Hz), 7.85 (2H, m), 7.96 (1H, s), 8.20 (1H, s)

MS^{m}/z: 508 (M+1)

### Example 84

### Ethyl 6-[3-({[(5-chloro-2-thienyl)sulfonyl]amino}carbonyl)-3-methylazetidin-1-yl]-5-cyano-2-methylnicotinate

### (a) 1-tert-Butyl 3-methyl azetidine-1,3-dicarboxylate

1-(tert-Butoxycarbonyl)azetidine-3-carboxylic acid (2.42 g, 12.0 mmol) was dissolved in MeOH (30 mL) and TMSCHN₂ (30.1 ml, 60.1 mmol) was added drop-wise at room temperature (reaction became warm and gas was evolved). TMSCHN₂ was added until a persistent yellow color was produced indicating excess reagent. AcOH was added drop-wise to quench the excess TMSCHN₂ and then the reaction mixture was concentrated under reduced pressure and azeotroped with toluene (3×20 mL) to remove any trace MeOH and AcOH. The crude material was used without any further purification assuming 100% yield.

¹H NMR (400 MHz, CDCl₃): δ 1.44 (9H, s), 3.29-3.39 (1H, m), 3.75 (3H, s), 4.07-4.13 (4H, m).

### (b) 1-ten-Butyl 3-methyl 3-methylazetidine-1,3-dicarboxylate

DIPA (1.71 ml, 12.1 mmol) was dissolved in THF (60 mL) and cooled to 0° C. Butyl lithium (6.97 ml, 11.2 mmol) was added drop-wise and the system stirred at 0° C. for 1 h. The reaction mixture was cooled to −78° C. and a solution of 1-tert-butyl 3-methyl azetidine-1,3-dicarboxylate (2.000 g, 9.29 mmol) in THF (30 mL) was added drop-wise over 30 minutes. The reaction mixture was stirred at −780C for 2 h and then iodomethane (0.928 ml, 14.9 mmol) in THF (10 mL) was added in one portion and the reaction mixture stirred for 2 h. The system was allowed to warm to room temperature overnight. The reaction mixture was quenched with sat. aqueous NH₄Cl (40 mL) and extracted into EtOAc (2×100 mL). The combined organics were washed with brine (1×100 mL), dried (MgSO₄) and concentrated under reduced pressure to afford the crude material. Flash chromatography (5-10% EtOAc/Hexanes) gave 1-tert-Butyl 3-methyl 3-methylazetidine-1,3-dicarboxylate as a solid. Yield: 0.682 g (32%).

¹H NMR (400 MHz, CDCl₃): δ 1.47 (9H, s), 1.56 (3H, s), 3.66-3.72 (2H, m), 3.78 (3H, s), 4.21-4.28 (2H, m).

### (c) Methyl 3-methylazetidine-3-carboxylate hydrochloride

1-tert-Butyl 3-methyl 3-methylazetidine-1,3-dicarboxylate (0.682 g, 3.0 mmol) was suspended in THF (15 mL) and HCl (15 ml, 59 mmol) (4 M in 1,4-dioxane) was added and the reaction mixture stirred at room temperature until complete consumption of starting material was observed by tlc analysis. The reaction mixture was concentrated under reduced pressure to afford the crude material, which was used without any further purification assuming 100% yield.

### (d) Ethyl 5-cyano-6-(3-(methoxycarbonyl)-3-methylazetidin-1-yl)-2-methylnicotinate

Methyl 3-methylazetidine-3-carboxylate hydrochloride (0.49 g, 2.97 mmol) and ethyl 6-chloro-5-cyano-2-methylnicotinate (0.56 g, 2.5 mmol) were suspended in DMF (10 mL) and DIPEA (2.2 ml, 12 mmol) was added. The reaction mixture was stirred at room temperature for 2 h and then concentrated under reduced pressure to afford the crude material. The crude material was partitioned between EtOAc (100 mL) and sat. aqueous NH₄Cl (70 mL). The organics were washed with water (2×50 mL), brine (1×50 mL), dried (MgSO₄) and concentrated under reduced pressure to afford the crude material, Flash chromatography (15-25% EtOAc/Hexanes) gave ethyl 5-cyano-6-(3-(methoxycarbonyl)-3-methylazetidin-1-yl)-2-methylnicotinate as a solid. Yield: 0.752 g (96%).

¹H NMR (400 MHz, CDCl₃): δ 1.37 (3H, t J=7.1 Hz), 1.64 (3H, s), 2.71 (3H, s), 3.78 (3H, s), 4.16-4.18 (2H, m), 4.31 (2H, q, J=7.1 Hz), 4.67-4.69 (2H, m), 8.27 (1H, s).

MS^{m}/z: 318 (M+1).

### (e) 1-(3-cyano-5-(ethoxycarbonyl)-methylpyridin-2-yl)-3-methylazetidine-3-carboxylic acid

Potassium trimethylsilanolate (0.024 g, 0.19 mmol) was added to a stirred room temperature solution of ethyl 5-cyano-6-(3-(methoxycarbonyl)-3-methylazetidin-1-yl)-2-methylnicotinate (0.050 g, 0.16 mmol) in THF (20 mL). The reaction mixture was stirred until HPLC analysis showed complete conversion of starting material. The reaction mixture was poured into 1 N HCl and extracted into DCM. The organics were dried (MgSO₄) and concentrated under reduced pressure to afford the crude product, which was used without any further purification assuming 100% yield.

¹H NMR (400 MHz, CDCl₃): δ 1.37 (3H, t, J=7.1 Hz), 1.69 (3H, s), 2.71 (3H, s), 4.16-4.24 (2H, m), 4.31 (2H, q, J=7.1 Hz), 4.69-4.77 (2H, m), 8.28 (1H, s).

### (f) Ethyl 6-[3-{[(5-chloro-2-thienyl)sulfonyl]amino}carbonyl)-3-methylazetidin-1-yl]-5-cyano-2-methylnicotinate

1-(3-Cyano-5-(ethoxycarbonyl)-6-methylpyridin-2-yl)-3-methylazetidine-3-carboxylic acid (0.20 g, 0.66 mmol), EDCI (0.16 g, 0.86 mmol) and HOBT (0.12 g, 0.S6 mmol) were dissolved in DCM (20 mL) at room temperature. The reaction mixture was stirred at room temperature for 30 minutes and then 5-chlorothiophene-2-sulfonamide (0.18 g, 0.92 mmol) and DIPEA (d 0.742) (0.69 ml, 4.0 mmol) were added. The reaction mixture was stirred at room temperature until complete consumption of starting material was observed by HPLC analysis. The reaction mixture was diluted with DCM (50 mL) and washed with saturated NH₄Cl (1×30 mL). The combined organics were dried (MgSO₄) and concentrated under reduced pressure to afford the crude material. Flash chromatography (gradient elution 3:7 EtOAc/hexanes, 0.5% AcOH to 7:3 EtOAc/hexanes, 0.5% AcOH) gave ethyl 6-[3-({[(5-chloro-2-thienyl)sulfonyl]amino}carbonyl)-3-methylazetidin-1-yl]-5-cyano-2-methylnicotinate as a solid. Yield: 0.283 g (89%).

¹H NMR (400 MHz, CDCl₃): δ 1.38 (3H, t J=7.1 Hz), 1.64 (3H, s), 2.71 (3H, s), 4.16 (2H, d, J=9.5 Hz), 4.32 (2H, q, J=7.1 Hz), 4.60 (2H, d, J=9.5 Hz), 6.98 (1H, d, J=4.1 Hz), 7.73 (1H, d, J=4.1 Hz), 8.29 (1H, s), 8.64 (1H, br s).

MS^{m}/z: 483 (M+1).

### Example 85

### 1-[6-amino-3-chloro-5-(5-ethyl-1,3-oxazol-2-yl pyridin-2-yl]-N-[(5-chloro-2-thienyl)sulfonyl]piperidine-4-carboxamide

### (a) 1-(6-Azido-3-chloro-5-(5-ethyl-1,3-oxazol-2-yl)pyridin-2-yl)piperidine-4-carboxylic acid

A solution of methyl 1-(6-azido-3-chloro-5-(5-ethyl-1,3-oxazol-2-yl)pyridin-2-yl)piperidine-4-carboxylate (0.300 g, 0.77 mmol), see example 58, in MeOH (4.0 mL) was treated with 2 M aqueous NaOH (1.0 mL, 2.0 mmol) at room temperature until complete consumption of starting material was observed by TLC. The reaction was acidified to pH 2 with 2 M aqueous HCl and extracted into EtOAc (3×50 mL). The combined extracts were washed with brine (50 mL), dried (MgSO₄) and concentrated to produce 1-(6-azido-3-chloro-5-(5-ethyl-1,3-oxazol-2-yl)pyridin-2-yl)piperidine-4-carboxylic acid as a solid. Yield: 0.26 g (90%)

### (b) 1-(6-Azido-3-chloro-5-(5-ethyl-1,3-oxazol-2-yl)pyridin-2-yl)-N-(5-chlorothiophen-2-ylsulfonyl)piperidine-4-carboxamide

1-(6-Azido-3-chloro-5-(5-ethyl-1,3-oxazol-2-yl)pyridin-2-yl)piperidine-4-carboxylic acid (0.256 g, 0.68 mmol), EDCI (0.17 g, 0.88 mmol) and HOBT (0.12 g, 0.88 mmol) were dissolved in DCM (10 mL) at room temperature. The reaction mixture was stirred at room temperature for 30 minutes and then 5-chlorothiophene-2-sulfonamide (0.19 g, 0.95 mmol) and DIPEA (0.71 ml, 4.1 mmol) were added. The reaction mixture was stirred at room temperature until complete consumption of starting material was observed by HPLC analysis. The reaction mixture was diluted with DCM (50 mL) and washed with saturated NH₄Cl (1×30 mL). The combined organics were dried (MgSO₄) and concentrated under reduced pressure to afford the crude material. Flash chromatography (gradient elution 3:7 EtOAc/hexanes, 0.5% AcOH to 7:3 EtOAc/hexanes, 0.5% AcOH) gave 1-(6-azido-3-chloro-5-(5-ethyl-1,3-oxazol-2-ylpyridin-2-yl)-N-(5-chlorothiophen-2-ylsulfonyl)piperidine-4-carboxamide as a solid. Yield: 0.287 g (76%).

MS^{m}/z: 556 (M+1).

### (c) 1-(6-Amino-3-chloro-5-(S-ethyl-1,3-oxazol-2-yl)pyridin-2-yl)-N-(5-chlorothiophen-2-ylsulfonyl)piperidine-4-carboxamide

1-(6-Azido-3-chloro-5-(5 ethyl-1,3-oxazol-2-yl)pyridin-2-yl)-N-(5-chlorothiophen-2-ylsulfonyl)piperidine-4-carboxamide (0.287 g, 0.516 mmol), was suspended in THF (10 mL) and water (2.5 mL) at room temperature. Zn dust (0.337 g, 5.16 mmol) was added followed by saturated aqueous NH₄Cl (0 mL). The reaction mixture was stirred at room temperature until complete consumption of starting material was observed by HPLC analysis. The reaction mixture was diluted with EtOAc (50 mL) and washed with saturated NH₄Cl (1×30 mL). The combined organics were dried (MgSO₄) and concentrated under reduced pressure to afford the crude material. Flash chromatography (gradient elution 3:7 EtOAc/hexanes, 0.5% AcOH to 7:3 EtOAc/hexanes, 0.5% AcOH) gave 1-(6-amino-3-chloro-5-(5-ethyl-1,3-oxazol-2-yl)pyridin-2-yl)-N-(5-chlorothiophen-2-ylsulfonyl)piperidine-4-carboxamide as a solid. Yield: 0.197 g (72%).

¹H NMR (400 MHz, CDCl₃): δ 1.29 (3H, t, J=7.6 Hz), 1.79-1.97 (4H, m), 2.36-2.44 (1H, m), 2.72 (2H, q, J=7.6 Hz), 2.78-2.88 (2H, m)₇ 3.95-4.03 (2H, m), 6.78 (1H, s), 6.96 (1H, d, J=4.2 Hz), 7.70 (1H, d, J=4.2 Hz), 7.91 (1H, s).

MS^{m}/z: 530 (M+1).

### Example 86

### Ethyl 6-[3-({[(3-bromo-5-chloro-2-thienyl)sulfonyl]amino}carbonyl)azetidin-1-yl]-5-cyano-2-methylnicotinate

Prepared according to method A starting from 3-bromo-5-chlorothiophene-2-sulfonamide (0.103 g, 0.32 mmol). Yield: 0.034 g (29%).

¹H NMR (500 MHz, d₆-DMSO) δ 1.29 (3H, t, J=7.1 Hz,), 2.61 (3H, s), 3.32 (1H, m, overlapped by water), 4.23 (2H, q, J=7.1 Hz), 4.28 (2H, m), 4.40 (2H, m), 7.29 (1H, s), 8.27 (1H, s)

MS^{m}/z: 549 (M+1)

### Example 87

### Ethyl 6-(3-{[(2,1,3-benzothiadiazol-4-ylsulfonyl)amino]carbonyl}azetidin-1-yl)-5-cyano-2-methylnicotinate

Prepared according to method A starting from 2,1,3-benzothiadiazole-4-sulfonamide (0.091 g, 0.32 mmol). Yield: 0.063 g (62%).

¹H NMR (500 MHz, d₆-DMSO) δ 1.28 (31, t, J=7.1 Hz), 2.58 (3H, s), 3.47 (1H, m), 4.14 (2H, m), 4.22 (2H, q, J=7.1 Hz), 4.35 (2H, m), 7.87 (1H, m), 8.24 (1H, s), 8.33 (2H, m),

MS^{m}/z: 487 (M+1)

### Example 88

### Ethyl 5 cyano-6-[3-({[(2,5-dimethyl-3-furyl)sulfonyl]amino}carbonyl)azetidin-1-yl]-2-methylnicotinate

Prepared according to method A starting from 2,5-dimethyl-furan-3-sulfonamide (0.086 g, 0.32 mmol). Yield: 0.036 g (38%).

¹H NMR (400 MHz, d₆-DMSO) δ 1.25 (3H, t, J=7.1 Hz), 2.19 (3H, s); 2.46 (3H, s), 2.57 (3H, s), 3.54 (1H, m),4.19 (4H, m), 4.38 (2H, t, J=9.0 Hz), 6.26 (1H, s), 8.24 (1H, s), 12.21 (1H, s)

MS^{m}/z: 447 (M+1)

### Example 89

### Ethyl 6-[3-({[(6-chloroimidazo[2,1-b][1,3]thiazol-5-yl)sulfonyl]amino}carbonyl)azetidin-1-yl]-5-cyano-2-methylnicotinate

Prepared according to method A starting from 6-chloroimidazo[2,1-b][1,3]thiazole-5-sulfonamide (0.094 g, 0.32 mmol). Yield: 0.058 g (54%).

¹H NMR (500 MHz, d₆-DMSO) δ 1.29 (4F, t, J=7.1 Hz), 2.60 (3H, s), 4.22 (4H, m), 4.36 (2H, m), 7.47 (1H, m), 7.98 (1H, d, J=4.4 Hz), 8.25 (1H, s)

MS^{m}/z: 509 (M+1)

### Example 90

### Ethyl 5-cyano-6-(3-{[(2,3-dihydro-1-benzofuran-5-ylsulfonyl)amino]carbonyl}azetidin-1-yl)-2-methylnicotinate

Prepared according to method A starting from 2,3-dihydro-1-benzofuran-5-sulfonamide (0.072 g, 0.32 mmol). Yield: 0.048 g (49%).

¹H NMR (500 MHz, d₆-DMSO) δ 1.29 (3H, t, J=7.1 Hz), 2.60 (3H, s), 3.27 (2H, t, J=8.8 Hz), 3.56 (1H, m), 4.23 (4H, m), 4.40 (2H, t, J=8.7 Hz), 4.66 (21, t, J=8.9 Hz), 6.95 (1H, d, J=8.5 Hz), 8.27 (1H, s), 7.71 (1H, dd, J=8.5, 2.1 Hz), 7.78 (1H, s),

MS^{m}/z: 471 (M+1)

### Example 91

### Ethyl 5-cyano-6-[3-({[(4-fluorophenyl)sulfonyl]amino}carbonyl)azetidin-1-yl]-2-methylnicotinate

Prepared according to method A starting from 4-fluoro-benzenesulfonamide (0.073 g, 0.32 mmol). Yield: 0.036 g (38%).

¹H NMR (400 MHz, d₆-DMSO) δ 1.25 (3H, t, J=7.2 Hz), 2.56 (3H, s), 3.53 (1H, m), 4.18 (4H, m), 4.36 (2H, t, J=8.8 Hz), 7.43 (2H, t, J=8.8 Hz), 7.97 (2H, dd, J=8.9, 5.2 Hz), 8.23 (1H, s)

MS^{m}/z: 447 (M+1)

### Example 92

### Ethyl 6-[3-({[(5-chloro-3-thienyl)sulfonyl]amino}carbonyl)azetidin-1-yl]-cyano-2-methylnicotinate

Prepared according to method A starting from 5-chloro-thiophene-3-sulfonamide (0.065 g, 0.32 mmol). Yield: 0.071 g (72%).

¹H NMR (500 MHz, d₆-DMSO) 61.29 (3H, t, J=7.1 Hz), 2.61 (3H, s), 3.48 (1H, m), 4.23 (2H, q, J=7.1 Hz), 4.25 (2H, m), 4.40 (2H, m), 7.19 (1H, m), 7.54 (1H, m), 8.28 (1H, s).

MS^{m}/z: 469 (M+1)

### Example 93

### Ethyl 5-cyano-6-[3-({[(5-isoxazol-5-yl-2-thienyl)sulfonyl]amino}carbonyl)azetidin-1-yl]-2-methylnicotinate

Prepared according to method A starting from 5-chloro-thiophene-3-sulfonamide (0.065 g, 0.32 mmol). Yield: 0.071 g (72%).

¹H NMR (500 MHz, d₆-DMSO) a 1.29 (3H, t, J=7.1 Hz), 2.61 (3H, s), 3.48 (1H, m), 4.23 (2H, q, J=7.1 Hz), 4.25 (2H, m), 4.40 (2H, m), 7.19 (1H, m), 7.54 (1H, m), 8.28 (1H, s)

MS^{m}/z: 469 (M+1)

### Example 94

### Ethyl 6-[3-({[(3-chlorophenyl)sulfonyl]amino}carbonyl)azetidin-1-yl]-5-cyano-2-methylnicotinate

Prepared according to method A starting from 3-chloro-benzenesulfonamide (0.043 g, 0.32 mmol). Yield: 0.032 g (33%).

¹H NMR (500 MHz, d₆-DMSO) δ 1.29 (3H, t, J=7.1 Hz), 8.27 (1R^{X}, s), 7.87 (2H, m), 7.75 (1H, d, J=7.9 Hz), 7.63 (1H, t, J=8.0 Hz), 4.39 (2H, m), 4.23 (4H, m), 3.52 (1H, m), 2.60 (3H, s)

MS^{m}/z: 463 (M+1)

### Example 95

### Ethyl 5-cyano-6-[3-({[(2-fluorophenyl)sulfonyl]amino}carbonyl)azetidin-1-yl]-2-methylnicotinate

Prepared according to method A starting from 2-fluoro-benzenesulfonamide (0.074 g, 0.32 mmol). Yield: 0.016 g (17%).

¹H NMR (500 MHz, d₆-DMSO) δ 1.29 (3H, t, J=7.1 Hz), 8.27 (1H, s), 7.92 (1H, t, J=7.4 Hz), 7.72 (1H, m), 7.40 (2H, dd, J=26.4, 10.8 Hz), 4.41 (2H, m), 4.22:(4H, m), 3.55 (1H, m), 2.60 (3H, s)

MS^{m}/z: 447 (M+1)

### Example 96

### Ethyl 5-cyano-6-[3-({[(5-isoxazol-3-yl-2-thienyl)sulfonyl]amino}carbonyl)azetidin-1-yl]-2-methylnicotinate

Prepared according to method A starting from 5-isoxazol-3-ylthiophene-2-sulfonamide (0.087 g, 0.32 mmol). Yield: 0.090g (86%).

¹H NMR (400 MHz, d₆-DMSO) δ 1.25 (3H, t, J=7.1 Hz), 2.56 (3H, s), 3.44 (1H, m), 4.18 (4H, m), 4.35 (2H, m), 7.02 (1H m), 7.63 (2H, m), 8.22 (1H, s), 8.67 (1H, s)

MS^{m}/z: 502 (M+1)

### Example 97

### Ethyl 5-cyano-6-[3-({[(3-fluorophenyl)sulfonyl]amino}carbonyl)azetidin-1-yl]-2-methylnicotinate

Prepared according to method A starting from 3-fluorobenzenesulfonamide (0.076 g, 0.32 mmol). Yield: 0.055 g (59%).

¹H NMR (500 MHz, d₆-DMSO) δ 1.29 (3H, t, J=7.1 Hz), 2.60 (3H, s), 3.58 (1H, dd, J=14.4, 3.2 Hz), 4.23 (4H, m), 4.40 (2H, t, J=8.6 Hz), 7.58 (1H, m), 7.69 (2H, m), 7.78 (1H, d, J=7.8 Hz), 8.27 (1H, s)

MS^{m}/z: 447 (M+1)

### Example 98

### Ethyl 5-cyano-2-methyl-6-(3-{[(phenylsulfonyl)amino]carbonyl}azetidin-1-yl)nicotinate

Prepared according to method A starting from benzenesulfonamide (0.060 g, 0.38 mmol). Yield: 0.075 g (70%).

1H NMR (400 MHz, d₆-DMSO) δ 1.24 (3H, t; J=7.2 Hz), 2.55 (3H, s), 3.47-3.57 (1H, m), 4.11-4.22 (2H, m), 4.18 (2H, q, J=7.2), 4.30-4.40 (2H, m), 7.56-7.62 (2H, m), 7.64-7.69 (1H, m), 7.87-7.92 (2H, m), 8.23 (1H, s)

MS^{m}/z: 429 (M+1)

### Example 99

### Ethyl 6-[3-({[(4-bromo-5-chloro-2-thienyl)sulfonyl]amino}carbonyl)azetidin-1-yl]-5-cyano-2-methylnicotinate

Prepared according to method A starting from 4-bromo-5-chlorothiophene-2-sulfonamide (0.105 g, 0.38 mmol). Yield: 0.139 mg (100%).

¹H NMR (400 MHz, d₆-DMSO) δ 1.23 (3H, t, J=7.2 Hz), 2.54 (3H, s), 3.33 (1H, m), 4.16 (4H, m), 4.32 (2H, m), 7.47 (1H, s), 8.20 (1H, s)

MS^{m}/z: 548 (M+1)

### Example 100

### Ethyl 6-[3-({[(5-bromo-6-chloropyridin-3-yl)sulfonyl]amino}carbonyl)azetidin-1-yl]-5-cyano-2-methylnicotinate

Prepared according to method A starting from 5-bromo-6-chloropyridine-3-sulfonamide (0.115 g, 0.38 mmol). Yield: 0.016 g (12%).

¹H NMR (400 MHz, d₆DMSO) δ 1.25 (3H, t, J=7.1 Hz), 2.56 (3H, s) 3.3 (1H, m, overlapped by water), 4.18 (4H, m), 4.32 (2H, m), 8.21 (1H, s), 8.42 (1H, s), 8.68 (1H, s)

MS^{m}/z: 543 (M+1)

### Example 101

### Ethyl 6-[3-({[(5-bromo-2-thienyl)sulfonyl]amino}carbonyl)azetidin-1-yl]-cyano-2-methylnicotinate

Prepared according to method A starting from 5-Bromo-thiophene-2-sulfonic acid amide (0.097 g, 0.38 mmol). Yield: 0.132 g (100%).

¹H NMR (400 MHz, d₆-DMSO) δ 1.25 (3H, t, J=7.1 Hz,), 2.57 (3H, s), 3.45 (1H, m), 3.45 (1H, ddd, J=14.4, 8.8, 5.7 Hz), 4.19 (4H, m), 4.36 (2H, t, J=8.7 Hz), 7.47 (1H, d, J=3.8 Hz), 8.23 (1H, s)

MS^{m}/z: 514 (M+1)

### Example 102

### Ethyl 5-cyano-2-methyl-6-[3-({[(5-pyridin-2-yl-2-thienyl)sulfonyl]amino}carbonyl)azetidin-1-yl]nicotinate

Prepared according to method A starting from 5-pyridin-2-yl-thiophene-2-sulfonic acid amide (0.073 g, 0.38 mmol). Yield: 0.045 g (35°% o).

¹H NMR (400 MHz, d₆-DMSO) δ 1.24 (3H, t, J=7.1 Hz), 2.54 (3H, s), 3.58 (11I, dd, J=14.2, 3.1 Hz), 4.19 (4H, m), 4.38 (2H, t, J=8.8 Hz), 7.37 (1H, dd, J=7.3, 5.0 Hz), 7.81 (2H, dd, J=16.7, 4.0 Hz), 7.88 (1H, dd, J=15.5, 1.6 Hz), 8.03 (1H, d, J=8.1 Hz), 8.23 (1H, s),), 8.55 (1H, d, J=4.6 Hz)

MS^{m}/z: 512 (M+1)

### Example 103

### Ethyl 5-cyano-6 [3-({[(2,5-dichloro-3-thienyl sulfonyl]amino}carbonyl)azetidin-1-yl]-2-methylnicotinate

Prepared according to method A starting from 2,5-dichloro-thiophene-3-sulfonic acid amide (0.082 g, 0.38 mmol). Yield: 0.027 g (21%).

¹H NMR (400 MHz, d₆-DMSO) δ 1.25 (3H, t, J=7.2 Hz), 2.57 (3H, s), 3.41 (1H, m), 4.19 (4H, m), 4.36 (2H, m), 7.25 (1H, s), 8.23 (1H, s),

MS^{m}/z: 503 (M+1)

### Example 104

### Ethyl 5-cyano-6-[3-({[(4,5-dichloro-2-thienyl)sulfonyl]amino}carbonyl)azetidin-1-yl]-2-methylnicotinate

Prepared according to method A starting from 4,5-dichlorothiophene-2-sulfonamide (0.108 g, 0.38 mmol). Yield: 0.094 g (75%).

¹H NMR (400 MHz, d₆-DMSO) δ 1.25 (3H, t, J=7.1 Hz), 2.57 (3H, s), 3.36 (1H, m), 4.19 (4H, m), 4.35 (2H, q, J=7.1 Hz), 7.54 (1H, s), 8.23 (1H, s)

MS^{m}/z: 503 (M+1)

### Example 105

### Ethyl 5-cyano-2-methyl-6-{3-[({[3-(trifluoromethyl)phenyl]sulfonyl}amino)carbonyl]azetidin-1-yl}nicotinate

Prepared according to method A starting from 3-(trifluoromethyl)benzenesulfonamide (0.092 g, 0.38 mmol). Yield: 0.009 g (7%).

¹H NMR (400 MHz, d₆-DMSO) δ 1.25 (3H, t, J=7.2 Hz,), 2.55 (3H, s), 3.57 (1H, ddd, J=14.5, 8.9, 5.7 Hz), 4.18 (4H, m), 4.36 (2H, t, J=9.0 Hz), 7.88 (1H, t, J=7.9 Hz), 8.10 (1H, d, J=7.9 Hz), 8.15 (1H, s), 8.22 (1H, d, J=7.5 Hz), 8.23 (1H, s),

MS^{m}/z: 497 (M+1)

### Example 106

### Ethyl 6-(3-{[(1-benzothien-3-ylsulfonyl)amino]carbonyl}azetidin-1-yl)-5-cyano-2-methylnicotinate

Prepared according to method A starting from 1-benzothiophene-3-sulfonamide (0.081 g, 0.38 mmol). Yield: 0.013 g (11%).

¹H NMR (400 MHz, d₆DMSO) δ 1.24 (3H, t, J=7.1 Hz), 2.53 (3H, s), 3.48 (1H, m), 4.09 (2H, m), 4.17 (3H, q, J=7.1 Hz), 4.33 (2H, m), 7.49 (2H, m), 8.10 (21, t, J=8.9 Hz), 8.20 (1H, s), 8.63 (1H, m)

MS^{m}/z: 485 (M+1)

### Example 107

### Ethyl 6-[3-({[(2-chlorophenyl)sulfonyl]amino}carbonyl)azetidin-1-yl]-5-cyano-2-methylnicotinate

Prepared according to method A starting from 2-chlorobenzenesulfonamide (0.075 g, 0.38 mmol). Yield: 0.092 g (80%).

¹H NMR (400 MHz, d₆-DMSO) δ 1.24 (3H, t, J=7.1 Hz), 2.56 (3H, s), 3.50 (1H, m), 4.18 (4H, m), 4.37 (2H, m), 7.51 (1H, m), 7.59 (2H, s), 8.05 (1H, d, J=7.9 Hz), 8.22 (1H, s),

MS^{m}/z: 463 (M+1)

### Example 108

### Ethyl 5-cyano-6-[3-({[(2,5-dimethyl-3-thienyl)sulfonyl]amino}carbonyl)azetidin-1-yl]-2-methylnicotinate

Prepared according to method A starting from 2,5-dimethylthiophene-3-sulfonamide (0.085 g, 0.38 mmol). Yield: 0.019 g (17%).

¹H NMR (400 MHz, d₆-DMSO) δ 1.25 (3H, t, J=7.2 Hz), 2.33 (3H, s), 2.57 (6H, s), 3.55 (1H, m), 4.19 (4H, m), 4.38 (2H, m), 6.93 (1H, s), 8.24 (1H, s),

MS^{m}/z: 463 (M+1)

### Example 109

### Ethyl 5-cyano-4-[3-({[(3-methoxyphenyl)sulfonyl]amino}carbonyl)azetidin-1-yl]-2-methylnicotinate

Prepared according to method A starting from 3-methoxybenzenesulfonamide (0.083 g, 0.38 mmol). Yield: 0.011 g (10%1/).

¹H NMR (400 MHz, d₆-DMSO) δ 1.25 (3H, t, J=7.2 Hz), 2.58 (3H, s), 3.53 (1H, m), 3.79 (3H, s), 4.18 (4H, m), 4.36 (2H, t, J=9.0 Hz), 7.23 (1H, d, J 8.1 Hz), 7.36 (1H, s), 7.48 (2H, dt, J=15.8, 8.0 Hz), 8.23 (1H, s)

MS^{m}/z: 459 (M+1)

### Example 110

### Ethyl 5-cyano-2-methyl-6-(3-{[(3-thienylsulfonyl)amino]carbonyl}azetidin-1-yl)nicotinate

Prepared according to method A starting from thiophene-3-sulfonamide (0.066 g, 0.38 mmol). Yield: 0.059 g (54%).

¹H NMR (400 MHz, d₆-DMSO) δ 1.25 (3H, t, J=7.1 Hz), 2.50 (3H, s), 3.56 (1H, m), 4.19 (4H, q, J=7.1 Hz), 4.37 (2H, t, J=8.9 Hz), 7.40 (1H, d, J=5.2 Hz), 7.73 (1H, dd, J=5.1, 3.1 Hz), 8.24 (1H, s), 8.39 (1H, d, J=1.8 Hz), 12.30 (1H, s)

MS^{m}/z: 435 (M+1)

### Example 111

### Ethyl 5-cyano-2-methyl-6-(3-{[(2-thienylsulfonyl)amino]carbonyl}azetidin-1-yl)nicotinate

Prepared according to method A starting from starting from thiophene-2-sulfonamide (0.087 g, 0.38 mmol). Yield: 0.088 g (81%).

¹H NMR (400 MHz, d₆-DMSO) δ 1.25 (3H t, J=7.2 Hz), 2.56 (3H, s), 3.48 (1H, dd, J=14.2, 3.1 Hz), 4.19 (4H, q, J=7.0 Hz), 4.36 (2H, t, J=8.9 Hz), 7.12 (1H, t, J=4.4 Hz), 7.69 (1H, t, J=4.4 Hz), 7.92 (1H, m), 8.23 (1H, s),

MS^{m}/z: 435 (M+1)

### Example 112

### 1-[4-Amino-3-chloro-5-(5-ethyl-1,3-oxazol-2-yl)pyridin-2-yl]-N-[(5-chloro-2-thienyl)sulfonyl]piperidine-4-carboxamide

### (a) 5,6-Dichloro-N-(2-hydroxybutyl)nicotinamide

5,6-Dichloronicotinic acid (20.0 g, 104 mmol), EDCI (26.0 g, 135 mmol) and HOBt (18.3 g, 135 mmol) were dissolved in DCM (500 mL) at r.t. The reaction mixture was stirred at r.t for 90 minutes and then 1-aminobutan-2-ol (15.0 g, 156 mmol) and DIPEA (54.4 mL, 313 mmol) were added. The reaction mixture was stirred at r.t for 18 h. The reaction mixture was diluted with DCM (400 mL) and the combined organics were washed with saturated NH₄Cl (2×100 mL), saturated NaHCO₃ (2×100 mL), dried (MgSO₄) and concentrated under reduced pressure to afford 5,6-dichloro-N-(2-hydroxybutyl)nicotinamide as a solid, which was used crude assuming a 100% conversion.

### (b) 5,6-Dichloro-N-(2-oxobutyl)nicotinamide

Oxalyl chloride (16.3 mL, 187 mmol) was dissolved in DCM (500 mL) and cooled to −78° C. DMSO (26.3 mL, 374 mmol) was added drop-wise and stirred at −78° C. for 10 minutes. 5,6-Dichloro-N-(2-hydroxybutyl)nicotinamide (30 g, 94 mmol) was dissolved in DCM/DMSO (3:1) and added slowly to the solution. The solution was stirred at −78° C. for 30 minutes. TEA (65.2 mL, 467 mmol) was added to the solution and stirred for 30 minutes. The solution was warmed to r.t and stirred for 3 h. The reaction mixture was diluted with DCM (200 mL) and the combined organics were washed with water (2×200 mL), brine (2×200 mL), dried (MgSO₄) and concentrated under reduced pressure to afford 5,6-dichloro-N-(2-oxobutyl)nicotinamide as a solid, which was used crude assuming a 100% conversion.

### (c) 2,3-dichloro-5-(5-ethyl-1,3-oxazol-2-yl)pyridine

5,6-Dichloro-N-(2-oxobutyl)nicotinamide (26.7 g, 78 mmol) and POCl₃ (59.6 g, 389 mmol) were dissolved in DMF (500 mL) and heated at 90° C. for 30 minutes. The reaction mixture was poured onto ice. Solid NaHCO₃ was added in portions until the pH was raised to pH>8. The reaction mixture was diluted with water (500 mL) and the combined aqueous were washed with EtOAc (3×400 mL), dried (MgSO₄) and concentrated under reduced pressure to afford the crude product as a solid. Flash chromatography (EtOAc/hexanes, 1/9) gave 2,3-dichloro-5-(5-ethyl-1,3-oxazol-2-yl)pyridine as a solid. Yield: 7.08 g (37%).

¹H NMR (400 MHz, CDCl₃): δ 1.33 (2H, t, J=7.5 Hz), 2.78 (2H, q, J=7.5 Hz), 6.91 (1H, s), 8.35 (1H, d, J=1.9 Hz) 8.29 (1H, d, J=1.9 Hz).

MS^{m}/z: 244 (M+1).

### (d) 2,3-dichloro-5-(5-ethyl-1,3-oxazol-2-yl)-4-(methylthio)pyridine

n-Butyllithium (2.5 M in hexanes, 7.14 mL, 17 mmol) was added drop-wise to diisopropylamine (2.62 mL, 19 mmol) in THF (5 mL) at 0° C. The solution was stirred at 0° C. for 30 minutes and then cooled to −78° C. 2,3-dichloro-5-(5-ethyl-1,3-oxazol-2-yl)pyridine (3.50 g, 14 mmol) in THF (30 mL) was added to the solution and the reaction was stirred at −78° C. for 1 h. S-methyl methanesulfonothioate (1.77 mL, 19 mmol) was added and the solution warmed to r.t. The reaction mixture was stirred for 16 h. The reaction mixture was diluted with saturated NH₄Cl (100 mL). The solution was washed with EtOAc (3×50 mL). The combined organics were washed with brine (1×50 mL), dried (MgSO₄) and concentrated under reduced pressure to afford the crude product as a solid. Flash chromatography (15% EtOAc/hexanes to 20% EtOAc/hexanes) gave 2,3-dichloro-5-(5-ethyl-1,3-oxazol-2-yl)-4-(methylthio)pyridine as a solid. Yield: 2.71 g (65%).

¹H NMR (400 MHz, CDCl₃): δ 1.33 (2H, t, J=7.6 Hz), 2.35 (3H, s), 2.79 (2H, q, J=7.6 Hz), 6.98 (1H, s), 8.58 (1H, s).

MS^{m}/z: 290 (M+1).

### (e) Methyl 1-[3-chloro-5-(5-ethyl-1,3-oxazol-2-yl)-4-(methylthio)pyridin-2-yl]piperidine-4-carboxylate

2,3-dichloro-5-(5-ethyl-1,3-oxazol-2-yl)-4-(methylthio)pyridine (3.11 g, 11 mmol), methyl piperidine-4-carboxylate (2.00 g, 14 mmol) and DIPEA (3.75 mL, 22 mmol) were dissolved in DMA (50 mL) and heated to 120° C. for 2 h. The reaction mixture was cooled to r.t and concentrated under reduced pressure. The crude material was dissolved in EtoAc (100 mL), washed with NH₄Cl (2×60 mL), dried (MgSO₄) and concentrated under reduced pressure to afford the crude product as a solid. Flash chromatography (1:5 EtOAc/hexanes to 1:3 EtOAc/hexanes) gave methyl 1-[3-chloro-5-(5-ethyl-1,3-oxazol-2-yl)-4-(methylthio)pyridin-2-yl]piperidine-4-carboxylate as a solid. Yield: 4.26 g (88%).

¹H NMR (400 MHz, CDCl₃): δ 1.33 (2H, t, J=7.6 Hz), 1.88-2.06 (4H, m), 2.32 (3H, s), 2.51-2.58 (1H, m), 2.76 (3H, q, J=7.6 Hz), 2.93-2.99 (2H, m), 3.72 (3H, s), 3.81-3.92 (2H, m), 6.91 (1H, s), 8.43 (1H, s).

MS^{m}/z: 396 (M+1).

### (f) Methyl 1-[3-chloro-5-(5-ethyl-1,3-oxazol-2-yl)-4-(methylsulfinyl)pyridin-2-yl]piperidine-4-carboxylate

Methyl 1-[3-chloro-5-(5-ethyl-1,3-oxazol-2-yl)-4-(methylthio)pyridin-2-yl]piperidine-4-carboxylate (2.12 g, 5.4 mmol) was dissolved in DMF (500 mL) and 3-chlorobenzenecarboperoxoic acid (2.64 g, 10.7 mmol) was slowly added at r.t. The solution was stirred at r.t for 4 h. 3-chlorobenzenecarboperoxoic acid (1.32 g, 5.35 mmol) was slowly added at r.t for 3 h. Saturated Na₂S₂O₃ (30 mL) was added and the solution was stirred for 5 minutes. The reaction mixture was diluted with CH₂Cl₂ (40 mL) and the combined organics were separated and washed with NaOH (1M, 2×40 mL), brine (1×30 mL), dried (MgSO₄) and concentrated under reduced pressure to afford the crude product. Flash chromatography (1:2 EtOAc/hexanes) gave methyl 1-[3-chloro-5-(5-ethyl-1,3-oxazol-2-yl)-4-(methylsulfinyl)pyridin-2-yl]piperidine-4-carboxylate as a solid. Yield: 2.71 g (65%).

¹H NMR (400 MHz, CDCl₃): δ 1.30 (1H, t, J=7.5 Hz), 1.83-2.08 (4H, m) 2.52-2.61 (1R^{X}, m), 2.75 (2H, q, J=7.5 Hz), 2.93-3.00 (1,H m), 3.04-3.13 (1H, m), 3.23 (3H, s), 3.72 (3H, s), 3.86-4.01 (2H, m), 6.87 (1H, s), 8.51 (1H, s).

MS^{m}/z: 412 (M+1).

### (g) Methyl 1-[4-azido-3-chloro-5-(5-ethyl-1,3-oxazol-2-yl)pyridin-2-yl]piperidine-4-carboxylate

Methyl 1-[3-chloro-5-(5-ethyl-1,3-oxazol-2-yl)-4-(methylsulfinyl)pyridin-2-yl]piperidine-4-carboxylate (0.150 g, 0.36 mmol) and sodium azide (0.026 g, 0.40 mmol) were dissolved in DMA (1 mL) and stirred at r.t for 48 h. The reaction mixture was diluted with EtOAc (40 mL) and the combined organics were separated and washed with water (2×40 mL), brine (1×30 mL), dried (MgSO₄) and concentrated under reduced pressure to afford methyl 1-[4-azido-3-chloro-5-(5-ethyl-1,3-oxazol-2-yl)pyridin-2-yl]piperidine-4-carboxylate as a solid, which was used crude assuming a 100% conversion

### (h) 1-(3-Chloro-5-(5-ethyl-1,3-oxazol-2-yl)-4-(methylsulfinyl)pyridin-2-yl)piperidin-4-carboxylic acid

Methyl 1-(4-azido-3-chloro-5-(5-ethyl-1,3-oxazol-2-yl)pyridin-2-yl)piperidine-4-carboxylate (0.170 g, 0.435 mmol), and lithium hydroxide (1 M, 4.35 mL, 4.35 mmol) were dissolved in THF (2 mL) and stirred at room temperature 22 h. The reaction mixture was concentrated under reduced pressure. H₂O (10 mL) was added to the reaction mixture and HCl (conc.) was added drop-wise until the pH was lowered to pH 2. The solution was washed with EtOAc (4×40 mL), dried (MgSO₄), and concentrated under reduced pressure to afford 1-(3-chloro-5-(5-ethyl-1,3-oxazol-2-yl)-4-(Methylsulfinyl)pyridin-2-yl)piperidine-4-carboxylic acid as a solid, which was used crude assuming a 100% yield.

¹H NMR (400 MHz, CDCl₃): δ 1.31 (3H, t J=7.6 Hz), 1.88-2.02 (2H, m), 2.03-2.13 (21, m), 2.56-2.67 (1H, m), 2.77 (2H, q, J=7.6 Hz), 2.95-3.07 (2H, m), 3.87-3.97 (2H, m), 6.93 (1H, s), 8.58 (1H, s).

MS^{m}/z: 377 (M+1).

### (i) 1-(4-Azido-3-chloro-5-(5-ethyl-1,3-oxazol-2-yl)pyridin-2-yl)N-(5-chlorothiophen-2-ylsulfonyl)piperidine-4-carboxamide

1-(3-chloro-5-(5-ethyl-1,3-oxazol-2-yl)-4-(methylsulfinyl)pyridin-2-yl)piperidine-4-carboxylic acid (0.160 g, 0.420 mmol), EDCI (0.098 g, 0.510 mmol) and HOBT (0.069 g, 0.510 mmol) were dissolved in DCM (2 mL) at room temperature. The reaction mixture was stirred at room temperature for 10 minutes and then 5-chlorothiophene-2-sulfonamide (0.084 g, 0.420 mmol) and TEA (0.300 mL, 2.10 mmol) were added. The reaction mixture was stirred at room temperature for 16 h. The reaction mixture was diluted with EtOAc (40 mL) and the combined organics were washed with saturated NH₄Cl (2×30 mL), dried (MgSO₄) and concentrated under reduced pressure to afford the crude product. Flash chromatography (30% EtOAc in Hexanes with 0.5% AcOH) gave 1-(4-azido-3-chloro-5-(5-ethyl-1,3-oxazol-2-yl)pyridin-2-yl)-N-(5-chlorothiophen-2-ylsulfonyl)piperidine-4-carboxamide as a solid. Yield: 0.165 g (70%).

¹H NMR (400 MHz, CDCl₃): δ 1.31 (31H, J=7.5 Hz), 1.83-1.99 (4H, m), 2.35-2.46 (1H, m), 2.77 (2H, q, J=7.5 Hz), 2.80-2.90 (2H, m), 3.89-4.00 (2H, m), 6.91-6.98 (2H, m), 7.67-7.73 (1H, m), 8.55 (1H, s).

MS^{m}/z: 556 (M+1).

### (j) 1-[4-Amino-3-chloro-5-(5-ethyl-1,3-oxazol-2-yl)pyridin-2-yl]-N-[(5-chloro-2-thienyl)sulfonyl]piperidine-4-carboxamide

1-(4-azido-3-chloro-5-(5-ethyl-1,3-oxazol-2-yl)pyridin-2-yl)-N-(5-chlorothiophen-2-ylsulfonyl)piperidine-4-carboxamide (0.100 g, 0.180 mmol) was dissolved in THF (0.900 mL) and cooled to 0° C. Zinc dust (0.059 g, 0.900 mmol) was added. NH₄Cl (0.900 mL) was added slowly to the solution. The solution was warmed to room temperature for 0.5 h. The reaction mixture was filtered (celite) and diluted with EtOAc (40 mL) and the combined organics were washed with saturated with NH₄OAc (1×30 mL) and brine (1×30 mL), dried (MgSO₄) and concentrated under reduced pressure. Trituration (17% DCM in Hexanes and 17% Et₂O in Hexanes) gave 1-[4-amino-3-chloro-5-(5-ethyl-1,3-oxazol-2-yl)pyridin-2-yl]-N-[(5-chloro-2-thienyl)sulfonyl]piperidine-4-carboxamide as a solid. Yield: 0.087 g (91%).

¹H NMR (400 MHz, d₆-DMSO): δ 1.19-1.28 (3H, m), 1.53-1.68 (2H, m), 1.76-1.85 (2H, m), 2.69-2.83 (4H, m), 3.66-3.77 (2H, m), 7.05 (1H, s), 7.20-7.30 (1H, m), 7.63-7.68 (1H, m), 8.41 (1H, s).

MS^{m}/z: 531 (M+1).

### Example 113

### tert-Butyl 5-chloro-6-[4-({[(5-chloro-2-thienyl)sulfonyl]amino}carbonyl)piperidin-1-yl]nicotinate

### (a) 5-Chloro-6-(4-(methoxycarbonyl)piperidin-1-yl)nicotinic acid

A suspension of 5,6-dichloronicotinic acid (50.0 g, 260 mmol) and methyl piperidine-4-carboxylate (46.6 g, 325 mmol) in DMA (350 mL) was heated to 120° C. until complete consumption of 5,6-dichloronicotinic acid was observed by HPLC analysis. The reaction mixture was concentrated under reduced pressure, diluted with DCM (100 mL), washed with 1N HCl (400 mL), brine (400 mL), dried (MgSO₄) and concentrated under reduced pressure to afford 5-chloro-6-(4-(methoxycarbonyl)piperidin-1-yl)nicotinic acid, which was used without further purification. Yield: 75.1 g (96%).

### (b) tert-Butyl 5-chloro-6-(4-(methoxycarbonyl)piperidin-1-yl)nicotinate

A solution of 5-chloro-6-(4-(methoxycarbonyl)piperidin-1-yl)nicotinic acid (2.01 g, 6.73 mmol) and tert-butyl N,N′-diisopropylcarbamimidate (21.6 g, 107 mmol) in THF (100 mL) was heated to reflux for 1 h. After cooling to room temperature, the resulting precipitate was removed by filtration through silica gel and discarded. The supernatant was concentrated. Flash chromatography (5% EtOAc/hexanes) furnished tert-butyl 5-chloro-6-(4-(methoxycarbonyl)piperidin-1-yl)nicotinate. Yield: 1.91 g (80%).

¹H NMR (400 MHz, CDCl₃): δ 1.57 (9H, s), 1.85-1.95 (2H, m), 2.01-2.05 (2H, m), 2.52-2.60 (1H, m), 2.96-3.03 (2H, m), 3.71 (3H, s), 4.00-4.05 (2H, m), 8.05 (1H, d, J=2.7 Hz), 8.69 (1H, d, J=2.7 Hz).

MS^{m}/z: 355 (M+1).

### (c) Potassium 1-(5-(tert-butoxycarbonyl)-3-chloropyridin-2-yl)piperidine-4-carboxylate

To a solution of tert-butyl 5-chloro-6-(4-(methoxycarbonyl)piperidin-1-yl)nicotinate (0.303 g, 0.854 mmol) in Et₂O (25 mL) was added potassium trimethylsilanoate (0.128 g, 0.897 mmol). After stirring at room temperature for 2 h, a potassium 1-(5-(tert-butoxycarbonyl)-3-chloropyridin-2-yl)piperidine-4-carboxylate was collected by filtration and washed with Et₂O. Yield: 0.222 g (69%).

### (d) ter-Butyl 5-chloro-6-[4-({[(5-chloro-2-thienyl)sulfonyl]amino}carbonyl)piperidin-1-yl]nicotinate

A solution of potassium 1-(5-(tert-butoxycarbonyl)-3-chloropyridin-2-yl)piperidine-4-carboxylate (0.222, 0.586 mmol), EDCI (0.187 g, 0.977 mmol), HOBt (0.132 g, 0.977 mmol), DIPEA (0.340 mL, 1.95 mmol), 5-chlorothiophene-2-sulfonamide (0.193 g, 0.977 mmol) in DCM (15 mL) was stirred at room temperature for 2 days. The mixture was concentrated, diluted with EtOAc (50 mL), washed with saturated NH₄Cl (25 mL), brine (25 mL), dried (MgSO₄), and concentrated. Reverse phase preparative HPLC furnished tert-Butyl 5-chloro-6-[4-({[(5-chloro-2-thienyl)sulfonyl]amino}carbonyl)piperidin-1-yl]nicotinate as a solid. Yield: 0.150 g (42%).

¹H NMR (400 MHz, CDCl₃): δ 1.58 (9H, s), 1.84-1.91 (2H, m), 1.93-2.02 (2H, m), 2.47-2.54 (1H, m), 3.00-3.07 (2H, m), 4.06-4.09 (2H, m) 6.96 (1H, d, J=4.1 Hz), 7.69 (1H, d, J=4.1 Hz), 8.14 (1H, d, J=1.9 Hz), 8.67 (1H, d, J=1.9 Hz), 8.71 (1H, br s).

MS^{m}/z: 521 (M+1).

### Example 114

### N-[(5-chloro-2-thienyl)sulfonyl]-1-[5-(5-ethyl-t ,3-oxazol-2-yl)-3-(isopropylamino)pyridin-2-yl]piperidine-4-carboxamide

### (a) Methyl 1-(5-(5-ethyloxazol-2-yl)-3-(isopropylamino)pyridin-2-yl)piperidine-4-carboxylate

To a solution of methyl 1-(3-amino-5-(5-ethyloxazol-2-yl)pyridin-2-yl)piperidine-4-carboxylate (0.238 g, 0.722 mmol), see example 121, and acetone (0.054 g, 0.939 mmol) in DCM (5.0) mL was added TiCl₄ (1.0 M in DCM, 0.794 mmol). A precipitated formed and the heterogeneous mixture was stirred for 20 h. Sodium cyanoborohydride (0.136 g, 2.17 mmol) was added the reaction mixture was stirred for 24 h. The mixture was diluted with Et₂O (100 mL), washed with water (50 mL), brine (50 mL), dried (MgSO₄), passed through silica gel and concentrated. Flash chromatography (15% EtOAc/hexanes) furnished methyl 1-(5-(5-ethyloxazol-2-yl)-3-(isopropylamino)pyridin-2-yl)piperidine-4-carboxylate as a solid. Yield 0.086 g (32%).

¹H NMR (400 MHz, CDCl₃): δ 1.27 (6H, d, J=6.3 Hz), 1.30 (3H, t, J=7.6 Hz), 1.81-1.91 (2H, m), 2.05-2.08 (2H, m), 2.48-2.55 (1H, m), 2.72-2.82 (4H, m), 3.39-3.42 (2H, m), 3.62-3.69 (1H, m), 3.73 (3H, s), 4.14 (1H, d, J=7.1 Hz), 6.81 (1H, s), 7.34 (1H, s), 8.27 (1H, s).

MS^{m}/z: 373 (M+1).

### (b) 1-(5-(5-Ethyloxazol-2-yl)-3-(isopropylamino)pyridin-2-)piperidine-4-carboxylic acid

A solution of methyl 1-(5-(5-ethyloxazol-2-yl)-3-(isopropylamino)pyridin-2-yl)piperidine-4-carboxylate (0.086 g, 0.23 mmol) in THF (10 mL) was treated with 1 M LiOH (10 mL) with vigorous stirring for 3 h. The reaction mixture was acidified to pH 3 with 1 M HCl and extracted with EtOAc (3×50 mL). The combined extracts were washed with brine (25 mL), dried MgsSO₄) and concentrated to yield 1-(5-(5-ethyloxazol-2-yl)-3-(isopropylamino)pyridin-2-yl)piperidine-4-carboxylic acid. Yield 0.081 g (100%).

### (c) N-[(5-chloro-2-thienyl)sulfonyl]-1-[5-(5-ethyl-1,3-oxazol-2-yl)-3-(isopropylamino)pyridin-2-yl]piperidine-4-carboxamide

A solution of 1-(5-(5-ethyloxazol-2-yl)-3-(isopropylamino)pyridin-2-yl)piperidine-4-carboxylic acid (0.0081, 0.585 mmol), EDCI (0.059 g, 0.31 mmol), HOBt (0.042 g, 0.31 mmol), DIPEA (0.74 mL, 0.71 mmol), 5-chlorothiophene-2-sulfonamide (0.061 g, 0.31 mmol) in DCM (10 mL) was stirred at room temperature for 24 h. The mixture was concentrated, diluted with EtOAc (50 mL), washed with saturated NH₄Cl (25 mL), brine (25 mL), dried (MgSO₄), and concentrated. Flash chromatography (50% EtOAc/hexanes with 1% AcOH) furnished N-[(5-chloro-2-thienyl)sulfonyl]-1-[5-(5-ethyl-1 ,3-oxazol-2-yl)-3-(isopropylamino)pyridin-2-yl]piperidine-4-carboxamide as a solid.

¹H NMR (400 MHz, CDCl₃): δ 1.25 (6H, d, J=6.3 Hz), 1.30 (3H, t, J=7.5 Hz), 1.80-1.90 (2H, m), 1.95-1.98 (2H, m), 2.33-2.40 (1H, m), 2.66-2.78 (4H, m), 3.40-3.51 (2H, m), 3.60-3.68 (1H, m), 6.49 (1W, s), 6.97 (1H, d, J=4.1 Hz), 7.32 (1H, s), 7.71 (1n, d, J=4.1 Hz), 8.23 (1H, s).

MS^{m}/z: 539 (M+1).

### Example 115

### N-[(5-chloro-2-thienyl)sulfonyl]-1-[3-(dimethylamino)-5-(S-ethyl-1,3-oxazol-2-yl pyridin-2-yl]piperidine-4-carboxamide

### (a) Methyl 1-(3-(dimethylamino)-5-(5-ethyl-1,3-oxazol-2-ylpyridin-2-yl)piperidine-4-carboxylate

A solution of methyl 1-(3-amino-5-(5-ethyl-1,3-oxazol-2-yl)pyridin-2-yl)piperidine-4-carboxylate, see example 121, (0.660 g, 2.00 mmol) and iodomethane (0.249 mL, 4.00 mmol) in DMF (5.0 mL) was treated with Cs₂CO₃ (1.30 g, 4.00 mmol) and heated to 80° C. in a sealed tube for 3 h. Additional iodomethane (0.249 mL, 4.00 mmol) was added and the mixture was heated for 3 h at 80° C. The reaction mixture was diluted with EtOAc (100 mL), washed with water (50 mL), brine (4×50 mL), dried (MgSO₄) and concentrated. Flash chromatography (30% EtOAc/hexanes) furnished methyl 1-(5-(5-ethyl-1,3-oxazol-2-yl)-3-(methylamino)pyridin-2-yl)piperidine-4-carboxylate as an oil. Yield 0.127 g (35%).

¹H NMR (400 MHz, CDCl₃): δ 1.32 (3W, t, J=7.6 Hz), 1.82-1.92 (2H, m), 2.02-2.05 (2H, m), 2.48-2.55 (1H, m), 2.72-2.80 (8H, m), 4.22-4.26 (2H, m) 6.80 (1H, s), 7.62 (1H, s), 8.47 (1H, s).

MS^{m}/z: 359 (M+1).

### (b) N-[(5-chloro-2-thienyl)sulfonyl]-1-[3-(dimethylamino)-5-(5-ethyl-1,3-oxazol-2-yl)pyridin-2-yl]piperidine-4-carboxamide

Utilizing the methodology employed in Example 114 sections b and c, methyl 1-(3-(dimethylamino)-5-(5-ethyloxazol-2-yl)pyridin-2-yl)piperidine-4-carboxylate was converted to N-[(5-chloro-2-thienyl)sulfonyl]-1-[3-(dimethylamino)-5-(5-ethyl-1,3-oxazol-2-yl)pyridin-2-yl]piperidine-4-carboxamide.

¹H NMR (400 MHz, CDCl₃): δ 1.31 (3H, t, J=7.5 Hz), 1.79-1.92 (4H, m), 2.32-2.40 (1R^{X}, m), 2.52-2.59 (2H, m), 2.72-2.77 (8H, m), 4.24-4.27 (2H, m), 6.86 (1H, s), 6.96 (1H, d, J=4.1 Hz), 7.58 (1H, d, J=1.9 Hz), 7.70 (1H, d, J=4.1 Hz), 8.42 (1H, d, J=1.9 Hz).

MS^{m}/z: 524 (M+1).

### Example 116

### N-[(5-chloro-2-thienyl)sulfonyl]-1-[5-(5-ethyl-1,3-oxazol-2-yl)-3-(methylamino)pyridin-2-yl]piperidine-4-carboxamide

### (a) Methyl 1-(5-(5-ethyl-1,3-oxazol-2-yl)-3-(methylamino)pyridin-2-yl)piperidine-4-carboxylate

A solution of methyl 1-(3-amino-5-(5-ethyl-1,3-oxazol-2-yl)pyridin-2-yl)piperidine-4-carboxylate (0.660 g, 2.00 mmol), see example 121, and iodomethane (0.249 mL, 4.00 mmol) in DMF (5.0 mL) was treated with Cs₂CO₃ (1.30 g, 4.00 mmol) and heated to 80° C. in a sealed tube for 3 h. Additional iodomethane (0.249 mL, 4.00 mmol) was added and the mixture was heated for 3 h at 80° C. The reaction mixture was diluted with EtOAc (100 mL), washed with water (50 mL), brine (4×50 mL), dried (MgSO₄) and concentrated. Flash chromatography (30% EtOAc/hexanes) furnished methyl 1-(5-(5-ethyl-1,3-oxazol-2-yl)-3-(methylamino)pyridin-2-yl)piperidine-4-carboxylate as an oil. Yield 0.274 g (80%).

¹H NMR (400 MHz, CDCl₃): δ 1.31 (3H, t, J=7.6 Hz), 1.82-1.92 (2H, m), 2.05-2.08 (2H, m), 2.48-2.56 (1H, s), 2.73-2.85 (4H, m), 2.91 (3H, d, J=5.2 Hz), 3.41-3.44 (2H, m), 3.72 (3H, s), 6.82 (1H, s), 7.34 (1H, s), 8.32 (1H, s).

MS^{m}/z: 345 (M+1).

### (b) N-[(5-chloro-2-thienyl)sulfonyl]-1-[5-(5-ethyl-1,3-oxazol-2-yl)-3-(methylamino)pyridin-2-yl]piperidine-4-carboxamide

Utilizing the methodology employed in Example 114 sections b and c, methyl 1-(5-(5-ethyloxazol-2-yl)-3-(methylamino)pyridin-2-yl)piperidine-4-carboxylate was converted to N-[(5-chloro-2-thienyl)sulfonyl]-1-[5-(5-ethyl-1,3-oxazol-2-yl)-3-(methylamino)pyridin-2-yl]piperidine-4-carboxamide.

¹H NMR (400 MHz, CDCl₃): δ 1.31 (3H, t, J=7.5 Hz), 1.80-1.95 (4H, m), 2.36-2.41 (1H, m), 2.61-2.67 (1H, m), 2.76 (2H, q, J=7.5 Hz), 2.87 (3H, s), 3.40-3.43 (24, m), 4.23 (1H, br s), 6.87 (1H, s), 6.96 (1H, d, J=4.0 Hz), 7.30 (1H, s), 7.70 (1H, d, J=4.0 Hz), 8.24 (1H, s).

MS^{m}/z: 510 (M+1).

### Example 117

### Ethyl 6-[4-({[(5-chloro-2-thienyl)sulfonyl]amino}carbonyl)piperidin-1-yl]-5-cyano-2-methylnicotinate

### Ethyl 6-chloro-5-cyano-2-methylnicotinate (0.20 g, 0.89 mmol), N-(5-chlorothiophen-2-ylsulfonyl)piperidine-4-carboxamide hydrochloride (0.41 g. 1.3 mmol), see example 158, and DIPEA (0.62 ml, 3.6 mmol) were combined in DMA (2.0 ml). The reaction was heated at 160° C. for 30 minutes. The reaction was then cooled and dissolved in EtOAc (75 ml) and washed with aqueous NH₄Cl (2×40 ml) followed by brine (40 ml). The organic phase was dried (MgSO₄) and concentrated in vacuo to provide a crude solid. This solid was purified by washing with MeOH followed by EtOAc to provide the desired product, ethyl 6-[4-({[(5-chloro-2-thienyl)sulfonyl]amino}carbonyl)piperidin-1-yl]-5-cyano-2-methylnicotinate, as a white solid. Yield: 198 mg (45%).

¹H NMR (400 MHz, d₆-DMSO): δ 1.30 (3H, t, J=7.1 Hz), 1.50-1.59 (4H, m), 1.80 (2H, d, J=11.0 Hz), 2.42-2.56 (1H, obs), 2.63 (3H, s), 3.15 (2H, d, J=11.9 Hz), 4.24 (2H, q, J=7.1 Hz), 4.49 (2H, d, J=13.5 Hz), 7.28 (1H, d, J=4.1 Hz), 7.67 (1H, d, J=4.1 Hz), 8.32 (1H, s).

MS^{m}/z: 497 (M+1).

### Example 118

### Ethyl 5-cyano-2-methyl-6-[3-({[(5-methylisoxazol-4-yl)sulfonyl]amino}carbonyl)azetidin-1-yl]nicotinate

Prepared according to method A starting from 5-methylisoxazole-4-sulfonamide (0.061 g, 0.38 mmol). Yield: 0.0012 g (1.1%).

¹H NMR (400 MHz, d₆-DMSO) δ 1.25 (3H, t, J=7.1 Hz), 2.57 (3H, s), 3.34-3.24 (3H, s, overlapped by water), 3.51 (1H, m), 4.19 (2H, q, J=7.1 Hz), 4.29 (2H, m), 4.37 (2H, m), 4.46 (1H, s), 8.23 (1H, s)

MS^{m}/z: 434.1 (M+1)

### Example 119

### 1-[3-Chloro-5-(5-ethyl-1,3-oxazol-2-yl)-4-(methylsulfinyl)pyridin-2-yl]-N-[(5-chloro-2-thienyl)sulfonyl]piperidine-4-carboxamide

### (a) 1-(3-Chloro-5-(5-ethyl-1,3-oxazol-2-yl)-4-methylsulfinyl)pyridin-2-yl)piperidine-4-carboxylic acid

Methyl 1-(3-chloro-5-(5-ethyl-1,3-oxazol-2-yl)-4-(methylsulfinyl)pyridin-2-yl)piperidine-4-carboxylate (0.100 g, 0.240 mmol), example 112, and lithium hydroxide (1 M, 2.40 mL, 2.40 mmol) were dissolved in THF (2 mL) and stirred at room temperature for 45 min. The reaction mixture was concentrated under reduced pressure. H₂O (10 mL) was added to the reaction mixture and HCl (conc.) was added drop-wise until the pH was lowered to pH 2. The solution was washed with EtOAc (3×40 mL), dried (MgSO₄), and concentrated under reduced pressure to afford 1-(3-chloro-5-(5-ethyl-1,3-oxazol-2-yl)-4-(methylsulfinyl)pyridin-2-yl)piperidine-4-carboxylic acid as a solid, which was used crude assuming a 100% yield.

### (b) 1-[3-Chloro-5-(5-ethyl-1,3-oxazol-2-yl)-4-(methylsulfinyl)pyridin-2-yl]-N-[(5-chloro-2-thienyl)sulfonyl]piperidine-4-carboxamide

1-(3-chloro-5-(5-ethyl-1,3-oxazol-2-yl)-4-(methylsulfinyl)pyridin-2-yl)piperidine-4-carboxylic acid (0.083 g, 0.210 mmol), EDCI (0.048 g, 0.250 mmol) and HOBT (0.034 g, 0.250 mmol) were dissolved in DCM (2 mL) at room temperature. The reaction mixture was stirred at room temperature for 10 minutes and then 5-chlorothiophene-2-sulfonamide (0.045 g, 0.230 mmol) and DIPEA (0.150 mL, 1.00 mmol) were added. The reaction mixture was stirred at room temperature for 16 h The reaction mixture was diluted with EtOAc (40 mL). The combined organics were washed with 50% saturated NaHCO₃ in brine (30 mL), dried (MgSO₄) and concentrated under reduced pressure to afford the crude product. Flash chromatography (80% EtOAc in Hexanes with 0.5% AcOH) gave 1-(3-chloro-5-(5-ethyloxazol-2-yl)-4-(methylsulfinyl)pyridin-2-yl)-N-(5-chlorothiophen-2-ylsulfonyl)piperidine-4-carboxamide as a solid. Yield: 0.060g (50%).

¹H NMR (400 MHz, CDCl₃): δ 1.23-1.33 (4H, m), 1.49-167 (2H, m), 1.75-1.99 (4H, m), 2.33-2.46 (1H, m), 2.71-2.93 (4H, m), 3.27 (3H, s), 3.81-4.00 (2H, m), 6.89-6.98 (2H, m), 7.63-7.71 (1H, m), 8.48-8.54 (1H, m).

MS^{m}/z: 577 (M+1).

### Example 120

### Ethyl 6-[4-({[(5-chloro-2-thienylsulfonyl]amino}carbonyl)piperidin-1-yl]-2,4-dimethylnicotinate

### (a) Ethyl 6-chloro-2,4-dimethylnicotinate

POCl₃ (2.5 ml, 27 mmol) was added to ethyl 2,4-dimethyl-6-oxo-1,6-dihydropyridine-3-carboxylate [Chem. Pharm. Bull. Japan 1980, 28, 2244] (1.33 g, 6.8 mmol) and the mixture was heated at 110° C. for 4 hours. The reaction was cooled and poured into ice and the excess POCl₃ was allowed to react. The mixture was then extracted with EtOAc (2×100 ml) and the organic phase was washed with water (50 ml) and brine (50 ml). The solution was dried (MgSO₄), concentrated in vacuo and purified through a short plug of silica (10% EtOAc in hexanes) to provide ethyl 6-chloro-2,4-dimethylnicotinate. Yield: 1.30 g (89%).

¹H NMR (400 MHz, CDCl₃): δ 1.40 (3H, t, J=7.1 Hz), 2.33 (3H, s), 2.53 (3H, s), 4.42 (2H, q, J=7.1 Hz), 7.05 (1H, s).

MS^{m}/z: 214 (M+1).

### (b) Ethyl 6-(4-(methoxycarbonyl)piperidin-1-yl)-2,4-dimethylnicotinate Ethyl 6-chloro-2,4-dimethylnicotinate (1.04 g. 4.9 mmol), methyl piperidine-4-carboxylate (1.4 g, 9.7 mmol) and DIPEA (2.3 ml, 15 mmol) were combined in DMA (8 ml) and heated at 110° C. for 14 hours. The reaction was cooled and partitioned between saturated aqueous NH₄Cl (100 ml) and EtOAc (200 ml). The organic phase was washed with additional NH₄Cl (2×75 ml), water (3×75 ml) and brine (50 ml). The organic phase was then dried (MgSO₄), concentrated in vacuo and purified by column chromatography (15% to 20% EtOAc in hexanes) to provide ethyl 6-(4-(methoxycarbonyl)piperidin-1-yl)-2,4-dimethylnicotinate. Yield: 1.15g (74%).

¹H NMR (400 MHz, CDCl₃): δ 1.37 (3H, t, J=7.1 Hz), 1.68-1.78 (2H, m), 1.96-1.99 (2H, m), 2.30 (3H, s), 2.46 (3H, s), 2.52-2.58 (1H, m), 2.93-2.99 (2H, m), 3.70 (3H, s), 4.28-4.36 (4H, m), 6.28 (1H, s).

MS^{m}/z: 321 (M+1).

### (c) 1-(5Ethoxycarbonyl)-4,6-dimethylpyridin-2-yl)piperidine-4-carboxylic acid

Ethyl 6-(4-(methoxycarbonyl)piperidin-1-yl)-2,4-dimethylnicotinate (0.25 g, 0.78 mmol) was dissolved in MeOH (5 ml) and THF (1 ml). An solution of 2M NaOH (1.0 ml, 2 mmol) was added. The reaction was monitored by TLC and after 2 hours at room temperature the reaction was complete. The reaction was concentrated and then saturated NH₄Cl was added (20 ml) followed by a small amount of aqueous HCl to bring the pH to 6. The reaction was extracted with EtOAc (3×50 ml). The organics were combined and washed with brine (30 ml), dried (MgSO₄) and concentrated in vacuo. The acid was used without further purification.

### (d) Ethyl 6-[4-({[(5-chloro-2-thienyl)sulfonyl]amino}carbonyl)piperidin-1-yl]-2,4-dimethylnicotinate

1-(5-(Ethoxycarbonyl)-4,6-dimethylpyridin-2-yl)piperidine-4-carboxylic acid (0.090 g, 0.29 mmol), 5-chlorothiophene-2-sulfonamide (0.075 g, 0.38 mmol), HOBT (0.052 g, 0.38 mmol) and EDCI (0.073 g, 0.38 mmol) were combined in DCM (4 ml) and DIPEA (0.16 ml, 0.88 mmol) was added. The reaction was allowed to stir 14 hours and was then partitioned between EtOAc (75 ml) and aqueous NH₄Cl (60 ml). The organic phase was washed with NH₄Cl (50 ml) and brine (40 ml) and dried (MgSO₄). The solution was then concentrated in vacuo and purified by column chromatography (30% EtOAc/hexanes to 50% EtOAc/hexanes, then add 0.2% AcOH) to provide ethyl 6-[4-({[(5-chloro-2-thienyl)sulfonyl]amino}carbonyl)piperidin-1-yl]-2,4-dimethylnicotinate. Yield: 0.050 g (35%).

¹H NMR (400 MHz, CDCl₃): δ 1.37 (3H, t, J=7.1 Hz), 1.64-1.74 (2H, m), 1.87-1.90 (2H, m), 2.29 (3H, s), 2.39-2.45 (1H, m), 2.44 (3H, s), 2.84-2.91 (2H, m), 4.32-4.38 (4H, m), 6.27 (1H, s), 6.69 (1H, d, J=4.1 Hz), 7.69 (1H, d, J=4.1 Hz).

MS^{m}/z: 486 (M+1).

### Example 121

### 1-[3-(Acetylamino)-5-(5-ethyl-1,3-oxazol-2-yl)pyridin-2-yl]-N-[(5-chloro-2-thienyl)sulfonyl]piperidine-4-carboxamide

### (a) Methyl 1-(3-nitropyridin-2-yl)piperidine-4-carboxylate

A solution of 2-chloro-3-nitropyridine (26.9 g, 170 mmol), methyl piperidine-4-carboxylate (29.2 g, 204 mmol) and DIPEA (32.9 g, 255 mmol) in DMA (100 mL) was heated to 110° C. for 4 h. After cooling to room temperature, the mixture was diluted with EtOAc (600 mL), washed with water (300 mL), saturated NaHCO₃ (300 mL), brine (300 mL), dried (MgSO₄), passed through silica gel and concentrated to furnish methyl 1-(3-nitropyridin-2-yl)piperidine-4-carboxylate as an oil. Yield: 45.0 g (100%).

¹H NMR (400 MHz, C)Cl₃): δ 1.82-1.91 (2H, m), 1.99-2.06 (2H, m), 02.57-2.65 (1H, m), 3.09-3.16 (2H, m), 3.72 (3H, s), 3.79-3.83 (2H, m), 6.73-4.77 (1H, m), 8.12-8.15 (1H, m), 8.32-8.34 (1H, m).

MS^{m}/z: 266 (M+1).

### (b) Methyl 1-(5-bromo-3-nitropyridin-2-yl)piperidine-4-carboxylate

To a solution of methyl 1-(3-nitropyridin-2-yl)piperidine-4-carboxylate (45.0 g, 170 mmol) in CH₃CN (500 mL) was added NBS (30.2 g, 170 mmol) and the reaction mixture was stirred at room temperature for 30 minutes. After concentration, the mixture was diluted with EtoAc (600 mL), washed with saturated NaHCO₃ (2×300 mL), 10% NaHS₂O₃ (2×300 mL) brine (300 mL), dried (MgSO₄), passed through silica gel and concentrated. Flash chromatography (10% EtOAc/hexanes) furnished methyl 1-(5-bromo-3-nitropyridin-2-yl)piperidine-4-carboxylate as a solid. Yield: 53.9 g (92%).

¹H NMR (400 MHz, CDCl₃): δ 1.79-1.89 (2H, m), 1.98-2.02 (2H, m), 2.57-2.64 (1H, m), 3.08-3.15 (2H, m), 3.71 (3H, s), 3.74-3.78 (2H, m), 8.24-8.25 (1H, m), 8.33-8.34 (1H, m).

MS^{m}/z: 346 (M+1).

### (c) Methyl 1-(5-(5-ethyl-1,3-oxazol-2-yl)-3-nitropyridin-2-yl)piperidine-carboxylate

To a solution of 5-ethyl-1,3-oxazole (3.95 g, 40.7 mmol) in THF (mL) cooled to −78° C. under N₂, was added drop-wise over 15 minutes BuLi (1.62 M in pentane, 25.1 mL, 40.7 mmol). ZnCl₂ (16.6 g, 122 mmol) was added in one portion and the reaction mixture was stirred for 10 minutes and then warmed to room temperature. Methyl 1-(5-bromo-3-nitropyridin-2-yl)piperidine-4-carboxylate (10.0 g, 29.1 mmol) was dissolved in THF (40 mL) and added to the reaction mixture. The N₂ atmosphere was replaced with Argon and Pd(PPh₃)₄ (1.68 g, 1.45 mmol) was added. The mixture was heated to 60° C. for 24 h, concentrated, diluted with EtOAc (300 mL), washed with saturated NH₄Cl (2×100 mL), brine (100 mL), dried (MgSO₄) and concentrated. Flash chromatography (15% EtOAc/hexanes) furnished methyl 1-(5-(5-ethyl-1,3-oxazol-2-yl)-3-nitropyridin-2-yl)piperidine-4-carboxylate as a solid. Yield: 2.56 g (24%).

MS^{m}/z: 361 (M+1).

### (d) Methyl 1-(3-amino-5-(5-ethyl-1,3-oxazol-2-yl)pyridin-2-yl)piperidine-4-carboxylate

To a biphasic mixture of methyl 1-(5-(5-ethyl-1,3-oxazol-2-yl)-3-nitropyridin-2-yl)piperidine-4-carboxylate (2.56 g, 7.10 mmol) in THF (70 mL) and H₂O (20 mL) cooled to 0° C. was added zinc dust (3.72 g, 56.8 mmol) and saturated NH₄Cl (100 mL). The reaction mixture was stirred for 30 minutes, diluted with EtOAc (300 mL), washed with saturated NH₄Cl (2×100 mL), brine (100 mL), dried (MgSO₄) and concentrated. Flash chromatography (30% EtOAc/hexanes) furnished methyl 1-(3-amino-5-(5-ethyl-1,3-oxazol-2-yl)pyridin-2-yl)piperidine-4-carboxylate as a solid. Yield: 1.10 g (47%).

¹H NMR (400 MHz, CDCl₃): δ 1.30 (3H, t, J-7.5 Hz), 1.83-1.93 (2H, m), 2.05-2.09 (2H, m), 2.48-2.56 (1H, m), 2.74 (2H, q, J=7.5 Hz), 2.80-2.86 (2H, m), 3.50-3.53 (2H, m), 3.72 (3H, s), 3.83 (2H, br s), 6.81 (1H, s), 7.51 (1H, d, J=2.0 Hz), 8.39 (1H, d, J=2.0 Hz).

MS^{m}/z: 331 (M+1).

### (e) Methyl 1-(3-acetamido-5-(5-ethyl-1,3-oxazol-2-yl)pyridin-2-yl)piperidine-4-carboxylate

To a solution of methyl 1-(3-amino-5-(5-ethyl-1,3-oxazol-2-yl)pyridin-2-yl)piperidine-4-carboxylate (0.106 g, 0.320 mmol) and DIPEA (0.067 mL, 0.39 mmol) in DCM (1.0 mL) cooled to 0° C., was added acetyl chloride (0.023 mL, 0.39 mmol). The reaction mixture was warmed to room temperature and stirred for 1 h. After concentration, the mixture was diluted with EtOAc (60 mL), washed with saturated NH₄Cl (2×30 mL), brine (30 mL), dried (MgSO₄) and concentrated. Flash chromatography (50% EtOAc/hexanes) furnished methyl 1-(3-acetamido-5-(5-ethyl-1,3-oxazol-2-yl)pyridin-2-yl)piperidine-4-carboxylate as a solid. Yield: 0.095 g, (80%).

MS^{m}/z: 373 (M+1).

### (f) 1-[3-(Acetylamino)-5-(5-ethyl-1,3-oxazol-2-yl)pyridin-2-yl]-N-[(5-chloro-2-thienyl)sulfonyl]piperidine-4-carboxamide

Utilizing the methodology employed in Example 114 sections b and c, methyl 1-(3-acetamido-5-(5-ethyloxazol-2-yl)pyridin-2-yl)piperidine-4-carboxylate was converted to 1-[3-(Acetylamino)-5-(5-ethyl-1,3-oxazol-2-yl)pyridin-2-yl]-N-[(5-chloro-2-thienyl)sulfonyl]piperidine-4-carboxamide.

¹H NMR (400 MHz, CDCl₃): δ 1.30 (3H, t, J=7.5 Hz), 1.85-1.95 (2H, m), 2.01-2.05 (2H, m), 2.25 (3H, s), 2.37-2.44 (1H, m), 2.75 (2H, q, J=7.5 Hz), 2.81-2.87 (2H, m), 3.27-3.30 (2H, m), 6.86 (1H, s), 6.97 (1H, d, J=4.1 Hz), 7.71 (1H, d, J=4.1 Hz), 8.60 (1H, br s), 8.69 (1H, d, J=1.9 Hz) 9.04 (1H, s).

MS^{m}/z: 539 (M+1).

### Example 122

### 1-[3-Chloro-5-(5-ethyl-1,3-oxazol-2-yl)-4-(hydroxymethyl)pyridin-2-yl]-N-[(5-chloro-2-thienyl)sulfonyl]piperidine-4-carboxamide

### (a) 2,3-Dichloro-5-(5-ethyl-1,3-oxazol-2-yl)isonicotinaldehyde

2-(5,6-Dichloropyridin-3-yl)-5-ethyl-1,3-oxazole (1.000 g, 4.11 mmol), see example 112, was dissolved in THF (50 mL) and cooled to −78° C. LDA (3.43 mL, 6.17 mmol) was added drop-wise and the reaction mixture stirred for 1 h at −78° C. DMF (0.952 mL, 12.30 mmol) was added in drop-wise one portion and the system slowly warmed to room temperature and stirred at room temperature overnight. The reaction mixture was poured onto saturated aqueous NH₄Cl (80 mL) and extracted into EtOAc (2×75 mL). The combined organics were dried (MgSO₄) and concentrated under reduced pressure to afford the crude material. Flash chromatography (eluant 1:9 EtOAc/hexanes) gave 2,3-dichloro-5-(5-ethyl-1,3-oxazol-2-yl)isonicotinaldehyde as a solid. Yield: 0.620g (56%).

¹H NMR (400 MHz, CDCl₃): δ 1.32 (3H, t, J=7.5 Hz), 2.78 (2H, q, J=7.5 Hz), 6.94 (1H, s), 8.92 (1H, s), 10.42 (1H, s).

MS^{m}/z: 270 (M+1).

### (b) (2,3-Dichloro-5-(5-ethyl-1,3-oxazol-2-yl)pyridin-4-yl)methanol

2,3-Dichloro-5-(5-ethyl-1,3-oxazol-2-yl)isonicotinaldehyde (0.160 g, 0.590 mmol) was dissolved in MeOH (5 mL) and sodium borohydride (0.022 g, 0.590 mmol) was added portion-wise. The reaction mixture was stirred at room temperature until complete consumption of starting material was observed by HPLC analysis. The solvent was concentrated under reduced pressure and the residue partitioned between EtOAc (40 mL) and saturated aqueous NH₄Cl (30 mL). The organics were separated, dried (MgSO₄) and concentrated under reduced pressure to afford the crude (2,3-dichloro-5-(5-ethyl-1,3-oxazol-2-yl)pyridin-4-yl)methanol, which was used without further purification. Yield: 0.157 g (97%).

### (c) 1-[3-Chloro-5-(5-ethyl-1,3-oxazol-2-yl)-4-(hydroxymethyl)pyridin-2-yl]-N-[(5-chloro-2-thienyl)sulfonyl]piperidine-4-carboxamide tri-fluoro acetic acid salt

(2,3-Dichloro-5-(5-ethyl-1,3-oxazol-2-yl)pyridin-4-yl)methanol (0.157 g, 0.60 mmol), N-(5-chlorothiophen-2-ylsulfonyl)piperidine-4-carboxamide hydrochloride (0.300 g, 0.090 mmol) and DIPEA (0.30 mL, 2.00 mmol) were suspended in DMA (7 mL) and heated at 120° C. until complete consumption of starting material was observed by HPLC analysis. The reaction mixture was concentrated under reduced pressure to afford the crude material. The crude material was partitioned between DCM (50 mL) and 1N HCl (30 mL) and the organics separated, dried (MgSO₄) and concentrated under reduced pressure to afford the crude material. Reverse phase column chromatography gave 1-[3-chloro-5-(5-ethyl-1,3-oxazol-2-yl)-4-(hydroxymethyl)pyridin-2-yl]-N-[(5-chloro-2-thienyl)sulfonyl]piperidine-4-carboxamide tri-fluoro acetic acid salt as a solid. Yield: 0.002 g (1%).

¹H NMR (400 MHz, CDCl₃): δ 1.32 (3H, t, J=7.5 Hz), 1.85-2.00 (5H, m), 2.36-2.48 (1H, m), 2.78 (2H, q, J=7.5 Hz), 2.86-2.97 (2H, m), 3.89-2.99 (2H, m), 4.96 (2H, s), 6.88 (1H, s), 6.97 (1H, d, J=4.1 Hz), 7.71 (1H, d, J=4.1 Hz), 8.11 (1H, s), 8.70 (1H, s).

MS^{m}/z: 545 (M+1).

### Example 123

### 1-[3-amino-5-(5-ethyl-1,3-oxazol-2-yl)pyridin-2-yl]-N-[(5-chloro-2-thienyl)sulfonyl]piperidine-4-carboxamide

N-[(5-chloro-2-thienyl)sulfonyl]-1-[5-(5-ethyl-1,3-oxazol-2-yl)-3-nitropyridin-2-yl]piperidine-4-carboxamide (0.10 g, 0.19 mmol) see example 126, was dissolved in MeOH/THF (6 ml, 1:1) and zinc dust (0.10 g. 1.5 mmol) was added. A saturated solution of NH₄Cl (0.7 ml) was added slowly over 2 minutes with slight cooling using an ice water bath and the reaction was stirred 2 hours. The reaction was filtered and the solids were washed with MeOH (25 ml). The filtrate was concentrated and partitioned between EtOAc (75 ml) and saturated aqueous NH₄Cl (40 ml). The organic phase was dried (MgSO₄), concentrated and purified by column chromatography (30 to 50% EtOAc/hexanes then adding AcOH 0.5%) to provide 1-[3-amino-5-(5-ethyl-1,3-oxazol-2-yl)pyridin-2-yl]-N-[(5-chloro-2-thienyl)sulfonyl]piperidine-4-carboxamide as a solid. Yield: 0.023 g (24%).

¹H NMR (400 MHz, CDCl₃): δ 1.30 (3H, t, J=7.6 Hz), 1.85-1.92 (2H, m), 1.96-1.99 (2H, m), 2.34-2.40 (1H, m), 2.71-2.79 (4H, m), 3.54-3.57 (2H, m), 3.80 (2H, s), 6.82 (1H, s), 6.97 (1H, d, J=4.1 Hz), 7.50 (1H, d, J=1.8 Hz), 7.71 (1H, d, J=4.1 Hz), 8.30 (1H, bs), 8.36 (1H, d, J=1.8 Hz).

MS^{m}/z: 496 (M+1).

### Example 124

### 4-[3-chloro-5-(cyclopropylcarbonyl)pyridin-2-yl]-N-[(chloro-2-thienyl)sulfonyl]piperazine-1-carboxamide

### (a) tert-Butyl -(3-chloro-5-(cyclopropanecarbonyl)pyridin-2-yl)piperazine-1-carboxylate

To a solution of tert-butyl 4-(3-chloro-5-(methoxy(methyl)carbamoyl)pyridin-2-yl)piperazine-1-carboxylate (1.00 g, 2.60 mmol), see example 36, in THF (20 mL) cooled to 0° C. was added cyclopropyl magnesium bromide (0.5 M in THF, 10.4 mL, 5.20 mmol). The reaction mixture was stirred for 10 minutes at 0° C. and 1 h at room temperature. The reaction was quenched with saturated NH₄Cl (10 mL), diluted with EtOAc (200 mL), washed with saturated NH₄Cl (2×50 mL), brine (50 mL), dried (MgSO₄) and concentrated. Flash chromatography (15% EtOAc/hexanes) furnished tert-butyl 4-(3-chloro-5-(cyclopropanecarbonyl)pyridin-2-yl)piperazine-1-carboxylate as a solid. Yield: 0.800g (84%).

¹H NMR (400 MHz, CDCl₃): δ 1.03-1.08 (2H, m), 1.23-1.26 (2H, m), 1.49 (9H, s), 2.51-2.57 (1H, m), 3.53-3.60 (8H, m), 8.15 (1H, d, J=2.0 Hz), 8.81 (1H, d, J=2.0 Hz).

MS^{m}/z: 366 (M+1).

### (b) (5-Chloro-6-(piperazin-1-yl)pyridin-3-yl)(cyclopropyl)methanone dihydrochloride

tert-Butyl 4-(3-chloro-5-(cyclopropanecarbonyl)pyridin-2-yl)piperazine-1-carboxylate (0.360 mg, 0.984 mmol) was suspended in MeOH (10 mL) and EtOAc (10 mL). HCl (4.9 mL, 19.68 mmol) solution in 1,4-dioxane was added and the reaction mixture stirred at room temperature for 4 h. The reaction mixture was concentrated under reduced pressure to afford the crude material, which was used without any further purification assuming 100% yield.

### (c) 4-[3-chloro-5-(cyclopropylcarbonyl)pyridin-2-yl]-N-[(5-chloro-2-thienyl)sulfonyl]piperazine-1-carboxamide

(5-Chloro-6-(piperazin-1-yl)pyridin-3-yl)(cyclopropyl)methanone dihydrochloride (102 mg, 301 mmol) and 2,2,2-trichloroethyl 5-chlorothiophen-2-ylsulfonylcarbamate (112 mg, 301 mmol) were suspended in DMA (7 mL). DIPEA (0.262 mL, 1.506 mmol) and DMAP (0.002 mg, 0.015 mmol) were added and the system heated at 100° C. for 5 h. The reaction mixture was diluted with EtOAc (50 mL) and washed sequentially with saturated NH₄Cl (2×40 mL), brine (1×40 mL), dried (MgSO₄) and concentrated under reduced pressure to afford the crude material. Flash chromatography (gradient elution 1:9 EtOAc/hexanes, 0.5% AcOH to 3:7 EtOAc/hexanes, 0.5% AcOH) gave 4-[3-chloro-5-(cyclopropylcarbonyl)pyridin-2-yl]-N-[(5-chloro-2-thienyl)sulfonyl]piperazine-1-carboxamide as a solid. Yield: 0.065 g (44%).

¹H NMR (400 MHz, CDCl₃): δ 1.09-1.12 (2H, m), 1.26-1.29 (2H, m), 2.52-2.58 (1H, m), 3.63 (8H, s), 6.95 (1H, d, J=4.2 Hz), 7.68 (1H, d, J=4.2 Hz), 8.21 (1H, d, J=1.9 Hz), 8.85 (1H, d, J=1.9 Hz).

MS^{m}/z: 489 (M+1).

### Example 125

### N-[({1-[3-cyano-5-(5-ethyl-1,3-oxazol-2-yl)-6-methylpyridin-2-yl]azetidin-3-yl}amino)carbonyl]-4-methylbenzenesulfonamide

### (a) Ethyl 5-cyano-2-methyl-6-oxo-1-{[2-(trimethylsilyl)ethoxy]methyl}-1,6-dihydropyridine-3-carboxylate

The sodium salt of ethyl 5-cyano-2-methyl-6-oxo-1,6-dihydropyridine-3-carboxylate (8.81 g, 38.6 mmol) was distributed equally into 8 Smith process vials. To each vial was added DCM (3 mL), [2-(chloromethoxy)ethyl](trimethyl)silane (1.78 g, 10.7 mmol), and then DIPEA (2.07 g, 16.0 mmol). Each vial was heated in a microwave oven, single node heating, at 120° C. for 10 minutes. Extra [2-(chloromethoxy)ethyl] (trimethyl)silane (0.445 g, 2.68 mmol) was added to each vial and the single node heating was continued at 120° C. for 10 minutes. The reaction mixtures were combined and vacuum filtered. Purification by flash chromatography on SiO₂ with heptane/EtOAc 4:1 or 3:1 afforded the pure product. Yield: 8.376 g (58%).

¹H NMR (400 MHz, CDCl₃): 5-0.18 (9H, s), 0.75 (2H, t, J=8.0 Hz), 1.19 (3H, t J=7.2 Hz), 2.78 (3H, s), 3.52 (2H, t, J=8.0 Hz), 4.13 (2H, q, J=7.2 Hz), 5.46 (2H, s), 8.16 (1H, s)

MS^{m}/z: 335 (M−1).

### (b) 5-cyano-2-methyl-6-oxo-1-{[2-(trimethylsilyl)ethoxy]methyl}-1,6-dihydropyridine-3-carboxylic acid

Ethyl 5-cyano-2-methyl-6-oxo-1-{[2-(trimethylsilyl)ethoxy]methyl}-1,6-dihydropyridine-3-carboxylate (8.371 g, 24.9 mmol) was dissolved in THF (50 mL) and 1M LiOH (100 mL) was added. The reaction mixture was stirred at rt for 3 h. The conversion was complete according to LC/MS. 4M HCl was added to pH 2-3. The WATER phase was extracted with EtOAc (3×100 mL). The organic phases were combined and dried with sodium sulphate and evaporated. To give a crude material. Yield: 8.35 g (109%). The isomeric ethyl 5-cyano-2-methyl-6-{[2-(trimethylsilyl)ethoxy]methoxy}nicotinate was formed as the main product according to LC/MS, which showed a product/by-product ratio of 25:75. No attempt was made to separate the isomers.

MS^{m}/z: 307 (M−1).

### (c) 5-cyano-N-(2-hydroxybutyl)-2-methyl-6-oxo-1-{[2-(trimethylsilyl)ethoxy]methyl}-1,6-dihydropyridine-3-carboxamide

A mixture (7.67 g, 24.9 mmol) of 5-cyano-2-methyl-6-oxo-1-{[2-(trimethylsilyl)ethoxy]methyl}-1,6-dihydropyridine-3-carboxylic acid and the isomer ethyl 5-cyano-2-methyl-6-{[2-(trimethylsilyl)ethoxy]methoxy}nicotinate, in a ratio of 25:75 according to LC/MS, was dissolved in DCM (125 mL). EDCI (6.2 g, 27.4 mmol) and HOBt (5.04 g, 37.3 mmol) were added and the reaction mixture was stirred at rt for 40 minutes. 1-aminopropan-2-ol (2.44 g, 27.7 mmol) in DIPEA (16.1 g, 124.4 mmol) was added and stirring at rt was continued for 1.5 h. According to LC/MS only the minor isomer had been converted at this point. Stirring at rt was continued for 16 h further without any change in LC/MS. The organic phase was extracted with 10% potassium carbonate (2×125 mL), brine (2×125 mL), dried with sodium sulphate and evaporated. This gave 12.21 g crude product. Purification by flash chromatography on Si-gel with heptane/EtOAc fractions, first 1:2, then 1:4, eluted afforded 5-cyano-N-(2-hydroxybutyl)-2-methyl-6-oxo-1-{[2-(trimethylsilyl)ethoxy]methyl}-1,6-dihydropyridine-3-carboxamide.

Yield: 3.28 g (35%). When all product had been eluted, elution was done with heptane/EtOAc 1:4+1% formic acid. In this way, 2.46 g of ethyl 5-cyano-2-methyl-6-{[2-(trimethylsilyl)ethoxy]methoxy}nicotinate was recovered.

¹H NMR (400 MHz, CDCl₃): 6-0.13 (s, 9H), 0.87-0.77 (m, 5H), 1.44-1.31 (m, 2H), 2.58 (s, 3H), 3.15-3.06 (m, 1H), 3.46-3.38 (m, 1H), 3.60-3.50 (m, 4H), 5.41 (s, 2H), 7.26-7.21 (m, 1H), 7.77 (s, 1H)

MS^{m}/z: 378 (M−1).

### (d) 5-cyano-2-methyl-6-oxo-N -2-oxobutyl)-1-{[2-(trimethylsilyl)ethoxy]methyl}-1,6-dihydropyridine-3-carboxamide

Oxalyl chloride (0.39 g, 3.05 mmol) was dissolved in DCM (2 mL) under an atmosphere of nitrogen and the solution was cooled to −78° C. DMSO (0.37 g, 4.69 mmol) in DCM (1 mL) was added dropwise and the mixture was stirred at −78° C. for less than 5 minutes. 5-cyano-N-(2-hydroxybutyl)-2-methyl-6-oxo-1-{[2-(trimethylsilyl)ethoxy]methyl}-1,6-dihydropyridine-3-carboxamide (0.89 g, 2.35 mmol) in DCM (2 mL) was added during 2 minutes and stirring at −78° C. was continued for 1 h. TEA (1.19 g, 11.7 mmol) was added. After stirring for 15 minutes the cooling bath removed and the reaction mixture was stirred at ambient temperature for 15 minutes. Water (10 mL) was added and the water phase was extracted with DCM (3×15 mL). The organic phases were combined and dried with sodium sulphate and evaporated to give the crude product which was used without further purification. Yield: 0.780g (88%).

¹H NMR (500 MHz, CDCl₃): 6-0.12 (s, 9H), 0.81 (t, J=8.2 Hz, 2H), 0.97 (t, J=7.4 Hz, 2H), 2.40 (q, J=7.4 Hz, 2H), 2.63 (s, 3H), 3.55 (t J=8.2 Hz, 2H), 4.09 (d, J=5.3 Hz, 2H), 5.45 (s, 2H), 7.50 (t, J=5.3 Hz, 1H), 7.86 (s, 1H)

MS^{m}/z: 376 (M−1).

### (e) 5-(ethyl-1,3-oxazol-2-yl)-6-methyl-2-oxo-1-{[2-(trimethylsilyl)ethoxy]methyl}-1,2-dihydropyridine-3-carbonitrile

5-cyano-2-methyl-6-oxo-N-(2-oxobutyl)-1-{[2-(trimethylsilyl)ethoxy]methyl}-1,6-dihydropyridine-3-carboxamide (2.761 g, 7.31 mmol) was dissolved in THF (9.6 mL) and the solution was transferred equally into 3 Smith process vials. To each vial was added (Methoxycarbonylsulfamoyl)triethylammonium hydroxide, inner salt (1.162 g, 4.88 mmol). The vials were sealed and heated in a microwave oven, single node heating, at 80° C. for 2 minutes. LC/MS on each vial showed complete conversion. The reaction mixtures were combined and evaporated to give 6.431 g of a crude material. Filtration through a Si-plug (10 g) with Heptane/EtOAc 1:1 (100 mL) afforded 5-(5-ethyl-1,3-oxazol-2-yl)-6-methyl-2-oxo-1-{[2-(trimethylsilyl)ethoxy]methyl}-1,2-dihydropyridine-3-carbonitrile. Yield: 1.766 g (67%).

¹H NMR (500 MHz, CDCl₃): 6-0.20 (s, 9H), 0.74 (t, J=8.0 Hz, 2H), 1.09 (t, J=7.5 Hz, 3F), 2.55 (q, J=7.5 Hz, 2H), 2.82 (s, 3H), 3.52 (t J=8.0 Hz, 2H), 5.46 (5, 2H), 6.62 (s, 1H), 8.09 (s, 1H)

MS^{m}/z: 358 (M−1).

### (f) 5-(5-ethyl-1,3-oxazol-2-yl)-6-methyl-2-ox-1,2-dihydropyridine-3-carbonitrile

A TFA/DCM mixture (1:1, 10 mL) was added to (5-(5-ethyl-1,3-oxazol-2-yl)-6-methyl-2-oxo-1-{[2-(trimethylsilyl)ethoxy]methyl}-1,2-dihydropyridine-3-carbonitrile (1.682 g, 4.68 mmol) and the reaction mixture was stirred at rt for 4 h. According to LC/MS the reaction was complete. The reaction mixture was evaporated. DCM (10 mL) was added and the mixture was dried with sodium sulphate and evaporated. This gave 0.263 g crude material. Purification by flash chromatography on Si-gel with DCM/MeOH (69:1, then 39:1) afforded the title compound. Yield: 0.263 g (82%).

¹H NMR (300 MHz, DMSO-d₆): δ 1.24 (br t, J=7.5 Hz, 3H), 2.68 (s, 3H), 2.73 (br q, J=7.5 Hz, 2H), 7.00 (br s, 1H), 8.51 (s, 1H), 12.97 (s, 1H)

MS^{m}/z: 230 (M+1).

### (g) 2-chloro-5-(5-ethyl-1,3-oxazol-2-yl)-4-methylnicotinonitrile

5-(5-ethyl-1,3-oxazol-2-yl)-6-methyl-2-oxo-1,2-dihydropyridine-3-carbonitrile (0.069 g, 0.30 mmol) was dissolved in DCM (0.8 mL) in a Smith process vial and oxalyl chloride (0.573 g, 4.51 mmol) and then DMF (0.022 g, 0.3 mmol) were added at 0° C. The reaction mixture was heated in the sealed vial on an oil bath at 50° C. for 2.5 h. LC/MS showed 33% wanted product and 45% starting material. Stirring at the same temperature was continued. After 1.5 h further, extra DMF (0.022 g, 0.30 mmol) was added. Stirring at the same temperature was performed for 7.5 h further. LC/MS showed 64% wanted product and 8% starting material. The reaction mixture was evaporated and mixed with a batch that was prepared in the following manner:

5-(5-ethyl-1,3-oxazol-2-yl)-6-methyl-2-oxo-1,2-dihydropyridine-3-carbonitrile (0.179 g, 0.78 mmol) was dissolved in 0CM (2.4 mL) in a Smith process vial and oxalyl chloride (1.486 g, 11.70 mmol) and then DMF (0.057 g, 0.78 mmol) were added at 0° C. The reaction mixture was heated in the sealed vial on an oil bath at 50° C. for 4 h. LC/MS showed 40% wanted product and 22% starting material. Extra DMF (0.057 g, 0.78 mmol) was added. Stirring at the same temperature was performed for 16 h further. LC/MS showed 35% wanted product and no starting material. The material was evaporated. Purification of the combined batches was done by flash chromatography on Si-gel with DCM/MeOH 199:1 as eluent to afford 2-chloro-5-(5-ethyl-1,3-oxazol-2-yl)-6-methylnicotinonitrile. Yield: 0.027 g (10%/0).

¹H NMR (400 MHz, CDCl₃): δ 1.34 (t, J=7.5 Hz, 3H), 2.80 (q, J=7.5 Hz, 2H), 3.00 (s, 3H), 6.97 (s, 1H), 8.52 (s, 1H)

### (h) ter-butyl {1-[3-cyano-5-(5-ethyl-1,3-oxazol-2-yl)-4-methylpyridin-2-yl]azetidin-3-yl}carbamate

2-Chloro-5-(5-ethyl-oxazol-2-yl)-6-methyl-nicotinonitrile (245 mg, 0.79 mmol), azetidin-3-yl-carbamic acid tert-butyl ester (160 mg, 0.93 mmol), and triethylamine (0.27 ml, 1.97 mmol) were mixed in 99.5% ethanol (4 ml) and heated at 120° C. for 10 min using a Emrys Optimizer microwave oven from Personal Chemistry. The solvent was evaporated and the crude mixture was dissolved in DCM (3 ml) and filtered through a 5g /25 ml silica gel column and concentrated to give the title compound as a yellow solid. Yield: 0.170g (53%).

1H NMR (400 MHz, CDCl₃) δ 1.27 (3H, t, J=7.5 Hz), 1.44 (9H, s), 2.71 (2H, q, J=7.5 Hz), 2.75 (3H, s), 4.15 (2H, dd, J=9.8, 4.9 Hz), 4.58 (1H, broad s), 4.66 (2H, m), 4.98 (1H, broad s), 6.80 (1H, s), 8.18 (1H, s)

MS^{m}/z: 384 (M+1)

### (i) N-[({1-[3-cyano-5-(5-ethyl-1,3-oxazol-2-yl)-6-methylpyridin-2-yl]azetidin-3-yl}amino)carbonyl]-4-methylbenzenesulfonamide

tert-butyl {1-[3-cyano-5-(5-ethyl-1,3-oxazol-2-yl)-6-methylpyridin-2-yl]azetidin-3-yl}carbamate (0.140 g, 0.365 mmol) was stirred in a mixture of TFA (1.5 ml) and DCM (4 ml) in room temp for 30 minutes. The solvents were evaporated and the crude material was dissolved in triethylamine (0.3 ml) and DCM (3 ml).

Carbonyldiimidazole (0.044 g, 0.27 mmol), 4-toluenesulfon amide (0.047 g, 0.27 mmol) and triethylamine (0.15 ml, 11.1 mmol) was stirred in DCM (4 ml) at room temp for 30 min. Half of the deprotected amine was added slowly to the mixture and the reaction was stirred at 40° C. over night. The reaction mixture was purified by preparative HPLC using Kromasil C8 10μ 250 mm×21.2 id. Eluent A: 100% CH₃CN Eluent B: 95% 0.1M NH₄₀ac(aq) and 5% CH₃CN. Conditions used: Flow 20 ml/minutes, isocratic 10 minutes 20% CH₃CN, gradient 20 minutes 20% to 50% CH₃CN. Freeze-drying gave the title compound as a white solid. Yield: 0.012 g (9%).

¹H NMR (400 MHz, CDDL3) δ 1.25 (3H, t, J=7.5 Hz), 2.39 (31H, s), 2.69 (2H, q, J=7.7 Hz), 2.72 (3H, s), 4.10 (2H, d, J=5.4 Hz), 4.60 (3H, m), 6.78 (1H, s), 7.30 (2H, d, J=8.1 Hz), 7.78 (2H, d, J=8.1 Hz), 8.15 (1H, s)

MS^{m}/z: 481 (M+1)

### Example 126

### N-[(5-chloro-2-thienyl)sulfonyl]-1-[5-(5-ethyl-1,3-oxazol-2-yl)-3-nitropyridin-2-yl]piperidine-4-carboxamide

### (a) 1-(5-(5-ethyloxazol-2-yl %3-nitropyridin-2-yl)piperidine-4-carboxylic acid

Methyl 1-(5-(5-ethyloxazol-2-yl)-3-nitropyridin-2-yl)piperidine-4-carboxylate (0.080 g, 0.210 mmol) and sodium hydroxide (2 M, 3 mL, 6.0 mmol) were dissolved in THF (10 mL) and MeOH (40 mL) and stirred at room temperature 16 h. The reaction mixture was concentrated under reduced pressure to afford 1-(5-(5-ethyloxazol-2-yl)-3-nitropyridin-2-yl)piperidine-4-carboxylic acid as a solid, which was used crude assuming a 100% yield.

### (b) N-[(5-chloro-2-thienyl)sulfonyl]-1-[5-(5-ethyl-1,3-oxazol-2-yl)-3-nitropyridin-2-yl]piperidine-4-carboxamide

1-(5-(5-ethyloxazol-2-yl)-3-nitropyridin-2-yl)piperidine-4-carboxylic acid (0.880 g, 2.5 mmol), EDCI (0.630 g, 3.30 mmol) and HOBT (0.450 g, 3.30 mmol) were dissolved in DMA (14 mL) at room temperature. The reaction mixture was stirred at room temperature for 30 minutes and then 5-chlorothiophene-2-sulfonamide (0.700 g, 3.60 mmol) and DIPEA (1.3 ml, 7.60 mmol) were added. The reaction mixture was stirred at room temperature for 16 h. The reaction mixture was diluted with EtOAc (50 mL) and washed with H₂O (3×30 mL). The combined organics were dried (MgSO₄) and concentrated under reduced pressure to afford the crude product. Flash chromatography (50% EtoAc in hexanes then 50% EtOAc in hexanes with 0.5% AcOH) gave N-[(5-chloro-2-thienyl)sulfonyl]-1-[5-(5-ethyl-1,3-oxazol-2-yl)-3-nitropyridin-2-yl]piperidine-4-carboxamide as a solid. Yield: 0.230g (17%).

¹H NMR (400 MHz, CDCl₃): δ 1.31 (3H, t, J=7.6 Hz), 1.80-1.90 (2H, m), 1.93-1.98 (2H, m), 2.47-2.55 (1H, m), 2.75 (2H, q, J=7.6 Hz), 3.10-3.17 (2H, m), 3.92-3.95 (2H, m), 6.83 (1H, s), 6.97 (1H, d, J=4.1 Hz), 7.70 (1H, , J=4.1 Hz), 8.66 (1H, d, J=2.0 Hz), 8.90 (1H, d, J=2.0 Hz).

MS^{m}/z: 526 (M+1).

### Example 127

### N-[(5-chloro-2-thienyl)sulfonyl]-1-[3-cyano-5-(5-ethyl-1,3-oxazol-2-yl)-6-methylpyridin-2-yl]azetidine-3-carboxamide

### (a) 1-[3-Cyano-5-(5-ethyl-1,3-oxazol-2-yl)-6-methylpyridin-2-yl]azetidine-3-carboxylic acid

2-chloro-5-(5-ethyl-1,3-oxazol-2-yl)-6-methylnicotinonitrile (0.028 g, 0.11 mmol), see example 125, was dissolved in dry EtOH (2 mL) in a Smith process vial. Azetidine-3-carboxylic acid (0.023 g, 0.23 mmol) and TEA (0.114 g, 1.13 mmol) were added and the sealed vial was heated in a microwave oven, single node heating, at 120° C. for 20 minutes. LC/MS showed full conversion. The reaction mixture was evaporated. 1M HCl (2 mL) was added. The mixture was extracted with DCM (3×2 mL) by using a phase separator. The organic phase were combined, dried with sodium sulphate and evaporated. This gave 0.033 g crude product. Purification by flash chromatography on Si-gel with DCM/MeOH 39:1+1% formic acid as eluent gave the pure product. Yield: 0.026 g (74%).

¹H NMR (300 MHz, CDCl₃): δ 1.31 (t, J=7.5 Hz, 3H), 2.80-2.70 (m, 5H), 3.70-3.57 (m, 1H), 4.68-4.57 (m, 4H), 6.88 (br s, 1H), 8.20 (s, 1H)

MS^{m}/z: 313 (M+1).

### (b) N-[(S-chloro-2-thienyl)sulfonyl]-1-[3-cyano-5-(S-ethyl-1,3-oxazol-2-yl)-6-methylpyridin-2-yl]azetidine-3-carboxamide

To a stirred solution of 1-[3-cyano-5-(5-ethyl-1,3-oxazol-2-yl)-6-methylpyridin-2-yl]azetidine-3-carboxylic acid (0.082 g, 0.210 mmol) in DCM (5 mL) was added EDCI (0.052 g, 0.273 mmol) and HOBT (0.0426 g, 0.315 mmol). After 30 minutes a solution of 5-chlorothiophene-2-sulfonamide (0.052 g, 0.273 mmol) and DIPEA (0.0814 g, 0.630 mmol). The reaction mixture was stirred at room temperature over night followed by filtration through a silica plug (5g) using 30 mL MeOH/DCM (8°/,). Flash chromatography (gradient 2-4% methanol/DCM) gave N-[(5-chloro-2-thienyl)sulfonyl]-1-[3-cyano-5-(5-ethyl-1,3-oxazol-2-yl)-6-methylpyridin-2-yl]azetidine-3-carboxamide as a solid. Yield: 0.048 g (46.5%/).

¹H NMR (400 MHz, CDCl₃): δ 1.23 (3H:, t, J=7.7 Hz), 2.66 (2H, q, J=7.7 Hz), 2.67 (31H, s), 3.38-3.4×(1H, m), 4.38-4.44 (3H, m), 6.74 (1H, s), 6.87 (1H, d, f4.0 Hz), 7.58 (1H, d, J=4.0 Hz), 8.10 (s)

MS^{m}/z: 492 (M+1).

### Example 128

### N-[(5-chloro-2-thienyl)sulfonyl]-1-[3-cyano-5-(5-ethyl-1,3-oxazol-2-yl)-6-methylpyridin-2-yl]piperidine-4-carboxamide

### (a) 1-[3-Cyano-5-(5-ethyl-1,3-oxazol-2-yl)-6-methylpyridin-2-yl]piperidine-4-carboxylic acid

2-chloro-5-(5-ethyl-1,3-oxazol-2-yl)-6-methylnicotinonitrile (0.056 g, 0.23 mmol), see example 125, was dissolved in dry EtOH (4 mL) in a Smith process vial. Piperidine-3-carboxylic acid (0.051 g, 0.40 mmol) was added and the sealed vial was heated in a microwave oven, single node heating, at 120° C. for 20 minutes. LC/MS showed the reaction to be incomplete. TEA (0.233 g, 2.30 mmol) and extra Piperidine-3-carboxylic acid (0.015 g, 0.11 mmol) were added and the sealed vial was then heated in a microwave oven, single node heating, at 100° C. for 20 minutes. LC/MS showed the reaction to be complete. The reaction mixture was evaporated. 1 M HCl (3 mL) was added and the mixture was extracted with DCM (3×3 mL) by using a phase separator. This gave the crude product which was used without further purification. Yield: 0.086 g (110%)

¹H NMR (300 MHz, CDCl₃): δ 1.17 (t, J=7.5 Hz, 3H), 1.78-1.62 (m, 2H), 1.99-1.86 (m, 2H), 2.51-2.39 (m, 1H), 2.67-2.57 (m, 51), 3.16-3.04 (m, 21), 4.43-4.32 (m 2H), 6.70 (br s, 1H), 8.11 (s, 1R)

MS^{m}/z: 341 (M+1).

### (b) N-[(5-chloro-2-thienyl)sulfonyl]-1-[3-cyano-5-(5-ethyl-1,3-oxazol-2-yl)-6-methylpyridin-2-yl]piperidin e-4-carboxamide

To a stirred solution of 1-[3-cyano-5-(5-ethyl-1,3-oxazol-2-yl)-6-methylpyridin-2-yl]piperidine-4-carboxylic acid (0.130 g, 0.306 mmol) in D-CM was added EDCI (0.0761 g, 0.397 mmol) and HOBT (0.0619 g, 0.458 mmol). After 30 minutes a solution of 5-chlorothiophene-2-sulfonamide (0.079 g, 0.397 mmol) and DIPEA (0.118 g, 0.916 mmol). The reaction mixture was stirred at room temperature over night followed by filtration through a silica plug (5g) using 30 mL MeOH/DCM (8%). Precipitation of 0.0396 g of the crude material from warm ethanol gave N-[(5-chloro-2-thienyl)sulfonyl]-1-[3-cyano-5-(5-ethyl-1,3-oxazol-2-yl)-6-methylpyridin-2-yl]piperidine-4-carboxamide as solid. Yield: 0.017 g (10.4%).

¹H NMR (400 MHz, CDCl₃): δ 1.28 (3H, t, J=7.4 Hz), 1.75-1.84 (2H, m), 1.90-1.98 (2H, m), 2.43-2.53 (1H, m), 2.74 (2H, q, J=7.4 Hz), 3.08-3.19 (2H, m), 4.50-4.60 (2H, m), 6.82 (1H, s), 6.94 (1H, d, J=4.4), 7.68 (1H, d, J=4.4), 8.15 (1H, s), 8.27 (1H, s)

MS^{m}/z: 520 (M+1).

### Example 129

### 1-[3-Chloro-5-(5-ethyl-1,3-oxazol-2-yl)-4-methylpyridin-2-yl]-N-[(5-chloro-2-thienyl)sulfonyl]piperidine-4-carboxamide

### (a) 2-(5,6-Dichloro-4-methylpyridin-3-yl)-5-ethyl-1,3-oxazole

2-(5,6-Dichloropyridin-3-yl)-5-ethyl-1,3-oxazole (0.500 g, 2.06 mmol) was dissolved in THF (100 mL) and cooled to −78° C. LDA (2.0 mL, 3.60 mmol) was added dropwise and the reaction mixture allowed to slowly warm to 0° C. MeI (0.289 mL, 4.63 mmol) was added in one portion and the system stirred at room temperature for 2 h. The reaction mixture was poured onto saturated NH₄Cl (50 mL) and extracted into EtOAc (100 mL). The combined organics were dried (MgSO₄) and concentrated under reduced pressure to afford the crude material, which was passed through a silica plug to give 2-(5,6-dichloro-4-methylpyridin-3-yl)-5-ethyl-1,3-oxazole as a solid. Yield: 0.200g (38%).

MS^{m}/z: 257 (M+1).

### (b) 1-(3-Chloro-5-(5-ethyl-1,3-oxazol-2-yl)-4-methylpyridin-2-yl)-N-(5-chlorothiophen-2-ylsulfonyl)piperidine-4-carboxamide

Crude 2-(5,6-dichloro-4-methylpyridin-3-yl)-5-ethyl-1,3-oxazole (0.200 g, 0.778 mmol), N-(5-chlorothiophen-2-ylsulfonyl)piperidine-4-carboxamide hydrochloride (0.403 g, 1.17 mmol), see example 158, and DIPEA (0.406 mL, 2.33 mmol) were suspended in DMF (5 mL) and heated at 120° C. until complete consumption of starting material was observed by HPLC analysis. The reaction mixture was concentrated under reduced pressure to afford the crude material. The crude material was partitioned between DCM (30 mL) and 1N HCl (20 mL) and the organics separated and dried (MgSO₄) and concentrated under reduced pressure to afford the crude material, which was purified by reverse phase prep HPLC to afford 1-[3-chloro-5-(5-ethyl-1,3-oxazol-2-yl)-4-methylpyridin-2-yl]-N-[(5-chloro-2-thienylsulfonyl]piperidine-4-carboxamide as a solid. Yield: 0.050g (12%).

¹H NMR (400 MHz, CDCl₃): δ 1.31 (3H, t, J=7.5 Hz), 1.86-1.97 (4H, m), 2.33-2.43 (1H, m), 2.70 (3H, s), 2.73-2.85 (4H, m), 3.82-3.91 (2H, m), 6.90 (1H, s), 6.97 (1H, d, J=4.2 Hz), 7.71 (1H, d, J=4.2 Hz), 8.50 (1H, br s), 8.60 (1H, s).

MS^{m}/z: 529 (M+1).

### Example 130

### Ethyl 6-[3-({[(5-chloro-2-thienyl)sulfonyl]amino}carbonyl)azetidin-1-yl]-5-cyano-2-methylnicotinate

### 1-[3-cyano-5-(ethoxycarbonyl)-6-methylpyridin-2-yl]azetidine-3-carboxylic acid (0.258 g, 0.890 mmol), see example 56, EDCI (0.180 g, 1.16 mmol) and HOBT (0.156 g, 1.57 mmol) was dissolved in DCM and stirred for 30 minutes. 5-chlorothiophene-2-sulfonamide (0.264 g, 1.33 mmol) followed by DIPEA (0.47 mL, 2.67 mmol) was added. The reaction was stirred for 18 h followed by removal of solvents in vacuo. The crude product was purified by flash chromatography (0-100% EtOAc/heptane followed by MeOH/DCM 0-40%). The isolated product was dissolved in DCM and filtered through an ion exchanger (Isolute CBA 1 g) followed by an additional amount of DCM (5 column volumes) The fractions were combined and the solvents was removed in vacuo to give ethyl 6-[3-({[(5-chloro-2-thienyl)sulfonyl]amino}carbonyl)azetidin-1-yl]-5-cyano-2-methylnicotinate. Yield: 0.061 g (15%).

¹H NMR (400 MHz, CDCl₃): 1.29 (3H, t, J=7.0 Hz), 2.60 s, 31), 3.55-3.68 (1H, m), 4.22 (2H, q, J=7.0 Hz), 4.26-4.28 (2H, m), 4.37-4.46 (2H, m), 7.22 (1H, d, f-0.2), 7.68 (1H, d, J=4.2), 8.25 (1H, s).

MS^{m}/z: 469 (M+1)

### Example 131

### N-[(5-chloro-2-thienyl)sulfonyl]-1-[5-(5-ethyl-1,3-oxazol-2-yl)-3-methylpyridin-2-yl]piperidine-4-carboxamide

### (a) Methyl 1-(5-bromo-3-methylpyridin-2-yl)piperidine-4-carboxylate

### 5-Bromo-2-chloro-3-methylpyridine (1.23 g, 5.96 mmol), methyl piperidine-4-carboxylate (1.7 g, 12 mmol) and DIPEA (1.6 ml, 9.0 mmol) where combined in DMA (2 ml) and heated for 36 h. The reaction was cooled and diluted with EtOAc (75 ml) and washed with 0.5 N HCl (30 ml), water (2×40 ml), brine (30 ml) and dried (MgSO₄). The crude reaction mixture was concentrated in vacuo and purified by column chromatography (15% EtOAc/hexanes) to provide methyl 1-(5-bromo-3-methylpyridin-2-yl)piperidine-4-carboxylate. Yield: 0.49 g (26%).

¹H NMR (400 MHz, CDCl₃): δ 1.82-1.92 (2H, m), 2.00-2.05 (2H, m), 2.25 (3H, s), 2.45-2.52 (1H, m), 2.77-2.83 (2H, m), 3.39-3.42 (2H, m), 3.71 (3H, s), 7.51 (1H, d, J=2.2 Hz), 8.16 (1H, d, J=2.2 Hz).

MS^{m}/z: 313/315 (M+1, Br pattern).

### (b) Methyl 1-(5-(5-ethyl-1,3-oxazol-2-yl)-3-methylpyridin-2-yl)piperidine-4-carboxylate

5-Ethyl-1,3-oxazole (0.21 g, 2.2 mmol) was dissolved in dry THF (2.5 ml) and cooled to −78° C. A 2.4 M solution of nBuLi in hexanes (0.90 ml, 2.2 mmol) was added slowly over 15 min at −78° C. and after 5 min, solid zinc chloride (0.66 g, 4.8 mmol) was added in one portion. The external bath was removed and the reaction was allowed to warm to room temperature over 15 minutes and the reaction was then allowed to stir at room temperature 15 minutes. Methyl 1-(5-bromo-3-methylpyridin-2-yl)piperidine-4-carboxylate (0.45 g, 1.4 mmol) was added to the reaction in a solution of dry THF (1.5 ml). The reaction was then purged with argon gas and tetrakis(triphenylphosphine)palladium(0) (0.083 g, 0.072 mmol) was added. The reaction was purged with argon and heated at 60° C. for 16 h. The reaction was cooled and partitioned between EtOAc (75 ml) and saturated aqueous NH₄Cl (50 ml). The organic phase was separated and washed with NH₄Cl (40 ml), brine (40 ml) and dried (MgSO₄). The solution was concentrated in vacuo and purified by column chromatography (15 to 35% EtOAc/hexanes) to provide methyl 1-(5-(5-ethyl-1,3-oxazol-2-yl)-3-methylpyridin-2-yl)piperidine-4-carboxylate. Yield: 0.18 g (38%).

¹H NMR (400 MHz, CDCl₃): δ 1.30 (3H, t, J=7.6 Hz), 1.84-1.94 (21, m), 2.02-2.06 (2H, m), 2.32 (3H, s), 2.48-2.56 (1H, m), 2.74 (2H, q, J=7.6 Hz), 2.85-2.92 (2H, m), 3.56-3.59 (2H, m), 3.72 (3H, s), 6.80 (1H, s), 7.97 (1H, s), 8.72 (1H, d, J=1.9 Hz). MS^{m}/z: 330 (M+1).

### (c) 1-(5-(5-ethyloxazol-2-yl)-3-methylpyridin-2-yl piperidine-4-carboxylic acid

Methyl 1-(5-(5-ethyloxazol-2-yl)-3-methylpyridin-2-yl)piperidine-4-carboxylate (0.050 g, 0.15 mmol) was dissolved in MeOH (2 ml) and 2N NaOH (0.3 ml, 0.6 mmol) was added and the reaction was allowed to stir 14 h. The reaction was neutralized with aqueous HCl to near pH 7 and was then concentrated in vacuo. The residue was partitioned between EtOAc (50 ml) and saturated NH₄Cl (40 ml). The organic phase was dried (MgSO₄) and concentrated in vacuo to provide 1-(5-(5-ethyloxazol-2-yl)-3-methylpyridin-2-yl)piperidine-4-carboxylic acid which was used without further purification.

### (d) N-[(5-chloro-2-thienyl)sulfonyl]-1-[5-(5-ethyl-1,3-oxazol-2-yl)-3-methylpyridin-2-yl]piperidine-4-carboxamide

1-(5-(5-Ethyloxazol-2-yl)-3-methylpyridin-2-yl)piperidine-4-carboxylic acid (0.050 g, 0.16 mmol), EDCI (0.040 g, 0.21 mmol), HOBT (0.028 g, 0.21 mmol) and 5-chlorothiophene-2-sulfonamide (0.055 g, 0.32 mmol) were combined in DMA (1.5 ml). DIPEA (0.24 ml, 1.4 mmol) was added and the reaction was allowed to stir 14 hr. The reaction was concentrated to remove most of the solvent and the residue was then partitioned between ET(OAc (75 mL) and saturated aqueous NH₄Cl (40 ml). The organic phase was washed with water (2×30 ml) and then brine (20 ml). The organic phase was dried (MgSO₄) and concentrated. The crude reaction mixture was purified by column chromatography (30 to 50% EtOAc/hexanes, then added 0.5% AcOH). N-[(5-chloro-2-thienyl)sulfonyl]-1-[5-(5-ethyl-1,3-oxazol-2-yl)-3-methylpyridin-2-yl]piperidine-4-carboxamide was isolated as a solid. Yield: 0.050g (64%).

¹H NMR (400 MHz, CDCl₃): δ 1.30 (3H, t, J=7.6 Hz), 1.83-1.97 (4H, m), 2.29 (3H, s), 2.35-2.43 (1H, m), 2.75 (2H, q, J=7.6 Hz), 2.79-2.86 (2H, m), 3.58-3.61 (2H, m), 6.82 (1H, s), 6.97 (1H, d, J=4.1 Hz), 7.71 (1H, d, J=4.1 Hz), 7.97 (1H, s), 8.71 (1H, d₃J=1.9 Hz).

MS^{m}/z: 495 (M+1).

### Example 132

### 1-[3-Chloro-5-(5-ethyl-1,3-oxazol-2-yl)pyridin-2-yl]-N-[(5-chloro-2-thienyl)sulfonyl]piperidine-4-carboxamide

1-(3-Chloro-5-(5-ethyloxazol-2-yl)pyridin-2-yl)piperidine-4-carboxylic acid (0.200 g, 0.60 mmol), see example 112, EDCI (0.148 g, 0.77 mmol) and HOBT (0.105 g, 0.77 mmol) were suspended in DCM (10 mL) at room temperature. The reaction mixture was stiffed at room temperature for 30 minutes and then 5-chlorothiophene-2-sulfonamide (0.177 g, 0.89 mmol) and DIPEA (0.311 mL, 1.79 mmol) was added drop-wise. The reaction mixture was stirred at room temperature until complete consumption of the starting material was observed by HPLC analysis. The reaction mixture was diluted with DCM (50 mL) and washed with saturated NH₄Cl (1×30 mL). The combined organics were dried (MgSO₄) and concentrated under reduced pressure to afford the crude material. Flash chromatography (gradient elution 3:7 EtOAc/hexanes, 0.5% AcOH to 7:3 EtOAc/hexanes, 0.5% AcOH) gave 1-[3-Chloro-5-(5-ethyl-1,3-oxazol-2-yl)pyridin-2-yl]-N-[(5-chloro-2-thienyl)sulfonyl]piperidine-4-carboxamide as a solid. Yield: 0.213 g (69%).

¹H NMR (400 MHz, CDCl₃): δ 1.30 (3H, t, J=7.6 Hz), 1.84-2.00 (4H, m), 2.38-2.47 (1H, m), 2.75 (2H, q, J=7.6 Hz), 2.84-2.96 (2H, m), 3.96-4.03 (2H, m), 6.83 (1H, s), 6.97 (1H, d, J=4.2 Hz), 7.71 (1H, d, J=4.2 Hz), 8.14 (1H, d, J=1.7 Hz), 8.73 (1H, d, J=1.7 Hz).

MS^{m}/z: 515 (M+1).

### Example 133

### 1-[3-Chloro-5-(5-propyl-1,3-oxazol-2-yl)pyridin-2-yl]-N-[(5-chloro-2-thienyl)sulfonyl]piperidine-4-carboxamide

Starting with 2-propyloxirane in place of 2-butyloxirane and employing the same methodology used to generate 1-[5-(5-butyl-1,3-oxazol-2-yl)-3-chloropyridin-2-yl]-N-[(5-chloro-2-thienyl)sulfonyl]piperidine-4-carboxamide, 1-[3-Chloro-5-(5-propyl-1,3-oxazol-2-yl)pyridin-2-yl]-N-[(5-chloro-2-thienylsulfonyl]piperidine-4-carboxamide was produced as a solid.

¹H NMR (400 MHz, CDCl₃): δ 1.01 (3H, t, J=7.4 Hz), 1.68-1.77 (2H, m), 1.86-1.96 (4H, m), 2.40-2.47 (1H, m), 2.69 (2H, t, J=7.4 Hz), 2.84-2.90 (2H, m), 3.96-4.00 (2H, m), 6.85 (1H, m), 6.97 (1H, d, J=4.1 Hz), 7.71 (1H, d, J=4.1 Hz), 8.12 (1H, s), 8.71 (1H, s), 8.77 (1H, br s).

MS^{m}/z: 530 (M+1).

### Example 134

### 1-[5-(5-Butyl-1,3-oxazol-2-yl)-3-chloropyridin-2-yl]-N-[(5-chloro-2-thienyl)sulfonyl]piperidine-4-carboxamide

### (a) 5-Chloro-6-(4-(methoxycarbonyl)piperidin-1-yl)nicotinic acid

A suspension of 5,6-dichloronicotinic acid (25.0 g, 130 mmol), methyl piperidine-4-carboxylate (23.3 g, 163 mmol) and DIPEA (45.4 mL, 260 mmol) in DMA (200 mL) was heated to 120° C. until complete consumption of starting material was observed by HPLC analysis. The reaction mixture was concentrated under reduced pressure and partitioned between DCM (500 mL) and 1M HCl (250 mL). The organic layer was washed with brine (250 mL), dried (MgSO₄) and concentrated under reduced pressure to afford 5-chloro-6-(4-(methoxycarbonyl)piperidin-1-yl)nicotinic acid.

¹H NMR (400 MHz, CDCl₃): δ 1.86-1.95 (2H, m), 2.03-2.07 (2H, m), 2.55-2.62 (1H, m), 3.03-3.09 (2H, m), 3.72 (3H, s), 4.12-4.15 (2H, m), 8.15 (1H, s), 8.79 (1H, s).

MS^{m}/z: 299 (M+1).

### (b) 1-Aminohexan-2-ol

To a solution of concentrated ammonium hydroxide (70 mL) and MeOH (100 mL), 2-butyloxirane (8.31 g, 83.0 mmol) was added drop-wise. The resulting solution was stirred at room temperature for 3 d and concentrated. Dilution with MeOH (50 mL) followed by concentration was performed 3 times to afford 1-aminohexan-2-ol which was used crude assuming 100% conversion.

### (c) Methyl 1-(3-chloro-5-(2-hydroxyhexylcarbamoyl)pyridin-2-yl)piperidine-4-carboxylate

A solution of 5-chloro-6-(4-(methoxycarbonyl)piperidin-1-yl)nicotinic acid (4.00 g, 13.4 mmol), EDCI (3.34 g, 17.4 mmol) and HOBT (2.35 g, 17.4 mmol) were dissolved in DCM (100 mL) and stirred at room temperature. After 30 minutes, 1-aminohexan-2-ol (3.14 g, 26.8 mmol) and DIPEA (7.00 mL, 40.2 mmol) were added. The reaction was stirred overnight, concentrated, diluted with EtOAc (250 mL), washed with saturated NH₄Cl (2×100 mL), saturated NaHCO₃ (2×100 mL), brine (100 mL), dried (MgSO₄), passed through silica gel and concentrated. Flash chromatography (60% EtOAc/hexanes) produced methyl 1-(3-chloro-5-(2-hydroxyhexylcarbamoylpyridin-2-ylpiperidine-4-carboxylate. Yield: 3.4 g (64%).

¹H NMR (400 MHz, CDCl₃): δ 0.91 (3H, t, J=6.9 Hz), 1.34-1.38 (4H, m), 1.49-1.52 (2H, m), 1.85-1.95 (2H, m), 2.00-2.05 (2H, m), 2.44 (1H, br s), 2.52-2.58 (1H, m), 2.94-3.01 (2H, m), 3.24-3.31 (1H, m), 3.67-3.73 (4H, m), 3.81 (1H, br s), 3.97-4.00 (2H, m), 6.54-6.56 (1H, m), 7.99 (1E, s), 8.51 (1H, s).

MS^{m}/z: 398 (M+1).

### (d) Methyl 1-(3-chloro-5-(2-oxohexylcarbamoyl)pyridin-2-yl)piperidine-4-carboxylate

To a solution of oxalyl chloride (1.28 g, 10.1 mmol) in DCM (50 mL) at −78° C. was added drop-wise DMSO (1.43 mL, 20.1 mmol). After stirring for 5 minutes, a solution of methyl 1-(3-chloro-5-(2-hydroxyhexylcarbamoylpyridin-2-yl)piperidine-4-carboxylate (2.00 g, 5.03 mmol) in DCM (50 mL) was added drop-wise to the reaction mixture. After stirring for 20 minutes, TEA (3.50 mL, 25.1 mmol) was added the reaction was stirred for an additional 30 minutes. The cooling bath was removed and stirring was continued for 30 minutes. The reaction mixture was quenched with water (50 mL). The organic layer and DCM extracts (2×100 mL) were washed with brine, dried (MgSO₄), and concentrated to furnish methyl 1-(3-chloro-5-(2-oxohexylcarbamoyl)pyridin-2-yl)piperidine-4-carboxylate as a solid. Yield: 1.99 g (100%).

MS^{m}/z: 396 (M+1).

### (e) Methyl 1-(5-(5-butyl-1,3-oxazol-2-yl)-3-chloropyridin-2-yl)piperidine-4-carboxylate

A solution of 1-(3-chloro-5-(2-oxohexylcarbamoyl)pyridin-2-yl)piperidine-4-carboxylate (1.00 g, 2.53 mmol) and POCl₃ (1.16 mL, 12.6 mmol) in DMF (50 mL) was heated to 90° C. for 30 minutes. After cooling to room temperature, the reaction mixture was poured onto ice and quenched with saturated NaHCO₃ 100 mL. The combined organic layers from extractions with EtOAc (4×100 mL) were washed w/brine (2×100 mL), dried (MgSO₄), passed through silica gel and concentrated. Flash chromatography (10% EtOAc/hexanes) furnished methyl 1-(5-(5-butyl-1,3-oxazol-2-yl)-3-chloropyridin-2-yl)piperidine-4-carboxylate as a solid. Yield: 0.82 g (86%)

¹H NMR (400 MHz, CDCl₃): δ 0.96 (3H, t, J=7.3 Hz), 1.30-1.38 (2H, m), 1.45-1.64 (2H, m), 1.87-1.97 (2H, m), 2.02-2.06 (2H, m), 2.51-2.59 (1H, m), 2.71 (2H, t, J=7.5 Hz), 2.94-3.00 (2H, m), 3.93 (3H, s), 3.93-3.96 (21, m), 6.82 (1H, s), 8.14 (1H, s), 8.74 (1H, s).

MS^{m}/z: 378 (M+1).

### (f) 1-(5-(5-Butyl-1,3-oxazol-2-yl)-3-chloropyridin-2-yl)piperidine-4-carboxylic acid

A biphasic mixture of 1-(5-(5-butyl-1,3-oxazol-2-yl)-3-chloropyridin-2-yl)piperidine-4-carboxylate (0.400 g, 1.06 mmol) dissolved in THF (50 mL) and aqueous LiOH (1 M, 50 mL) was stirred at room temperature for 20 h. The pH was adjusted to 2 with concentrated HCl. The combined organic layers from extractions with EtOAc (3×75 mL) were dried (MgSO₄), passed through silica gel and concentrated to produce 1-(5-(5-butyl-1,3-oxazol-2-yl)-3-chloropyridin-2-yl)piperidine-4-carboxylic acid as a solid. Yield: 0.277 g (72%).

MS^{m}/z: 364 (M+1).

### (g) 1-[5-(S-Butyl-1,3-oxazol-2-yl)-3-chloropyridin-2-yl]-N-[(5-chloro-2-thienyl)sulfonyl]piperidine-4-carboxamide

A solution of 1-(5-(5-butyl-1,3-oxazol-2-yl)-3-chloropyridin-2-yl)piperidine-4-carboxylic acid (0.277 g, 0.761 mmol), EDCI (0.190 g, 0.990 mmol), and HOBT (0.134 g, 0.990 mmol) in DCM (10 mL) was stirred at room temperature for 30 minutes. After addition of 5-chlorothiophene-2-sulfonamide (0.196 g, 0.990 mmol) and DIPEA (0.398 mL, 2.28 mmol), the reaction mixture was stirred at room temperature for 20 h. Following concentration, the mixture was diluted with EtOAc (100 mL), washed with saturated NH₄Cl (2×50 mL), saturated NaHCO₃ (2×50 mL), brine (50 mL), dried (MgSO₄) and concentrated. Flash chromatography (40% EtOAc/hexanes with 0.5% AcOH) furnished 1-[5-(5-Butyl-1,3-oxazol-2-yl)-3-chloropyridin-2-yl]-N-[(5-chloro-2-thienyl)sulfonyl]piperidine-4-carboxamide as a solid. Yield: 0.368 g (86%).

¹H NMR (400 MHz, CDCl₃): δ 0.96 (3H, t, J=7.3 Hz), 1.38-1.47 (2H, m), 1.64-1.72 (2H, m), 1.84-1.97 (4H, m), 2.40-2.48 (1H, m), 2.71 (2H, t, J=7.6 Hz), 2.84-2.91 (2H, m), 3.69-4.00 (2H, m), 6.84 (1H, s), 6.97 (1H, d, J=4.2 Hz), 7.71 (1H, d, J=4.2 Hz), 8.12 (1R^{X}, s), 8.72 (1H, s), 3.88 (1H, br s).

MS^{m}/z: 543 (M+1).

### Example 135

5-Chloro-N-[({1-[3-cyano-5-(5-ethyl-1,3-oxazol-2-yl)-6-methylpyridin-2-yl]azetidin-3-yl}amino)carbonyl]thiophene-2-sulfonamide

{1-[3-Cyano-5-(5-ethyl-1,3-oxazol-2-yl)-6-methyl-pyridin-2-yl]-azetidin-3-yl}-carbamic acid tert-butyl ester (0.140 g, 0.365 mmol), see example 125, was stirred in a mixture of TFA (1.5 mL) and DCM (4 mL) at room temperature for 30 minutes. The solvents were evaporated and the crude material was dissolved in triethylamine (0.3 mL) and DCM (3 mL). Carbonyldiimidazole (0.044 g, 0.27 mmol), 5-chlorothiophene-2-sulfonamide (0.054 g, 0.27 mmol) and triethylamine (0.15 mL, 1.1 mmol) was stirred in DCM (4 mL) at room temperature for 30 min. Half of the deprotected amine was added slowly to the mixture and the reaction was stirred at 40° C. over night. The reaction mixture was purified by preparative HPLC using Kromasil C8 10μ 250 mm×21.2 id. Eluent A: 100% CH₃CN, Eluent B: 95% 0.1M NH₄OAc(aq) and 5% acetonitrile. Conditions used: Flow 20 ml /minutes, isocratic 10 minutes 20% acetonitrile, gradient 20 minutes 20% to 50% CH₃CN. Freeze-drying gave the title compound as a white solid. Yield: 0.052 g (55%).

¹H NMR (400 MHz, CDCL3) δ 1.21 (3H, t, J=7.6 Hz), 2.64 (5H, m), 4.08 (2H, d, J=5.4 Hz,), 4.57 (3H, s), 6.73 (1H, s,), 6.85 (1H, d, J=3.8 Hz,), 7.48 (1H, d, J=4.0 Hz,), 8.09 (1H, s,)

MS^{m}/z: 507 (M+1)

### Example 136

### N-[(S5-chloro-2-thienyl)sulfonyl]-4-[3-cyano-5-(5-ethyl-1,3-oxazol-2-yl)-6-methylpyridin-2-yl]piperazine-1-carboxamide

### (a) ter-Butyl 4-[3-cyano-5-(ethoxycarbonyl)-6-methylpyridin-2-yl]piperazine-1-carboxylate

Potassium carbonate (0.62 g, 4.5 mmol) was added to a solution of Ethyl 5-cyano-2-methyl-6-piperazin-1-ylnicotinate (1.03 g, 4.5 mmol), see example 11, in THF (2 mL) and water (5 mL). The reaction mixture was cooled to followed by addition of di-tert-butyl dicarbonate (0.98 g, 4.5 mmol). The reaction mixture was stirred for 10 minutes at 0° C. followed by 16 h at room temperature. DCM (10 mL) was added and the phases were separated. The aqueous phase was further extracted with DCM (10 mL) twice and the combined organics were dried over sodium sulphate, filtered and evaporated to give tert-Butyl 4-[3-cyano-5-(ethoxycarbonyl)-6-methylpyridin-2-yl]piperazine-1-carboxylate. Yield 1.32 g (94 I/O).

¹H NMR (300 MHz, CDCl₃) δ 1.31 (3H, t J=7.1 Hz), 1.42 (9H, s), 2.65 (3H, s), 3.45-3.54 (4H, m), 3.79-3.88 (41H, m), 4.25 (2H, q, J-7.1 Hz), 8.27 (1H, s)

### (b) 6-[4-(tert-Butoxycarbonyl)piperazin-1-yl]-5-cyano-2-methylnicotinic acid

To a solution of tert-Butyl 4-[3-cyano-5-(ethoxycarbonyl)-6-methylpyridin-2-yl]piperazine-1-carboxylate (1.33 g, 3.53 mmol) in THF (7 mL) was added 1M (water) LiOH (7 mL). The reaction mixture was heated at 60° C. for 5 h followed by cooling to 0° C. Acidification with HCl (1M) followed by addition of DCM (15 mL). The phases were separated and the aqueous phase was extracted twice with DCM (15 mL). The combined organic phases were dried over sodium sulphate, filtered and the solvents were removed in vacuo to give tert-Butyl 4-[3-cyano-5-(ethoxycarbonyl)-6-methylpyridin-2-yl]piperazine-1-carboxylate as a solid. Yield: 0.914 g (75%0).

¹H NMR (300 MHz, CDCl₃) δ 1.50 (91t, s), 2.76 (3H, s), 3.56-3.64 (4H, m), 3.90-4.00 (4H, m), 8.45 (1H, s)

### (c) tert-Butyl 4-(3-cyano-{[(2-hydroxybutyl)amino]carbonyl}-6-methylpyridin-2-yl)piperazine-1-carboxylate

To a solution of tert-Butyl 4-[3-cyano-5-(ethoxycarbonyl)-6-methylpyridin-2-yl]piperazine-1-carboxylate (0.91 g, 2.64 mmol) in DCM (25 mL) at 0° C. was added HOBT (0.54 g, 3.96 mmol) and EDCI (0.759 g, 3.96 mmol). The reaction mixture was stirred at room temperature for 30 minutes followed by addition of 1-aminobutan-2-ol (0.35 g, 3.96 mmol) and DIPEA (1.42 mL, 7.9 mmol). The reaction mixture was stirred for 14 h followed by filtration and removal of solvents in vacuo. The crude material was dissolved in ethyl acetate and was extracted twice with ammonium chloride (sat.)(30 mL), twice with sodium bicarbonate (30 mL) and twice with brine (30 mL). The organic layer-was dried over sodium sulphate and the solvents were removed in vacuo to give ) tert-Butyl 4-(3-cyano-5-{[(2-hydroxybutyl)amino]carbonyl}-6-methylpyridin-2-yl)piperazine-1-carboxylate. Yield: 1.06 g (96%).

¹H NMR (300 MHz, CDCl₃) δ 0.94-1.0 (3H, m), 1.46 (9H, s), 1.46-1.60 (2H, m), 2.52 (3H, s), 3.18-3.29 (1H, m), 3.50-3.55 (4H, m), 3.55-3.65 (1H, m), 3.65-3.77 (1H, m),3.75-3.80 (4H, m), 7.85 (1H, s)

### (d) tert-Butyl 4-(3-cyano-6-methyl-5-{[(2-oxobutyl)amino]carbonyl}pyridin-2-yl)piperazine-1-carboxylate

Oxalyl chloride (0.206 g, 1.63 mmol) was dissolved in DCM (2.5 mL) and a solution of DMSO (0.195 g, 2.5 mmol) in DCM (1 mL) was added at −78° C. After 5 minutes a solution of (rac)-tert-butyl 4-(3-cyano-5-{[(2-hydroxybutyl)amino]carbonyl}-6-methylpyridin-2-yl)piperazine-1-carboxylate (0.522 g, 1.25 mmol) in DCM (2.2 mL) was added. The reaction mixture was stirred at −78° C. for 30 minutes before addition of TEA (0.632 g, 6.25 mmol). The reaction mixture was heated to room temperature followed by addition of water (10 mL) and the phases were separated. The aqueous phase was extracted with DCM (2×10 mL) and the combined organic phases were extracted with brine (30 mL), dried over sodium sulphate, filtered and the solvents were removed in vacuo to give tert-Butyl 4-(3-cyano-6-methyl-5-{[(2-oxobutyl)amino]carbonyl}pyridin-2-yl)piperazine-1-carboxylate. Yield: 0.498 g (96%).

¹H NMR (300 MHz, CDCl₃) δ 1.04 (3H, t, f7.4 Hz), 1.40 (9H, s), 2.46 (2H, q, J=7.4 Hz), 2.52 (3H, s), 2.58-2.66 (1H, m), 3.43-3.52 (4H, m), 3.65-3.78 (4H, m), 4.15-4.22 (2H, m), 7.85 (1H, s)

MS^{m}/z: 416 (M+1)

### (e) tert-Butyl 4-[3-cyano-5-(5-ethyl-1,3-oxazol-2-yl)-6-methylpyridin-2-yl]piperazine-1-carboxylate

A solution of tert-butyl 4-(3-cyano-6-methyl-5-{[(2-oxobutyl)amino]carbonyl}pyridin-2-yl)piperazine-1-carboxylate (468 mg, 1.13 mmol), DMAP (catalytical amount) and pyridine (1.78 g, 22.5 mmol) in DCM (5 mL) was cooled to 0° C. followed by addition of trichloroacetyl chloride (1.84 g, 10.1 mmol). The reaction mixture was stirred at 0° C. for two hours followed by stirring at room temperature for 16 h. The reaction mixture was extracted with saturated sodium bicarbonate solution, the organics isolated and the solvents were removed in vacuo. The residue was in methanol (5 mL) and cooled to 0° C. followed by addition of potassium carbonate (0.166 g, 1.20 mmol). After 30 minutes the reaction mixture was partitioned between brine (10 mL) and ethyl acetate (10 mL). The organic layer was isolated and the aqueous layer was further extracted with ethyl acetate (3×10 mL). The combined organic phases were dried over sodium sulphate, filtered and the solvents were removed in vacuo to give the crude product. Flash chromatography (20% EtOAc/hexanes) gave tert-butyl 4-[3-cyano-5-(5-ethyl-1,3-oxazol-2-yl)-6-methylpyridin-2-yl]piperazine-1-carboxylate. Yield: 0.207 g(46%)

1H NMR (300 MHz, CDCl₃) δ 1.31 (3H, t, J=7.6 Hz), 1.49 (9, s), 2.75 (2H, q, J=7.6), 2.80 (3H, s), 3.55-3.63 (4H, m), 3.79-3.83 (41, m), 6.84 (1H, s), 8.30 (1H, s).

### (f) N-[(5-chloro-2-thienyl)sulfonyl]-4-[3-cyano-5-(5-ethyl-1,3-oxazol-2-yl)-6-methylpyridin-2-yl]piperazine 1-carboxamide

4-[3-Cyano-5-(5-ethyl-1,3-oxazol-2-yl)-6-methyl-pyridin-2-yl]-piperazine-1-carboxylic acid tert-butyl ester (0.062 g, 0.156 mmol) was stirred in a mixture of TFA (1.5 mL) and DCM (3 mL) in room temperature for 40 minutes. The solvents were evaporated. Carbonyldiimidazole (0.028 g, 0.172 mmol), 5-chlorothiophene-2-sulfonamide (0.040 g, 0.203 mmol) and DIPEA (0.25 ml, 1.4 mmol) was stirred in DCM (4 ml) at room temp for 1 h. The mixture was added do the deprotected amine and the reaction was stirred at 40° C. over night. The reaction mixture was purified by preparative HPLC using Kromasil C8 10μ 250 mm×21.2 id. Eluent A: 100% CH₃CN, Eluent B: 95% 0.1M NH₄OAc(aq) and 5% CH₃CN. Conditions used: Flow 20 ml minutes, isocratic 10 minutes 20% CH₃CN, gradient 20 minutes 20% to 50% CH₃CN. Freeze-drying gave the title compound as a white solid. Yield: 0.032 g (36%).

¹H NMR (500 MHz, CDCl₃) δ 1.33 (3H, t, J=7.5 Hz), 2.77 (2H, q, J=7.2 Hz,), 2.83 (3H, s), 3.66 (4H, t, J-4.9 Hz), 3.88 (4H, t, J=5.1 Hz), 6.87 (1H ,s), 6.93 (1H, d, J=4.2 Hz), 7.62 (1H, d, J=3.9 Hz), 8.34 (1H, s)

MS^{m}/z: 521 (M+1)

### Example 137

### 1-[3-Chloro-5-(5-ethyl-1,3-oxazol-2-yl)pyridin-2-yl]-N-[(5-chloro-2-thienyl)sulfonyl]azetidine-3-carboxamide

### (a) 1-(3-Chloro-5-(5-ethyloxazol-2-yl)pyridin-2-yl)azetidine-3-carboxylic acid

2-(5,6-Dichloropyridin-3-yl)-5-ethyloxazole (0.401 mg, 1.65 mmol), azetidine-3-carboxylic acid (0.250 mg, 2.47 mmol) and DIPEA (0.86 mL, 4.95 mmol) were suspended in DMF (5 mL) and heated at 120° C. until complete consumption of starting material was observed by HPLC analysis. The reaction mixture was concentrated under reduced pressure to afford the crude material. The crude material was partitioned between DCM (50 mL) and 1N HCl (40 mL) and the organics separated, dried (MgSO₄) and concentrated under reduced pressure to afford the crude material, which was used without any further purification assuming 100% yield.

### (b) 1-[3-Chloro-5-(5-ethyl-1,3-oxazol-2-yl)pyridin-2-yl]-N-[(5-chloro-2-thienyl)sulfonyl]azetidine-3-carboxamide

1-(3-chloro-5-(5-ethyl-1,3-oxazol-2-yl)pyridin-2-yl)azetidine-3-carboxylic acid (0.374 g, 1.21 mmol), EDCI (0.303 g, 1.58 mmol) and HOBT (0.213 g, 1.58 mmol) were suspended in DCM (5 mL) at room temperature. The reaction mixture was stirred at room temperature for 30 minutes and then 5-chlorothiophene-2-sulfonamide (0.360 g, 1.82 mmol) and DIPEA (0.635 mL, 3.64 mmol) was added drop-wise. The reaction mixture was stirred at room temperature until complete consumption of the starting material was observed by HPLC analysis. The reaction mixture was diluted with DCM (50 mL) and washed with saturated NH₄Cl (1×30 mL). The combined organics were dried (MgSO₄) and concentrated under reduced pressure to afford the crude material. Flash chromatography (gradient elution 3:7 EtOAc/hexanes, 0.5% AcOH to 7:3 EtOAc/hexanes, 0.5% AcOH) gave 1-[3-chloro-5-(5-ethyl-1,3-oxazol-2-yl)pyridin-2-yl]-N-[(5-chloro-2-thienyl)sulfonyl]azetidine-3-carboxamide as a solid. Yield: 0.270g (46%).

¹H NMR (400 MHz, CDCl₃): δ 1.30 (3H, t, J=7.6 Hz), 2.74 (2H, q, J -7.6 Hz), 3.37-3.46 (1H, m), 4.39-4.50 (4H, m), 6.80 (1H, s), 6.98 (1H, d, J=4.1 Hz), 7.73 (1H, d, J=4.1 Hz), 8.03 (1H, s), 8.65 (1H, s).

MS^{m}/z: 487 (M+1).

### Example 138

### Ethyl 5-chloro-6-[4-({[(5-chloro-2-thienyl)sulfonyl]amino}carbonyl)piperidin-1-yl]-2,4-dimethylnicotinate

### (a) Ethyl 5-chloro-4-(4-(methoxycarbonyl)piperidin-1-yl)-2,4-dimethylnicotinate

Ethyl 6-(4-(methoxycarbonyl)piperidin-1-yl)-2,4-dimethylnicotinate (0.86 g, 2.7 mmol), see example 120, was dissolved in DCM (4 ml) and N-chlorosuccinimide (0.55 g, 4.1 mmol) was added and the reaction was allowed to stir 48 hours at room temperature. The reaction showed a small amount of starting material remained and was partitioned between EtOAc (70 ml) and saturated aqueous NH₄Cl (50 ml). The organic phase was washed with water (40 ml), brine (40 ml) and was then dried (MgSO₄) and concentrated in vacuo. The reaction mixture was purified by column chromatography (15% EtOAc/hexanes) to provide ethyl 5-chloro-6-(4-(methoxycarbonyl)piperidin-1-yl)-2,4-dimethylnicotinate. Yield: 0.70 g (73%).

¹H NMR (400 MHz, CDCl₃): δ 1.38 (3H, t, J=7.1 Hz), 1.86-1.96 (2H, m), 1.98-2.05 (2H, m), 2.33 (3H, s), 2.41 (3H, s), 2.46-2.54 (1H, m), 2.85-2.91 (2H, m), 3.71 (3H, s), 3.76-3.80 (2H, m), 4.38 (2H, q, J=7.1 Hz).

MS^{m}/z: 355 (M+1),

### (b) Ethyl 5-chloro-6-[4-({[(5-chloro-2-thienylsulfonyl]amino}carbonyl)piperidin-1-yl]-2,4-dimethylnicotinate

Following the general procedure used for Example 7 and substituting ethyl 6-(4-(methoxycarbonyl)piperidin-1-yl)-2,4-dimethylnicotinate with ethyl 5-chloro-6-(4-(methoxycarbonyl)piperidin-1-yl)-2,4-dimethylnicotinate in step (c) of the sequence, and then continuing through the sequence using the same general procedures, then the desired product ethyl 5-chloro-6-[4-({[(5-chloro-2-thienyl)sulfonyl]amino}carbonyl)piperidin-1-yl]-2,4-dimethylnicotinate was obtained.

¹H NMR (400 MHz, CDCl₃): δ 1.38 (3H, t J=7.1 Hz), 1.86-1.92 (4H, m), 2.32 (3H, s), 2.36-2.40 (I, m, obs), 2.40 (3H, s), 2.78-2.85 (2H, m), 3.81-3.85 (2H, m), 4.38 (2H, q, J=7.1 Hz), 6.97 (1H, d, J=4.1 Hz), 7.71 (1H, d, J=4.1 Hz), 8.11 (1H, bs). MS^{m}/z: 520 (M+1).

### Example 139

### 1-[3-Chloro-5-(5-ethyl-1,3-oxazol-2-yl)-4-methoxypyridin-2-yl]-N-[(5-chloro-2-thienyl)sulfonyl]piperidine-4-carboxamide

### (a) Methyl 1-(3-chloro-5-(5-ethyloxazol-2-yl)-4-methoxypyridin-2-yl)piperidine-4-carboxylate

1-(3-chloro-5-(5-ethyl-1,3-oxazol-2-yl)-4-methoxypyridin-2-yl)piperidine-4-carboxylic acid (0.120 g, 0.280 mmol), see example 141 and Sodium methoxide (0.017 g, 0.310 mmol) were dissolved in MeOH (1 mL) at room temperature. The reaction mixture was stirred at room temperature for 16 h. The reaction mixture was concentrated then diluted with EtOAc (40 mL) and the combined organics were washed with H₂O (1×30 mL), dried (MgSO₄) and concentrated under reduced pressure to afford methyl 1-(3-chloro-5-(5-ethyloxazol-2-yl)-4-methoxypyridin-2-yl)piperidine-4-carboxylate as a solid which was used crude.

MS^{m}/z: 380 (M+1).

### (b) 1-(3-chloro-5-(5-ethyloxazol-2-yl)-4-methoxypyridin-2-yl)piperidine-4-carboxylic acid

Methyl 1-(3-chloro-5-(5-ethyloxazol-2-yl)-4-methoxypyridin-2-yl)piperidine-4-carboxylate (0.080 g, 0.210 mmol), and lithium hydroxide (1 M, 10.0 mL, 10.0 mmol) were dissolved in THF (10 mL) and stirred at room temperature 3 h. The reaction mixture was heated at 70° C. for 30 minutes. The reaction mixture was concentrated under reduced pressure. H₂O (10 mL) was added to the reaction mixture and HCl (conc.) was added dropwise until the pH was lowered to pH 2. The solution was washed with EtOAc (4×50 mL), dried (MgSO₄), and concentrated under reduced pressure to afford 1-(3-chloro-5-(5-ethyloxazol-2-yl)-4-methoxypyridin-2-yl)piperidine-4-carboxylic acid as a solid, which was used crude assuming a 100% yield.

MS^{m}/z: 366 (M+1).

### (c) 1-[3-Chloro-5-(5-ethyl-1,3-oxazol-2-yl)-4-methoxypyridin-2-yl]-N-[(5-chloro-2-thienyl)sulfonyl]piperidine-4-carboxamide

1-(3-chloro-5-(5-ethyloxazol-2-yl)-4-methoxypyridin-2-yl)piperidine-4-carboxylic acid (0.080 g, 0.22 mmol), EDCI (0.050 g, 0.260 mmol) and HOBT (0.035 g, 0.260 mmol) were dissolved in DCM (10 mL) at room temperature. The reaction mixture was stirred at room temperature for 30 minutes and then 5-chlorothiophene-2-sulfonamide (0.052 g, 0.260 mmol) and DIPEA (0.11 ml, 0.66 mmol) were added. The reaction mixture was stirred at room temperature for 16 h. The reaction mixture was diluted with EtOAc (50 mL) and washed with saturated NH₄Cl (2×30 mL). The combined organics were dried (MgSO₄) and concentrated under reduced pressure to afford the crude product. Flash chromatography (gradient elution 20% EtOAc in hexanes, 0.5% AcOH to 40% EtOAc in hexanes, 0.5% AcOH) gave 1-[3-Chloro-5-(5-ethyl-1,3-oxazol-2-yl)-4-methoxypyridin-2-yl]-N-[(5-chloro-2-thienyl)sulfonyl]piperidine-4-carboxamide as a solid. Yield: 0.115 g (92%).

¹H NMR (400 MHz, CDCl₃): δ 1.31 (3H, t, J=7.5 Hz), 1.85-1.97 (4H, m), 2.36-2.44 (1H, m), 2.75 (2H, q, J=7.5 Hz), 2.83-2.90 (2H, m), 3.93-3.97 (54, m), 6.90 (1H, s), 6.97 (1H, d, J=4.2 Hz), 7.71 (1H, d4J=4.2 Hz), 8.26 (1H, br s), 8.64 (1H, s).

MS^{m}/z: 545 (M+1).

### Example 140

### 1-[3-Chloro-5-(5-ethyl-1,3-oxazol-2-yl)-6-methoxypyridin-2-yl]-N-[(5-chloro-2-thienyl)sulfonyl]piperidine-4-carboxamide

### (a) 5-Ethyloxazole-4-carboxylic acid

Ethyl 5-ethyloxazole-4-carboxylate [European Journal of Med. Chem. 1987, 22, 283] (56.9 g, 336 mmol) was suspended in EtOH (700 ml) and a solution of NaOH (33.6 g, 841 mmol) in water (300 ml) was added with ice bath cooling and the system was stirred at room temperature for 14 h. The reaction mixture was concentrated under reduced pressure. The concentrated water solution was acidified to pH 1 with conc. HCl and extracted into DCM. The organics were dried (MgSO₄) and concentrated under reduced pressure to afford the crude material (45.2 g) which was used without further purification.

### (b) 5-Ethyloxazole

5-Ethyloxazole-4-carboxylic acid (45.1 g, 320 mmol) and copper(11) oxide (1.3 g, 16 mmol) were combined with quinoline (46 mL). The product was distilled from the reaction mixture under slightly reduced pressure at a distillation-head temperature less than 100° C. Distillation fractions containing clean product (as determined by NMR) were combined to provide 5-ethyloxazole as a clear liquid. Yield: 27g (87%).

¹H NMR (400 MHz, CDCl₃): δ 1.26 (3H, t, J=7.6 Hz), 2.69 (2H, q, J=7.6 Hz), 6.75 (1H, s), 7.76 (1H, s).

### (c) Methyl 1-(6-chloropyridin-2-yl)piperidine-4-carboxylate

2,6-Dichloropyridine (45.00 g, 304 mmol), methyl piperidine-4-carboxylate (43.1 mL, 319 mmol) and DIPEA (106 mL, 608 mmol) were suspended in DMF (350 mL) and heated at 120° C. until complete consumption of starting material was observed by HPLC analysis. The reaction mixture was concentrated under reduced pressure to afford the crude material. The crude material was partitioned between DCM (500 mL) and 1N HCl (250 mL) and the organics separated, dried (MgSO₄) and concentrated under reduced pressure to afford the crude material. Flash chromatography (eluant 10% EtOAc/Hexanes) gave methyl 1-(6-chloropyridin-2-yl)piperidine-4-carboxylate as a solid. Yield: 54.51 g (70%/0).

¹H NMR (400 MHz, CDCl₃): δ 1.68-1.82 (2H, m), 1.94-2.04 (2H, m), 2.50-2.60 (1H, m), 2.92-3.02 (2H, m), 4.15-4.25 (2H, m), 6.50 (1H, d, J=8.4 Hz), 6.57 (1H, d, J=7.5 Hz), 7.34-7.41 (1H, m).

MS^{m}/z: 255 (M+1).

### (d) Methyl 1-(6-chloro-5-iodopyridin-2-yl)piperidine-4-carboxylate

Methyl 1-(6-chloropyridin-2-yl)piperidine-4-carboxylate (24.16 g, 94.85 mmol) was dissolved in MeCN (400 mL) and N-Iodosuccinimide (21.34 g, 94.85 mmol) added. The reaction mixture was stirred at room temperature overnight. HPLC analysis showed incomplete reaction. More NIS was added until HPLC analysis showed complete reaction conversion. The reaction mixture was concentrated under reduced pressure and the residue partitioned between EtOAc (500 mL) and sat. aqueous NaHCO₃ (300 mL). The organics were dried (MgSO₄) and concentrated under reduced pressure to afford the crude material. Flash chromatography (eluant 10-20% EtOAc/Hexanes) gave methyl 1-(6-chloro-5-iodopyridin-2-yl)piperidine-4-carboxylate as a solid. Yield: 25.77 g (71%).

¹H NMR (400 MHz, CDCl₃): δ 1.68-1.81 (2H, m), 1.95-2.05 (2H, m), 2.52-2.62 (1H, m), 2.94-3.05 (2H, m), 3.71 (3H, s), 4.11-4.21 (2H, m), 6.32 (1H, d, J=8.7 Hz), 7.73 (1H, d, J=8.7 Hz).

MS^{m}/z: 381 (M+1).

### (e) Methyl 1-(3,6-dichloro-5-iodopyridin-2-yl)piperidine-4-carboxylate

Methyl 1-(6-chloro-5-iodopyridin-2-yl)piperidine-4-carboxylate (24.76 g, 65.05 mmol) and N-chlorosuccinimide (9.56 g, 71.56 mmol) were suspended in MeCN (500 mL) and stirred at reflux until complete consumption of starting material was observed by HPLC analysis. The reaction mixture was concentrated under reduced pressure and the residue partitioned between EtOAc (500 mL) and saturated aqueous NaHCO₃ (300 mL). The organics were dried (MgSO₄) and concentrated under reduced pressure to afford the crude material. Flash chromatography (eluant 7.5% EtOAc/Hexanes) gave methyl 1-(3,6-dichloro-5-iodopyridin-2-yl)piperidine-4-carboxylate as a solid. Yield: 12.93 g (48%).

¹H NMR (400 MHz, CDCl₃): δ 1.81-1.95 (2H, m), 1.99-2.07 (2H, m), 2.46-2.57 (1H, m), 2.86-2.98 (2H, m), 3.71 (3H, s), 3.81-3.90 (2H, m), 7.89 (1H, s).

MS^{m}/z: 415 (M+1).

### (f) Methyl 1-(3,6-dichloro-5-ethyloxazol-2-yl)pyridin-2-yl)piperidine-4-carboxylate

5-Ethyloxazole (0.351 g, 3.61 mmol) was suspended in THF (4 mL) and cooled to −78° C. Butyllithium (2.56 ml, 4.10 mmol) was added drop-wise maintaining the internal temperature below −60° C. The reaction mixture was stirred for 20 minutes and then ZnCl₂ (1.07 g, 7.83 mmol) was added in one portion. The reaction mixture was warmed to room temperature and placed under an argon balloon. The system was sonicated for five minutes. A solution of methyl 1-(3,6-dichloro-5-iodopyridin-2-yl)piperidine-4-carboxylate (1.00 g, 2.41 mmol) in THF (5 mL) was added along with Pd(PPh₃)₄ (0.278 g, 0.241 mmol) and the system heated to 60° C. until complete conversion to the starting material was observed by HPLC analysis. The reaction mixture was diluted with EtOAc (30 mL) and washed sequentially with sat. aqueous NH₄Cl (20 mL) and brine (20 mL). The organics were dried (MgSO₄) and concentrated under reduced pressure to afford the crude material. Flash chromatography (eluant 10-15% EtOAc/Hexanes) gave methyl 1-(3,6-dichloro-5-(5-ethyloxazol-2-yl)pyridin-2-yl)piperidine-4-carboxylate as a solid. Yield: 0.434 g (47% o).

¹H NMR (400 MHz, CDCl₃): δ 1.31 (3H, t, J=7.6 Hz), 1.85-1.96 (2H, m), 1.99-2.09 (2H, m), 2.52-2.61 (1H, m), 2.76 (2H, q, J=7.6 Hz), 2.97-3.07 (2H, m), 3.72 (3H, s), 3.99-4.09 (2H, m), 6.89 (1H, s), 8.15 (1H, s).

MS^{m}/z: 384 (M+1).

### (g) 1-(3-Chloro-5-(5-ethyl-1,3-oxazol-2-yl)-6-methoxypyridin-2-yl)piperidine-4-carboxylic acid

Methyl 1-(3,6-dichloro-5-(5-ethyl-1,3-oxazol-2-yl)pyridin-2-yl)piperidine-4-carboxylate (0.434 g, 1.13 mmol) was suspended in MeOH (20 ml) in a sealed tube and then sodium methoxide (0.610 g, 11.3 mmol) added. The system was heated at 75° C. until conversion of the starting material was observed by HPLC analysis. The reaction progressed to complete 6-Cl displacement but incomplete hydrolysis so 1 mL of water was added and the reaction continued. Complete product formation was observed by HPLC analysis. The reaction mixture was cooled to room temperature and diluted with DCM (100 mL) and water (50 mL). The aqueous was made acidic with conc. HCl and extracted into DCM. The combined organics were dried (MgSO₄) and concentrated under reduced pressure to afford the crude 1-(3-chloro-5-(5-ethyl-1,3-oxazol-2-yl)-6-methoxypyridin-2-yl)piperidine-4-carboxylic acid, which was used without further purification. Yield: 0.322 g (78%).

¹H NMR (400 MHz, CDCl₃): δ 1.29 (3H, t, J=7.5 Hz), 1.89-2.00 (2H, m), 2.02-2.11 (2H, m), 2.56-2.66 (1H, m), 2.73 (2H, q, J=7.5 Hz), 2.98-3.10 (2H, m), 4.02-4.11 (5H, m), 6.87 (1H, s), 8.07 (1H, s).

MS^{m}/z: 366 (M+1).

### (h) 1-[3-Chloro-5-(5-ethyl-1,3-oxazol-2-yl)-6-methoxypyridin-2-yl]-N-[(5-chloro-2-thienyl)sulfonyl]piperidine-4-carboxamide

1-(3-Chloro-5-(5-ethyl-1,3-oxazol-2-yl)-6-methoxypyridin-2-yl)piperidine-4-carboxylic acid (0.322 g, 0.88 mmol), EDCI (0.22 g, 1.1 mmol) and HOBT (0.15 g, 1.1 mmol) were dissolved in DCM (20 mL) at room temperature. The reaction mixture was stirred at room temperature for 30 minutes and then 5-chlorothiophene-2-sulfonamide (0.24 g, 1.2 mmol) and DIPEA (0.92 ml, 5.3 mmol) were added. The reaction mixture was stirred at room temperature until complete consumption of starting material was observed by HPLC analysis. The reaction mixture was diluted with DCM (50 mL) and washed with saturated NH₄Cl (30 mL). The combined organics were dried (MgSO₄) and concentrated under reduced pressure to afford the crude product. Flash chromatography (gradient elution 3:7 EtOAc/hexanes, 0.5% AcOH to 7:3 EtOAc/hexanes, 0.5% AcOH) gave 1-[3-Chloro-5-(5-ethyl-1,3-oxazol-2-yl)-6-methoxypyridin-2-yl]-N-[(5-chloro-2-thienyl)sulfonyl]piperidine-4-carboxamide as a solid. Yield: 0.356 g (78%).

¹H NMR (400 MHz, CDCl₃): δ 1.29 (3H, , J=7.5 Hz), 1.83-1.96 (4H, m), 2.48-2.51 (1H, m), 2.74 (2H, q, J=7.5 Hz), 2.82-2.92 (2H, m), 3.98-4.11 (5H, m), 6.88 (1H, s), 6.97 (1H, d, J=4.1 Hz), 7.71 (1H, d, J=4.1 Hz), 8.05 (1H, s).

MS^{m}/z: 545 (M+1).

### Example 141

### 1-[3-Chloro-4-(dimethylamino)-(5-ethyl-1,3-oxazol-2-yl)pyridin-2-yl]-N-[(5-chloro-2-thienyl)sulfonyl]piperidine-4-carboxamide

### (a) Methyl 1-(3-chloro-5-(5-ethyl-1,3-oxazol-2-yl)-4-(methylsulfonyl)pyridin-2-yl)piperidine-4-carboxylate

Methyl 1-(3-chloro-5-(5-ethyl-1,3-oxazol-2-yl)-4-(methylthio)pyridin-2-yl)piperidine-4-carboxylate (2.12 g, 5.35 mmol), see example 112, was dissolved in DMF (500 mL) and 3-Chloroperoxybenzoic acid (2.64 g, 10.7 mmol) was slowly added at room temperature. The solution was stirred at room temperature for 4 h. 3-Chloroperoxybenzoic acid (1.32 g, 5.35 mmol) was slowly added at room temperature for 3 h. Saturated Na₂S₂O₃ (30 mL) was added and the solution was stirred for 5 minutes. The reaction mixture was diluted with CH₂Cl₂ (40 mL) and the combined organics were separated and washed with NaOH (1M, 2×40 mL) and brine (1×30 mL), dried (MgSO₄) and concentrated under reduced pressure to afford the crude product as a solid. Flash chromatography (30% EtOAc in Hexanes) gave methyl 1-(3-chloro-5-(5-ethyl-1,3-oxazol-2-yl)-4-(methylsulfonyl)pyridin-2-yl)piperidine-4-carboxylate as a solid. Yield: 0.970g (42.3%).

¹H NMR (400 MHz, CDCl₃): δ 1.30 (3H, t, J=7.5 Hz), 1.87-2.00 (21, m), 2.02-2.12 (2H, m), 2.55-2.65 (1H, m), 2.76 (2H, q, J=7.5 Hz), 3.01-3.13 (2H, m), 3.38 (3H, s), 3.74 (3H, s), 3.90-4.00 (2H, m), 6.88 (1H, s), 8.40 (1H, s).

MS^{m}/z: 428 (M+1).

### (b) Methyl 1-(3-chloro-4-(dimethylamino)-5-(5-ethyl-1,3-oxazol-2-yl)pyridin-2-yl)piperidine-4-carboxylate

Methyl 1-(3-chloro-5-(5-ethyl-1,3-oxazol-2-yl)-4-(methylsulfonyl)pyridin-2-yl)piperidine-4-carboxylate (0.100 g, 0.230 mmol), DIPEA (0.41 mL, 2.30 mmol), and dimethylamine (1.5 mL), were dissolved into THF (2 mL) and heated at 60° C. for 30 h. The reaction mixture was concentrated under reduced pressure to afford methyl 1-(3-chloro-4-(dimethylamino)-5-(5-ethyl-1,3-oxazol-2-yl)pyridin-2-yl)piperidine-4-carboxylate as a solid. No purification was done.

### (c) 1-(3-chloro(dimethylamino)-5-(5-ethyl-1,3-oxazol-2-yl)pyridin-2-yl)piperidine-4-carboxylic acid

1-(3-chloro-4-(dimethylamino)-5-(5-ethyl-1,3-oxazol-2-yl)pyridin-2-yl)piperidine-4-carboxylate (0.130 g, 0.335 mmol), and lithium hydroxide (1 M, 6.60 mL, 6.60 mmol) were dissolved in THF (2 mL) and stirred at room temperature 1 h. NaOH (6 N, 1 mL) was added and the reaction mixture was stirred for 3 hours. The reaction mixture was concentrated under reduced pressure. H₂O (10 mL) was added to the reaction mixture and HCl (conc.) was added drop-wise until the pH was lowered to pH 2. The solution was washed with EtOAc (4×50 mL), dried (MgSO₄), and concentrated under reduced pressure to afford 1-(3-chloro-4-(dimethylamino)-5-(5-ethyloxazol-2-yl)pyridin-2-yl)piperidine-4-carboxylic acid as a solid, which was used crude assuming a 100% yield.

¹H NMR (400 MHz, CDCl₃): δ 1.23-1.34 (4H, m), 1.80-2.00 (2H, m), 2.01-2.11 (2H, m), 2.52-2.62 (1H, m), 2.69-2.80 (8H, m), 2.90-3.01 (2H, m), 3.82-3.92 (2H, m), 6.86 (1H, s), 8.29 (1H, s).

MS^{m}/z: 379 (M+1).

### (d) 1-[3-Chloro-4-(dimethylamino)-5-(5-ethyl-1,3-oxazol-2-yl)pyridin-2-yl]-N-[(5-chloro-2-thienyl)sulfonyl]piperidine-4-carboxamide

1-(3-chloro-4-(dimethylamino)-5-(5-ethyloxazol-2-yl)pyridin-2-yl)piperidine-4-carboxylic acid (0.040 g, 0.110 mmol), EDCI (0.024 g, 0.13 mmol) and HOBT (0.017 g, 0.130 mmol) were dissolved in DCM (2 mL) at room temperature. The reaction mixture was stirred at room temperature for 10 minutes and then 5-chlorothiophene-2-sulfonamide (0.021 g, 0.110 mmol) and TEA (0.074 mL, 0.530 mmol) were added. The reaction mixture was stirred at room temperature for 16 h. The reaction mixture was diluted with EtOAc (40 mL) and the combined organics were washed with saturated NH₄Cl (2×30 mL), dried (MgSO₄) and concentrated under reduced pressure to afford the crude product. Flash chromatography (30% EtOAc in Hexanes with 0.5% AcOH) gave 1-[3-Chloro-4-(dimethylamino)-5-(5-ethyl-1,3-oxazol-2-yl)pyridin-2-yl]-N-[(5-chloro-2-thienyl)sulfonyl]piperidine-4-carboxamide as a solid. Yield: 0.037 g (63%).

¹H NMR (400 MHz, CDCl₃): δ 1.28-1.36 (3H, n), 1.80-1.91 (4H, m), 2.23-2.34 (1H, m), 2.63-2.82 (10H, m), 3.80-3.90 (2H, m), 6.90-7.00 (2H, m), 7.68-7.73 (1H, s), 8.25 (1H, s).

MS^{m}/z: 558 (M+1).

### Example 142

### Ethyl 5-cyano-2-methyl-4-(3-{[(pyridin-3-ylsulfonyl)amino]carbonyl}azetidin-1-yl)nicotinate

Prepared according to method A starting from pyridine-3-sulfonamide (0.072 g, 0.38 mmol). Yield: 0.058 g (54%).

¹H NMR (400 MHz, d₆-DMSO) δ 1.25 (3H, t, J=7.2 Hz), 2.55 (3H, s), 3.57 (1H, ddd, J=14.6, 8.9, 5.7 Hz), 4.18 (4H, m), 4.36 (2H, t, J=8.9 Hz), 7.66 (1H, dd, J=8.1, 4.8 Hz), 8.23 (1H, s), 8.30 (1H, d, J=8.3 Hz), 8.85 (1H, d, J=3.8 Hz), 9.04 (1H, s)

MS^{m}/z: 430 (M+1)

### Example 143

### Ethyl 5-cyano-2-methyl-6-(3-{[({5-[1-methyl-5-(trifluoromethyl)-1H-pyrazol-3-yl]-2-thienyl}sulfonyl)amino]carbonyl}azetidin-1-yl)nicotinate

Prepared according to method A starting from 5-[1-methyl-5-(trifluoromethyl)-1H-pyrazol-3-yl]thiophene-2-sulfonamide (0.139 g, 0.32 mmol). Yield: 0.061 g (50%).

¹H NMR (500 MHz, d₆-DMSO) δ 1.29 (31, t, J=7.1 Hz), 2.60 (3H, s), 3.51 (1H, m), 4.04 (3H, s), 4.23 (2H, q, J=7.1 Hz), 4.27 (2H, m), 4.41 (2H, m), 7.17 (1H, s), 7.53 (1H, d, J=3.8 Hz), 7.76 (1R^{X}, s), 8.27 (1H, s)

MS^{m}/z: 583 (M+1)

### Example 144

### N-[(S-chloro-2-thienyl)sulfonyl]-1-[3-[(2,2-dimethylpropanoyl)amino]-5-(5-ethyl-1,3-oxazol-2-yl)pyridin-2-yl]piperidine-4-carboxamide

Beginning with methyl 1-(3-amino-5-(5-ethyl-1,3-oxazol-2-yl)pyridin-2-yl)piperidine-4-carboxylate and utilizing the same methodology which produced 11-[3-(Acetylamino)-5-(5-ethyl-1,3-oxazol-2-yl)pyridin-2-yl]-N-[(5-chloro-2-thienyl)sulfonyl]piperidine-4-carboxamide, see example 121, from acetyl chloride, N-[(5-chloro-2-thienyl)sulfonyl]-1-[3-[(2,2-dimethylpropanoyl)amino]-5-(5-ethyl-1,3-oxazol-2-yl)pyridin-2-yl]piperidine-4-carboxamide was generated from pivaloyl chloride.

¹H NMR (400 MHz, CDCl₃): δ 1.30 (3H, t, J=7.6 Hz), 1.34 (9H, s), 1.83-1.93 (2H, m), 2.02-2.05 (2H, m), 2.36-2.43 (1H, m), 2.71-2.82 (4H, m), 3.23-3.27 (2H, m), 6.89 (1H, s), 6.97 (1H, d, J=4.2 Hz), 7.70 (1H, d, J=4.2 Hz), 8.17 (1H, s), 9.13 (1H, s), 9.23 (1H, s), 9.23 (1H, br s).

MS^{m}/z: 581 (M+1).

### Example 145

### Ethyl 6-[3-({[(5-chloro-1,3-dimethyl-1H-pyrazol-4-yl)sulfonyl]-amino}carbonyl)azetidin-1-yl]-5-cyano-2-methylnicotinate

Prepared according to method A starting from 5-chloro-1,3-dimethyl-1H-pyrazole-4-sulfonamide (0.079 g, 0.38 mmol). Yield: 0.084 g (70%).

¹H NMR (400 MHz, d₆-DMSO) δ 1.24 (3H, t, J=7.1 Hz), 2.29 (3H, s), 2.56 (3H, s), 3.29 (1H, m, overlapped by water), 3.72 (3H, s), 4.18 (4H, m), 4.36 (2H, t, J=9.0 Hz), 8.23 (1H, s)

MS^{m}/z: 481 (M+1)

### Example 146

### Ethyl 5-cyano-2-methyl-6-{3-[({[3-(3-methyl-5-oxo-4,5-dihydro-1H-pyrazol-1-yl)phenyl]sulfonyl}amino)carbonyl]azetidin-1-yl}nicotinate

Prepared according to method A starting from starting from 3-(3-Methyl-5-oxo-4,5-dihydro-pyrazol-1-yl)-benzenesulfonamide (0.100 g, 0.38 mmol). Yield: 0.011 g (8%).

¹H NMR (400 MHz, d₆-DMSO) δ 1.21 (t, J=7.1 Hz, 3H), 2.06 (s, 313), 2.52 (s, 3H), 3.48 (mult, 1H), 4.14 (mult, 2H), 4.15 (q, J=7.1 Hz, 2), 4.32 (t, J=8.7 Hz, 2H), 5.35 (s, 1H), 7.60 (mult, 2H), 7.97 (mult, 1H), 8.19 (s, 1H), 8.26 (s, 1H)

MS^{m}/z: 525 (M+1)

### Example 147

### Ethyl 6-(3-{[({4-[(-chlorophenyl)sulfonyl]-3-methyl-2-thienyl}sulfonyl)amino]carbonyl}azetidin-1-yl)-5-cyano-2-methylnicotinate

Prepared according to method A starting from 4-(4-chloro-benzenesulfonyl)-3-methyl-thiophene-2-sulfonic acid amide (0.139 g, 0.38 mmol). Yield: 0.115 g (73%0).

¹H NMR (400 MHz, d₆-DMSO)) δ 1.22 (31H, t, J=7.1 Hz), 2.25 (3H, s), 2.54 (3H, s), 3.24 (1H, m, overlapped by water), 4.16 (4H, m), 4.28 (2H, m), 7.63 (2H, d, J=8.5 Hz), 7.84 (2H, d, J=8.5 Hz), 8.19 (1H, s), 8.55 (1H, s)

MS^{m}/z: 623 (M+1)

### Example 148

### Ethyl 5-cyano-2-methyl-6-{3-[({[2-(trifluoromethoxy)phenyl]sulfonyl}amino)carbonyl]azetidin-1-yl}nicotinate

Prepared according to method A starting from 2-trifluoromethoxy-benzenesulfonamide (0.097 g, 0.38 mmol). Yield: 0.114 g (89%).

¹H NMR (400 MHz, d₆-DMSO)) δ 1.25 (3H, t, J=7.1 Hz), 2.55 (3H, s), 3.48 (1H, m), 4.17 (2H, m), 4.18 (2H, q, J=7.1 Hz), 4.36 (2H, m), 7.53 (2H, m), 7.71 (1H, m), 8.02 (1H, d, J=7.5 Hz), 8.23 (1H, s)

MS^{m}/z: 513 (M+1)

### Example 149

### Ethyl 5-cyano-6-[3-({[(3,5-difluorophenyl sulfonyl]amino}carbonyl)azetidin-1-yl]-2-methylnicotinate

Prepared according to method A starting from 3,5-difluoro-benzenesulfonamide (0.075 g, 0.38 mmol). Yield: 0.096 g (83%).

¹H NMR (400 MHz, d₆-DMSO) δ 1.25 (3H>t, J=7.2 Hz), 2.56 (3H, s), 3.40 (1H, m), 4.18 (4H, m), 4.34 (2H, t, J=8.4 Hz), 7.48 (3H, m), 8.22 (1H, s)

MS^{m}/z: 465 (M+1)

### Example 150

### Ethyl 5-cyano-2-methyl-6-{3-[({[4-(trifluoromethoxy)phenyl]sulfonyl}amino)carbonyl]azetidin-1-yl}nicotinate

Prepared according to method A starting from 4-trifluoromethoxy-benzenesulfonamid (0.093 g, 0.38 mmol). Yield: 0.098 g (76%).

¹H NMR (400 MHz, d₆-DMSO) δ 1.25 (3H, , J=7.1 Hz), 2.56 (3H, s), 3.56 (1 in, ddd, J 14.5, 8.9, 5.7 Hz), 4.17 (2H, m), 4.18 (2H, q, J=7.1 Hz), 4.36 (2H, t, J=8.8 Hz), 7.59 (2H, d, J=8.5 Hz), 8.04 (2H, d, J=8.9 Hz), 8.23 (1H, s)

MS^{m}/z: 513 (M+1)

### Example 151

### Ethyl 6-[3-(2-{[5-chloro-2-thienyl)sulfonyl]amino}-2-oxoethyl)piperidin-1-yl]-S-cyano-2-methylnicotinate

### (a) Piperidin-3-ylacetic acid potassium salt

Potassium trimethylsilanoate (0.89 g, 5.2 mmol) and ethyl piperidin-3-ylacetate (0.87 g, 6.8 mmol) were stirred in DCM (50 mL) at r.t for 2 days. Concentration of the reaction mixture afforded solid piperidin-3-ylacetic acid as the potassium salt, which was used crude assuming complete conversion. Yield: 0.74 g (100%).

### (b) {1-[3-Cyano-5-(ethoxycarbonyl)-6-methylpyridin-2-yl]piperidin-3-yl}acetic acid

Ethyl 6-chloro-5-cyano-2-methylnicotinate (1.00 g, 4.45 mmol), piperidin-3-ylacetic acid (0.701 g, 4.90 mmol) and DIPEA (2.33 mL, 13.4 mmol) were dissolved in DMF (30 mL) and stirred at r.t for 3 days. The reaction mixture was diluted with EtOAc (100 mL), washed with saturated NH₄Cl (2×25 mL), saturated NaHCO₃ (2×25 mL), brine (25 m), dried (MgSO₄) and concentrated under reduced pressure to afford crude material. Flash chromatography (9:1 EtOAc/hexanes with 1% AcOH) gave {1-[3-cyano-5-(ethoxycarbonyl)-6-methylpyridin-2-yl]piperidin-3-yl}acetic acid as a solid. Yield: 0.791 g (54%).

¹H NMR (400 MHz, CDCl₃): δ 1.37 (3H, t, J=7.1 Hz), 1.39-1.44 (1H, m), 1.63-1.73 (1H, m), 1.78-1.85 (1H, m), 1.98-2.03 (1H, m), 2.16-2.24 (1H, m), 2.29-2.34 (1H, m), 2.40-2.46 (1H, m), 2.71 (3H, s), 3.08-3.13 (1H, m), 3.26-3.32 (1H, m), 4.31 (2H, q, J=7.1 Hz), 4.44-4.50 (1H, m), 4.52-4.56 (1H, m), 8.33 (1H, s).

MS^{m}/z: 330 (M−1).

### (c) Ethyl 6-[3-(2-{[(5-chloro-2-thienyl)sulfonyl]amino}-2-oxoethyl)piperidin-1-yl]-5-cyano-2-methylnicotinate

A solution of 2-(1-(3-cyano-5-(ethoxycarbonyl)-6-methylpyridin-2-yl)piperidin-3-yl)acetic acid (0.100 g, 0.302 mmol), EDCI (0.075 g, 0.392 mmol), and HOBT (0.053 g, 0.392 mmol), 5-chlorothiophene-2-sulfonamide (0.078 g, 0.392 mmol) and DIPEA (0.105 mL, 0.604 mmol) in DCM (7.0 mL) was stirred at room temperature for 20 h. Following concentration, the mixture was diluted with EtOAc (100 mL), washed with saturated NH₄Cl (2×50 mL), saturated NaHCO₃ (2×50 mL), brine (50 mL), dried (MgSO₄) and concentrated. Flash chromatography (20% EtOAc/hexanes with 0.5% AcOH) furnished ethyl 6-[3-(2-{[(5-chloro-2-thienyl)sulfonyl]amino}-2-oxoethyl)piperidin-1-yl]-5-cyano-2-methylnicotinate as a solid. Yield: 0.030g (19%).

¹H NMR (400 Mhz, CDCl₃): δ 1.38 (3H, t, J=7.0 Hz), 1.61-1.70 (1H, m), 1.74-1.78 (1H, m), 1.93-1.97 (1H, m), 2.20-2.23 (2H, m), 2.38-2.45 (1H, m), 2.72 (3H, s), 3.19-3.25 (1H, m), 3.34-3.40 (1H, m), 4.26-4.35 (4H, m), 6.96 (1H, t, J=3.9 Hz), 7.69 (1H, t, J=3.9 Hz), 8.34 (1H, s).

MS^{m}/z: 512 (M+1).

### Example 152

### Ethyl 5-cyano-6-{3-[({[5-(methoxycarbonyl)-2-furyl]sulfonyl}amino)carbonyl]azetidin-1-yl}-2-methylnicotinate

Prepared according to method A starting from 5-methoxycarbonyl-furan-2-sulfonamide (0.098 g, 0.38 mmol). Yield: 0.034 g (28%).

¹H NMR (400 MHz, d₆-DMSO) δ 1.25 (3H, t, J=7.1 Hz), 2.56 (3H, s), 3.3 (1H, m), 3.78 (3H, s), 4.18 (4H, m), 4.32 (2H, m), 6.84 (1H, m), 7.24 (1H, d, J=3.4 Hz), 8.21 (1H, s)

MS^{m}/z: 477 (M+1)

### Example 153

### Ethyl 5-cyano-6-{3-[({[4-(methoxycarbonyl)-5-methyl-2-furyl]sulfonyl}amino)carbonyl]azetidin-1-yl}-2-methylnicotinate

Prepared according to method A starting from 4-methoxycarbonyl-5-methyl-furan-2-sulfonamide (0.089 g, 0.38 mmol). Yield: 0.033 g (27%).

¹H NMR (400 MHz, d₆-DMSO) δ 1.22 (3H, t, J=7.2 Hz,), 2.49 (3H, s, overlapped by DMSO), 8.20 (1H, s), 2.54 (3H, s),), 3.32 (1H, m), 3.71 (3H, s, overlapped by water), 4.16 (4H, m), 4.33 (2H, m), 6.94 (1H, s)

MS^{m}/z: 491 (M+1)

### Example 154

### Ethyl 6-[3-({[(4-chlorophenyl)sulfonyl]amino}carbonyl)azetidin-1-yl]-5-cyano-2-methylnicotinate

Prepared according to method A starting from 4-chlorobenzenesulfonamide (0.198 g, 0.38 mmol)-Yield: 0.061 g (53%).

¹Hl NMR (400 MHz, d₆-DMSO) δ 1.22 (3H, t, J=7.2 Hz), 2.53 (3H, s), 3.48 (1H, m), 4.13 (2H, m), 4.16 (2H, q, J=7.1 Hz), 4.34 (2H, t, J=9.0 Hz), 7.63 (2H, d, J=8.7 Hz), 7.87 (2H, d, J=8.7 Hz), 8.20 (1H, s)

MS^{m}/z: 427 (M+1)

### Example 155

### Ethyl 5-cyano-6-[3-({[(3,4-dichlorophenyl)sulfonyl]amino}carbonyl)azetidin-1-yl]-2-methylnicotinate

Prepared according to method A starting from 3,4-dichlorobenzenesulfonamide (0.067 g, 0.38 mmol). Yield: 0.101 g(81%).

¹H NMR (400 Hz, , d₆-DMSO) δ 1.22 (3H, t, l=7.1 Hz), 2.53 (3H, s), 3.37 (1H, m), 4.13 (2H, mult), 4.16 (2H, q, J=7.1 Hz), 4.32 (2H, m), 7.76 (2H, s), 7.95 (1H, s), 8.20 (1H, s)

MS^{m}/z: 497 (M+1)

### Example 156

### Ethyl 5-cyano-6-[3-({[(3,4-dimethoxyphenyl)sulfonyl]amino}carbonyl)azetidin-1-yl]-2-methylnicotinate

Prepared according to method A starting from 3,4-dimethoxybenzenesulfonamide (0.063 g, 0.38 mmol). Yield: 0.088 g (72%) of the title compound was isolated.

¹H NMR (400 MHz, d₆DMSO) δ 1.22 (3H, t, J=7.1 Hz,), 3.49 (1H, m), 2.53 (3H, s), 3.76 (3H, s), 3.78 (3H, s), 8.21 (1H, s), ), 4.16 (2H, q, J=7.1 Hz), 4.14 (2H, m), 4.35 (2H, t, J=9.2 Hz), 7.10 (1H, d, J=8.7 Hz), 7.32 (1H, s), 7.48 (1H, d, J=10.7 Hz)

MS^{m}/z: 489 (M+1)

### Example 157

### Ethyl 5-cyano-2-methyl-6-{3-[({[2-methyl-5-(methylsulfonyl)phenyl]sulfonyl}amino)carbonyl]azetidin-1-yl}nicotinate

Prepared according to method A starting from 2-methyl-5-(methylsulfonyl)benzenesulfonamide (0.053 g, 0.38 mmol). Yield: 0.033 g (25%).

¹H NMR (400 MHz, d₆DMSO) δ 1.22 (3H, t, J=7.1 Hz), 2.53 (3H, s), 2.59 (3H, s), 3.16 (3H, s), 3.39 (1H, m), 4.14 (2H, m), 4.16 (2H, q, J=7.1 Hz), 4.33 (211 m), 7.57 (1H, d, J=8.1 Hz), 7.94 (1H, d, J=8.1 Hz), 8.19 (1H, s), 8.31 (1H, s)

MS^{m}/z: 521 (M+1)

### Example 158

### N-[(5-chloro-2-thienyl)sulfonyl]-1-[3-cyano-5-(cyclopropylcarbonyl)-6-methylpyridin-2-yl]piperidin e-4-carboxamide

### (a) 2-(1-Cyclopropylethylidene)-1,1-dimethylhydrazine

A solution of 1-cyclopropylethanone (26.7 mL, 285 mmol), 1,1-dimethylhydrazine (43.4 mL, 571 mmol) and 4-methylbenzenesulfonic acid hydrate (0.054 g, 0.29 mmol) in benzene (200 mL) was refluxed with a Dean Stark apparatus for 20 h. After cooling to room temperature, the reaction mixture was concentrated, diluted with DCM (400 mL), passed through silica gel and concentrated to yield 2-(1-cyclopropylethylidene)-1,1-dimethylhydrazine as an oil. Yield: 28g (78%).

MS^{m}/z: 127 (M+1).

### (b) 4-Cyclopropyl-4-(2,2-dimethylhydrazono)butan-2-one

To a solution of diisopropylamine (14.8 mL, 105 mmol) in THF (80 mL) cooled to 0° C. was added slowly BuLi (1.60 M in pentane, 65.4 mL, 105 mmol). The reaction mixture was stirred for 20 minutes and then cooled to −78° C. This solution was added drop-wise to a solution of 2-(1-cyclopropylethylidene)-1,1-dimethylhydrazine (12.0 g, 95.1 mmol) in THF (200 mL) cooled to −78° C. The reaction became heterogenous, was diluted with THF (100 mL) and stirred for 15 minutes. N-methoxy-N-methylacetamide (9.81 g, 95.1 mmol) was added drop-wise. The reaction was stirred for 15 minutes at −78° C., warmed to 0° C. for 15 minutes and then quenched with saturated NH₄Cl (100 mL). The combined organic layers from extractions with EtOAc (3×200 mL) were washed with brine, (200 mL), dried (MgSO₄), passed through silica gel and concentrated to furnish 4-cyclopropyl-4-(2,2-dimethylhydrazono)butan-2-one as an oil. Yield: 16.0 g (100%).

MS^{m}/z: 169 (M+1).

### (c) 3-(Cyclopropyl(2,2-dimethylhydrazono)methyl-4-(dimethylamino)but-3-en-2-one

A mixture of 4-cyclopropyl-4-(2,2-dimethylhydrazono)butan-2-one (16.2 g, 96.5 mmol) and 1,1-dimethoxy-N,N-dimethylmethanamine (19.3 mL, 145 mmol) was heated to 75° C. for 12 h. The mixture was concentrated, diluted with DCM (200 mL), passed through silica gel (10% MeOH/EtOAc elution) and concentrated to furnish 3-(cyclopropyl(2,2-dimethylhydrazono)methyl)-4-(dimethylamino)but-3-en-2-one. Yield: 11.9 g (55%).

MS^{m}/z: 224 (M+1).

### (d) 5-(Cyclopropanecarbonyl)-6-methyl-2-oxo 1,2-dihydropyridine-3-carbonitrile

To a solution of 2-cyanoacetamide (4.49 g, 53.4 mmol) in DMF (100 mL) was added in small portions NaH (55% in mineral oil, 2.33 g, 53.4 mmol) and the reaction was stirred for 30 minutes. To this mixture was added 3-(cyclopropyl(2,2-dimethylhydrazono)methyl)-4-(dimethylamino)but-3en-2-one (11.9 g, 53.4 mmol) and the resulting mixture was stirred at 80° C. for 20 h. Addition of HCl (2M, 150 mL) followed by cooling to room temperature produced 5-(cyclopropanecarbonyl)-6-methyl-2-oxo-1,2-dihydropyridine-3-carbonitrile as a precipitate which was collected and dried under vacuum. Yield 0.60 g (5.6%).

MS^{m}/z: 201 (M−1).

### (e) 2-Chloro-5-(cyclopropanecarbonyl)-6-methylnicotinonitrile

A suspension of 5-(cyclopropanecarbonyl)-6-methyl-2-oxo-1,2-dihydropyridine-3-carbonitrile (0.054 mg, 0.27 mmol) in POCl₃ (3.0 mL) was stirred at 100° C. for 20 h. Concentration and flash chromatography (5% EtOAc/hexanes) produced 2-chloro-5-(cyclopropanecarbonyl)-6-methylnicotinonitrile as a solid. Yield: 0.033 g (56%).

¹H NMR (400 MHz, CDCl₃): δ 1.20-1.26 (2H, m), 1.31-1.35 (2H, m), 2.65 (3H, s), 2.74-2.80 (1H, m), 8.08 (1H, s).

### (f) tert-Butyl 4-(5-chlorothiophen-2-ylsulfonylcarbamoyl)piperidine-1-carboxylate

A solution of 1-(tert-butoxycarbonyl)piperidine-4-carboxylic acid (7.00 g, 30.5 mmol) EDCI (7.02 g, 36.6 mmol) and HOBT (4.95 g, 36.6 mmol) in DCM (200 mL) was stirred at room temperature for 30 minutes. 5-chlorothiophene-2-sulfonamide (7.54 g, 38.2 mmol) and DIPEA (16.0 mL, 91.6 mmol) were added and the reaction mixture was stirred for 20 h. The reaction was diluted with 1)CM (500 mL), washed with saturated NH₄Cl (3×200 mL), dried (MgSO₄) and concentrated. Flash chromatography (25% EtOAc/hexanes with 1% AcOH) furnished tert-butyl 4-(5-chlorothiophen-2-ylsulfonylcarbamoyl)piperidine-1-carboxylate as a solid. Yield: 11.3 g (90%).

¹H NMR (400 MHz, CDCl₃): δ 1.45 (9H, s), 1.55-1.65 (2H, m), 1.79-1.82 (2H, m), 2.92-2.37 (1H, m), 2.73-2.80 (2H, m), 4.06-4.11 (2H, m), 6.96 (1H, d, J=4.1 Hz), 7.69 (1H, d, J=4.1 Hz), 8.11 (1H, br s).

### (g) N-(5-chlorothiophen-2-ylsulfonyl)piperidine-4-carboxamide hydrochloride

A suspension tert-butyl 4-(5-chlorothiophen-2-ylsulfonylcarbamoyl)piperidine-1-carboxylate (11.3 g, 27.6 mmol) in THF (500 mL) was treated with HCl (4M in 1,4-dioxane, 138 mL, 552 mmol) and the reaction mixture was stirred at room temperature for 20 h. Concentration furnished N-(5-chlorothiophen-2-ylsulfonyl)piperidine-4-carboxamide hydrochloride as a solid. Yield: 9.52 g (100%).

¹H NMR (400 MHz, DMSO-d₆): δ 1.58-1.68 (2H, m), 1.87-1.90 (2H, m), 2.52-2.59 (1H, m), 2.80-2.88 (2H, m), 3.22-3.25 (2H, m), 7.29 (1H, d, J=4.1 Hz), 7.67 (1H, d, J=4.1 Hz), 8.51 (1H, br s), 8.82 (1H, br s).

MS^{m}/z: 309 (M+1).

### (h) N-[(5-chloro-2-thienyl)sulfonyl]-1-[3-cyano-5-(cyclopropylcarbonyl)-6-methylpyridin-2-yl]piperidine-carboxamide

A solution of 2-chloro-5-(cyclopropanecarbonyl)-6-methylnicotinonitrile (0.030 g, 0.136 mmol), N-(5-chlorothiophen-2-ylsulfonyl)piperidine-4-carboxamide hydrochloride (0.0469 g, 0.136 mmol) and DIPEA (0.0947 mL, 0.544 mmol) in DMF (5 mL) was heated to 80° C. for 20 h. Following concentration, the mixture was diluted with EtOAc (100 mL), washed with saturated NH₄Cl (2×50 mL), brine (50 mL), dried (MgSO₄) and concentrated. Flash chromatography (50% EtOAc/hexanes with 1% AcOH) furnished N-[(5-chloro-2-thienyl)sulfonyl]-1-[3 cyano-5-(cyclopropylcarbonyl)-6-methylpyridin-2-yl]piperidine-4-carboxamide as a solid. Yield: 0.065 g (94%).

¹H NMR (400 MHz, CDCl₃): δ 1.06-1.09 (2H, m), 1.11-1.30 (21, m), 1.73-1.82 (12H, m), 1.94-1.97 (2H, m), 2.50-2.55 (4H, m), 3.07-3.20 (2H, m), 4.53-4.57 (2H, m), 6.96-6.98 (1H, m), 7.69-7.71 (1H, m), 8.06 (1H, s), 8.28 (1H, br s).

MS^{m}/z: 493 (M+1).

### Example 159

### Isopropyl 6-[3-({[(5-chloro-2-thienyl)sulfonyl]amino}carbonyl)azetidin-1-yl]-5-ethynyl-2-methylnicotinate

### (a) Sodium propan-2-olate

Isopropyl alcohol (5 mL) was cooled to 0° C. Sodium hydride (95%, 0.088 g, 3.48 mmol) was slowly added. The solution was used crude assuming a 100% conversion.

### (b) 1-[3-Cyano-5-(isopropoxycarbonyl)-6-methylpyridin-2-yl]azetidine-3-carboxylic acid

1-[3-cyano-5-(ethoxycarbonyl)-6-methylpyridin-2-yl]azetidine-3-carboxylic acid (0.400 g, 1.20 mmol) was dissolved in isopropyl alcohol (5 mL) and stirred at r.t for 10 minutes. Sodium propan-2-olate (0.286 g, 3.48 mmol) in isopropyl alcohol (5 mL) was added and the solution was stirred for 10 minutes. HCl (cone.) was added drop-wise to the mixture until the pH was lowered to pH 2. The reaction mixture was concentrated under reduced pressure. The reaction mixture was concentrated under reduced pressure. The aqueous was washed with EtOAc (3×40 mL), dried (MgSO₄) and concentrated under reduced pressure to afford the crude product as a solid. Flash chromatography (100% EtOAc to 100% EtOAc with 0.5% AcOH) yielded 1-[3-cyano-5-(isopropoxycarbonyl)-6-methylpyridin-2-yl]azetidine-3-carboxylic acid as a solid. Yield: 0.133 g (51.0%).

¹H NMR (400 MHz, CDCl₃): δ 1.34 (6H, d, J=6.2 Hz), 2.71 (3H, s), 3.59-3.67 (1H, m), 4.57-4.64 (4H, m), 5.15-5.24 (1H, m), 8.26 (1H, s).

MS^{m}/z: 304 (M+1).

### (c) Isopropyl 6-[3-({[(5-chloro-2-thienyl)sulfonyl]amino}carbonyl)azetidin-1-yl]-5-ethynyl-2-methylnicotinate

1-(3-cyano-5-(isopropoxycarbonyl)-6-methylpyridin-2-yl)azetidine-3-carboxylic acid (0.047 g, 0.153 mmol), EDCI (0.035 g, 0.184 mmol) and HOBT (0.025 g, 0.184 mmol) were dissolved in DCM (1 mL) at room temperature. The reaction mixture was stirred at room temperature for 30 minutes and then 5-chlorothiophene-2-sulfonamide (0.036 g, 0.184 mmol) and DIPEA (0.134 mL, 0.767 mmol) were added. The reaction mixture was stirred at room temperature for 16 h. The reaction mixture was diluted with EtOAc (40 mL). The combined organics were washed with saturated NH₄Cl (2×40 mL), dried (MgSO₄) and concentrated under reduced pressure to afford the crude product. Flash chromatography (30% EtOAc in hexanes then 30% EtOAc in hexanes with 0.5% AcOH) gave isopropyl 6-[3-({[(5-chloro-2-thienyl)sulfonyl]amino}carbonyl)azetidin-1-yl]-5-ethynyl-2-methylnicotinate as a solid. Yield: 0.033 g (44%).

¹H NMR (400 MHz, CDCl₃): δ 1.35 (6H, d, J=6.2 Hz), 2.70 (3H, s), 3.48-3.59 (1H, m), 4.54 (5H, d, J=7.3 Hz), 5.14-5.24 (1H, m), 6.99 (1H, d, J=4.1 Hz), 7.72 (1H, d, J=4.1 Hz), 8.25 (1H, s).

MS^{m}/z: 483 (M+1).

### Example 160

### Ethyl 6-{4-[({[(4-chlorophenyl)sulfonyl]amino}carbonyl)amino]piperidin-1-yl}-5-cyano-2-methylnicotinate

### (a) Ethyl 6-(4-(tert-butoxycarbonylamino)piperidin-1-yl)-5-cyano-2-methylnicotinate

Ethyl 6-chloro-5-cyanonicotinate (2.00 g, 8.90 mmol) and tert-butyl piperidin-4-ylcarbamate (1.78 g, 8.90 mmol) were dissolved in EtOH (50 mL) at room temperature. DIPEA (4.65 mL, 26.7 mmol) was added and the system heated at 94° C. for 4 h. The reaction mixture was cooled to room temperature and the solvent concentrated under reduced pressure. The material was partitioned between EtOAc (50 mL) and saturated aqueous NH₄Cl (2×30 mL). The organics were washed with brine (30 mL), dried (MgSO₄) and concentrated under reduced pressure to afford the crude product. No purification was done. Yield, 3.30 g (95.4%).

¹H NMR (400 MHz, CDCl₃): δ 1.37 (3H, t, J=7.1 Hz), 1.46 (11H, s), 2.05-2.14 (2H, m), 2.72 (3H, s), 3.15-3.26 (2H, m), 3.71-3.83 (1H, m), 4.32 (2H, q, J=7.1 Hz), 4.42-4.51 (1H, m), 4.58-4.67 (2H, m), 8.34 (1H, s).

MS^{m}/z: 389 (M+1).

### (b) Ethyl 6-(4-aminopiperidin-1-yl)-5-cyano-2-methylnicotinate dihydrochloride

Ethyl 6-(4-(tert-butoxycarbonylamino)piperidin-1-yl)-5-cyano-2-methylnicotinate (3.30 g. 8.50 mmol) was dissolved HCl (4 M in dioxane, 31.9 mL, 127 mmol). The reaction mixture was stirred at room temperature for 48 h and concentrated under reduced pressure to yield ethyl 6-(4-aminopiperidin-1-yl)-5-cyano-2-methylnicotinate dihydrochloride as a solid, which was used crude assuming 100% conversion.

¹H NMR (400 MHz, d₆-DMSO): δ 1.31 (3H, t, J=7.1 Hz), 1.53-1.68 (2H, m), 2.02-2.12 (2H, m), 2.65 (3H, s), 3.14-3.27 (2H, m), 3.30-3.43 (1H, m), 4.25 (2H, q, J=7.1 Hz), 4.50-4.60 (2H, m), 8.17-8.29 (2H, m), 8.37 (1H, s).

MS^{m}/z: 362 (M+1).

### (c) Ethyl 6{-4-[({[(4-chlorophenyl)sulfonyl]amino}carbonyl)amino]piperidin-1-yl}-5-cyano-2-methylnicotinate

Ethyl 6-(4-aminopiperidin-1-yl)-5-cyano-2-methylnicotinate dihydrochloride (0.200 g, 0.554 mmol) and DIPEA (0.482 mL, 2.77 mmol) were dissolved in CH₂Cl₂ (2 mL), at room temperature. The reaction mixture was cooled to 0° C. 4-chlorobenzenesulfonyl isocyanate (0.084 mL, 0.559 mmol), was slowly added and the system stirred for 16 h at room temperature. EtOAc (40 mL) was added and the combined organics were washed with saturated NaHCO₃ (1×30 mL) and saturated NH₄Cl (1×30 mL). The organics were then dried (MgSO₄) and concentrated under reduced pressure. Flash Chromatography (30% EtOAc in Hexanes to 50% EtOAc in hexanes with 0.1% AcOH) gave Ethyl 6-{4-[({[(4-chlorophenyl)sulfonyl]amino}carbonyl)amino]piperidin-1-yl}-5 cyano-2-methylnicotinate product as a solid. Yield: 0.182 g (65%).

¹H NMR (400 MHz, d₆DMSO): δ 1.30 (3H, t, J=7.1 Hz), 1.37-1.50 (2H, m), 1.77-1.86 (2H, m), 2.63 (3H, s), 3.14-3.25 (2H, m), 3.60-3.72 (1H, m), 4.24 (2H, q, J=7.1 Hz), 4.38-4.47 (2H, m), 6.62-6.69 (1H, m), 7.70 (2H, d, J=8.6 Hz), 7.91 (21, d, J=8.6 Hz), 8.33 (1H, s), 10.7 (1H, s).

MS^{m}/z: 506 (M+1).

### Example 161

### Ethyl 6-{4-[({[(5-chloro-2-thienyl)sulfonyl]amino}carbonyl)amino]piperidin-1-yl}-5-cyano-2-methylnicotinate

Ethyl 6-(4-aminopiperidin-1-yl)-5-cyano-2-methylnicotinate dihydrochloride (0.250 g, 0.692 mmol), see example 160, and 2,2,2-trichloroethyl [(5-chloro-2-thienyl)sulfonyl]carbamate (0.387 g, 1.04 mmol) were dissolved in DMA (2 mL) at room temperature. DIPEA (1.21 mL, 6.92 mmol) were added and the system heated to 100° C. for 1 h. The reaction mixture was cooled to room temperature and the solvent concentrated under reduced pressure. The material was partitioned between EtOAc (40 mL) and saturated aqueous NH₄Cl (2×40 mL). The organics were dried (MgSO₄) and concentrated under reduced pressure to afford the crude product. Flash Chromatography (30 to 50% EtOAc in Hexanes then 50% EtOAc in hexanes with 0.5% AcOH) gave ethyl 6-{4-[({[(5-chloro-2-thienyl)sulfonyl]amino}carbonyl)amino]piperidin-1-yl}-5-cyano-2-methylnicotinate. Yield: 0.011 g(3%).

¹H NMR (400 MHz, d₆-DMSO): δ 1.30 (31, t, J=7.1 Hz), 1.40-1.53 (2H, m), 1.80-1.90 (2H, m), 2.63 (3H, s), 3.17-3.27 (2H, m), 3.66-3.78 (1H, m), 4.24 (2H, q, J=7.1 Hz), 4.39-4.50 (2H, m), 6.67-6.76 (1H, m), 7.26 (1H, d, J=4.1 Hz), 7.62 (1H, d, J=4.1 Hz), 8.33 (1H, s), 10.9-11.0 (1H, s).

MS^{m}/z: 512 (M+1).

### Example 162

### Ethyl 6-[4-({[(5-chloro-3-thienyl)sulfonyl]amino}carbonyl)piperidin-1-yl]-5-cyano-2-methylnicotinate

The sulfoneamide 5-chlorothiophene-3-sulfonamide (0.25 mmol) was reacted in according to method B to give ethyl 6-[4-({[(5-chloro-3-thienyl)sulfonyl]amino}carbonyl)piperidin-1-yl]-5-cyano-2-methylnicotinate. Yield: 0.024 g (23%)

¹H NMR (400 MHz, d₆-DMSO) δ 1.30 (3H, J=7.2 Hz), 1.46-1.62 (2H, m), 1.80-1.92 (2H, m), 2.58-2.62 (1H, m), 2.63 (3H, s), 3.09-3.22 (2H, m), 4.24 (2H, q, J=7.1 Hz), 4.44-4.54 (2H, m), 7.25 (1H, d, J=4.0 Hz), 7.63 (1H, d, J=4.0 Hz), 8.32 (1H, s), 12.41-12.75 (1H, m)

MS^{m}/z: 497 (M+1).

### Example 163

### Ethyl 5-cyano-2-methyl-6-(4-{[(2-naphthylsulfonyl)amino]carbonyl}piperidin-1-yl)nicotinate

The sulfoneamide naphthalene-2-sulfonamide (0.25 mmol) was reacted in according to method B to give ethyl 5-cyano-2-methyl-6-(4-{[(2-naphthylsulfonyl)amino]carbonyl}piperidin-1-yl)nicotinate. Yield: 0.032 g (30%/0)

¹H NMR (400 MHz, d₆-DMSO) δ 1.28 (31 t, J=7.2 Hz), 1.38-1.53 (2H, m), 1.76-1.87 (2H, m), 2.60 (3H, s), 2.60-2.65 (1H, m), 3.06-3.17 (2H, m), 4.23 (2H, q, J=7.1 Hz), 4.40-4.52 (2H, m), 7.62-7.79 (2H, m), 7.87 (1H, dd, J=8.7, 1.8 Hz), 8.05 (1H, d, J=8.1 Hz), 8.14 (1H, d, J=8.7 Hz), 8.22 (1H, d, J=7.9 Hz), 8.29 (1H, s), 8.58 (1H, s), 12.18-12.40 (1H, m)

MS^{m}/z: 507 (M+1).

### Example 164

### Ethyl 5-cyano-2-methyl-6-[4-({[(4-methylphenyl)sulfonyl]amino}carbonyl)piperidin-1-yl]nicotinate

The sulfoneamide 4-methylbenzenesulfonamide (0.25 mmol) was reacted in according to method B to give ethyl 5-cyano-2-methyl-6-[4-({[(4-methylphenyl)sulfonyl]amino}carbonyl)piperidin-1-yl]nicotinate. Yield: 0.063 g (64%).

¹H NMR (400 MHz, d₆-DMSO) 51.29 (3H, t, J=7.1 Hz), 1.39-1.53 (2H, m), 1.76-1.87 (2H, m), 2.39 (3H, s), 2.47-2.51 (1H, m), 2.61 (3H, s), 3.07-3.18 (2H, m), 4.24 (2H, q, J=7.1 Hz), 4.42-4.50 (2H, m), 7.41 (2H, d, J=8.1 Hz), 7.78 (211d, J=8.1 Hz), 8.31 (1H, s), 12.06-12.15 (1H, m)

MS^{m}/z: 471 (M+1).

### Example 165

### Ethyl 5-cyano-2-methyl-6-[5{[(phenylsulfonyl)amino]carbonyl}hexahydropyrrolo[3,4-c]pyrrol-2(1H)-yl]nicotinate

### (a) tert-butyl 5-[3-cyano-5-(ethoxycarbonyl)-6-methylpyridin-2-yl]hexahydropyrrolo[3,4-c]pyrrole-2(1H)-carboxylate

Ethyl 6-chloro-5-cyano-2-methylnicotinate (0.090 g, 0.4 mmol) was dissolved in ethanol (2 mL, 98%) in a Smith process vial. ten-Butyl hexahydropyrrolo[3,4-c]pyrrole-2(1H)-carboxylate (0.093 g, 0.44 mmol) and triethylamine (0.202 g, 2.0 mmol) were added. The sealed vial was heated in a microwave oven, single node heating, at 120° C. for 20 minutes. The solvent was evaporated. Flash chromatography on Si-gel with heptane/ethyl acetate 3:1 as eluent gave the wanted product. Yield: 0.088 g (55%).

¹H NMR (400 MHz, CDCl₃): δ 1.35 (3H, t J=7.1 Hz), 1.44 (9H, s), 2.68 (31H, s), 2.92-3.02 (2H, m), 3.24-3.35 (2H, m), 3.56-3.69 (2H, m), 3.72-3.79 (2H, m), 4.03-4.13 (2H, m), 4.28 (2H, q, J=7.1 Hz), 8.30 (1H, s).

¹³C NMR (100 MHz, CDCl₃): δ 14.1, 25.5, 28.3, 40.6, 41.7, 49.2, 49.5, 52.15, 52.25, 60.6, 79.5, 86.9, 113.4, 118.7, 147.4, 154.2, 155.4, 164.6, 164.7

MS^{m}/z: 401 (M+1)

### (b) Ethyl 5-cyano-2-methyl-6-[5-{[(phenylsulfonyl)amino]carbonyl}hexahydropyrrolo[3,4-c]pyrrol-2(1H)-yl]nicotinate

tert-Butyl 5-[3-cyano-5-(ethoxycarbonyl)-6-methylpyridin-2-yl]hexahydropyrrolo[3,4-c]pyrrole-2(1H)-carboxylate (0.085 g, 0.21 mmol) was dissolved in TFA/DCM 1:1 (2 mL) and the reaction mixture was stirred at room temperature for 30 minutes. TLC showed the reaction to be finish. The reaction mixture was evaporated and the crude product dissolved in DCM (1 mL). Triethylamine (0.106 g, 1.05 mmol) was added at room temperature and benzenesulfonyl isocyanate (0.042 g, 0.23 mmol) was added at 0° C. The reaction mixture was stirred at 0° C. for 10 minutes and then at room temperature for 1.5 h. The solvent was evaporated. Purification was done by reverse phase HPLC. Phases: A: CH₃CN, B: 0.1 M NH₄OAc/CH₃CN 95:5. Start: A/B: 5:95. Increase stepwise to 10/90 after 2 minutes, to 20/80 after 5 minutes, to 30/70 after 10 minutes, to 40/60 after 15 minutes, and to 50/50 after 20 minutes. Flow: 20 mL/min. Column: Kromasil CS, 250 mm×20 ID. The relevant fractions were combined and concentrated in vacuo and the material was freeze dried. Yield: 0.075 g (74%).

¹H NMR (400 MHz, d₆₁DMSO): 1.32 (3H, t, J=7.1 Hz), 2.64 (3H, s), 2.95-3.08 (2H, m), 3.59-3.67 (4H, m), 3.96-4.05 (2H, m), 4.25 (2H, q, J=7.1 Hz), 7.57-7.63 (2H, m), 7.64-7.70 (1H, m), 7.91-7.95 (2H, m), 8.29 (1H, s), 10.71-10.75 (1H, s).

MS^{m}/z: 484 (M+1)

### Example 166

### Ethyl 5-cyano-2-methyl-6-{3-[({[5-(2-methyl-1,3-thiazol-4-yl)-2-thienyl]sulfonyl}amino)carbonyl]azetidin-1-yl}nicotinate

Prepared according to method A starting from 5-(2-methyl-1,3-thiazol-4-yl)thiophene-2-sulfonamide (0.098 g, 0.38 mmol). Yield: 0.103 g (77%).

¹H NMR (400 MHz, d₆-DMSO) δ 1.25 (3H, t, J=7.2 Hz,), 2.57 (3H, s), 2.58 (3H, s), 3.46 (1H, m), 4.19 (4H, m), 4.36 (2H, t, J=8.4 Hz), 8.23 (1H, s)

MS^{m}/z: 532 (M+1)

### Example 167

### Ethyl 6-[(1S,4S)-5-({[(5-chloro-2-thienyl)sulfonyl]amino}carbonyl)-2,5-diazabicyclo[2.2.1]hept-2-yl]-5-cyano-2-methylnicotinate

### (a) (1S,4S)-tert-Butyl 5-(3-cyano-5-(ethoxycarbonyl)-6-methylpyridin-2-yl)-2,5-diazabicyclo[2.2.1]heptane-2-carboxylate

A solution of ethyl 6-chloro-5-cyano-2-methylnicotinate (1.00 g, 4.45 mmol), (1S,4S)-tert-butyl 2,5-diazabicyclo[2.2.1]heptane-2-carboxylate (1.06 g, 5.34 mmol), and DIPEA (2.33 mL, 13.4 mmol) in DMF (10 mL) was stirred at room temperature for 1 h. The reaction mixture was diluted with EtOAc (100 mL), washed with saturated NH₄Cl (4×50 mL), brine (3×50 mL), dried (MgSO₄), passed through silica gel and concentrated to produce (1S,4S)-tert-butyl 5-(3-cyano-5-(ethoxycarbonyl)-6-methylpyridin-2-yl)-2,5-diazabicyclo[2.2.1]heptane-2-carboxylate as an oil. Yield: 1.71 g (99%/0).

MS^{m}/z: 387 (M+1).

### (b) Ethyl 6-((1S,4S)-2,5-diazabicyclo[2.2.1]heptan-2-yl)-5-cyano-2-methylnicotinate

A solution of (1S,4S)-tert-Butyl 5-(3-cyano-5-(ethoxycarbonyl)-6-methylpyridin-2-yl)-2,5-diazabicyclo[2.2.1]heptane-2-carboxylate (1.71 g, 4.42 mmol) in EtOH (40 mL) was treated with 4M HCl/dioxane (40 mL). After stirring at room temperature for 18 h, the mixture was concentrated, diluted with EtOAc (300 mL), washed with saturated NaHCO₃ (100 mL), brine (100 mL), dried (MgSO₄), and concentrated to furnish ethyl 6-((1S,4S)-2,5-diazabicyclo[2.2.1]heptan-2-yl)-5-cyano-2-methylnicotinate which was used crude. Yield: 1.13 g (89%).

### (c) Ethyl 6-[(1S,4S)-5-({[(5-chloro-2-thienyl)sulfonyl]amino}carbonyl)-2,5-diazabicyclo[2.2.1]hept-2-yl]-5-cyano-2-methylnicotinate

A solution of ethyl 6-((1S,4S)-2,5-diazabicyclo[2.2.1]heptan-2-yl)-5-cyano-2-methylnicotinate (0.128 g, 0.447 mmol), 2,2,2-trichloroethyl 5-chlorothiophen-2-ylsulfonylcarbamate (0.145 g, 0.447 mmol), DMAP (0.0027 g, 0.022 mmol), and DIPEA (0.39 mL, 2.23 mmol) in DMA (3 mL) was heated to 120° C. for 3 h. The mixture was diluted with EtOAc (100 mL), washed with saturated NH₄Cl₄ (3×50 mL), brine (50 mL), dried (MgSO₄) and concentrated. Flash chromatography (25% EtOAc/hexanes with 1% AcOH) followed by reverse phase preparative HPLC produced ethyl 6-[(1S,4S)-5-({[(5-chloro-2-thienyl)sulfonyl]amino}carbonyl)-2,5-diazabicyclo[2.2.1]hept-2-yl]-5-cyano-2-methylnicotinate. Yield: 0.070g (30%).

¹H NMR (400 MHz, CDCl₃): δ 1.38 (3H, t, J=7.1 Hz), 1.98-2.06 (2H, m), 2.70 (3H, s), 3.55 (2H, s), 3.80-3.83 (1H, m), 4.00-4.03 (1H, m), 4.32 (2H, d, J=7.1 Hz), 4.80 (1H, br s), 6.93 (1H, d, J=4.1 Hz), 7.64 (1H, d, J=4.1 Hz), 8.33 (1H, s).

MS^{m}/z: 510 (M+1

### Example 168

### Ethyl S-cyano-2-methyl-6-(4-{[(phenylsulfonyl)amino]carbonyl}piperidin-1-yl)nicotinate

The sulfoneamide benzenesulfonamide (0.25 mmol) was reacted in according to method B to give ethyl 5-cyano-2-methyl-6-(4-{[(phenylsulfonyl)amino]carbonyl}piperidin-1-yl)nicotinate. Yield: 0.047 g (49%) ¹H NMR (400 MHz, d-4-DMSO) 61.29 (3H, t, J=7.1 Hz), 1.38-1.54 (2H, m), 1.78-1.87 (2H, m), 2.47-2.54 (1H, m), 2.61 (3H, s),3.07-3.19 (2H, m), 4.24 (2H, q, J=7.1 Hz), 4.40-4.52 (2H, m), 7.54-7.76 (3H, m), 7.90 (2H, d, J=7.5 Hz), 8.31 (1H, s), 12.13-12.25 (1H, m)

MS^{m}/z: 457 (M+1).

### Example 169

### Ethyl 5-cyano-6-[4-({[(2,4-dichlorophenyl)sulfonyl]amino}carbonyl)piperidin-1-yl]-2-methylnicotinate

The sulfoneamide 2,4-dichlorobenzenesulfonamide (0.25 mmol) was reacted in according to method B to give ethyl 5-cyano-6-[4-({[(2,4-dichlorophenyl)sulfonyl]amino}carbonyl)piperidin-1-yl]-2-methylnicotinate. Yield: 0.033 g (30%).

¹H NMR (400 MHz, d₆₁DMSO) δ 1.25 (3H, t, J=7.1 Hz), 1.37-1.53 (2H, m), 1.79-1.89 (2H, m), 2.58 (3H, s), 2.60-2.65 (1H, m), 3.06-3.18 (2H, m), 4.20 (2H, q, J=7.1 Hz), 4.39-4.49 (2H, m), 7.63 (1H, d, J=8.7 Hz), 7.84 (1H, s), 8.02 (1H, d, J=8.7 Hz), 8.27 (1H, s), 12.62-12.77 (1H, m)

MS^{m}/z: 525 (M+1).

### Example 170

### Isopropyl 6-[4-({[(3-bromophenyl)sulfonyl]amino}carbonyl)piperidin-1-yl]-5-cyano-2-methylnicotinate

To 1-[3-cyano-5-(isopropoxycarbonyl)-6-methylpyridin-2-yl]piperidine-4-carboxylic acid (0.100 g, 0.302 mmol)), see example 45, were added TBTU (0.097 g, 0.302 mmol), dry DCM (2 mL), DIPEA (0.1 mL, 0.57 mmol) and the mixture was stirred at room temperature for 2.5 h. The mixture was added to 3-bromobenzenesulfonamide (0.085 g, 0.361 mmol), dry DCM (2 mL) was added and the reaction mixture was stirred at room temperature for 18 h. NaHCO₃ (aq) was added and the mixture was extracted with DCM (×3). The combined organic layer was run through a phase separator and solvents were removed in vacuo. The crude product was purified by preparative HPLC (Column: Kromasil C8 10 μm, 21.5×250 mm, Mobilephase A: 100% CH₃CN, Mobilephase B: 5% CH₃CN, 95% 0.1M NH₄OAc(aq), Gradient: 20=>50%).

The relevant fractions was collected and evaporated and freezedried yielding isopropyl 6-[4-({[(3-bromophenyl)sulfonyl]amino}carbonyl)piperidin-1-yl]-5-cyano-2-methylnicotinate as a solid. Yield: 0.132 g (80%)

¹H NMR (500 MHz, d₆-DMSO): 1.28 (6H, d), 1.47 (2H, m), 1.83 (2H, m), 2.61 (3H, s), 2.62 (1H, m), 3.13 (2H, m), 4.46 (2H, m), 5.06 (1H, m), 7.60 (1H, m), 7.89-8.00 (3H, m), 8.28 (1H, s), 12.34 (1H, s).

MS^{m}/z: 550 (M+1), 548 (M−1).

### Example 171

### Ethyl 5-cyano-2-methyl-6-{4-[({[4-(trifluoromethoxy)phenyl]sulfonyl}amino)carbonyl]piperidin-1-yl}nicotinate

The sulfoneamide 4-(trifluoromethoxy)benzenesulfonamide (0.25 mmol) was reacted in according to method B to give ethyl 5-cyano-2-methyl-6-{4-[({[4-(trifluoromethoxy)phenyl]sulfonyl}amino)carbonyl]piperidin-1-yl}nicotinate. Yield: 0.030 g (26%)

¹H NMR (400 MHz, d₆-DMSO) δ 1.29 (3H, , J=7.2 Hz), 1.41-1.56 (2H, m), 1.79-1.88 (2H, m), 2.56-2.60 (1H, m), 2.61 (3H, s), 3.08-3.20 (2H, m), 4.24 (2H, q, J=7.1 Hz), 4.40-4.52 (2H, m), 7.61 (21, d, J=8.5 Hz), 8.03 (2H, d, J=8.9 Hz), 8.31 (1H, s), 12.26-12.43 (1H, m)

MS^{m}/z: 541 (M+1).

### Example 172

### Ethyl 5-cyano-6-[3-({[(6-ethoxy-1,3-benzothiazol-2-yl)sulfonyl]amino}carbonyl)azetidin-1-yl]-2-methylnicotinate

Prepared according to method A starting from 6-ethoxy-benzothiazole-2-sulfonic acid amide

(0.110 g, 0.38 mmol). Yield: 0.142 g (100%).

¹H NMR (400 MHz, DMSO) δ 1.25 (3H, t, J=7.2 Hz), 1.33 (3H, t), 2.56 (3H, s), 3.28 (1H ,m, overlapped by water), 4.08 (2H, m), 4.18 (2H, q, J=7.1 Hz), 4.23 (2H, m), 4.35 (2H, m), 8.22 (1H, s), 7.10 (1H, dd, J=9.0, 2.3 Hz), 7.64 (1H, s), 7.89 (1H, d, J=9.1 Hz)

MS^{m}/z: 530 (M+1)

### Example 173

### Ethyl 5-cyano-2-methyl-6-(3-{2-oxo-2-[(phenylsulfonyl)amino]ethyl}piperidin-1-yl)nicotinate

A solution of 2-(1-(3-cyano-5-(ethoxycarbonyl)-6-methylpyridin-2-yl)piperidin-3-yl)acetic acid (0.100 g, 0.302 mmol), see example 151, EDCI (0.075 g, 0.392 mmol), and HOBT (0.053 g, 0.392 mmol), benzenesulfonamide (0.062 g, 0.392 mmol) and DIPEA (0.105 mL, 0.604 mmol) in DCM (7.0 mL) was stirred at room temperature for 20 h. Following concentration, the mixture was diluted with EtOAc (100 mL), washed with saturated NH₄Cl (2×50 mL), saturated NaHCO₃ (2×50 mL), brine (50 mL), dried (MgSO₄) and concentrated. Flash chromatography (20% EtOAc/hexanes with 0.5% AcOH) furnished ethyl 5-cyano-2-methyl-6-(3-{2-oxo-2-[(phenylsulfonyl)amino]ethyl}piperidin-1-yl)nicotinate as a solid. Yield: 0.030g (21%).

¹H NMR (400 MHz, CDCl₃): δ 1.30-1.35 (1H, m), 1.38 (3H, t, J=7.1 Hz), 1.59-1.63 (1H, m), 1.70-1.76 (1H, m), 1.87-1.91 (1H, m), 2.15-2.32 (2H, m), 2.33-2.40 (1H, m), 2.70 (31, s), 3.12-3.18 (1H, m), 3.29-3.36 (1H, m), 4.25-4.35 (4H, m), 7.52-7.57 (2H, m), 7.64-7.68 (1H, m), 8.06-8.08 (2H, m), 8.11 (1H, s), 8.32 (1H, s).

MS^{m}/z: 471 (M+1).

### Example 174

### Ethyl 5-cyano-6-(4-{[(2,3-dihydro-1,4-benzodioxin-6-ylsulfonyl)amino]carbonyl}piperidin-1-yl)-2-methylnicotinate

The 2,3-dihydro-1,4-benzodioxine-6-sulfonamide (0.25 mmol) was reacted in according to method B to give ethyl 5-cyano-6-(4-{[(2,3-dihydro-1,4-benzodioxin-6-ylsulfonyl)amino]carbonyl}piperidin-1-yl)-2-methylnicotinate. Yield: 0.078 g (72%)

¹H NMR (400 MHz, d₆-DMSO) 31.29 (3H, , J=7.2 Hz), 1.42-1.54 (2H, m), 1.78-1.86 (2H, m), 2.48-2.52 (1H, m), 2.62 (3H, s), 3.07-3.19 (2H, m), 4.24 (2H, q, J=7.1 Hz), 4.28-4.36 (4H, m), 4.43-4.53 (2H, m), 7.05 (1H, d, J=8.5 Hz), 7.31-7.38 (2H, m), 8.31 (1H, s), 11.99-12.11 (1H, m)

MS^{m}/z: 515 (M+1).

### Example 175

### Ethyl 5-cyano-6-[4-({[(4-methoxyphenyl)sulfonyl]amino}carbonyl)piperidin-1-yl]-2-methylnicotinate

The 4-methoxybenzenesulfonamide (0.25 mmol) was reacted in according to method B to give ethyl 5-cyano-6-[4-({[(4-methoxyphenyl)sulfonyl]amino}carbonyl)piperidin-1-yl]-2-methylnicotinate. Yield: 0.064g, 63%

¹H NMR (400 MHz, d₆-DMSO) δ 1.29 (3H, t, J=7.2 Hz), 1.39-1.55 (2H, m), 1.75-1.86 (2H, m), 2.50-2.58 (1H, m), 2.61 (3H, s), 3.07-3.18 (2H, m), 3.84 (3H, s), 4.24 (21, q, J=7.1 Hz), 4.41-4.52 (2H, m), 7.11 (2H, d, J=8.9 Hz), 7.83 (2H, d, J=9.1 Hz), 8.31 (1H, s), 11.97-12.10 (1H, m)

MS^{m}/z: 487 (M+1).

### Example 176

### Ethyl 6-(4-{[(2,1,3-benzoxadiazol-4-ylsulfonyl)amino]carbonyl}piperidin-1-yl)-5-cyano-2-methylnicotinate

The 2,1,3-benzoxadiazole-4-sulfonamide (0.25 mmol) was reacted in according to method B to give ethyl 6-(4-{[(2,1,3-benzoxadiazol-4-ylsulfonyl)amino]carbonyl}piperidin-1-yl)-5-cyano-2-methylnicotinate. Yield: 0.003 g (2%)

MS^{m}/z: 499 (M+1).

### Example 177

### Ethyl 5-cyano-2-methyl-6-[4-({[(3-nitrophenyl)sulfonyl]amino}carbonyl)piperidin-1-yl]nicotinate

The 3-nitrobenzenesulfonamide (0.25 mmol) was reacted in according to method B to give ethyl 5-cyano-2-methyl-6-[4-({[(3-nitrophenyl)sulfonyl]amino}carbonyl)piperidin-1-yl]nicotinate. Yield: 0.015 g, 14%.

1H NMR (400 MHz, d₆-DMSO) δ 1.29 (3H, t, J=7.2 Hz), 1.40-1.56 (2H, m), 1.79-1.90 (2H, m), 2.56-2.60 (1H, m), 2.61 (31H, s), 3.07-3.20 (2H, m), 4.24 (2H, q, J=7.1 Hz), 4.40-4.52 (21, m), 7.92 (1H, t, J=8.1 Hz), 8.31 (1H, s), 8.32 (1H, d, J=8.3 Hz), 8.49-8.55 (1H, m), 8.57-8.61 (1H, m), 12.26-12.86 (1H, m)

MS^{m}/z: 502 (M+1).

### Example 178

### Isopropyl 5-cyano-2-methyl-4-(4-{[(phenylsulfonyl)amino]carbonyl}piperidin-1-yl)nicotinate

To 1-[3-cyano-5-(isopropoxycarbonyl)-6-methylpyridin-2-yl]piperidine-4-carboxylic acid (0.100 g, 0.302 mmol)), see example 45, were added TBTU (0.097 g, 0.302 mmol), dry DCM (2 mL), DIPEA (00.1 mL, 0.57 mmol) and the mixture was stirred at room temperature for 2.5 h. The mixture was added to benzenesulfonamide (0.0566 g, 0.360 mmol), dry DCM (2 ml) was added and the reaction mixture was stirred at room temperature for 18 h. NaHCO₃ (aq) was added and the mixture was extracted with DCM (×3). The combined organic layer was run through a phase separator and solvents were removed in vacuo. The crude product was purified by preparative HPLC (Column: Kromasil C8 10 μm, 21.5×250 mm, Mobilephase A: 100% C₁₋₃CN, Mobilephase B: 5% CH₃CN, 95% 0.1 M NH₄OAc (aq), Gradient: 20=>50%).

tions was evaporated and freezedried yielding the isopropyl 5-cyano-2-methyl-6-(4-{[(phenylsulfonyl)amino]carbonyl}piperidin-1-yl)nicotinate as a solid. Yield: 0.098 g (69%). in NMR (500 MHz, d₆-DMSO): 1.28 (6H, d), 1.46 (2H, m), 1.81 (2H, m), 2.60 (1H, m), 2.60 (3H, s), 3.12 (2H, m), 4.45 (2H, m), 5.05 (1H, m), 7.60-7.91 (5H, m), 8.28 (1H, s), 12.18 (1H, s).

MS^{m}/z: 471 (M+1).

### Example 179

### Isopropyl 5-cyano-2-methyl-6-{3-[({[4-(trifluoromethyl)phenyl]sulfonyl}amino)carbonyl]azetidin-1-yl}nicotinate

4-(Trifluoromethyl)benzenesulfonamide (0.25 mmol) was reacted in according to method C to give isopropyl 5-cyano-2-methyl-6-{3-[({[4-(trifluoromethyl)phenyl]sulfonyl}amino)carbonyl]azetidin-1-yl}nicotinate. Yield: 0.089 g (58%)

¹H NMR (400 MHz, d₆-DMSO): δ 1.30 (d, J=6.3 Hz, 6H), 2.61 (s, 3H), 3.59-3.68 (m, 1H), 4.18-4.27 (m, 2H), 4.37-4.46 (m, 2H), 5.01-5.12 (m, 1H), 8.06 (d, J=8.4 Hz, 2H), 8.19 (d, J=8.4 Hz, 2H), 8.26 (s, 1H), 12.67 (br s, 1H).

MS^{m}/z: 511 (M+1)

### Example 180

### Isopropyl 6-[4-({[(4-chlorophenyl)sulfonyl]amino}carbonyl)piperidin-1-yl]-5-cyano-2-methylnicotinate

To 1-[3-cyano-5-(isopropoxycarbonyl)-6-methylpyridin-2-yl]piperidine-4-carboxylic acid (0.100 g, 0.302 mmol)), see example 45, were added TBTU (0.097 g, 0.302 mmol), dry DCM (2 mL), DIPEA (0.11 mL, 0.57 mmol) and the mixture was stirred at room temperature for 2.5 h. The mixture was added to 4-chlorobenzenesulfonamide (0.0690 g, 0.360 mmol), dry DCM (2 ml) was added and the reaction mixture was stirred at room temperature for 18 h. NaHCO₃ (aq) was added and the mixture was extracted with DCM (x3). The combined organic layer was run through a phase separator and solvents were removed in vacuo. The crude product was purified by preparative HPLC (Column: Kromasil C8 10 μm, 21.5×250 mm, Mobilephase A: 100% CH3CN, Mobilephase B: 5% CH₃CN, 95% 0.1M NH₄OAc (aq), Gradient: 20=>50%).

The relevant fractions was collected, evaporated and freezedried yielding isopropyl 6-[4-({[(4-chlorophenyl)sulfonyl]amino}carbonyl)piperidin-1-yl]-5-cyano-2-methylnicotinate as a solid. Yield: 0.113 g;(74%)

¹H-NMR (500 MHz δ 6-DMSO): 1.28 (6H, d), 1.46 (2H, m), 1.82 (2H, m), 2.60 (1H, m), 2.60 (3H, s), 3.12 (2H, m), 4.45 (2H, m), 4.74 (2H, s), 5.06 (1H, m), 7.70 (2H, m), 7.90 (2H, m), 8.28 (1H, s), 12.29 (1H, s).

MS^{m}/z: 506 (M+1).

### Example 181

### Ethyl 5-cyano-6-[4-({[(3-cyanophenyl)sulfonyl]amino}carbonyl)piperidin-1-yl]-2-methylnicotinate

The 3-cyanobenzenesulfonamide (0.25 mmol) was reacted in according to method B to give ethyl 5-cyano-6-[4-({[(3-cyanophenyl)sulfonyl]amino}carbonyl)piperidin-1-yl]-2-methylnicotinate. Yield: 0.022 (22%)

¹H NMR (400 MHz, D₆-DMSO) δ 1.29 (3H, t, J=7.2 Hz), 1.40-1.56 (2H, m, Hz), 1.78-1.90 (2H, m, Hz), 2.57-2.61 (1H, m, Hz), 2.62 (3H, s, Hz), 3.07-3.20 (2H, m, Hz), 4.24 (2H, q, J=7.1 Hz), 4.47 (2H, d, J=42.3 Hz), 7.84 (1H, t, J=8.0 Hz), 8.19 (2H, t, J=8.8 Hz), 8.29 (1H, s, Hz), 8.31 (1H, s, Hz), 12.39-12.59 (1H, m, Hz)

MS^{m}/z: 482 (M+1).

### Example 182

### Isopropyl 5-cyano-2-methyl-6-(3-{[(2-naphthylsulfonyl)amino]carbonyl}azetidin-1-yl)nicotinate

Naphthalene-2-sulfonamide (0.25 mmol) was reacted in according to method C to give isopropyl 5-cyano-2-methyl-6-(3-{[(2-naphthylsulfonyl)amino]carbonyl}azetidin-1-yl)nicotinate. Yield: 0.040g (27%)

¹H NMR (400 MHz, d₆-DMSO): δ 1.29 (d, J=6.3 Hz, 6H), 2.59 (m, 3H), 3.54-3.65 (m, 1H), 4.14-4.25 (m, 2H), 4.35-4.45 (m, 2H), 5.00-5.11 (m, 1H), 7.68-7.79 (m, 2H), 7.90-7.95 (m, 1H), 8.05-8.10 (m, 1H), 8.14-8.19 (m, 1H), 8.22-8.27 (m, 21), 8.63 (s, 1H), 12.40-12.61 (br s, 1H).

MS^{m}/z: 492 (M+1)

### Example 183

### Ethyl 5-cyano-2-methyl-6-{4-[({[2-(trifluoromethoxy)phenyl]sulfonyl}amino)carbonyl]piperidin-1-yl}nicotinate

The 2-(trifluoromethoxy)benzenesulfonamide (0.25 mmol) was reacted in according to method B to give ethyl 5-cyano-2-methyl-6-{4-[({[2-(trifluoromethoxy)phenyl]sulfonyl}amino)carbonyl]piperidin-1-yl}nicotinate. Yield: 0.012 g (11%).

¹H NMR (400 MHz, d₆-DMSO) 61.29 (3H, t, J=7.2 Hz), 1.41-1.56 (2H, m), 1.79-1.89 (2H, m), 2.50-2.52 (1H, m), 2.61 (3H, s), 3.10-3.21 (2H, m), 4.23 (2H, q, J=7.1 Hz), 4.40-4.53 (2H, m), 7.53-7.64 (2H, m), 7.75-7.88 (1H, m), 8.04 (1H, d, J=7.7 Hz), 8.31 (1H, s), 12.54-12.67 (1H, m)

MS^{m}/z: 541 (M+1).

### Example 184

### Isopropyl 5-cyano-6-[4-({[(4-methoxyphenyl)sulfonyl]amino}carbonyl)piperidin-1-yl]-2-methylnicotinate

To 1-[3-cyano-5-(isopropoxycarbonyl)-6-methylpyridin-2-yl]piperidine-4-carboxylic acid (0.100 g, 0.302 mmol)), see example 45, were added TBTU (0.097 g, 0.302 mmol), dry DCM (2 mL), DIPEA (00.1 mL, 0.57 mmol) and the mixture was stirred at room temperature for 2.5 h. The mixture was added to 4-methoxybenzenesulfonamide (0.0674 g, 0.360 mmol), dry DCM (2 mL) was added and the reaction mixture was stirred at room temperature for 18 h. NaHCO₃ (aq) was added and the mixture was extracted with DCM (×3). The combined organic layer was run through a phase separator and solvents were removed in vacuo. The crude product was purified by preparative HPLC (Column: Kromasil C8 10 μm, 21.5×250 mm, Mobilephase A: 100% CH₃CN, Mobilephase B: 5% CH₃CN, 95% 0.1M NH₄OAc(aq), Gradient: 20=>50%).

The relevant fractions was collected, evaporated and freeze dried yielding isopropyl 6-[4-(({[(4-chlorophenyl)sulfonyl]amino}carbonyl)piperidin-1-yl]-5-cyano-2-methylnicotinate as a solid. Yield: 0.146 g (48%)

¹H NMR (500 MHz, d₆-DMSO): δ1.28 (6H, d), 1.45 (2H, m), 1.80 (2H, m), 2.58 (1H, m), 2.60 (3H, s), 3.11 (2H, m), 3.84 (3H, s), 4.44 (2H, m), 5.06 (1H, m), 7.12 (2H, m), 7.83 (2H, m), 8.28 (1H, s), 12.02 (1H, s)

MS^{m}/z: 501 (M+1).

The below two compounds were made by similar methods described above.

### Example 185

### Ethyl 5-cyano-2-methyl-6-(3-{2-oxo-2-[(phenylsulfonyl)amino]ethyl}azetidin-1-yl)nicotinate

¹H NMR (500 MHz, d₆-DMSO): δ 1.29 (3H, t, J=7.3 Hz), 2.59 (3H, s), 2.69 (2H, d), 2.90 (1H, m), 3.92 (2H, m), 4.22 (2H, q, J=7.11 Hz), 4.34 (2H, m), 7.62 (2H, m), 7.71 (1H, m), 7.92 (2H, m), 8.24 (1H, s), 12.22 (1H, s).

MS^{m}/z: 443 (M+1).

### Example 186

### Ethyl 6-[3-(2-{[(5-chloro-2-thienyl)sulfonyl]amino}-2-oxoethyl)azetidin-1-yl]-5-cyano-2-methylnicotinate

¹H NMR (500 MHz, d₆-DMSO): δ 1.29 (3H, t J=7.1 Hz), 2.60 (3H, s), 2.61 (2H, d), 2.94 (In, m), 3.97 (2H, m), 4.23 (2H, q, J=7.0 Hz), 4.37 (21, m), 7.16 (1H, m), 7.52 (1H, m), 8.25 (1H, s), 11.95 (1H, s).

MS^{m}/z: 483 (M+1).

1. A compound of formula I or a pharmaceutically acceptable salt thereof: 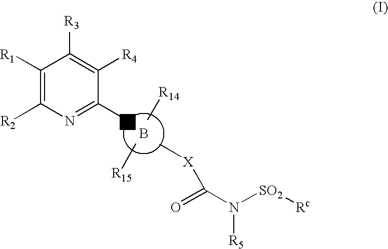
wherein:
R₁ represents R₆OC(O), R₇C(O), R₁₆SC(O), R₁₇S, R₁₈C(S) or a group selected from 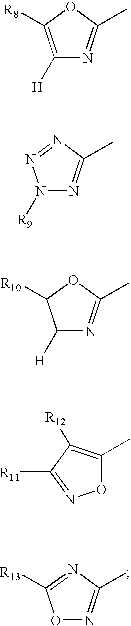
R₂ represents H, CN, NO₂, (C₁-C₁₂)alkyl optionally interrupted by oxygen and/or optionally substituted by OH, aryl, cycloalkyl, heterocyclyl or one or more halogen atoms; or
R₂ represents (C₃-C₆)cycloalkyl, hydroxy(C₁-C₁₂)alkyl, (C₁-C₁₂)alkylC(O), (C₁-C₁₂)alkylthioC(O), (C₁-C₁₂)alkylC(S), (C₁-C₁₂)alkoxy, (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl,
aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(2)}R^{b(2)} in which R^{a(2)} and R^{b(2)} independently represent H, (C₁-C₁₂)alkyl, (C₁-C₁₂)alkylC(O) or R^{a(2)} and R^{b(2)} together with the nitrogen atom represent piperidine, pyrrolidine, azetidine or aziridine; or
R₁+R₂ together (with two carbon atoms of the pyridine ring) may form a 5-membered or 6-membered cyclic lactone;
R₃ represents H, CN, NQ, halogen, (C₁-C₁₂)alkyl optionally interrupted by oxygen and/or optionally substituted by OH, aryl, cycloalkyl, heterocyclyl or one or more halogen atoms; or
R₃ represents (C₃-C₆)cycloalkyl, hydroxy(C₁-C₁₂)alkyl, (C₁-C₁₂)alkylC(O), (C₁-C₁₂)alkoxy, (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl,
aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(3)}R^{b(3)} in which R^{a(3)} and R^{b(3)} independently represent H, (C₁-C₁₂)alkyl, (C₁-C₁₂)alkylC(O) or R^{a(3)} and R_{b(3)} together with the nitrogen atom represent piperidine, pyrrolidine, azetidine or aziridine;
R₄ represents H, CN, NO₂, halogen (C₁-C₁₂)alkyl optionally interrupted by oxygen and/or optionally substituted by OH, COOH, aryl, cycloalkyl, heterocyclyl or one or more halogen atoms; or
R₄ represents (C₃-C₆)cycloalkyl, hydroxy(C₁-C₁₂)alkyl, (C₁-C₁₂)alkylC(O), (C₁-C₁₂)alkylcycloalkyl, (C₁-C₁₂)alkoxy wherein the alkoxygroup may optionally be substituted by OH and/or COOH; or
R₄ represents (C₁-C₁₂)alkylthioC(O), (C₁-C₁₂)alkylC(S), (C₁-C₁₂)alkoxyC(O), (C₃-C₆)cycloalkoxy, aryl, arylC(O), aryl(C₁-C₁₂)alkylC(O), heterocyclyl, heterocyclylC(O), heterocyclyl(C₁-C₁₂)alkylC(O), (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl,
aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)Cycloalkyl(C₁-C₁₂)alkylsulfonyl or a group of formula NR^{a(4)}R^{b(4)} in which R^{a(4)} and R^{b(4)} independently represent H, (C₁-C₁₂)alkyl, (C₁-C₁₂)alkylC(O) or R^{a(4)} and R^{b(4)} together with the nitrogen atom represent piperidine, pyrrolidine, azetidine or aziridine;
R₅ represents H or (C₁-C₁₂)alkyl;
R₆ represents (C₁₋₁₂)alkyl optionally interrupted by oxygen, (with the proviso that any such oxygen must be at least 2 carbon atoms away from the ester-oxygen connecting the R₆ group) and/or optionally substituted by OH, aryl, cycloalkyl, heterocyclyl or one or more halogen atoms; or
R₆ represents (C₃₋₆)cycloalkyl, hydroxy(C₂-C₁₂)alkyl, aryl or heterocyclyl;
R₇ represents (C₁-C₁₂)alkyl optionally interrupted by oxygen, and/or optionally substituted by OH, aryl, cycloalkyl, heterocyclyl or one or more halogen atoms; or
R₇ represents (C₃-C₆)cycloalkyl, hydroxy(C₁-C₁₂)alkyl, (C₁-C₁₂)alkoxy, (C₃-C₆)cycloalkoxy, aryl or heterocyclyl;
R₈ represents H, (C₁-C₁₂)alkyl optionally interrupted by oxygen, and/or optionally substituted by aryl, cycloalkyl, heterocyclyl or one or more halogen atoms; or
R₈ represents (C₃-C₆)cycloalkyl, hydroxy(C₁-C₁₂)alkyl,(C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)Cycloalkyl(C₁-C₁₂)alkylsulfinyl or (C₃-C₆)cycloalkyl(C₁-C₁₂)alkylsulfonyl;
R₉ represents H, (C₁-C₁₂)alkyl optionally interrupted by oxygen, and/or optionally substituted by aryl, cycloalkyl, heterocyclyl or one or more halogen atoms; or
R₉ represents (C₃-C₆)cycloalkyl, hydroxy(C₁-C₁₂)alkyl, aryl or heterocyclyl;
R₁₀ represents (C₁-C₁₂)alkyl optionally interrupted by oxygen, and/or optionally substituted by aryl, cycloalkyl, heterocyclyl or one or more halogen atoms; or
R₁₀ represents (C₃-C₆)cycloalkyl, hydroxy(C₁-C₁₂)alkyl,(C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁&₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆Cycloalkyl(C₁-C₁₂)alkylsulfinyl or (C₃-C₆)cycloalkyl(C₁-C₁₂)alkylsulfonyl;
R₁₁ represents H, (C₁-C₁₂)alkyl optionally interrupted by oxygen, and/or optionally substituted by aryl, cycloalkyl, heterocyclyl or one or more halogen atoms; or
R₁₁ represents (C₃-C₆)cycloalkyl, hydroxy(C₁-C₁₂)alkyl, (C₁-C₁₂)alkoxy, (C₃-C₆)Cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;
R₁₂ represents H, (C₁-C₁₂)alkyl optionally interrupted by oxygen, and/or optionally substituted by aryl, cycloalkyl, heterocyclyl or one or more halogen atoms; or
R₁₂ represents (C₃-C₆)cycloalkyl, hydroxy(C₁-C₁₂)alkyl, C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁₋₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;
R₁₃ represents H, (C₁-C₁₂)alkyl optionally interrupted by oxygen, and/or optionally substituted by aryl, cycloalkyl, heterocyclyl or one or more halogen atoms; or
R₁₃ represents (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl;
R₁₄ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{d}; wherein R^{d} represents aryl, cycloalkyl, heterocyclyl or (C₁-C₁₂)alkyl optionally substituted by one or more of halogen atoms, OH, aryl, cycloalkyl and heterocyclyl; or
R₁₄ represents aryl, cycloalkyl, heterocyclyl, one or more halogen atoms, (C₃-C₆)cycloalkyl, hydroxy(C₁-C₁₂)alkyl, (C₁-C₁₂)alkoxy, (C₃-C₆)cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl or (C₃-C₆)cycloalkyl(C₁-C₁₂)alkylsulfonyl, a group of formula NR^{a(14)}R^{b(14)} in which R^{a(14)} and R^{b(14)} independently represent H, (C₁-C₁₂)alkyl, (C₁-C₁₂)alkylC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₁₂)alkyl optionally interrupted by oxygen and/or optionally substituted by one or more of OH, COOH and COOR^{d}; wherein R^{d} represents aryl, cycloalkyl, heterocyclyl or (C₁-C₁₂)alkyl optionally substituted by one or more of halogen atoms, OH, aryl, cycloalkyl and heterocyclyl; or
R₁₅ represents aryl, cycloalkyl, heterocyclyl, one or more halogen atoms, (C₃-C₆)cycloalkyl, hydroxy(C₁-C₁₂)alkyl, (C₁-C₁₂)alkoxy, (C₃-Q)cycloalkoxy, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylth, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(15)}R^{b(15)} in which R^{a(15)} and R^{b(15)} independently represent H, (C₁-C₁₂)alkyl, (C₁-C₁₂)alkylC(O) or R^{a(15)} and R^{b(15)} together with the nitrogen atom represent piperidine, pyrrolidine, azetidine or aziridine;
R₁₆ represents (C₁-C₁₂)alkyl optionally interrupted by oxygen and/or optionally substituted by OH, aryl, cycloalkyl, heterocyclyl or one or more halogen atoms, further R₁₆ represents (C₃-C₆)cycloalkyl, hydroxy(C₂-C₁₂)alkyl,(C₁-C₁₂)alkoxy, (C₃-C₆)cycloalkoxy, aryl or heterocyclyl;
R₁₇ represents (C₁-C₁₂)alkyl optionally interrupted by oxygen and/or optionally substituted by OH, aryl, cycloalkyl, heterocyclyl or one or more halogen atoms; or
R₁₇ represents (C₃-C₆)cycloalkyl, hydroxy(C₁-C₁₂)alkyl,(C₁-C₁₂)alkoxy, (C₃-C₆)cycloalkoxy, aryl or heterocyclyl;
R₁₈ represents (C₁-C₁₂)alkyl optionally interrupted by oxygen and/or optionally substituted by OH, aryl, cycloalkyl, heterocyclyl or one or more halogen atoms; or
R₁₈ represents (C₃-C₆)cycloalkyl, hydroxy(C₁-C₁₂)alkyl,(C₁-C₁₂)alkoxy, (C₃-C₆)cycloalkoxy, aryl or heterocyclyl;
R^{c} represents (C₃-C₈)cycloalkyl, aryl or heterocyclyl, and anyone of these groups optionally substituted with one or more halogen atoms and/or one or more of the following groups: OH, CN, NO₂ (C₁-C₁₂)alkyl, (C₁-C₁₂)alkoxyC(O), (C₁-C₁₂)alkoxy, halogen substituted (C₁-C₁₂)alkyl, (C₃-C₆)cycloalkyl, aryl, heterocyclyl, (C₁-C₁₂)alkylsulfinyl, (C₁-C₁₂)alkylsulfonyl, (C₁-C₁₂)alkylthio, arylsulfinyl, arylsulfonyl, arylthio, aryl(C₁-C₁₂)alkylthio, aryl(C₁-C₁₂)alkylsulfinyl, aryl(C₁-C₁₂)alkylsulfonyl, heterocyclyl(C₁-C₁₂)alkylthio, heterocyclyl(C₁-C₁₂)alkylsulfinyl, heterocyclyl(C₁-C₁₂)alkylsulfonyl, (C₃-C₆)cycloalkyl(C₁-C₁₂)alkylthio, (C₃-C₆)cycloalkyl(C₁-C₁₂)alkylsulfinyl, (C₃-C₆)cycloalkyl(C₁-C₁₂)alkylsulfonyl or a group of formula NR^{a(Rc)}R^{b(Rc)} in which R^{a(Rc)} and R^{b(Rc)} independently represent H, (C₁-C₁₂)alkyl, (C₁-C₁₂)alkylC(O) or R^{a(Rc)} and R^{b(Rc)} together with the nitrogen atom represent piperidine, pyrrolidine, azetidine or aziridine;
X represents a single bond, imino (—NH—), methylene (—CH₁₂—), iminomethylene (—CH₂—NH—) wherein the carbon is connected to the B-ring/ringsystem, methyleneimino (—NH—CH₂—) wherein the nitrogen is connected to the B-ring/ringsystem and any carbon and/or nitrogen in these groups may optionally be substituted with (C₁-C₆) alkyl; or
X represents a group (—CH₂—)ₙ wherein n=2-6, which optionally is unsaturated and/or substituted by one or more substituent chosen among halogen, hydroxyl or (C₁-C₆)alkyl.; and
B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions; the substituents R₁₄ and R₁₅ are connected to the B ring/ring system in such a way that no quarternary ammonium compounds are formed (by these connections).

2. A compound according to claim 1 wherein:
R₁ represents R₆OC(O), R₇C(O), R₁₆SC(O), R₁₇S, R₁₈C(S) or a group selected from 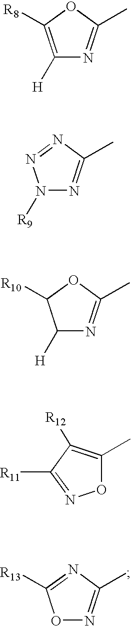
R₂ represents H, CN, NO₂, (C₁-C₆)alkyl optionally interrupted by oxygen and/or optionally substituted by OH, aryl, cycloalkyl, heterocyclyl or one or more halogen atoms; or
R₂ represents (C₃-C₆)cycloalkyl, hydroxy(C₁-C₆)alkyl, (C₁-C₆)alkylC(O), (C₁-C₆)alkoxy, (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₃-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁₋₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(2)}R^{b(2)} in which R^{a(2)} and R^{b(2)} independently represent H, (C₁-C₆)alkyl, (C₁-C₆)alkylC(O) or R^{a(2)} and R^{b(2)} together with the nitrogen atom represent piperidine, pyrrolidine, azetidine or aziridine; or
R₁+R₂ together (with two carbons from the pyridine ring) may form a 5-membered or 6-membered cyclic lactone;
R₃ represents H, CN, NO₂, halogen, (C₁-C₆)alkyl optionally interrupted by oxygen and/or optionally substituted by OH, aryl, cycloalkyl, heterocyclyl or one or more halogen atoms; or
R₃ represents (C₃-C₆)cycloalkyl, hydroxy(C₁-C₆)alkyl, (C₁-C₆)alkylC(O), (C₁-C₆)alkoxy, (C₁₋₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁₋₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents H, CN, NO₂, halogen, (C₁-C₆)alkyl optionally interrupted by oxygen and/or optionally substituted by OH, COOH, aryl, cycloalkyl, heterocyclyl or one or more halogen atoms; or
R₄ represents (C₃-C₆)cycloalkyl, hydroxy(C₁-C₆)alkyl, (C₁-C₆)alkylC(O), (C₁-C₆)alkoxy wherein the alkoxy group may optionally be substituted by OH and/or COOH; or
R₄ represents (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;
R₅ represents H or (C₁-C₆)alkyl;
R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen atoms;
R₆ represents (C₃-C₆)cycloalkyl, hydroxy(C₂-C₆)alkyl, aryl or heterocyclyl;
R₇ represents (C₁-C₆)alkyl optionally interrupted by oxygen, and/or optionally substituted by OH, aryl, cycloalkyl, heterocyclyl or one or more halogen atoms;
R₇ represents (C₃-C₆)cycloalkyl, hydroxy(C₁-C₆)alkyl, (C₁-C₆)alkoxy, (C₃-C₆)cycloalkoxy, aryl or heterocyclyl;
R₈ represents H, (C₁-C₆)alkyl optionally interrupted by oxygen, and/or optionally substituted by aryl, cycloalkyl, heterocyclyl or one or more halogen atoms; or
R₈ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₈)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;
R₉ represents H, (C₁-C₆)alkyl optionally interrupted by oxygen, and/or optionally substituted by aryl, cycloalkyl, heterocyclyl or one or more halogen atoms; or
R₉ represents (C₃-C₆)cycloalkyl, hydroxy(C₁-C₆)alkyl, aryl or heterocyclyl;
R₁₀ represents (C₁-C₆)alkyl optionally interrupted by oxygen, and/or optionally substituted by aryl, cycloalkyl, heterocyclyl or one or more halogen (F, Cl, Br, I) atoms; further R₁₀ represents (C₃-C₆)cycloalkyl, hydroxy(C₁-C₆)alkyl,(C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;
R₁₁ represents H, (C₁-C₆)alkyl optionally interrupted by oxygen, and/or optionally substituted by aryl, cycloalkyl, heterocyclyl or one or more halogen atoms; or
R₁₁, represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁₋₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₈)alkylsulfinyl, heterocyclyl(C₁₋₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;
R₁₂ represents H, (C₁-C₆)alkyl optionally interrupted by oxygen, and/or optionally substituted by aryl, cycloalkyl, heterocyclyl or one or more halogen atoms; or
R₁₂ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)ₑCycloalkyl(C₁-C₆)alkylsulfonyl;
R₁₃ represents H, (C₁-C₆)alkyl optionally interrupted by oxygen, and/or optionally substituted by aryl, cycloalkyl, heterocyclyl or one or more halogen atoms; or
R₁₃ represents (C₃-C₆)cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{d}; wherein R^{d} represents aryl, cycloalkyl, heterocyclyl or (C₁-C₆)alkyl optionally substituted by one or more of halogen atoms, OH, aryl, cycloalkyl and heterocyclyl; or
R₁₄ represents aryl, cycloalkyl, heterocyclyl, one or more halogen atoms, (C₃-C₆)Cycloalkyl, hydroxy(C₁-C₆)alkyl, (C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)Cycloalkyl(C₁-C₆)alkylsulfonyl or a group of formula NR^{a(14)}R^{b(14)} in which R^{a(14)} and R^{b(14)} independently represent H, (C₁-C₆)alkyl, (C₁-C₆)alkylC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{d}; wherein R^{d} represents aryl, cycloalkyl, heterocyclyl or (C₁-C₆)alkyl optionally substituted by one or more of halogen atoms, OH, aryl, cycloalkyl and heterocyclyl; further R₁₅ represents aryl, cycloalkyl, heterocyclyl, one or more halogen atoms, (C₃-C₆)cycloalkyl, hydroxy(C₁-C₆)alkyl,
(C₁-C₆)alkoxy, (C₃-C₆)cycloalkoxy, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(15)}R^{b(15)} in which R^{a(15)} and R^{b(15)} independently represent H, (C₁-C₆)alkyl, (C₁-C₆)alkylC(O) or R^{a(15)} and R^{b(15)} together with the nitrogen atom represent piperidine, pyrrolidine, azetidine or aziridine;
R₁₆ represents (C₁-C₆)alkyl optionally interrupted by oxygen and/or optionally substituted by OH, aryl, cycloalkyl, heterocyclyl or one or more halogen atoms; or
R₁₆ represents (C₃-C₆)cycloalkyl, hydroxy(C₂-C₆)alkyl, (C₁-C₆)alkoxy, (C₃-C₆)cycloalkoxy, aryl, or heterocyclyl;
R₁₇ represents (C₁-C₆)alkyl optionally interrupted by oxygen and/or optionally substituted by OH, aryl, cycloalkyl, heterocyclyl or one or more halogen atoms; or
R₁₇ represents (C₃-C₆)cycloalkyl, hydroxy(C₁-C₆)alkyl, C₁-C₆)alkoxy, (C₃-C₆)cycloalkoxy, aryl or heterocyclyl;
R₁₈ represents (C₁-C₆)alkyl optionally interrupted by oxygen and/or optionally substituted by OH, aryl, cycloalkyl, heterocyclyl or one or more halogen atoms; or
R₁₈ represents (C₃-C₆)cycloalkyl, hydroxy(C₁-C₆)alkyl, (C₁-C₆)alkoxy, (C₃-C₆)cycloalkoxy, aryl or heterocyclyl;
R^{c} represents (C₃-C₈)cycloalkyl, aryl or heterocyclyl, and anyone of these groups optionally substituted with one or more halogen atoms and/or one or more of the following groups, OH, CN, NQ, (C₁-C₆)alkyl, (C₁-C₆)alkoxyC(O), (C₁-C₆)alkoxy, halogen substituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl, heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl, (C₃-C₆)cycloalkyl(C₁-C₆)alkylsulfonyl or a group of formula NR^{a(Rc)}R^{b(Rc)} in which R^{a(Rc)} and R^{b(Rc)} independently represent H, (C₁-C₆)alkyl, (C₁-C₆)alkylC(O) or R^{a(Rc)} and R^{b(Rc)} together with the nitrogen atom represent piperidine, pyrrolidine, azetidine or aziridine;
X represents a single bond, imino (—NH—), methylene (—CH₂—), iminomethylene (—CH₂—NH—) wherein the carbon is connected to the B-ring/ringsystem, methyleneimino (—NH—CH₂—) wherein the nitrogen is connected to the B-ring/ringsystem and any carbon and/or nitrogen in these groups may optionally be substituted with (C₁-C₆) alkyl; or
X may represent a group (—CH₁₂—)ₙ wherein n=2-6, which optionally is unsaturated and/or substituted by one or more substituent chosen among halogen, hydroxyl or (C₁-C₆)allyl; and
B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions; the substituents R₁₄ and R₁₅ are connected to the B ring/ring system in such a way that no quarternary ammonium compounds are formed (by these connections).

3. A compound according to claim 2 wherein:
R₁ represents R₆OC(O), R₇C(O), or a group selected from 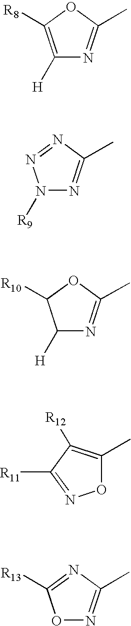
R₂ represents H, CN, NO₂, (C₁-C₆)alkyl optionally interrupted by oxygen and/or optionally substituted by OH, aryl, cycloalkyl, heterocyclyl or one or more halogen atoms; or
R₂ represents (C₃-C₆)cycloalkyl, hydroxy(C₁-C₆)alkyl, (C₁-C₆)alkylC(O), (C₁-C₆)alkoxy, (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(2)}R^{b(2)} in which R^{a(2)} and R^{b(2)} independently represent H, (C₁₋₆)alkyl, (C₁₋₆)alkylC(O) or R^{a(2)} and R^{b(2)} together with the nitrogen atom represent piperidine, pyrrolidine, azetidine or aziridine;
R₃ represents H, CN, NO₂, halogen, (C₁-C₆)alkyl optionally interrupted by oxygen and/or optionally substituted by OH, aryl, cycloalkyl, heterocyclyl or one or more halogen atoms; or
R₃ represents (C₃-C₆)cycloalkyl, hydroxy(C₁-C₆)alkyl, (C₁-C₆)alkylC(O), (C₁-C₆)alkoxy, (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O), (C₁-C₆)alkylsulfinyl, or a group of formula NR^{a(3)}R^{b(3)} in which R^{a(3)} and R^{b(3)} independently represent H, (C₁-C₆)alkyl, (C₁-C₆)alkylC(O) or R^{a(3)} and R^{b(3)} together with the nitrogen atom represent piperidine, pyrrolidine, azetidine or aziridine;
R₄ represents H, CN, NO₂, halogen, (C₁-C₆)alkyl optionally interrupted by oxygen and/or optionally substituted by OH, COOH, aryl, cycloalkyl, heterocyclyl or one or more halogen atoms; or
R₄ represents (C₃-C₆)cycloalkyl, hydroxy(C₁-C₆)alkyl, (C₁-C₆)alkylC(O), (C₁₋₆)alkoxy wherein the alkoxygroup may optionally be substituted by OH and/or COOH; or
R₄ represents, (C₁-C₆)alkylthioC(O), (C₁-C₆)alkylC(S), (C₁-C₆)alkoxyC(O), (C₃-C₆)cycloalkoxy, aryl, arylC(O), aryl(C₁-C₆)alkylC(O), heterocyclyl, heterocyclylC(O), heterocyclyl(C₁-C₆)alkylC(O) or a group of formula NR^{a(4)}R^{b(4)} in which R^{a(4)} and R^{b(4)} independently represent H, (C₁-C₆)alkyl, (C₁-C₆)alkylC(O) or R^{a(4)} and R^{b(4)} together with the nitrogen atom represent piperidine, pyrrolidine, azetidine or aziridine;
R₅ represents H or (C₁-C₆)alkyl;
R₆ represents (C₁-C₆)alkyl optionally interrupted by oxygen, (with the proviso that any such oxygen must be at least 1 carbon atom away from the ester-oxygen connecting the R₆ group) and/or optionally substituted by OH, aryl, cycloalkyl, heterocyclyl or one or more halogen atoms; or
R₆ represents (C₃-C₆)cycloalkyl, hydroxy(C₂-C₆)alkyl, aryl or heterocyclyl;
R₇ represents (C₁-C₆)alkyl optionally interrupted by oxygen, and/or optionally substituted by OH, aryl, cycloalkyl, heterocyclyl or one or more halogen atoms; or
R₇ represents (C₃-C₆)cycloalkyl, hydroxy(C₁-C₆)alkyl,(C₁-C₆)alkoxy, (C₃-C₆)cycloalkoxy, aryl or heterocyclyl;
R₈ represents H, (C₁-C₆)alkyl optionally interrupted by oxygen, and/or optionally substituted by aryl, cycloalkyl, heterocyclyl or one or more halogen atoms; or
R₈ represents (C₃-C₆)cycloalkyl, hydroxy(C₁-C₆)alkyl, (C₁-C₆)alkoxy, (C₃-C₆)cycloalkoxy, aryl or heterocyclyl;
R₉ represents H, (C₁-C₆)alkyl optionally interrupted by oxygen, and/or optionally substituted by aryl, cycloalkyl, heterocyclyl or one or more halogen atoms; or
R₉ represents (C₃-C₆)cycloalkyl, hydroxy(C₁-C₆)alkyl, aryl or heterocyclyl;
R₁₀ represents (C₁-C₆)alkyl optionally interrupted by oxygen, and/or optionally substituted by aryl, cycloalkyl, heterocyclyl or one or more halogen atoms; or
R₁₀ represents (C₃-C₆)cycloalkyl, hydroxy(C₁-C₆)alkyl,(C₁-C₆)alkoxy, (C₃-C₆)cycloalkoxy, aryl or heterocyclyl;
R₁₁ represents a, (C₁-C₆)alkyl optionally interrupted by oxygen, and/or optionally substituted by aryl, cycloalkyl, heterocyclyl or one or more halogen atoms; or
R₁₁ represents (C₃-C₆)cycloalkyl, hydroxy(C₁-C₆)alkyl,(C₁-C₆)alkoxy, (C₃-C₆)cycloalkoxy, aryl or heterocyclyl;
R₁₂ represents H, (C₁-C₆)alkyl optionally interrupted by oxygen, and/or optionally substituted by aryl, cycloalkyl, heterocyclyl or one or more halogen atoms; or
R₁₂ represents (C₃-C₆)cycloalkyl, hydroxy(C₁-C₆)alkyl, (C₁-C₆)alkoxy, (C₃-C₆)cycloalkoxy, aryl or heterocyclyl;
R₁₃ represents H, (C₁-C₆)alkyl optionally interrupted by oxygen, and/or optionally substituted by aryl, cycloalkyl, heterocyclyl or one or more halogen atoms; or
R₁₃ represents (C₃-C₆)cycloalkyl, hydroxy(C₁-C₆)alkyl, (C₁-C₆)alkoxy, (C₃-C₆)cycloalkoxy, aryl or heterocyclyl;
R₁₄ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{d}; wherein R^{d} represents aryl, cycloalkyl, heterocyclyl or (C₁-C₆)alkyl optionally substituted by one or more of halogen atoms, OH, aryl, cycloalkyl and heterocyclyl; or
R₁₄ represents aryl, cycloalkyl, heterocyclyl, one or more halogen atoms, (C₃-C₆)cycloalkyl, hydroxy(C₁-C₆)alkyl,(C₁-C₆)alkoxy, (C₃-C₆)Cycloalkoxy, aryl, heterocyclyl or a group of formula NR^{a(14)}R^{b(14)} in which R^{a(14)} and R^{b(14)} independently represent H,
(C₁-C₆)alkyl, (C₁-C₆)alkylC(O) or R^{a(14)} and R^{b(14)} together with the nitrogen atom represent piperidine, pyrrolidine, azetidine or aziridine;
R₁₅ represents H, OH with the proviso that the OH group must be at least 2 carbon atoms away from any heteroatom in the B ring/ring system, (C₁-C₆)alkyl optionally interrupted by oxygen and/or optionally substituted by one or more of OH, COOH and COOR^{d}; wherein R^{d} represents aryl, cycloalkyl, heterocyclyl or (C₁-C₆)alkyl optionally substituted by one or more of halogen atoms, OH, aryl, cycloalkyl and heterocyclyl; or
R₁₅ represents aryl, cycloalkyl, heterocyclyl, one or more halogen atoms, (C₃-C₆)cycloalkyl, hydroxy(C₁-C₆)alkyl,(C₁-C₆)alkoxy, (C₃-C₆)cycloalkoxy, aryl, heterocyclyl or a group of formula NR^{a(15)}R^{b(15)} in which R^{a(15)} and R^{b(15)} independently represent H,
(C₁-C₆)alkyl, (C₁-C₆)alkylC(O) or R^{a(15)} and R^{b(15)} together with the nitrogen atom represent piperidine, pyrrolidine, azetidine or aziridine;
R^{c} represents (C₃-C₈)cycloalkyl, aryl or heterocyclyl, and anyone of these groups optionally substituted with one or more halogen atoms and/or one or more of the following groups: CN, NO₂, (C₁-C₆)alkyl, (C₁-C₆)alkoxy, halosubstituted (C₁-C₆)alkyl, (C₃-C₆)cycloalkyl, aryl, heterocyclyl, (C₁-C₆)alkylsulfinyl, (C₁-C₆)alkylsulfonyl, (C₁-C₆)alkylthio, arylsulfinyl, arylsulfonyl, arylthio, aryl(C₁-C₆)alkylthio, aryl(C₁-C₆)alkylsulfinyl, aryl(C₁-C₆)alkylsulfonyl,
heterocyclyl(C₁-C₆)alkylthio, heterocyclyl(C₁-C₆)alkylsulfinyl, heterocyclyl(C₁-C₆)alkylsulfonyl, (C₃-C₆)cycloalkyl(C₁-C₆)alkylthio, (C₃-C₆)cycloalkyl(C₁-C₆)alkylsulfinyl or (C₃-C₆)cycloalkyl(C₁-C₆)alkylsulfonyl;
X represents a single bond, imino (—NH—), methylene (—CH₂—), iminomethylene (CH₂—NH—) wherein the carbon is connected to the B-ring/ringsystem, methyleneimino (—NH—CH₂—) wherein the nitrogen is connected to the B-ring/ringsystem and any carbon and/or nitrogen in these groups may optionally be substituted with (C₁-C₆) alkyl; or
X may represent a group (—CH₂—)ₙ wherein n-2-6, which optionally is unsaturated and/or substituted by one or more substituent chosen among halogen, hydroxyl or (C₁-C₆)alkyl; and
B is a monocyclic or bicyclic, 4 to 11-membered heterocyclic ring/ring system comprising one or more nitrogen and optionally one or more atoms selected from oxygen or sulphur, which nitrogen is connected to the pyridine-ring (according to formula I) and further the B-ring/ring system is connected to X in another of its positions; the substituents R₁₄ and R₁₅ are connected to the B ring/ring system in such a way that no quarternary ammonium compounds are formed (by these connections).

4. A compound according to claim 1 wherein:
R₁ is chosen from a group consisting of methoxycarbonyl, ethoxycarbonyl, (n-propyl)-oxycarbonyl, (iso-propyl)-oxycarbonyl, (n-butyl)-oxycarbonyl, (tert-butyl)-oxycarbonyl, (3-methyl-butyl)-oxycarbonyl, (2,2-dimethyl-propyl)oxycarbonyl, n-propylcarbonyl, (cyclo-propyl)-carbonyl, 3-methylisoxazol-5-yl, 2-ethyl-2H-tetrazol-5-yl, 5-ethyl-4,5-dihydro-1,3-oxazol-2-yl, 5-methyl-1,3-oxazol-2-yl, 5-ethyl-1,3-oxazol-2-yl, 5-propyl-1,3-oxazol-2-yl and 5-butyl-1,3-oxazol-2-yl;
R₂ is chosen from a group consisting of H, methyl, ethyl, isopropyl, trifluoromethyl, methoxy, phenyl, amino and methylamino;
R₃ is chosen from a group consisting of H, amino, methyl, methylamino, dimethylamino, methoxy, methylsulfinyl and hydroxymethyl;
R₄ is chosen from a group consisting of H, methyl, chloro, cyano, amino, methylamino, dimethylamino, isopropylamino, acetylamino, (2,2-dimethylpropanoyl)amino and nitro;
R₅ is chosen from a group consisting of H and methyl;
R₁₄ is chosen from a group consisting of H, methyl, t-butyl carboxylate, 2-carboxyethyl and 3-tert-butoxy-3-oxopropyl;
R₁₅ is H;
R^{c} is chosen from a group consisting of phenyl, 2-methylphenyl, 3-methylphenyl, 4-methylphenyl, 3-(trifluoromethyl)phenyl, 4-(trifluoromethyl)phenyl, 2-(trifluoromethoxy)phenyl, 4-(trifluoromethoxy)phenyl, 2-fluorophenyl, 3-fluorophenyl, 4-fluorophenyl, 2-chlorophenyl, 3-chlorophenyl, 4-chlorophenyl, 3-bromophenyl, 3-cyanophenyl, 4-cyanophenyl, 3-methoxyphenyl, 4-methoxyphenyl, 3-nitrophenyl, 3-(3-methyl-5-oxo-4,5-dihydro-1H-pyrazol-t-yl)phenyl, 2,4-dichlorophenyl, 3,4-dichlorophenyl, 3,5-difluorophenyl, 3,4-dimethoxyphenyl, 2-methyl-5-(methylsulfonyl)phenyl, 2-thienyl, 3-thienyl, 5-bromo-2-thienyl, 5-chloro-2-thienyl, 5-chloro-3-thienyl, 2,5-dichloro-3-thienyl, 2,5-dimethyl-3-thienyl, 4,5-dichloro-2-thienyl, 3-bromo-5-chloro-2-thienyl, 4-bromo-5-chloro-2-thienyl, 5-pyridin-2-yl-2-thienyl, 5-isoxazol-3-yl-2-thienyl, 5-isoxazol-5-yl-2-thienyl, 5-[1-methyl-5-(trifluoromethyl)-1H-pyrazol-3-yl]-2-thienyl, 5-(2-methyl-1,3-thiazol-4-yl)-2-thienyl, 5-chloro-3-methyl-1-benzothien-2-yl, 2,4-dimethyl-1,3-thiazol-5-yl, 2,5-dimethyl-3-furyl, 5-(methoxycarbonyl)-2-furyl, 4-(methoxycarbonyl)-5-methyl-2-furyl, 5-methylisoxazol-4-yl, 5-chloro-1,3-dimethyl-1H-pyrazol-4-yl, pyridin-3-yl, 5-bromo-6-chloropyridin-3-yl, 2-naphtyl, 2,3-dihydro-1,4-benzodioxin-6-yl, 4-(1H-tetrazol-5-yl)phenyl, 2,1,3-benzoxadiazol-4-yl, 2,1,3-benzothiadiazol-4-yl, 6-ethoxy-1,3-benzothiazol-2-yl, 1-benzothien-3-yl, 2,3-dihydro-1,4-benzodioxin-6-yl, 6-chloroimidazo[2,1-b][1,3]thiazol-5-yl and 2,3-dihydro-1-benzofuran-5-yl;
B is chosen from the group consisting of 4-piperazin-1-ylene, 4-piperidin-1-ylene, 3-piperidin-1-ylene, 3-azetidin-1-ylene, 3-pyrrolidin-1-ylene, 4-(1,4-diazepan)-1-ylene, 5-hexahydropyrrolo[3,4-]pyrrol-2(1H)-ylene and 5-(2,5-diazabicyclo[2.2.1]hept)-2-ylene, and the substituents R₁₄ and R₁₅ are connected to the B ring/ring system, in such a way that no quarternary ammonium compounds are formed (by these connections).

5. A compound according to claim 1 which is of the formula (Ia):

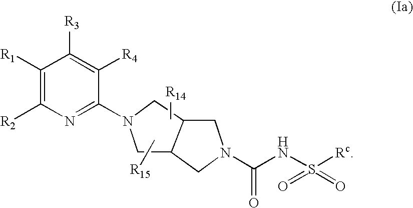

6. A compound according to claim 1 which is of the formula (Ib):

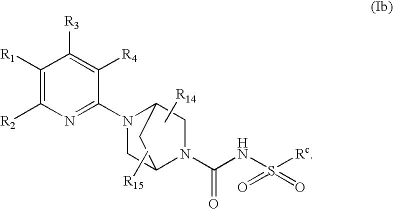

7. A compound according to claim 1 which is of the formula (Ic):

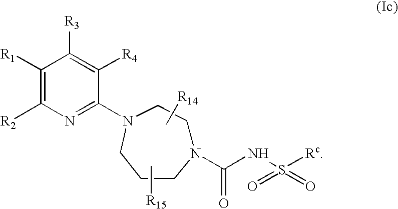

8. A compound according to claim 1 which is of the formula (Id):

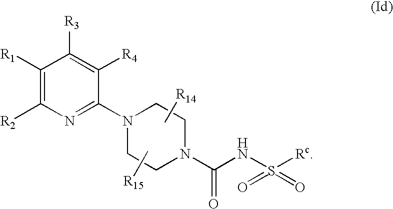

9. A compound according to claim 1 which is of the formula (Ie):

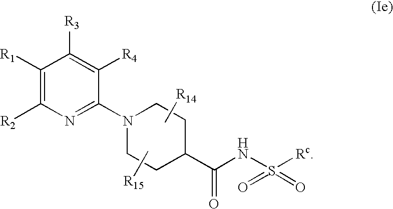

10. A compound according to claim 1 which is of the formula (If):

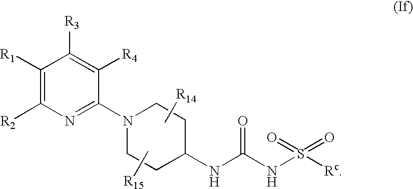

11. A compound according to claim 1 which is of the formula (Ig):

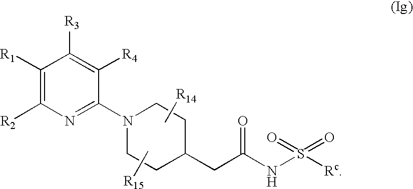

12. A compound according to claim 1 which is of the formula (Ih):

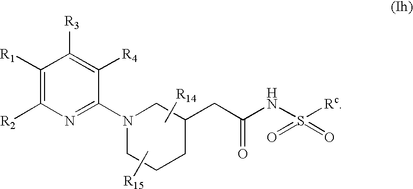

13. A compound according to claim 1 which is of the formula (Ii):

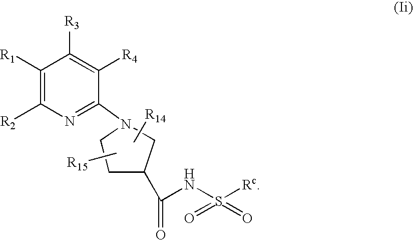

14. A compound according to claim 1 which is of the formula (Ij):

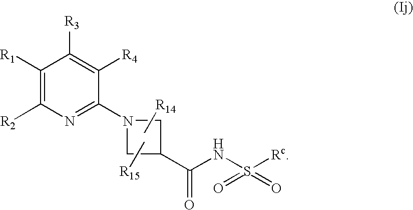

15. A compound according to claim 1 which is of the formula (Ik):

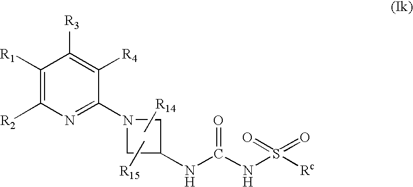

16. A compound according to claim 1 which is of the formula (Il):

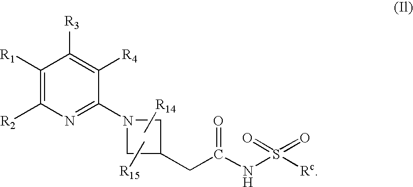

17. A compound according to claim 1 wherein R₁ represents R₆OC(O).

18. A compound according to claim 17 which is of the formula (Iaa):

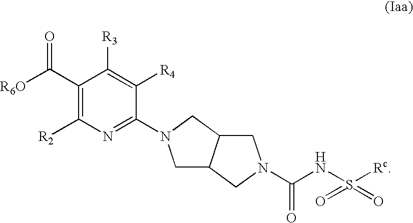

19. A compound according to claim 17 which is of the formula (Ibb):

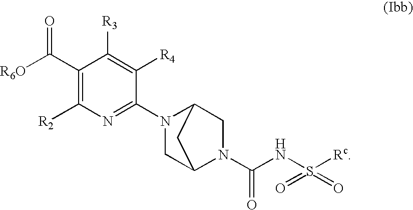

20. A compound according to claim 17 which is of the formula (Icc):

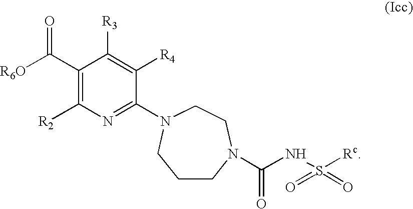

21. A compound according to claim 17 which is of the formula (Idd):

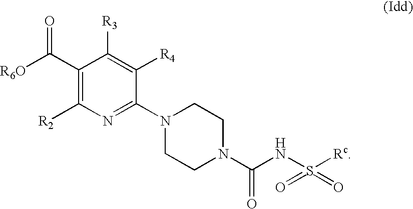

22. A compound according to claim 17 which is of the formula (Ide):

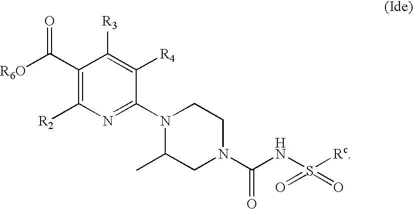

23. A compound according to claim 17 which is of the formula (Idf):

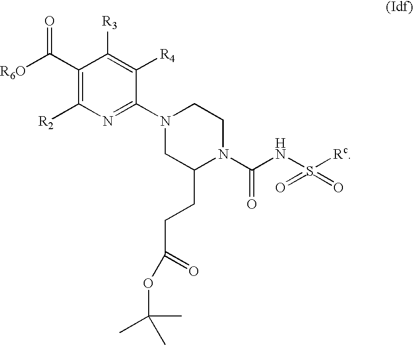

24. A compound according to claim 17 which is of the formula (Idg):

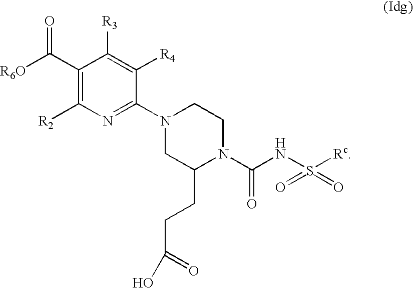

25. A compound according to claim 17 which is of the formula (Iee):

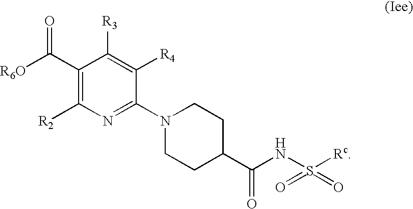

26. A compound according to claim 17 which is of the formula (Ief):

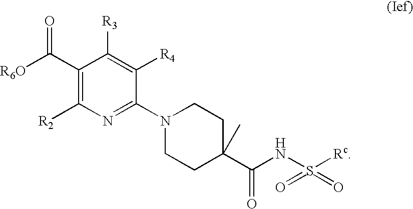

27. A compound according to claim 17 which is of the formula (Iff):

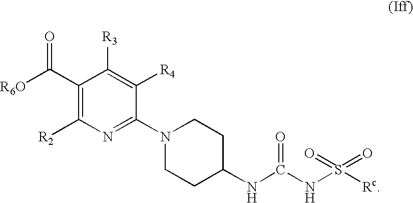

28. A compound according to claim 17 which is of the formula (Igg):

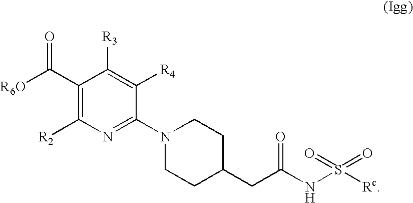

29. A compound according to claim 17 which is of the formula (Ihh):

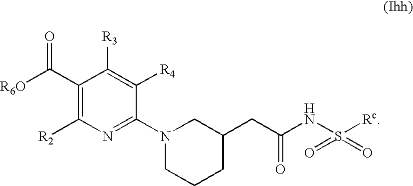

30. A compound according to claim 17 which is of the formula (Ijj):

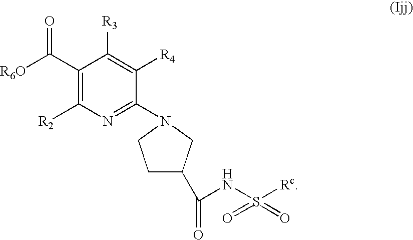

31. A compound according to claim 17 which is of the formula (Ikk):

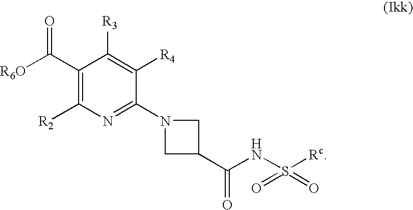

32. A compound according to claim 17 which is of the formula (Ikl):

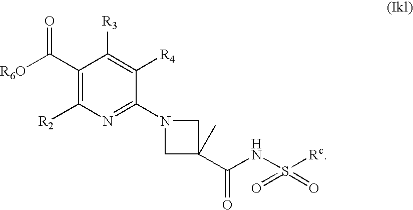

33. A compound according to claim 17 which is of the formula (Ill):

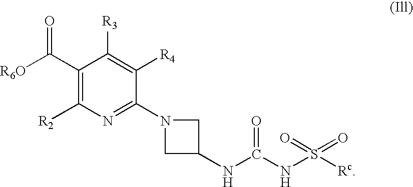

34. A compound according to claim 17 which is of the formula (Ilm):

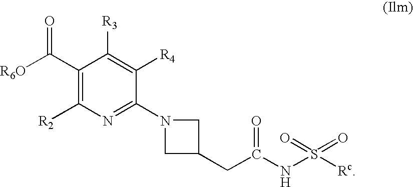

35. A compound according to claim 1 wherein R₁ represents R₇C(O).

36. A compound according to claim 35 which is of the formula (Imm):

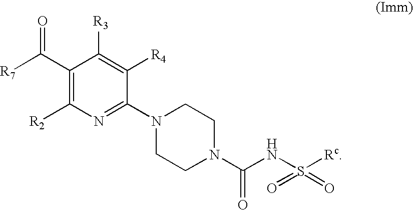

37. A compound according to claim 1 wherein R₁ represents a group selected from

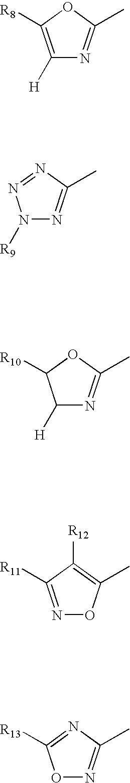

38. A compound according to claim 37 which is of the formula (Inn):

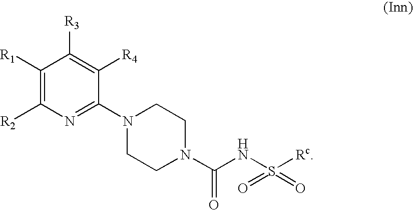

39. A compound selected from:
Ethyl 5-chloro-6-[4-({[(2-methylphenyl)sulfonyl]amino}carbonyl)piperazin-1-yl]nicotinate,
Ethyl 5-chloro-6-[4-({[(4-methylphenyl)sulfonyl]amino}carbonyl)piperazin-1-yl]nicotinate,
Ethyl 5-cyano-6-[4-({[(4-fluorophenyl)sulfonyl]amino}carbonyl)piperazin-1-yl]-2-(trifluoromethyl)nicotinate,
Ethyl 5-chloro-6-[4-({[(4-chlorophenyl)sulfonyl]amino}carbonyl)piperazin-1-yl]nicotinate,
Ethyl 5-chloro-6-[4-({[(5-chloro-2-thienyl)sulfonyl]amino}carbonyl)piperazin-1-yl]nicotinate,
Ethyl-6-(4-{[phenylsulfonyl)amino]carbonyl}piperazine-1-yl)-2-(trifluoromethyl)nicotinate,
Ethyl 5-cyano-6-(4-([phenylsulfonyl)amino]carbonyl piperazin-1-yl)-2-(trifluoromethyl)nicotinat,
Ethyl 6-[4-({[(2-chlorophenyl)sulfonyl]amino}carbonyl)piperazin-1-yl]-5-cyano-2-(trifluoromethyl)nicotinate,
Ethyl 5-cyano-6-[4-({[(4-methylphenyl)sulfonyl]amino}carbonyl)piperazin-1-yl]-2-(trifluoromethyl)nicotinate,
Ethyl 5-chloro-6-(4-{[(phenylsulfonyl)amino]carbonyl}piperazin-1-yl)nicotinate,
Ethyl 5-cyano-2-methyl-6-(4-{[(phenylsulfonyl)amino]carbonyl}piperazin-1-yl)nicotinate,
Ethyl 6-[4-({[(5-chloro-2-thienyl)sulfonyl]amino}carbonyl)piperazin-1-yl]-5-cyano-2-(trifluoromethyl)nicotinate,
Ethyl 5-chloro-6-[4-({[(4-fluorophenyl)sulfonyl]amino}carbonyl)piperazin-1-yl]nicotinate,
Ethyl 5-chloro-6-[4-({[(2-chlorophenyl)sulfonyl]amino}carbonyl)piperazin-1-yl]nicotinate,
Ethyl 6-[4-({[(4-chlorophenyl)sulfonyl]amino}carbonyl)piperazin-1-yl]-5-cyano-2-(trifluoromethyl)nicotinate,
Ethyl 5-cyano-6-[4-({[(2-methylphenyl)sulfonyl]amino}carbonyl)piperazin-1-yl]-2-(trifluoromethyl)nicotinate,
Ethyl 6-[4-({[(5-chloro-2-thienyl)sulfonyl]amino}carbonyl)piperazin-1-yl]-5-cyano-2-methylnicotinate,
Isopropyl 5-chloro-6-[4-({[(5-chloro-2-thienyl)sulfonyl]amino}carbonyl)piperazin-1-yl]nicotinate,
Butyl 5-chloro-6-[4-({[(5-chloro-2-thienyl)sulfonyl]amino}carbonyl)piperazin-1-yl]nicotinate,
Methyl 5-chloro-6-[4-({[(5-chloro-2-thienyl)sulfonyl]amino}carbonyl)piperazin-1-yl]nicotinate,
Propyl 5-chloro-6-[4-({[(5-chloro-2-thienyl)sulfonyl]amino}carbonyl)piperazin-1-yl]nicotinate,
3-Methylbutyl 5-chloro-6-[4-({[(5-chloro-2-thienyl)sulfonyl]amino}carbonyl)-piperazin-1-yl]nicotinate,
Ethyl 5-chloro-6-(4-{[phenylsulfonyl)amino]carbonyl}piperidin-1-yl)nicotinate,
Ethyl 5-chloro-6-[4-({[(5-chloro-2-thienyl)sulfonyl]amino}carbonyl)piperidin-1-yl]nicotinate,
Ethyl 5-chloro-6-[3-({[(phenylsulfonyl)amino]carbonyl}amino)azetidin-1-yl]nicotinate,
Ethyl 5-chloro-6-[3-({[(5-chloro-2-thienyl)sulfonyl]amino}carbonyl)azetidin-1-yl]nicotinate,
Ethyl 5-chloro-6-[3-({[(phenylsulfonyl)amino]carbonyl}amino)azetidin-1-yl]nicotinate,
Ethyl 5-chloro-6-[3-({[(5-[chloro-2-thienyl)sulfonyl]amino}carbonyl)pyrrolidin-1-yl]nicotinate,
Ethyl 6-[3-(3-tert-butoxy-3-oxopropyl)-4-({[(5-chloro-2-thienyl)sulfonyl]amino}carbonyl)piperazin-1-yl]-5 cyano-2-(trifluoromethyl)nicotinate,
3-{1-({[(5-Chloro-2-thienyl)sulfonyl]amino}carbonyl)-4-[3-cyano-5-[ethoxy(hydroxy)methyl]-6-(trifluoromethyl)pyridin-2-yl]piperazin-2-yl}propanoic acid,
Ethyl 6-(3-(3-tert-butoxy-3-oxopropyl)-4-{[(phenylsulfonyl)amino]carbonyl}piperazin-1-yl)-5-cyano-2-(trifluoromethyl)nicotinate,
3-(4-[3-Cyano-5-(ethoxycarbonyl)-6-(trifluoromethyl)pyridin-2-yl]-1-{[(phenylsulfonyl)amino]carbonyl}piperazin-2-yl)propanoic acid,
Ethyl 6-(3-(3-tert-butoxy-3-oxopropyl)-4-{[(phenylsulfonyl)amino]carbonyl}piperazin-1-yl)-5-chloronicotinate,
3-(4-[3 Chloro-5-(ethoxycarbonyl)pyridin-2-yl]-1-{[(phenylsulfonyl)amino]carbonyl}piperazin-2-yl)propanoic acid,
Ethyl 5-chloro-6-[4-({[(phenylsulfonyl)amino]carbonyl}amino)piperidin-1-yl]nicotinate,
4-(5-Butyryl-3-chloropyridin-2-yl)-N-[(5-chloro-2-thienyl)sulfonyl]piperazine-1-carboxamide,
4-[3-Chloro-5-(2-ethyl-2H-tetrazol-5-yl)pyridin-2-yl]-N-[(5-chloro-2-thienyl)sulfonyl]piperazine-1-carboxamide,
4-[3-Chloro-5-(5-ethyl-4,5-dihydro-1,3-oxazol-2-yl)pyridin-2-yl]-N-(phenylsulfonyl)piperazine-1-carboxamide,
4-[3-Chloro-5-(5-methyl-1,3-oxazol-2-yl)pyridin-2-yl]-N-(phenylsulfonyl)piperazine-1-carboxamide,
4-[3-Chloro-5-(5-methyl-1,3-oxazol-2-yl)pyridin-2-yl]-N-[(5-chloro-2-thienyl)sulfonyl]piperazine-1-carboxamide,
4-[3-Chloro-5-(5-ethyl-1,3-oxazol-2-yl)pyridin-2-yl]-N-(phenylsulfonyl)piperazine-1-carboxamide,
4-[3-Chloro-5-(5-ethyl-1,3-oxazol-2-yl)pyridin-2-yl]-N-[(5-chloro-2-thienyl)sulfonyl]piperazine-1-carboxamide,
4-[3-Chloro-5-(3-methylisoxazol-5-yl)pyridin-2-yl]-N-[(5-chloro-2-thienyl)sulfonyl]piperazine-1-carboxamide,
4-[3-Chloro-5-(5-ethyl-1,2,4-oxadiazol-3-yl)pyridin-2-yl]-N-[(5-chloro-2-thienyl)sulfonyl]piperazine-1-carboxamide,
Isopropyl 5-cyano-2-methyl-6-[4-({[(4-methylphenyl)sulfonyl]amino}carbonyl)piperidin-1-yl]nicotinate,
Isopropyl 5-cyano-2-methyl-6-(4-{[(2-naphthylsulfonyl)amino]carbonyl}piperidin-1-yl)nicotinate,
Ethyl 6-{3-[({[(4-chlorophenyl)sulfonyl]amino}carbonyl)amino]azetidin-1-yl}-5-cyano-2-methylnicotinate,
Ethyl 6-{3-[({[(5-chloro-2-thienyl)sulfonyl]amino}carbonyl)amino]azetidin-1-yl}-5-cyano-2-methylnicotinate,
Ethyl 6-[4-({[(5-chloro-2-thienyl)sulfonyl]amino}carbonyl)piperidin-1-yl]-5-cyano-2-isopropylnicotinate,
Ethyl 6-[4-({[(5-chloro-2-thienyl)sulfonyl]amino}carbonyl)piperidin-1-yl]-5-cyano-2-phenylnicotinate,
Ethyl 6-[4-({[(5-chloro-2-thienyl)sulfonyl]amino}carbonyl)piperidin-1-yl]-5-cyano-2-ethylnicotinate,
tert-Butyl 6-[4-({[(5-chloro-2-thienyl)sulfonyl]amino}carbonyl)piperidin-1-yl]-5-cyano-2-methylnicotinate,
2,2-Dimethylpropyl 6-{3-[({[(5-chloro-2-thienyl)sulfonyl]amino}carbonyl)amino]azetidin-1-yl}-5-cyano-2-methylnicotinate,
2,2-Dimethylpropyl 6-[4-({[(5-chloro-2-thienyl)sulfonyl]amino}carbonyl)piperidin-1-yl]-5-cyano-2-methylnicotinate,
Isopropyl 5-cyano-2-methyl-6-[4-({[(5-methyl-2-thienyl)sulfonyl]amino}carbonyl)piperidin-1-yl]nicotinate,
Ethyl 5-cyano-2-methyl-6-[3-({[(3-methylphenyl)sulfonyl]amino}carbonyl)azetidin-1-yl]nicotinate,
Ethyl 5-cyano-2-methyl-6-[3-({[(phenylsulfonyl)amino]carbonyl}amino)azetidin-1-yl]nicotinate,
1-[3-Chloro-5-(5-ethyl-1,3-oxazol-2-yl)-6-(methylamino)pyridin-2-yl]-N-[(5-chloro-2-thienyl)sulfonyl]piperidine-4-carboxamide,
Ethyl 5-cyano-2-methyl-6-(4-{2-oxo-2-[(phenylsulfonyl)amino]ethyl}piperidin-1-yl)nicotinate,
Ethyl 4-amino-5-chloro-6-[4-({[(5-chloro-2-thienyl)sulfonyl]amino}carbonyl)piperidin-1-yl]nicotinate,
Ethyl 6-[4-(2-{[(5-chloro-2-thienyl)sulfonyl]amino}-2-oxoethyl)piperidin-1-yl]-5-cyano-2-methylnicotinate,
Ethyl 6-[4-({[(5-chloro-2-thienyl)sulfonyl]amino}carbonyl)-1,4-diazepan-1-yl]-5-cyano-2-methylnicotinate,
Ethyl 6-[4-({[(5-chloro-2-thienyl)sulfonyl]amino}carbonyl)-2-methylpiperazin-1-yl]-5-cyano-2-methylnicotinate,
Ethyl 5-cyano-2-methyl-6-(4-{[(phenylsulfonyl)amino]carbonyl}-1,4-diazepan-1-yl)nicotinate,
1-[3-Chloro-5-(5-ethyl-1,3-oxazol-2-yl)-4-(methylamino)pyridin-2-yl]-N-[(5-chloro-2-thienyl)sulfonyl]piperidine-4-carboxamide,
Ethyl 6-[4-({[(5-chloro-2-thienyl)sulfonyl]amino}carbonyl)-4-methylpiperidin-1-yl]-5-cyano-2-methylnicotinate,
Ethyl 6-(3-{[({[(5-chloro-2-thienyl)sulfonyl]amino}carbonyl)amino]methyl}azetidin-1-yl)-5-cyano-2-methylnicotinate,
Ethyl 5-cyano-2-methyl-6-{3-[({[(phenylsulfonyl)amino]carbonyl}amino)methyl]azetidin-1-yl}nicotinate,
Ethyl 5-Cyano-6-[3-({[(4-cyanophenyl)sulfonyl]amino}carbonyl)azetidin-1-yl]-2-methylnicotinate,
Ethyl 6-(3-{[(2,1,3-benzoxadiazol-4-ylsulfonyl)amino]carbonyl}azetidin-1-yl)-5-cyano-2-methylnicotinate,
Ethyl 5-cyano-2-methyl-6-{3-[({[4-(1H-tetrazol-5-yl)phenyl]sulfonyl}amino)carbonyl]azetidin-1-yl}nicotinate,
Ethyl 5-cyano-6-[3-({[(4-methoxyphenyl)sulfonyl]amino}carbonyl)azetidin-1-yl]-2-methylnicotinate,
Ethyl 5-cyano-6-[3-({[(3-cyanophenyl)sulfonyl]amino}carbonyl)azetidin-1-yl]-2-methylnicotinate,
Ethyl 5-cyano-2-methyl-6-(3-{[(2-naphthylsulfonyl)amino]carbonyl}azetidin-1-yl)nicotinate,
Ethyl 5-cyano-6-[3-({[(2,4-dimethyl-1,3-thiazol-5-yl)sulfonyl]amino}carbonyl)azetidin-1-yl]-2-methylnicotinate,
Ethyl 5-cyano-6-(3-{[(2,3-dihydro-1,4-benzodioxin-6-ylsulfonyl)amino]carbonyl}azetidin-1-yl)-2-methylnicotinate,
Ethyl 5-cyano-2-methyl-6-[3-({methyl[(4-methylphenyl)sulfonyl]amino}carbonyl)azetidin-1-yl]nicotinate,
Ethyl 5-cyano-6-[3-({[(2,4-dichlorophenyl)sulfonyl]amino}carbonyl)azetidin-1-yl]-2-methylnicotinate,
Ethyl 6-[3-({[(5-chloro-3-methyl-1-benzothien-2-yl)sulfonyl]amino}carbonyl)azetidin-1-yl]-5-cyano-2-methylnicotinate,
Ethyl 5-cyano-2-methyl-6-[3-({[(4-methyl phenyl) sulfonyl]amino}carbonyl)azetidin-1-yl]nicotinate,
Ethyl 5-cyano-2-methyl-6-{3-[({[4-(trifluoromethyl)phenyl]sulfonyl}amino) carbonyl]azetidin-1-yl}nicotinate,
Ethyl 5-cyano-2-methyl-6-[3-({[(3-nitrophenyl)sulfonyl]amino}carbonyl)azetidin-1-yl]nicotinate,
Ethyl 6-[3-({[(3-bromophenyl)sulfonyl]amino}carbonyl)azetidin-1-yl]-5-cyano-2-methylnicotinate,
Ethyl 6-[3-({[(5-chloro-2-thienyl)sulfonyl]amino}carbonyl)-3-methylazetidin-1-yl]-5-cyano-2-methylnicotinate,
1-[6-amino-3-chloro-5-(5-ethyl-1,3-oxazol-2-yl)pyridin-2-yl]-N-[(5-chloro-2-thienyl)sulfonyl]piperidine-4-carboxamide,
Ethyl 6-[3-({[(3-bromo-5-chloro-2-thienyl)sulfonyl]amino}carbonyl)azetidin-1-yl]-5-cyan-2-methylnicotinate,
Ethyl 6-(3-{[(2,1,3-benzothiadiazol-4-ylsulfonyl)amino]carbonyl}azetidin-1-yl)-5-cyano-2-methylnicotinate,
Ethyl 5-cyano-6-[3-({[(2,5-dimethyl-3-furyl)sulfonyl]amino}carbonyl)azetidin-1-yl]-2-methylnicotinate,
Ethyl 6-[3-({[(6-chloroimidazo[2,1-b][1,3]thiazol-5-yl)sulfonyl]amino}carbonyl)azetidin-1-yl]-5-cyano-2-methylnicotinate,
Ethyl 5-cyano-6-(3-{[(2,3-dihydro-1-benzofuran-5-ylsulfonyl)amino]carbonyl}azetidin-1-yl)-2-methylnicotinate,
Ethyl 5-cyano-6-[3-({[(4-fluorophenyl)sulfonyl]amino}carbonyl)azetidin-1-yl]-2-methylnicotinate,
Ethyl 6-[3-({[(5-chloro-3-thienyl)sulfonyl]amino}carbonyl)azetidin-1-yl]-5-cyano-2-methylnicotinate,
Ethyl 5-cyano-6-[3-({[(5-isoxazol-5-yl-2-thienyl)sulfonyl]amino}carbonyl)azetidin-1-yl]-2-methylnicotinate,
Ethyl 6-[3-({[(3-chlorophenyl)sulfonyl]amino}carbonyl)azetidin-1-yl]-5-cyano-2-methylnicotinate,
Ethyl 5-cyano-6-[3-({[(2-fluorophenyl)sulfonyl]amino}carbonyl)azetidin-1-yl]-2-methylnicotinate,
Ethyl 5-cyano-4-[3-({[(5-isoxazol-3-yl-2-thienyl)sulfonyl]amino}carbonyl)azetidin-1-yl]-2-methylnicotinate,
Ethyl 5-cyano-6-[3-({[(3-fluorophenyl)sulfonyl]amino}carbonyl)azetidin-1-yl]-2-methylnicotinate,
Ethyl 5-cyano-2-methyl-6-(3-{[(phenylsulfonyl)amino]carbonyl}azetidin-1-yl)nicotinate,
Ethyl 6-[3-({[(4-bromo-5-chloro-2-thienyl)sulfonyl]amino}carbonyl)azetidin-1-yl]-5-cyano-2-methylnicotinate,
Ethyl 6-[3-({[(5-bromo-6-chloropyridin-3-yl)sulfonyl]amino}carbonyl)azetidin-1-yl]-5-cyano-2-methylnicotinate,
Ethyl 6-[3-({[(5-bromo-2-thienyl)sulfonyl]amino}carbonyl)azetidin-1-yl]-5-cyano-2-methylnicotinate,
Ethyl 5-cyano-2-methyl-6-[3-({[(5-pyridin-2-yl-2-thienyl)sulfonyl]amino}carbonyl)azetidin-1-yl]nicotinate,
Ethyl 5-cyano-6-[3-({[(2,5-dichloro-3-thienyl)sulfonyl]amino}carbonyl)azetidin-1-yl]-2-methylnicotinate,
Ethyl 5-cyano-6-[3-({[(4,5-dichloro-2-thienyl)sulfonyl]amino}carbonyl)azetidin-1-yl]-2-methylnicotinate,
Ethyl 5-cyano-2-methyl-6-{3-[({[3-(trifluoromethyl)phenyl]sulfonyl}amino)carbonyl]azetidin-1-yl}nicotinate,
Ethyl 6-(3-{[(1-benzothien-3-ylsulfonyl)amino]carbonyl}azetidin-1-yl)-5-cyano-2-methylnicotinate,
Ethyl 6-[3-({[(2-chlorophenyl)sulfonyl]amino}carbonyl)azetidin-1-yl]-5-cyano-2-methylnicotinate,
Ethyl 5-cyano-6-[3-({[(2,54-dimethyl-3-thienyl)sulfonyl]amino}carbonyl)azetidin-1-yl]-2-methylnicotinate,
Ethyl 5-cyano-6-[3-({[(3-methoxyphenyl)sulfonyl]amino}carbonyl)azetidin-1-yl]-2-methylnicotinate,
Ethyl 5-cyano-2-methyl-6-(3-{[(3-thienylsulfonyl)amino]carbonyl}azetidin-1-yl)nicotinate,
Ethyl 5-cyano-2-methyl-6-(3-{[(2-thienylsulfonyl)amino]carbonyl}azetidin-1-yl)nicotinate,
1-[4-Amino-3 chloro-5-(5-ethyl-1,3-oxazol-2-yl)pyridin-2-yl]-N-[(5 chloro-2-thienyl)sulfonyl]piperidine-4-carboxamide,
tert-Butyl 5-chloro-6-[4-({[(5-chloro-2-thienyl)sulfonyl]amino}carbonyl)piperidin-1-yl]nicotinate,
N-[(5-chloro-2-thienyl)sulfonyl]-1-[5-(5-ethyl-1,3-oxazol-2-yl)-3-(isopropylamino)pyridin-2-yl]piperidine-4-carboxamide,
N-[(5-chloro-2-thienyl)sulfonyl]-1-[3-(dimethylamino)-5-(5-ethyl-1,3-oxazol-2-yl)pyridin-2-yl]piperidine-4-carboxamide,
N-[(5-chloro-2-thienyl)sulfonyl]-1-[5-(5-ethyl-1,3-oxazol-2-yl)-3-(methylamino)pyridin-2-yl]piperidine-4-carboxamide,
Ethyl 6-[4-({[(5-chloro-2-thienyl)sulfonyl]amino}carbonyl)piperidin-1-yl]-5-cyano-2-methylnicotinate,
Ethyl 5-cyano-2-methyl-6-[3-({[(5-methylisoxazol-4-yl)sulfonyl]amino}carbonyl)azetidin-1-yl]nicotinate,
1-[3-Chloro-5-(5-ethyl-1,3-oxazol-2-yl)-4-(methylsulfinyl)pyridin-2-yl]-N-[(5-chloro-2-thienyl)sulfonyl]piperidine-4-carboxamide,
Ethyl 6-[4-({[(5-chloro-2-thienyl)sulfonyl]amino}carbonyl)piperidin-1-yl]-2,4-dimethylnicotinate,
1-[3-(Acetylamino)-5-(5-ethyl-1,3-oxazol-2-yl)pyridin-2-yl]-N-[(5-chloro-2-thienyl)sulfonyl]piperidine-4-carboxamide,
1-[3-Chloro-5-(5-ethyl-1,3-oxazol-2-yl)-4-(hydroxymethyl)pyridin-2-yl]-N-[(5-chloro-2-thienyl)sulfonyl]piperidine-4-carboxamide,
1-[3-amino-5-(5-ethyl-1,3-oxazol-2-yl)pyridin-2-yl]-N-[(5-chloro-2-thienyl)sulfonyl]piperidine-4-carboxamide,
4-[3-chloro-5-(cyclopropylcarbonyl)pyridin-2-yl]-N-[(5-chloro-2-thienyl)sulfonyl]piperazine-1-carboxamide,
N-[({1-[3-cyano-5-(5-ethyl-1,3-oxazol-2-yl)-6-methylpyridin-2-yl]azetidin-3-yl}amino)carbonyl]-4-methylbenzenesulfonamide,
N-[(5-chloro-2-thienyl)sulfonyl]-1-[5-(5 ethyl-1,3-oxazol-2-yl)-3-nitropyridin-2-yl]piperidine-4-carboxamide,
N-[(5-chloro-2-thienyl)sulfonyl]-1-[3-cyano-5-(5-ethyl-1,3-oxazol-2-yl)-6-methylpyridin-2-yl]azetidine-3-carboxamide,
N-[(5-chloro-2-thienyl)sulfonyl]-1-[3-cyano-5-(5-ethyl-1,3-oxazol-2-yl)-6-methylpyridin-2-yl]piperidine-4-carboxamide,
1-[3-Chloro-5-(5-ethyl-1,3-oxazol-2-yl)-4-methylpyridin-2-yl]-N-[(5-chloro-2-thienyl)sulfonyl]piperidine-4-carboxamide,
Ethyl 6-[3-({[(5-chloro-2-thienyl)sulfonyl]amino}carbonyl)azetidin-1-yl]-5-cyano-2-methylnicotinate,
N-[(5-chloro-2-thienyl)sulfonyl]-1-[5-(5-ethyl-1,3-oxazol-2-yl)-3-methylpyridin-2-yl]piperidine-4-carboxamide,
1-[3-Chloro-5-(5-ethyl-1,3-oxazol-2-yl)pyridin-2-yl]-N-[(5-chloro-2-thienyl)sulfonyl]piperidine-4-carboxamide,
1-[3-chloro-5-(5-propyl-1,3-oxazol-2-yl)pyridin-2-yl]-N-[(5-chloro-2-thienyl)sulfonyl]piperidine-4-carboxamide,
1-[5-(5-Butyl-1,3-oxazol-2-yl)-3-chloropyridin-2-yl]-N-[(5-chloro-2-thienyl)sulfonyl]piperidine-4-carboxamide,
5-Chloro-N-[({1-[3-cyano-5-(5-ethyl-1,3-oxazol-2-yl)-6-methylpyridin-2-yl]azetidin-3-yl}amino)carbonyl]thiophene-2-sulfonamide,
N-[(5-chloro-2-thienyl)sulfonyl]-4-[3-cyano-5-(5-ethyl-1,3-oxazol-2-yl)-6-methylpyridin-2-yl]piperazine-1-carboxamide,
1-[3-Chloro-5-(5-ethyl-1,3-oxazol-2-yl)pyridin-2-yl]-N-[(5-chloro-2-thienyl)sulfonyl]azetidine-3-carboxamide,
Ethyl 5-chloro-6-[4-({[(5-chloro-2-thienyl)sulfonyl]amino}carbonyl)piperidin-1-yl]-2,4-dimethylnicotinate,
1-[3-Chloro-5-(5-ethyl-1,3-oxazol-2-yl)-4-methoxypyridin-2-yl]-N-[(5-chloro-2-thienyl)sulfonyl]piperidine-4-carboxamide,
1-[3-Chloro-5-(5-ethyl-1,3-oxazol-2-yl)-6-methoxypyridin-2-yl]-N-[(5-chloro-2-thienyl)sulfonyl]piperidine-4-carboxamide,
1-[3-Chloro-4-(dimethylamino)-5-(5-ethyl-1,3-oxazol-2-yl)pyridin-2-yl]-N-[(5-chloro-2-thienyl)sulfonyl]piperidine-4-carboxamide,
Ethyl 5-cyano-2-methyl-6-(3-{[(pyridin-3-ylsulfonyl)amino]carbonyl}azetidin-1-yl)nicotinate,
Ethyl 5-cyano-2-methyl-6-(3-{[({5-[1-methyl-5-(trifluoromethyl)-1H-pyrazol-3-yl]-2-thienyl}sulfonyl)amino]carbonyl}azetidin-1-yl)nicotinate,
N-[(5-chloro-2-thienyl)sulfonyl]-1-[3-[(2,2-dimethylpropanoyl)amino]-5-(5-ethyl-1,3-oxazol-2-yl)pyridin-2-yl]piperidine-4-carboxamide,
Ethyl 6-[3-({[(5-chloro-1,3-dimethyl-1H-pyrazol-4-yl)sulfonyl]amino}carbonyl)azetidin-1-yl]-5-cyano-2-methylnicotinate,
Ethyl 5-cyano-2-methyl-6-{3-[({[3-(3-methyl-5-oxo-4,5-dihydro-1H-pyrazol-1-yl)phenyl]sulfonyl}amino)carbonyl]azetidin-1-yl}nicotinate,
Ethyl 6-(3-{[({4-[(4-chlorophenyl)sulfonyl]-3-methyl-2-thienyl}sulfonyl)amino]carbonyl}azetidin-1-yl)-5-cyano-2-methylnicotinate,
Ethyl 5-cyano-2-methyl-6-{3-[({[2-(trifluoromethoxy)phenyl]sulfonyl}amino)carbonyl]azetidin-1-yl}nicotinate,
Ethyl 5-cyano-6-[3-({[(3,5-difluorophenyl)sulfonyl]amino}carbonyl)azetidin-1-yl]-2-methylnicotinate,
Ethyl 5-cyano-2-methyl-6-{3-[({[4-(trifluoromethoxy)phenyl]sulfonyl}amino) carbonyl]azetidin-1-yl}nicotinate,
Ethyl 6-[3-(2-{[(5-chloro-2-thienyl)sulfonyl]amino}-2-oxoethyl)piperidin-1-yl]-5-cyano-2-methylnicotinate,
Ethyl 5-cyano-6-{3-[({[5-(methoxycarbonyl)-2-furyl]sulfonyl}amino) carbonyl]azetidin-1-yl}-2-methylnicotinate,
Ethyl 5-cyano-6-{3-[({[4-(methoxycarbonyl)-5-methyl-2-furyl]sulfonyl}amino)carbonyl]azetidin-1-yl}-2-methyl nicotinate,
Ethyl 6-[3-({[(4-chlorophenyl)sulfonyl]amino}carbonyl)azetidin-1-yl]-5-cyano-2-methylnicotinate,
Ethyl 5-cyano-6-[3-({[(3,4-dichlorophenyl)sulfonyl]amino}carbonyl)azetidin-1-yl]-2-methylnicotinate,
Ethyl 5-cyano-6-[3-({[(3,4-dimethoxyphenyl)sulfonyl]amino}carbonyl)azetidin-1-yl]-2-methylnicotinate,
Ethyl 5-cyano-2-methyl-6-{3-[({[2-methyl-5-(methylsulfonyl)phenyl]sulfonyl}amino)carbonyl]azetidin-1-yl}nicotinate,
N-[(5-chloro-2-thienyl)sulfonyl]-1-[3-cyano-5-(cyclopropylcarbonyl)-6-methylpyridin-2-yl]piperidine-4-carboxamide,
Isopropyl 6-[3-({[(5-chloro-2-thienyl)sulfonyl]amino}carbonyl)azetidin-1-yl]-5-ethynyl-2-methylnicotinate,
Ethyl 6-{4-[({[(4-chlorophenyl)sulfonyl]amino}carbonyl)amino]piperidin-1-yl}-5-cyano-2-methylnicotinate,
Ethyl 6-{4-[({[(5-chloro-2-thienyl)sulfonyl]amino}carbonyl)amino]piperidin-1-yl}-5-cyano-2-methylnicotinate,
Ethyl 6-[4-({[(5-chloro-3-thienyl)sulfonyl]amino}carbonyl)piperidin-1-yl]-5-cyano-2-methylnicotinate,
Ethyl 5-cyano-2-methyl-6-(4-{[(2-naphthylsulfonyl)amino]carbonyl}piperidin-1-yl)nicotinate,
Ethyl 5-cyano-2-methyl-6-[4-({[(4-methylphenyl)sulfonyl]amino}carbonyl)piperidin-1-yl]nicotinate,
Ethyl 5-cyano-2-methyl-6-[5-{[(phenylsulfonyl)amino]carbonyl}hexahydropyrrolo[3,4-c]pyrrol-2(1H)-yl]nicotinate,
Ethyl 5-cyano-2-methyl-6-{3-[({[5-(2-methyl-1,3-thiazol-4-yl)-2-thienyl]sulfonyl}amino)carbonyl]azetidin-1-yl}nicotinate,
Ethyl 6-[(1S,4S)-5-({[(5-chloro-2-thienyl)sulfonyl]amino}carbonyl)-2,5-diazabicyclo[2 0.2.1]hept-2-yl]-5-cyano-2-methylnicotinate,
Ethyl 5-cyano-2-methyl-6-(4-{[(phenylsulfonyl)amino]carbonyl}piperidin-1-yl)nicotinate,
Ethyl 5-cyano-6-[4-({[(2,4-dichlorophenyl)sulfonyl]amino}carbonyl)piperidin-1-yl]-2-methylnicotinate,
Isopropyl 6-[4-({[(3-bromophenyl)sulfonyl]amino}carbonyl)piperidin-1-yl]-5-cyano-2-methylnicotinate,
Ethyl 5-cyano-2-methyl-6-{4-[({[4-(trifluoromethoxy)phenyl]sulfonyl}amino) carbonyl]piperidin-1-yl}nicotinate,
Ethyl 5-cyano-6-[3-({[(6-ethoxy-1,3-benzothiazol-2-yl)sulfonyl]amino}carbonyl)azetidin-1-yl]-2-methylnicotinate,
Ethyl 5-cyano-2-methyl-6-(3-{2-oxo-2-[phenylsulfonyl)amino]ethyl}piperidin-1-yl)nicotinate,
Ethyl 5-cyano-6-(4-{[(2,3-dihydro-1,4-benzodioxin-6-ylsulfonyl)amino]carbonyl}piperidin-1-yl)-2-methylnicotinate,
Ethyl 5-cyano-6-[4-({[(4-methoxyphenyl)sulfonyl]amino}carbonyl)piperidin-1-yl]-2-methylnicotinate,
Ethyl 6-(4-{[(2,1,3-benzoxadiazol-4-ylsulfonyl)amino]carbonyl}piperidin-1-yl)-5-cyano-2-methylnicotinate,
Ethyl 5-cyano-2-methyl-6-[4-({[(3-nitrophenyl)sulfonyl]amino}carbonyl)piperidin-1-yl]nicotinate,
Isopropyl 5-cyano-2-methyl-6-(4-{[(phenylsulfonyl)amino]carbonyl}piperidin-1-yl)nicotinate,
Isopropyl 5-cyano-2-methyl-6-{3-[({[4-(trifluoromethyl)phenyl]sulfonyl}amino) carbonyl]azetidin-1-yl}nicotinate,
Isopropyl 6-[4-({[(4-chlorophenyl)sulfonyl]amino}carbonyl)piperidin-1-yl]-5-cyano-2-methylnicotinate,
Ethyl 5-cyano-6-[4-({[(3-cyanophenyl)sulfonyl]amino}carbonyl)piperidin-1-yl]-2-methylnicotinate,
Isopropyl 5-cyano-2-methyl-6-(3-{[(2-naphthylsulfonyl)amino]carbonyl}azetidin-1-yl)nicotinate,
Ethyl 5-cyano-2-methyl-6-{4-[({[2-(trifluoromethoxy)phenyl]sulfonyl}amino) carbonyl]piperidin-1-yl}nicotinate,
Isopropyl 5-cyano-6-[4-({[(4-methoxyphenyl)sulfonyl]amino}carbonyl)piperidin-1-yl]-2-methylnicotinate,
Ethyl 5-cyano-2-methyl-6-(3-{2-oxo-2-[(phenylsulfonyl)amino]ethyl}azetidin-1-yl)nicotinate; and
Ethyl 6-[3-(2-{[(5-chloro-2-thienyl)sulfonyl]amino}-2-oxoethyl)azetidin-1-yl]-5-cyano-2-methylnicotinate.

40. A compound of formula (II)

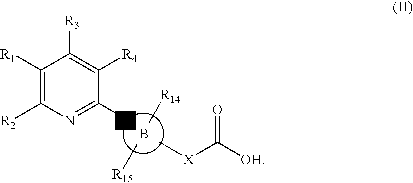

42. A pharmaceutical composition comprising a compound according to claim 1 and a pharmaceutically acceptable adjuvant, diluent, and/or carrier.

46. A method of treatment of a platelet aggregation disorder comprising administering to a patient suffering from such a disorder a therapeutically effective amount of a compound according to claim 1.

47. A method of inhibition of the P2Y₁₂ receptor in a cell comprising administering to a patient having such receptor an effective amount of a compound according to claim 1.
